(19) United States
(12) Patent Application Publication
(10) Pub. No.: US 2026/0151497 A1
(43) Pub. Date: Jun. 4, 2026

(54) LIGAND-DRUG CONJUGATES

(71) Applicant: Tripartite Therapeutics Inc., Taipei City (TW)

(72) Inventors: Young-Sun Lin, Taipei City (TW); Er-Yi Huang, Taipei City (TW); Hsueh-Yi Lu, Taipei City (TW)

(21) Appl. No.: 19/407,141

(22) Filed: Dec. 3, 2025

Related U.S. Application Data
(60) Provisional application No. 63/728,044, filed on Dec. 4, 2024.

(51) Int. Cl. A61K 47/68; A61K 47/64; A61P 35/00

(52) U.S. Cl. CPC A61K 47/68033; A61K 47/643; A61K 47/68037; A61K 47/6855; A61K 47/6889; A61P 35/00

(57) ABSTRACT

Disclosed are ligand drug conjugates (LDCs) for treating cancers and other diseases, and compounds useful for preparing the LDCs.

### CROSS REFERENCE TO RELATED APPLICATION

This application claims the benefit of and priority to U.S. Provisional Application No. 63/728,044 filed Dec. 4, 2024, the entire content of which is incorporated by reference.

### BACKGROUND

Ligand-drug conjugates (LDCs), especially antibody-drug conjugates (ADCs), are designed to deliver a therapeutic agent to a target.

An ADC is typically made up of three components: a monoclonal antibody as the targeting molecule, a cytotoxic agent or an immune adjuvant as the payload and a linker that crosslinks the payload to the antibody. The safety and efficacy of ADCs are important considerations. They are affected by factors including linker's physicochemical properties, payload's physicochemical properties, payload's pharmacological properties, location of chemical groups of the conjugations, payload release mechanism, and drug-antibody ratios (DAR).

Most current ADC payloads, such as Mertansine (DM1), Ravtansine (DM4), Monomethyl auristatin E (MMAE), γ-calicheamicin, and pyrrolobenzodiazepine (PBD) are hydrophobic. Their solubility and bioavailability remain as key challenges in the development of ADCs. The intrinsic property of payload hydrophobicity can lead to aggregation of an ADC, drug load limitations (that is, low DAR), antigen-independent toxicity due to Reticuloendothelial System (RES) clearance, and pharmacokinetics and pharmacodynamics (PK/PD) issues. Indeed, low DAR of 4 or less are found in most conventional ADCs, marketed or currently under clinical development, including trastuzumab emtansine sold under Kadcyla®, mirvetuximab soravtansine sold under Elahere®, lorvotuzumab mertansine (IMGN901; LM), HuC242-DM1, MLN2704, BT062, AVE9633, and BT062 (see Deslandes, 2014; and Maecker et al., 2023).

Several approaches have been exploited to construct next generation ADCs but remain unsuccessful (see Lyon et al., 2015; Yurkovetskiy et al., 2015; Burke et al., 2017; Su et al., 2018; Anami et al., 2018; Viricel et al., 2019; Simmons et al., 2020; Li et al., 2021; and Tedeschini et al., 2021; Evans et al., 2022; and Watanabe et al., 2024).

Further, current ADC technology faces another limitation: payload-mandated linker selection and/or development. For example, VCt linker is recruited for ADCs with MMAE as the payload, GGFG linker for ADCs with Dxd and the like.

There is a need to develop a universal linker for high DAR, potent ligand-drug conjugates to efficiently and safely deliver a payload(s) to targeted cells.

### SUMMARY OF THE INVENTION

The present invention provides potent ligand drug conjugates (LDCs) for treating cancers and other diseases.

In one aspect, the invention relates to compounds of formula I:

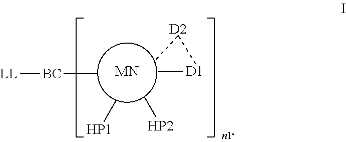

In formula I, LL is a ligand linker containing a coupling moiety capable of reacting with a ligand via a sulfhydryl, amino, glutamine, formyl, or keto group contained in the ligand; BC is absent or a bridging complex containing 1 to 20 monomeric units; MN is a multifunctional linker complex containing 1 to 20 monomeric units; D1 is a first drug moiety; D2 is absent or a second drug moiety; one of [symbol] is a covalent bond and the other [symbol] is absent; HP1 and HP2 are hydrophilic polymers independently selected from a polysaccharide, a PEG, and a derivative thereof, LL is bonded to BC or MN via a first connector; BC is bonded to MN via a second connector; HP1 is bonded to MN via a third connector; HP2 is bonded to MN via a fourth connector; D1 is bonded to MN via a fifth connector; D2 when present is bonded to MN or D1 via a sixth connector; n1 is 1 or 2; and each of the first to sixth connectors is independently selected from the group consisting of a hydrazone moiety, an oxime moiety, a carbonate moiety (—O—C(O)—O—), an azobenzene moiety, an amide bond, an arylsulfate bond, a glycoside bond, a beta-glucuronide bond, a beta-galactoside bond, an ester bond, a phosphate bond, a pyrophosphate bond, an ether bond, a thioether bond, a disulfide bond, a C—S bond, a C—O bond, a C—N bond, a C—C bond, an imine bond, a carbamate moiety (—O—C(O)—NH—), a urea moiety (—NH—C(O)—NH—), a 1,2,4-trioxolane (TRX) moiety, a triazole moiety

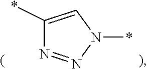 [SMILES: *c1cn(*)nn1]

a moiety derived from strain-promoted azide-alkyne cycloaddition (SPAAC), a moiety derived from strain-promoted alkyne-nitrone cycloaddition (SPANC), and a moiety derived from the trans-cyclooctene (TCO)-tetrazine click reaction.

A subset of the compounds of formula I are compounds of formula II:

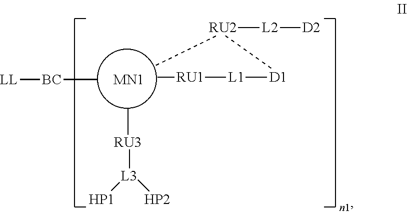

in which MN1 is a multifunctional linker complex containing 1 to 20 monomeric units; RU1 is a bond or a first releasable unit with or without a self-immolative spacer; L1 is a bond or a first bifunctional crosslinker; RU2 is a bond or a second releasable unit with or without a self-immolative spacer; one of [symbol] is a covalent bond and the other [symbol] is absent; L2 is a bond or a second bifunctional crosslinker; RU3 is a bond or a third releasable unit with or without a self-immolative spacer; L3 is a bond or a multifunctional crosslinker; RU1 when present is bonded to MN1 via a seventh connector; L1 when present is bonded to RU1 or MN1 via an eighth connector; D1 is bonded to L1, RU1, or MN1 via the fifth connector; RU2 when present is bonded to MN1 or D1 via a ninth connector; L2 when present is bonded to RU2, MN1 or D1 via a tenth connector; D2 when present is bonded to L2, RU2, MN1 or D1 via the sixth connector; RU3 when present is bonded to MN1 via an eleventh connector; L3 when present is bonded to RU3 or MN1 via a twelfth connector; HP1 is bonded to L3, RU3, or MN1 via the third connector; HP2 is bonded to L3, RU3, or MN1 via the fourth connector; each of the seventh to twelfth connectors is independently selected from the group consisting of a hydrazone moiety, an oxime moiety, a carbonate moiety (—O—C(O)—O—), an azobenzene moiety, an amide bond, an arylsulfate bond, a glycoside bond, a beta-glucuronide bond, a beta-galactoside bond, an ester bond, a phosphate bond, a pyrophosphate bond, an ether bond, a thioether bond, a disulfide bond, a C—S bond, a C—O bond, a C—N bond, a C—C bond, an imine bond, a carbamate moiety (—O—C(O)—NH—), a urea moiety (—NH—C(O)—NH—), a 1,2,4-trioxolane (TRX) moiety, a triazole moiety

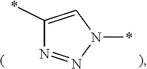 [SMILES: *c1cn(*)nn1]

a moiety derived from strain-promoted azide-alkyne cycloaddition (SPAAC), a moiety derived from strain-promoted alkyne-nitrone cycloaddition (SPANC), and a moiety derived from the trans-cyclooctene (TCO)-tetrazine click reaction; and at least one of the third to twelfth connectors is a releasable connector selected from the group consisting of a hydrazone moiety, an oxime moiety, a carbonate moiety, an azobenzene moiety, a disulfide bond, an amide bond, an arylsulfate bond, a glycoside bond, a beta-glucuronide bond, a beta-galactoside bond, an ester bond, a phosphate bond, a pyrophosphate bond, a carbamate moiety, a urea moiety, and a 1,2,4-trioxolane (TRX) moiety.

Another subset of the compounds of formula I are compounds of formula III:

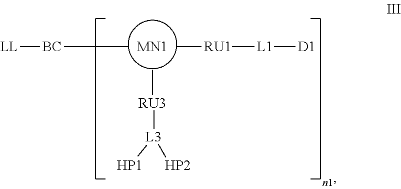

in which MN1 is a multifunctional linker complex containing 1 to 20 monomeric units; RU1 is a bond or a first releasable unit with or without a self-immolative spacer; L1 is a bond or a first bifunctional crosslinker; RU3 is a bond or a third releasable unit with or without a self-immolative spacer; L3 is a bond or a multifunctional crosslinker; RU1 when present is bonded to MN1 via the seventh connector; L1 when present is bonded to RU1 or MN1 via the eighth connector; D1 is bonded to L1, RU1, or MN1 via the fifth connector; RU3 when present is bonded to MN1 via the eleventh connector; L3 when present is bonded to RU3 or MN1 via the twelfth connector; HP1 is bonded to L3, RU3, or MN1 via the third connector; HP2 is bonded to L3, RU3, or MN1 via the fourth connector; each of the seventh, eighth, eleventh and twelfth connectors is independently selected from the group consisting of a hydrazone moiety, an oxime moiety, a carbonate moiety (—O—C(O)—O—), an azobenzene moiety, an amide bond, an arylsulfate bond, a glycoside bond, a beta-glucuronide bond, a beta-galactoside bond, an ester bond, a phosphate bond, a pyrophosphate bond, an ether bond, a thioether bond, a disulfide bond, a C—S bond, a C—O bond, a C—N bond, a C—C bond, an imine bond, a carbamate moiety (—O—C(O)—NH—), a urea moiety (—NH—C(O)—NH—), a 1,2,4-trioxolane (TRX) moiety, a triazole moiety

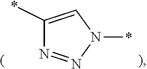 [SMILES: *c1cn(*)nn1]

a moiety derived from strain-promoted azide-alkyne cycloaddition (SPAAC), a moiety derived from strain-promoted alkyne-nitrone cycloaddition (SPANC), and a moiety derived from the trans-cyclooctene (TCO)-tetrazine click reaction; and at least one of the third, fourth, seventh, eighth, eleventh and twelfth connectors is a releasable connector selected from the group consisting of a hydrazone moiety, an oxime moiety, a carbonate moiety, an azobenzene moiety, a disulfide bond, an amide bond, an arylsulfate bond, a glycoside bond, a beta-glucuronide bond, a beta-galactoside bond, an ester bond, a phosphate bond, a pyrophosphate bond, a carbamate moiety, a urea moiety, and a 1,2,4-trioxolane (TRX) moiety.

The above described compounds including compounds of formula I, II, and III can have one or any combinations of the following features:
(i) wherein BC, MN or MN1 contains at least 2 monomeric units each being bonded to its adjacent monomeric unit via a covalent bond;
(ii) at least one of the fifth, seventh and eighth connectors is a releasable connector, and at least one of the sixth, ninth and tenth connectors is a releasable connector;
(iii) HP1 and HP2 are connected to a single monomeric unit contained in MN or MN1;
(iv) HP1 and HP2 are each connected to the monomeric unit via a C—N bond;
(v) each of HP1 and HP2, independently, is a pharmaceutically active moiety;
(vi) LL contains amino (NH₂), iodo (I), bromo (Br), a maleimide moiety, an N-hydroxysuccinimide (NHS) moiety, or an aminooxy moiety; and LL is bonded to BC, MN or MN1 via an amide bond, an arylsulfate bond, a glycoside bond, a beta-glucuronide bond, a beta-galactoside bond, an ester bond, a phosphate bond, a pyrophosphate bond, an ether bond, a thioether bond, a disulfide bond, a C—S bond, a C—O bond, a C—N bond, a C—C bond, an imine bond, a carbamate moiety (—O—C(O)—NH—), a urea moiety (—NH—C(O)—NH—), a 1,2,4-trioxolane (TRX) moiety, a triazole moiety

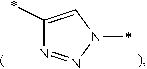 [SMILES: *c1cn(*)nn1]

a moiety derived from strain-promoted azide-alkyne cycloaddition (SPAAC), a moiety derived from strain-promoted alkyne-nitrone cycloaddition (SPANC), or a moiety derived from the trans-cyclooctene (TCO)-tetrazine click reaction;
(vii) LL is

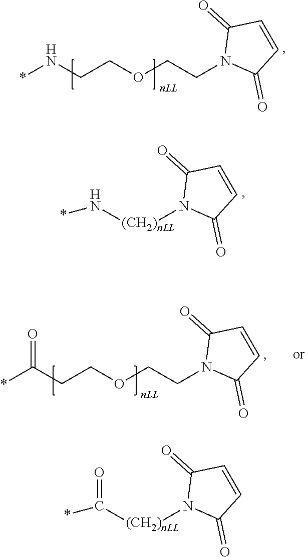

in which nLL is an integer from 0 to 10, preferably 1 or 2;
(viii) LL is

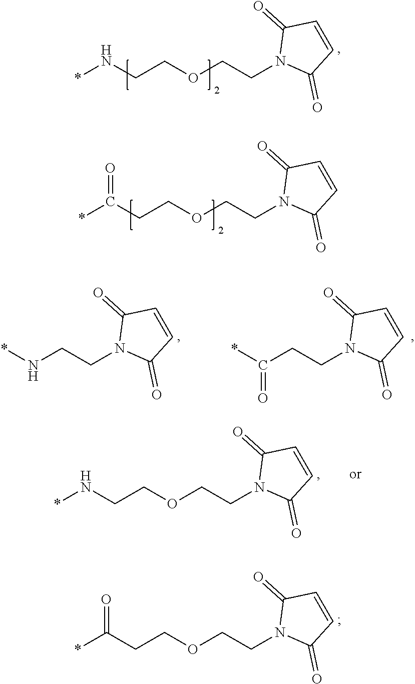

(ix) each of BC, MN, MN1, L1, L2, and L3, independently, contains one or more monomeric units derived from the group consisting of a natural amino acid, a non-natural amino acid, a monosaccharide, a bi- or multi-functional crosslinker, a polyamine, a polyalcohol, a polycarboxylic acid, a mercaptocarboxylic acid, a hydroxycarboxylic acid, a glycolic acid, a lactic acid, a diglycolic acid, an iminodiacetic acid, an aminoaldehyde, an aminoke to, an aminoalcohol, an aminothiol, a penicillamine, 2-(4-aminophenyl)-2-hydroxyacetic acid, pyrrolidine-2,4-dicarboxylic acid, 1-aminocyclopentane-trans-1,3-dicarboxylic acid, 3,6-bis-(4-aminobutyl)piperazine-2,5-dione, a cyclic peptide, and a diamino dicarboxylic acid;
(x) each of BC, MN, and MN1, independently, is an oligomer containing 1 to 10 monomeric unit selected from the group consisting of:

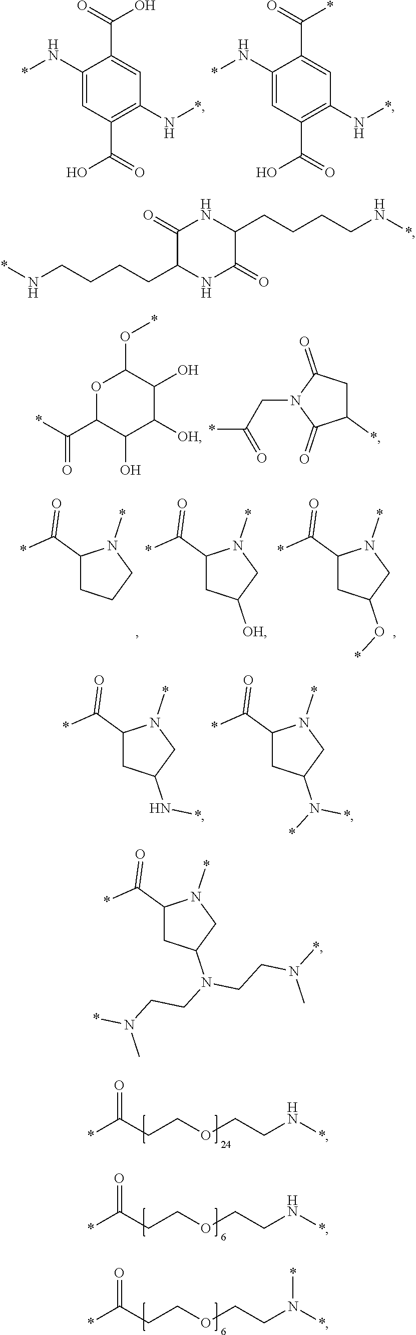

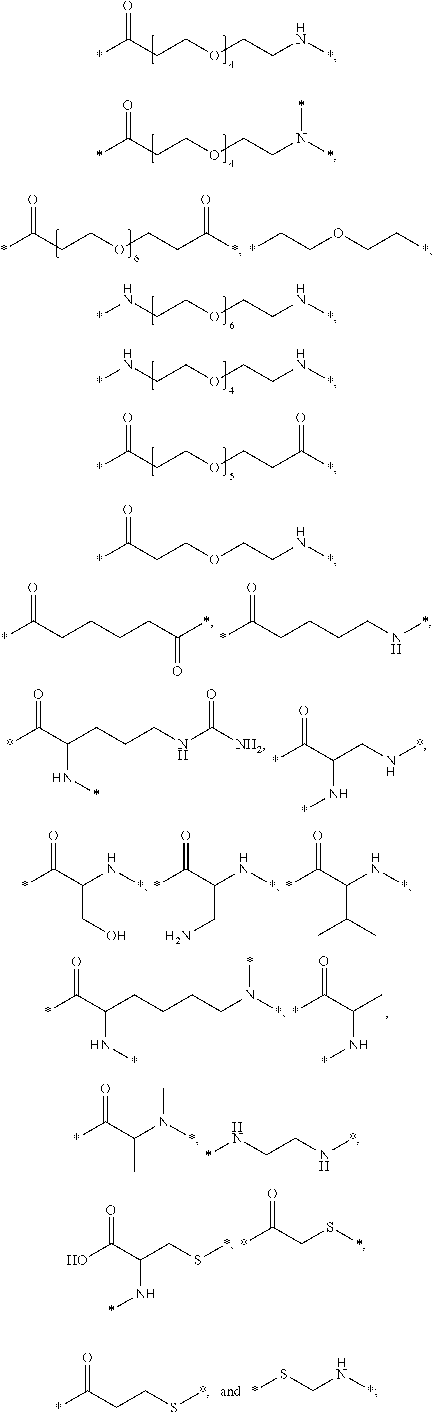

(xi) each of BC, MN, and MN1, independently, is the oligomer containing 1 to 8 monomeric units selected from the group consisting of:

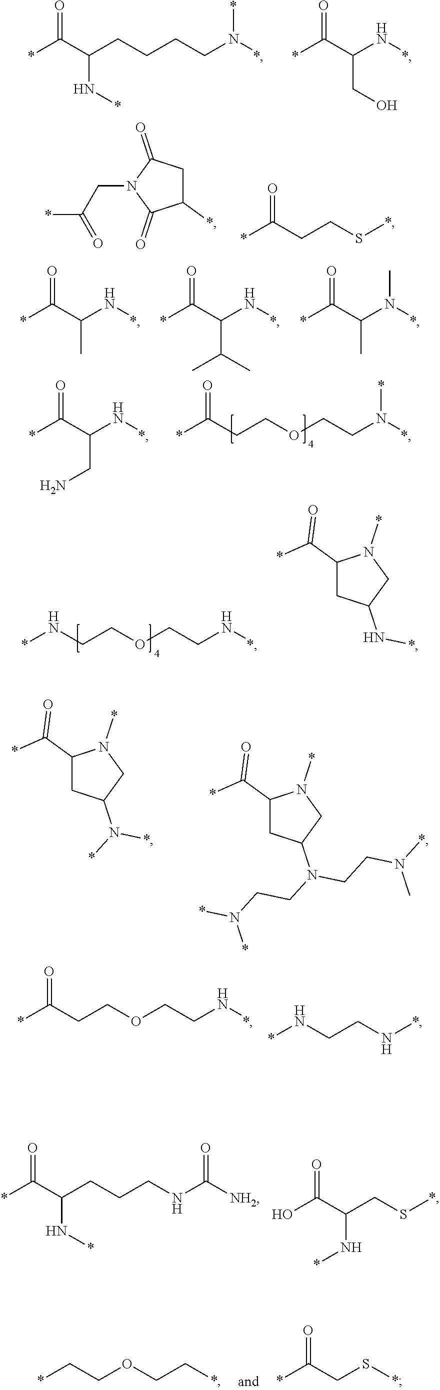

(xii) each of HP1 and HP2, independently, is a monosaccharide moiety, an oligosaccharide moiety, a PEG moiety, or a derivative thereof,
(xiii) each of HP1 and HP2, independently, is a glucose moiety, a galactose moiety, a mannose moiety, a ribose moiety, a maltose moiety, a lactose moiety, a cellobiose moiety, a maltotriose moiety, a cellotriose moiety, or a bleomycin moiety;
(xiv) each of RU1, RU2, and RU3, independently, is a serine moiety, a PEG moiety, a valine moiety, a citrulline moiety, an alanine moiety, an asparagine moiety, a 2,3-diaminopropionic acid (DAP) moiety, 4-aminobenzyl alcohol, 4-aminobenzyl-amine, an alanine-alanine-asparagine moiety, a glycine-glycine-phenylalanine-glycine moiety, or any combination thereof, and
(xv) each of D1 and D2 is a drug moiety derived from mertansine (DM1), ravtansine (DM4), N-methyl-L-Ala-maytansinol, monomethyl auristatin E, monomethyl auristatin F (MMAF), Eribulin (CAS No: 253128-41-5),7-ethyl-10-hydroxycamptothecin (SN38), Dxd (Exatecan derivative for ADC, CAS No: 1599440-33-1), ARV-771 (CAS No: 1949837-12-0), TLR7/8 agonist Ag (e.g., R848 (Resiquimod), 2-propylthiazolo[4,5-c]quinolin-4-amine (CL075, 3M002), 2-(ethoxymethyl)-1H-imidazo[4,5-c]quinolin-4-amine hydrochloride (CL097), gardiquimod, imiquimod, and the like (See, e.g., WO2022098954A1 and US 20230279014), a STING agonist (e.g., 2′,3′-cGAMP (cyclic guanosine monophosphate-adenosine monophosphate), c-di-GMP (cyclic dimeric GMP), c-di-AMP (cyclic dimeric AMP), 3′,3′-cGAM, 2′3′-c-di-AM(PS)2 (Rp,Rp) ammonium salt (CAS No. 1638750-96-5), Ulevostinag (MK-1454, CAS No. 2082743-96-0, See e.g., US 20210008190A1 and WO2020227159A2), a NOD1 agonist or NOD2 agonist (such as iE-DAP (gamma-D-glutamyl-meso-diaminopimelic acid), C12-iE-DAP (Lauroyl-gamma-D-glutamyl-meso-diaminopimelic acid), Tri-DAP (L-alanyl-gamma-D-glutamyl-meso-diaminopimelic acid), FK156 (d-lactyl-l-Ala-gamma-d-Glu-meso-DAP-Gly), FK565 (heptanoyl-gamma-d-Glu-meso-DAP-Ala), PGN-ECndss (soluble sonicated peptidoglycan from E. coli K12), MDP (muramyl dipeptide), murabutide, L18-MDP, M-TriLYS, N-Glycolyl-MDP, or the like), or any combination thereof.

The structures of control compounds 1-5 and exemplary compounds 6-46 of this invention are shown below:

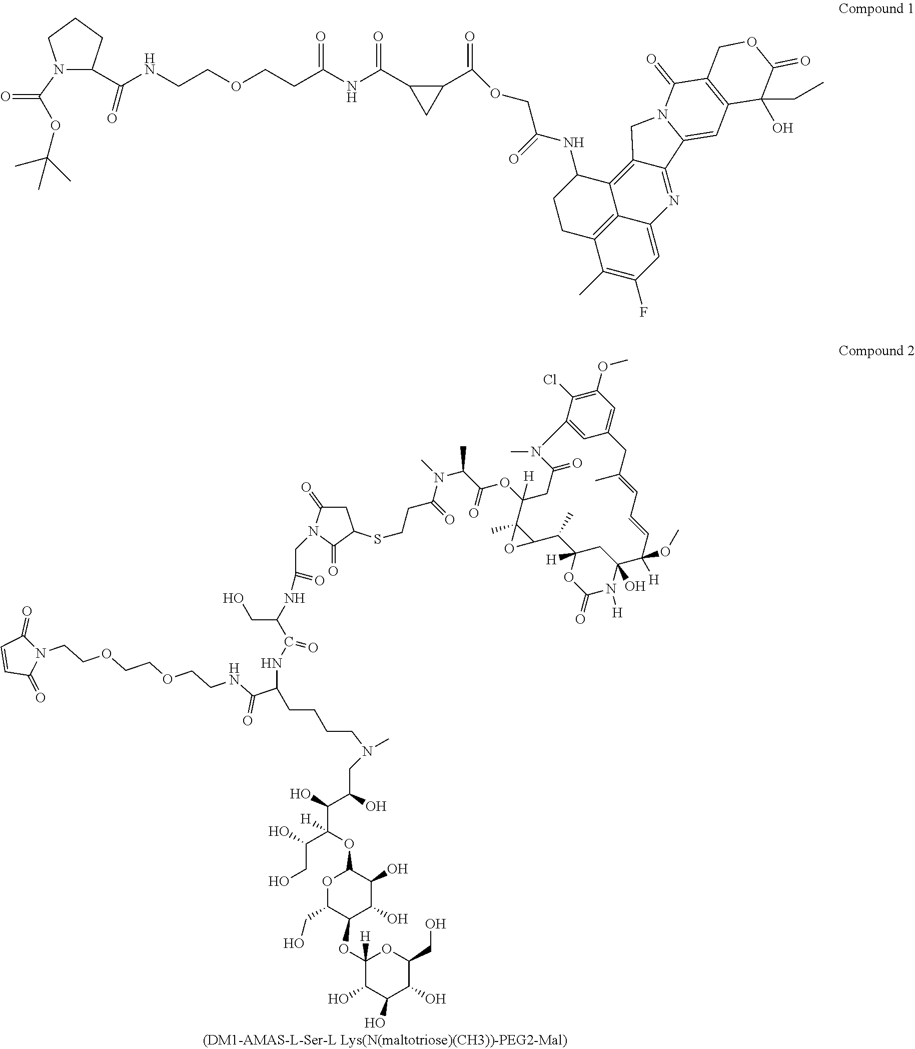

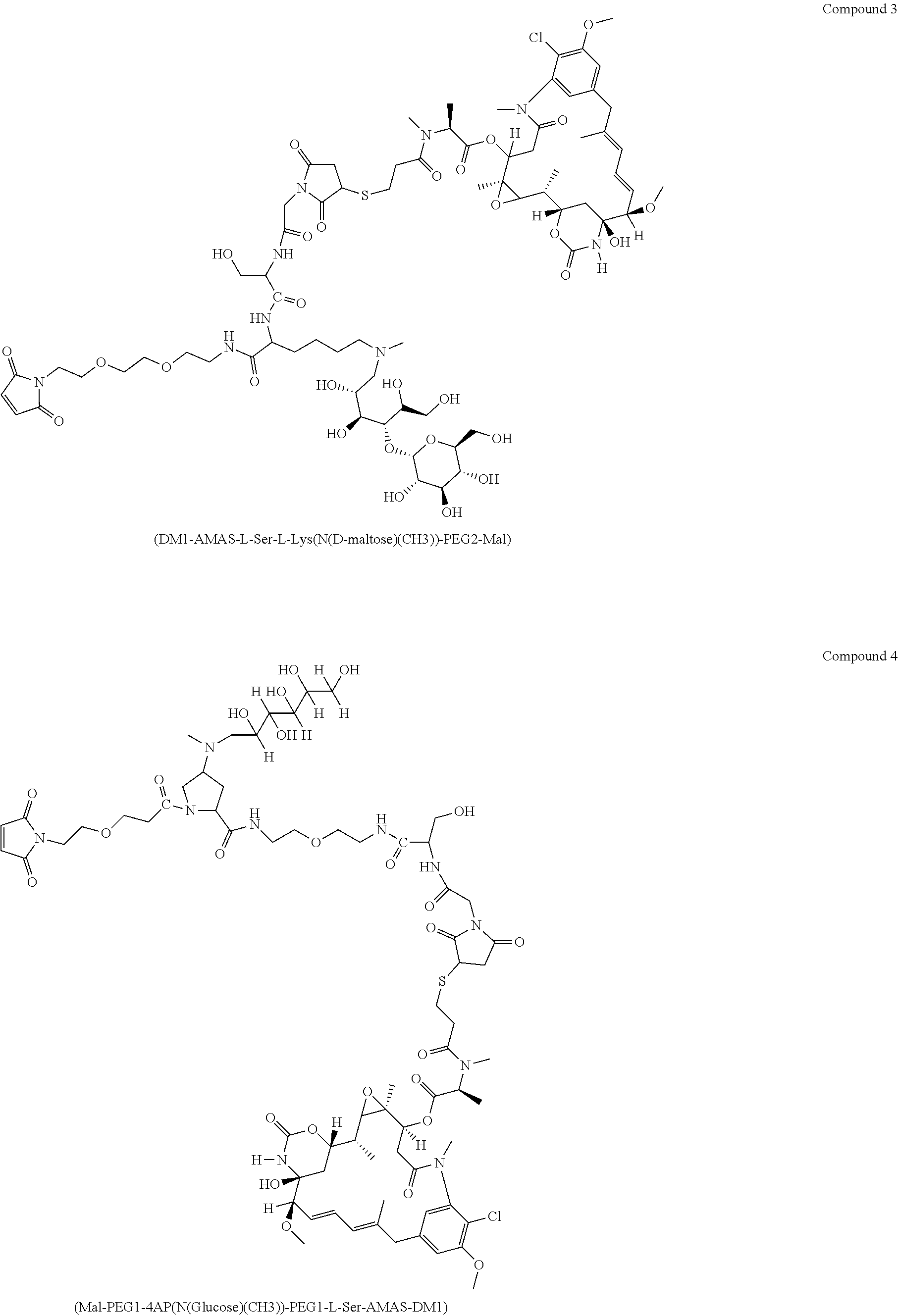

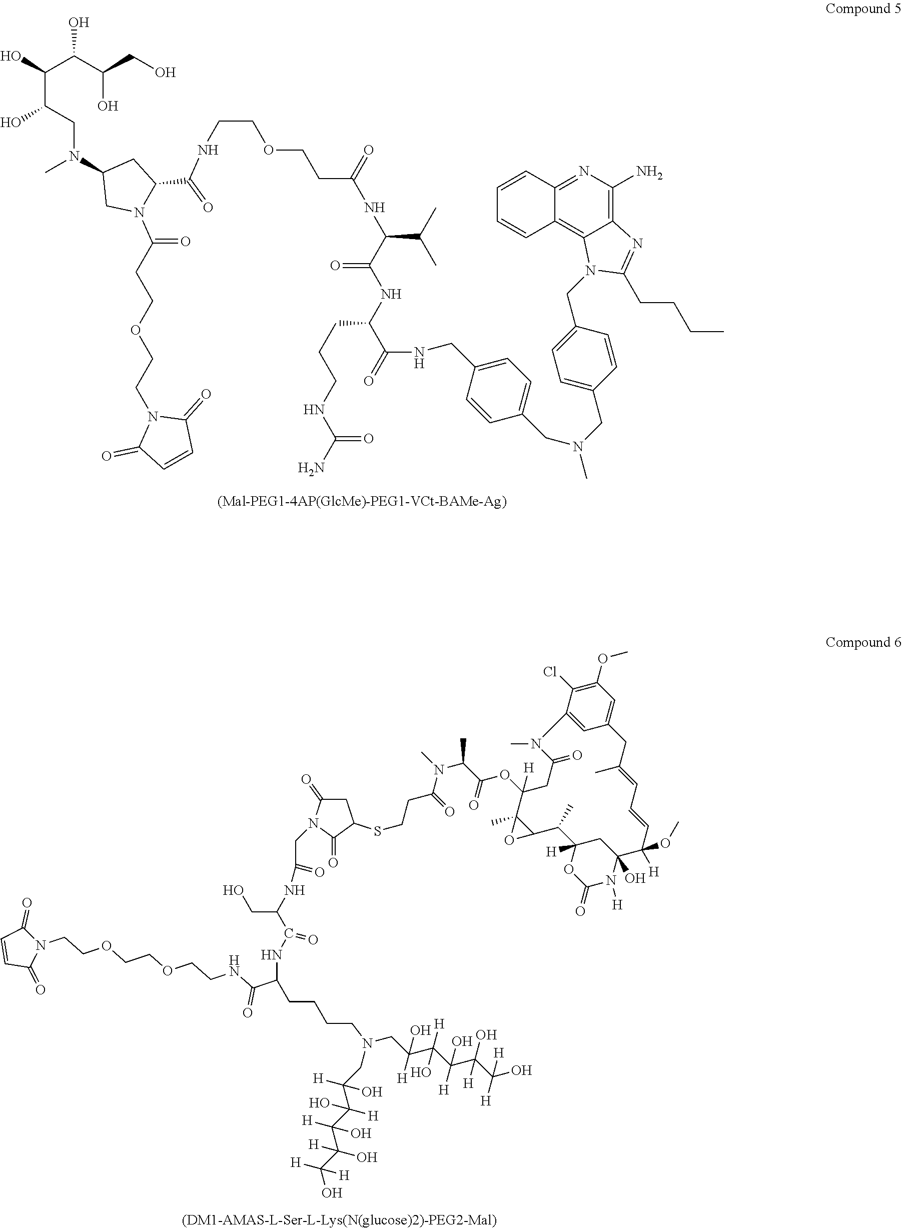 [SMILES: CCCCc1nc2c(N)nc3ccccc3c2n1Cc1ccc(CN(C)Cc2ccc(CNC(=O)[C@H](CCCNC(N)=O)NC(=O)[C@@H](NC(=O)CCOCCNC(=O)[C@H]3C[C@H](N(C)C[C@H](O)[C@@H](O)[C@H](O)[C@H](O)CO)CN3C(=O)CCOCCN3C(=O)C=CC3=O)C(C)C)cc2)cc1 ; COc1cc2cc(c1Cl)N(C)C(=O)CC(OC(=O)[C@H](C)N(C)C(=O)CCSC1CC(=O)N(CC(=O)NC(CO)C(=O)NC(CCCCN(CC(O)C(O)C(O)C(O)CO)CC(O)C(O)C(O)C(O)CO)C(=O)NCCOCCOCCN3C(=O)C=CC3=O)C1=O)[C@]1(C)OC1[C@H](C)[C@@H]1C[C@@](O)(NC(=O)O1)[C@H](OC)/C=C/C=C(\C)C2]

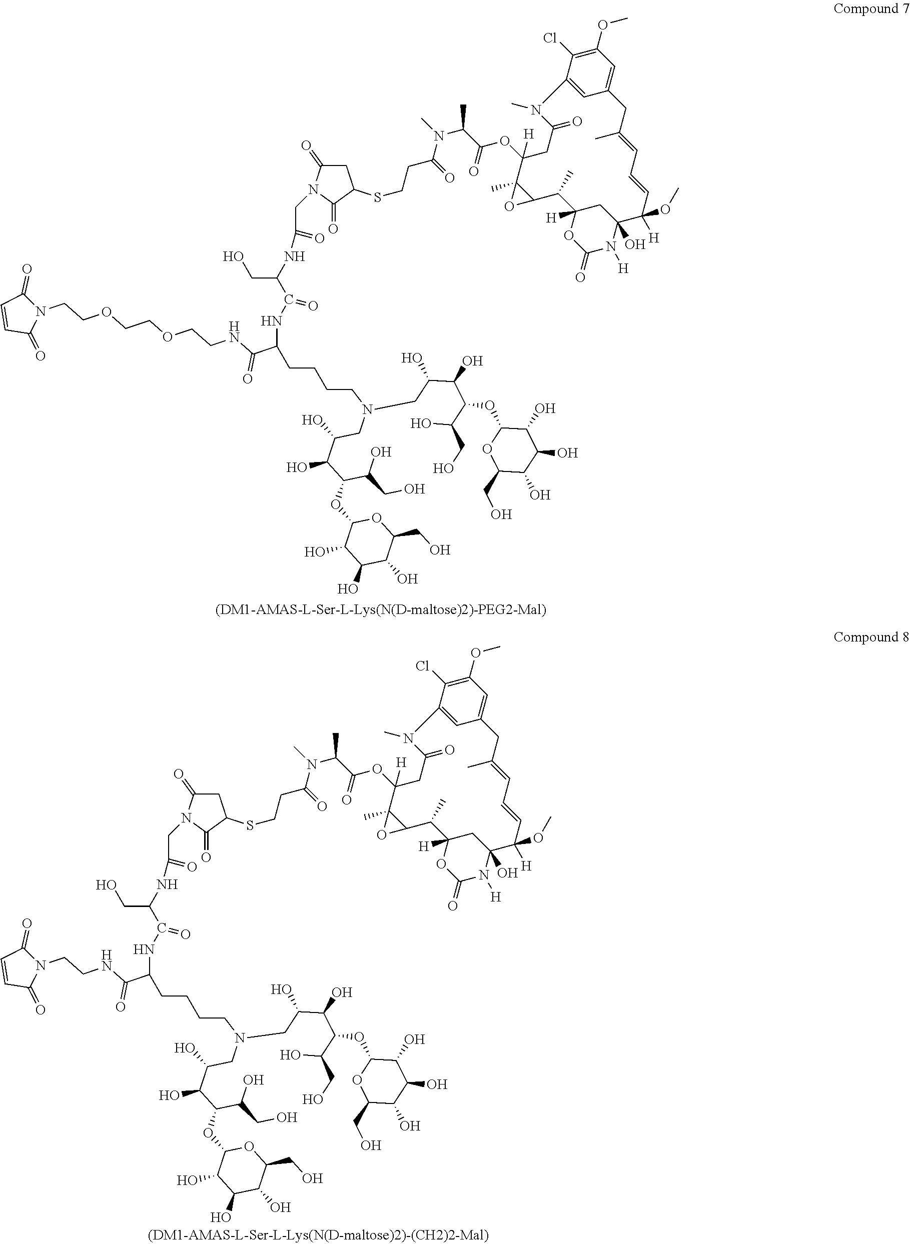

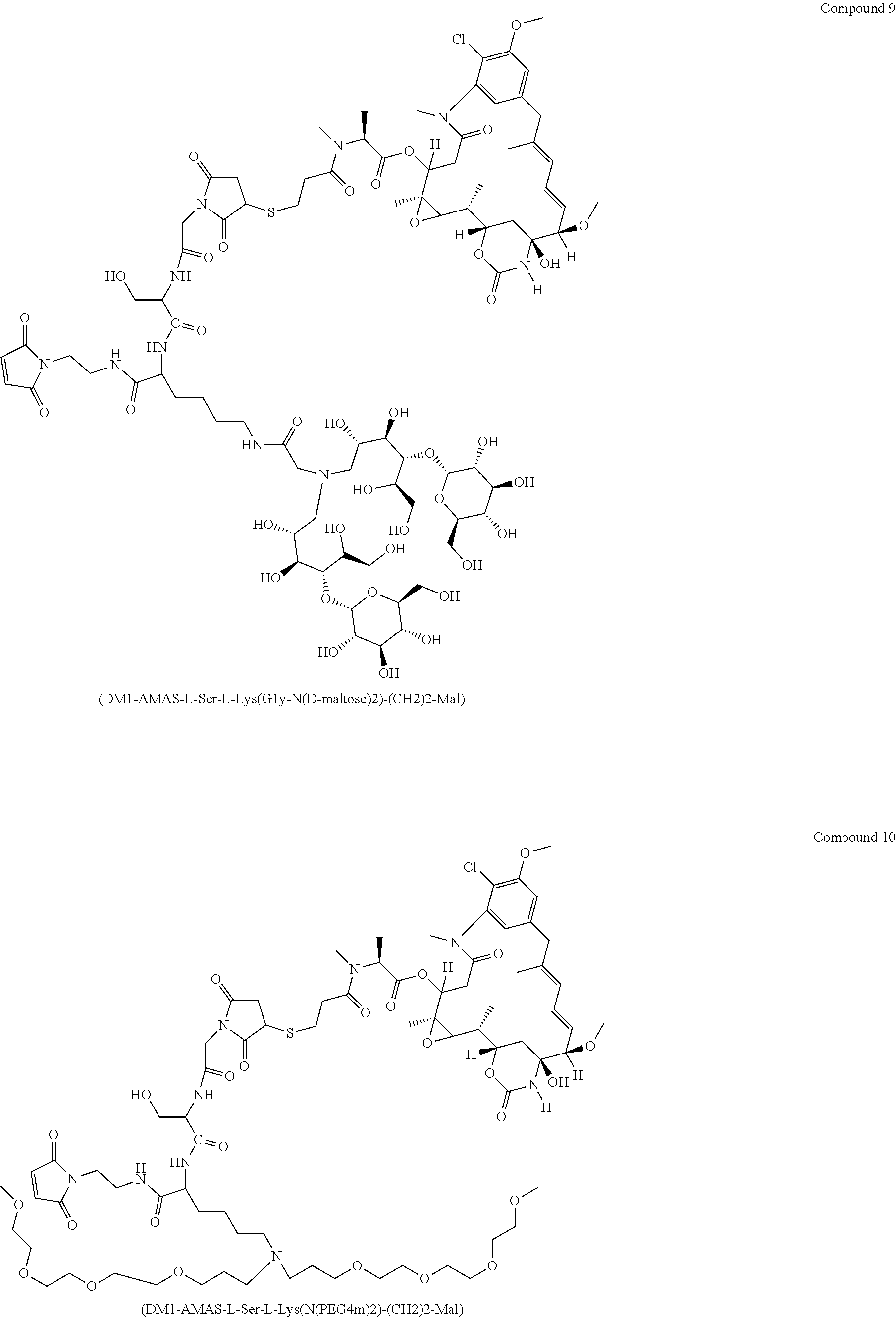

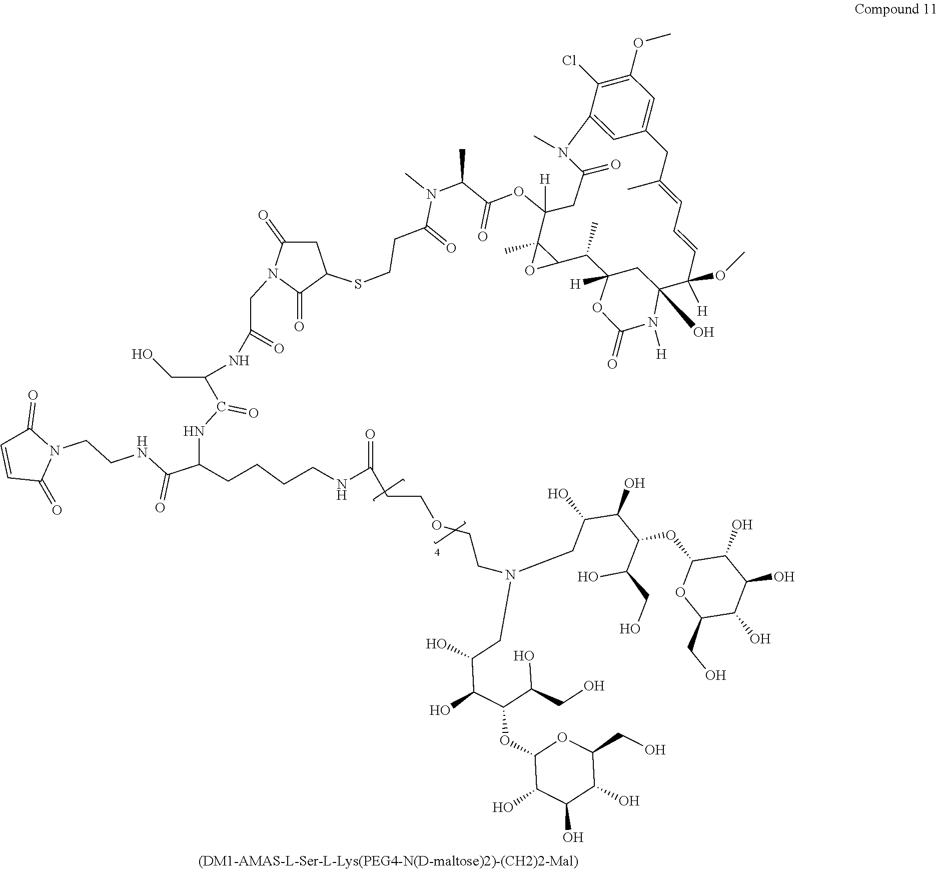

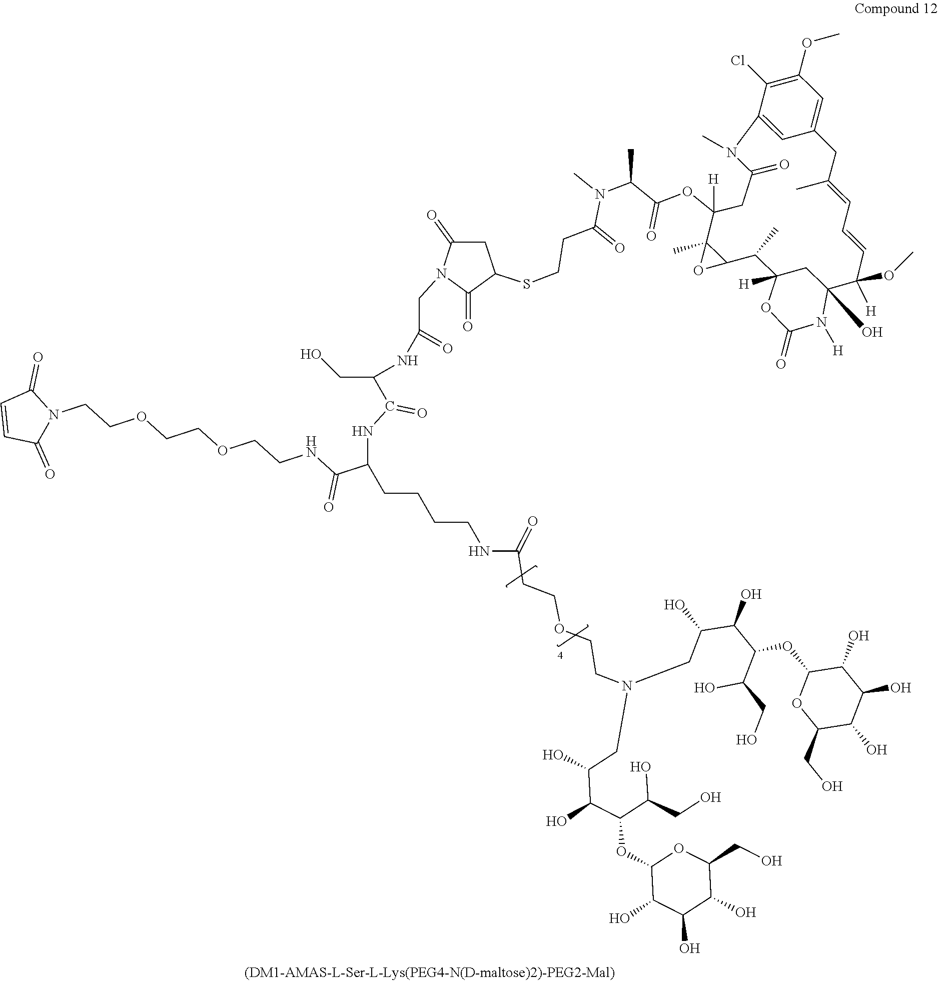

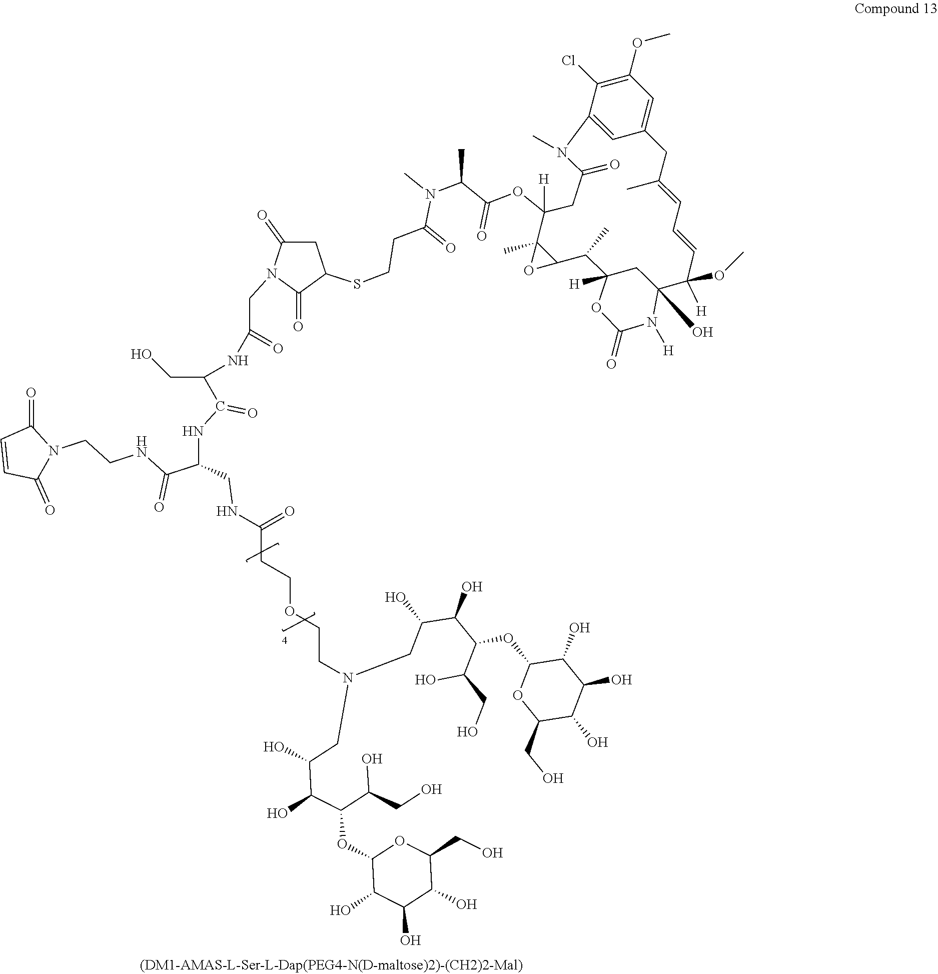

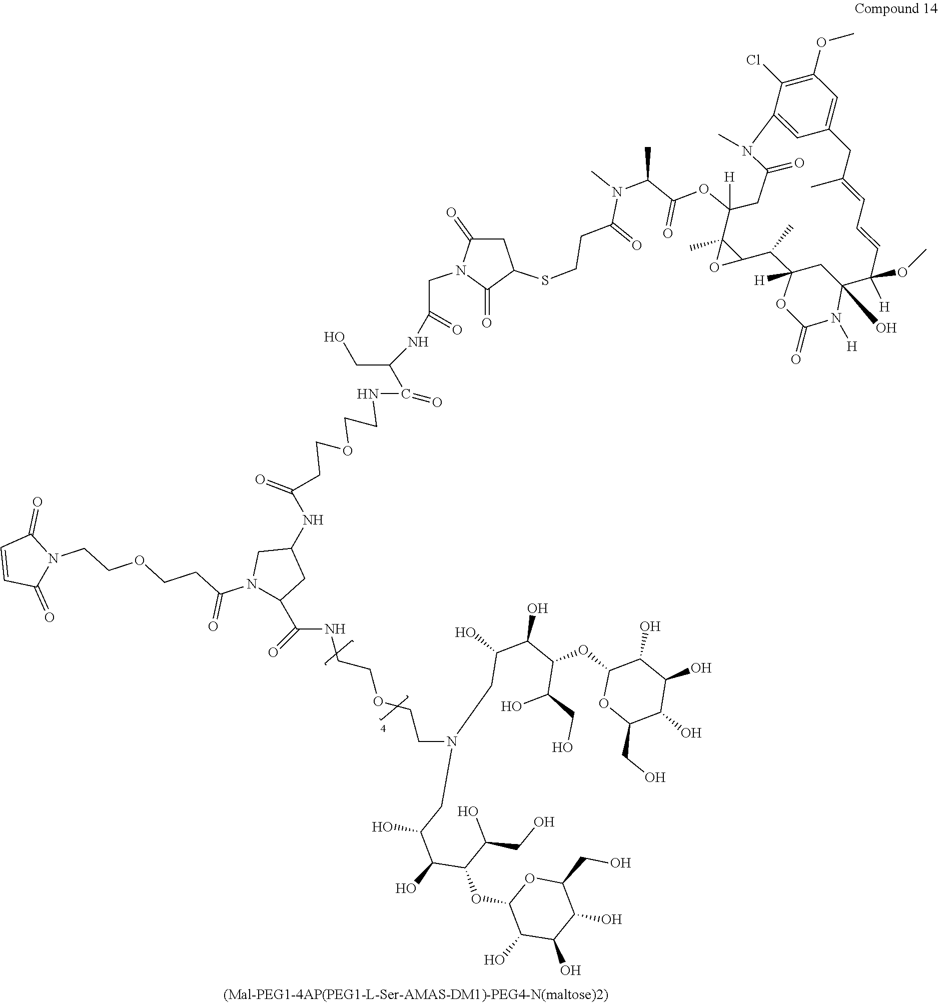

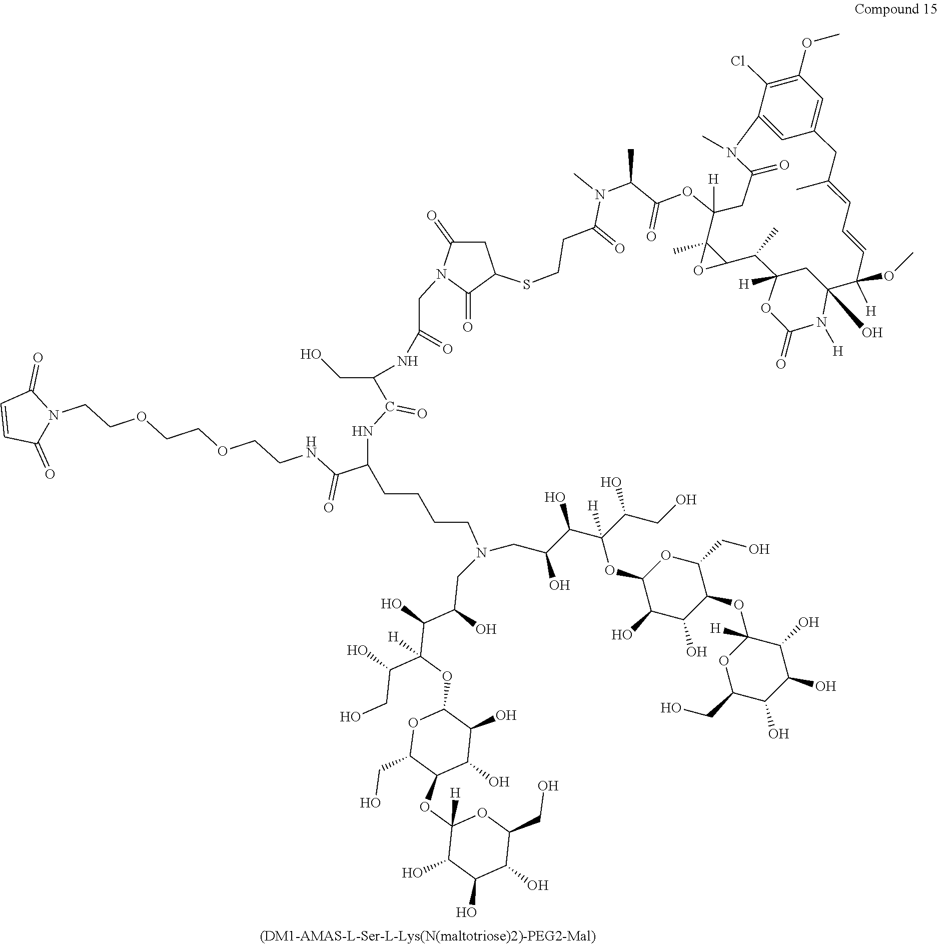 [SMILES: COc1cc2cc(c1Cl)N(C)C(=O)CC(OC(=O)[C@H](C)N(C)C(=O)CCSC1CC(=O)N(CC(=O)NC(CO)C(=O)NC(CCCCN(C[C@@H](O)[C@H](O)[C@@H](O[C@H]3O[C@@H](CO)[C@H](O[C@@H]4O[C@@H](CO)[C@H](O)[C@@H](O)[C@@H]4O)[C@@H](O)[C@@H]3O)[C@@H](O)CO)C[C@H](O)[C@@H](O)[C@H](O[C@H]3O[C@H](CO)[C@@H](O[C@H]4O[C@H](CO)[C@@H](O)[C@H](O)[C@H]4O)[C@H](O)[C@H]3O)[C@H](O)CO)C(=O)NCCOCCOCCN3C(=O)C=CC3=O)C1=O)[C@]1(C)OC1[C@H](C)[C@@H]1C[C@@](O)(NC(=O)O1)[C@H](OC)/C=C/C=C(\C)C2]

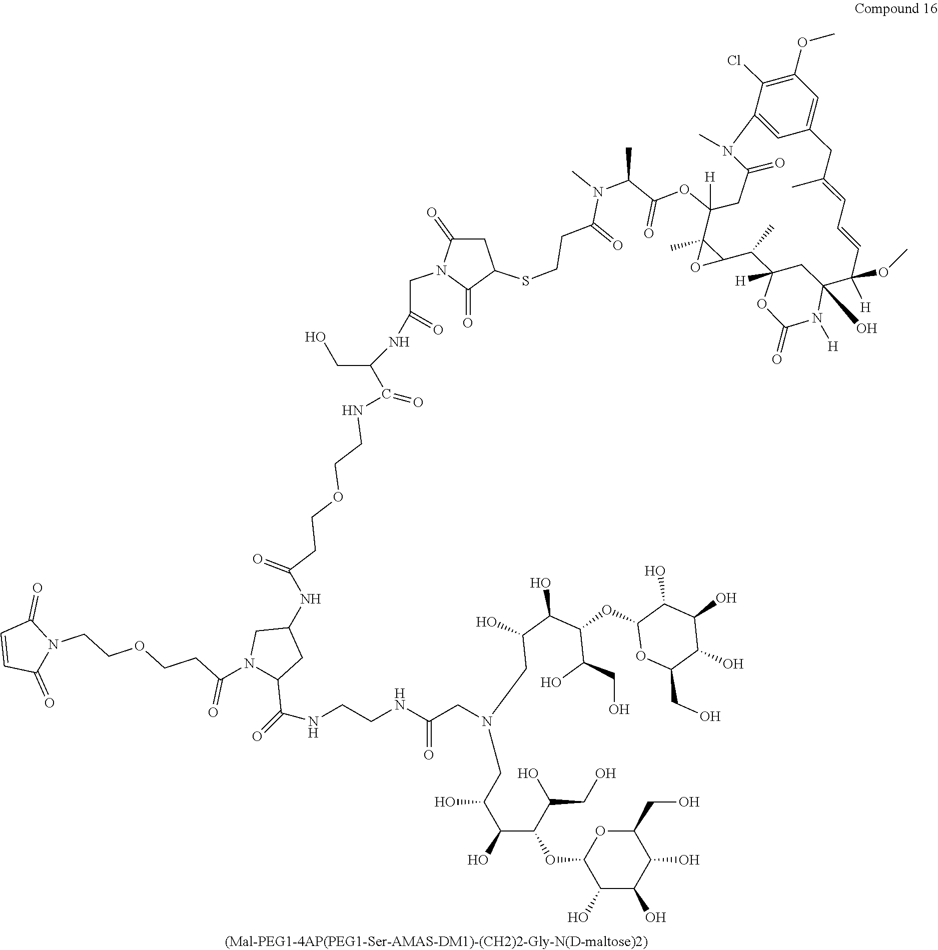

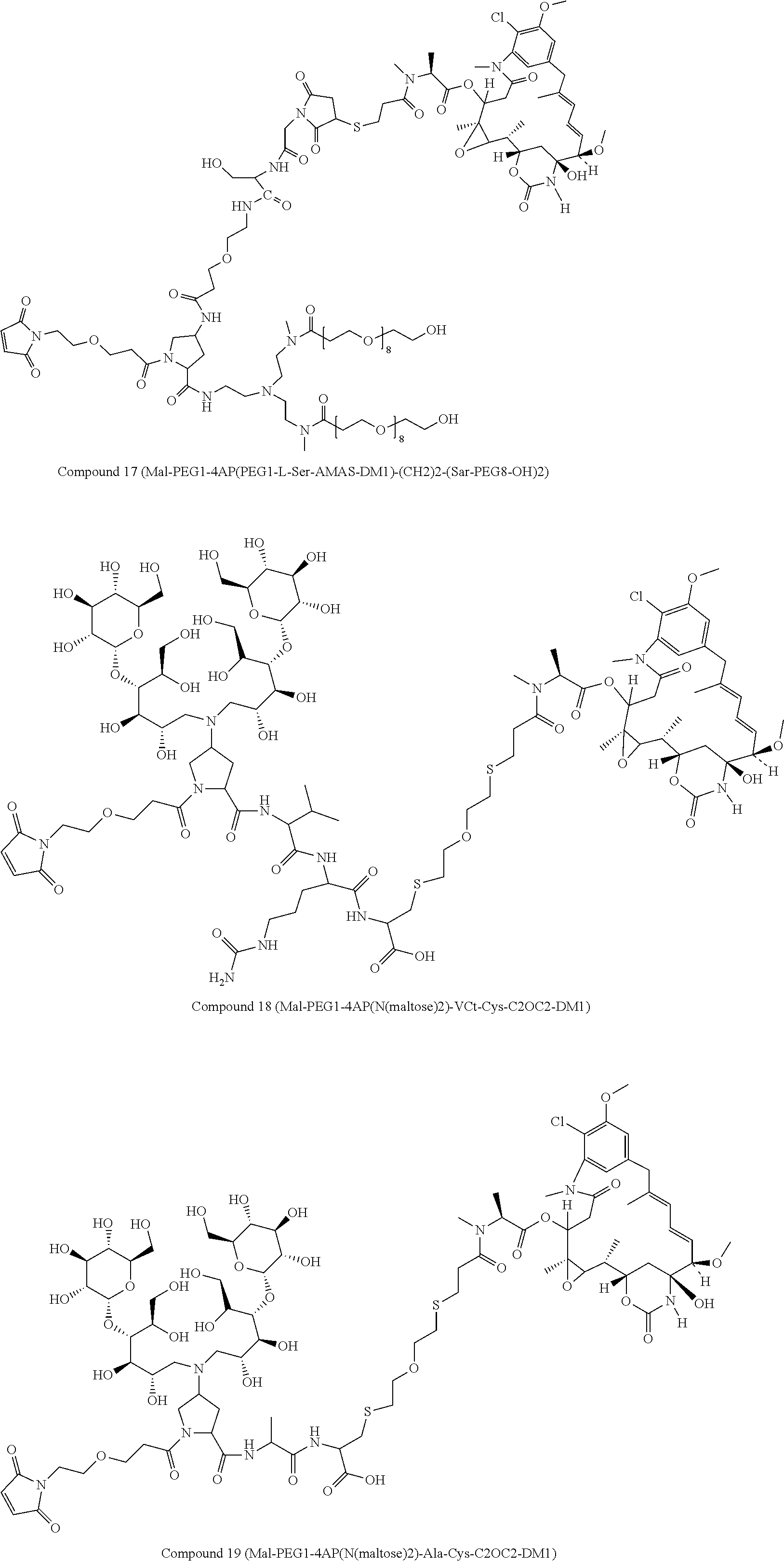

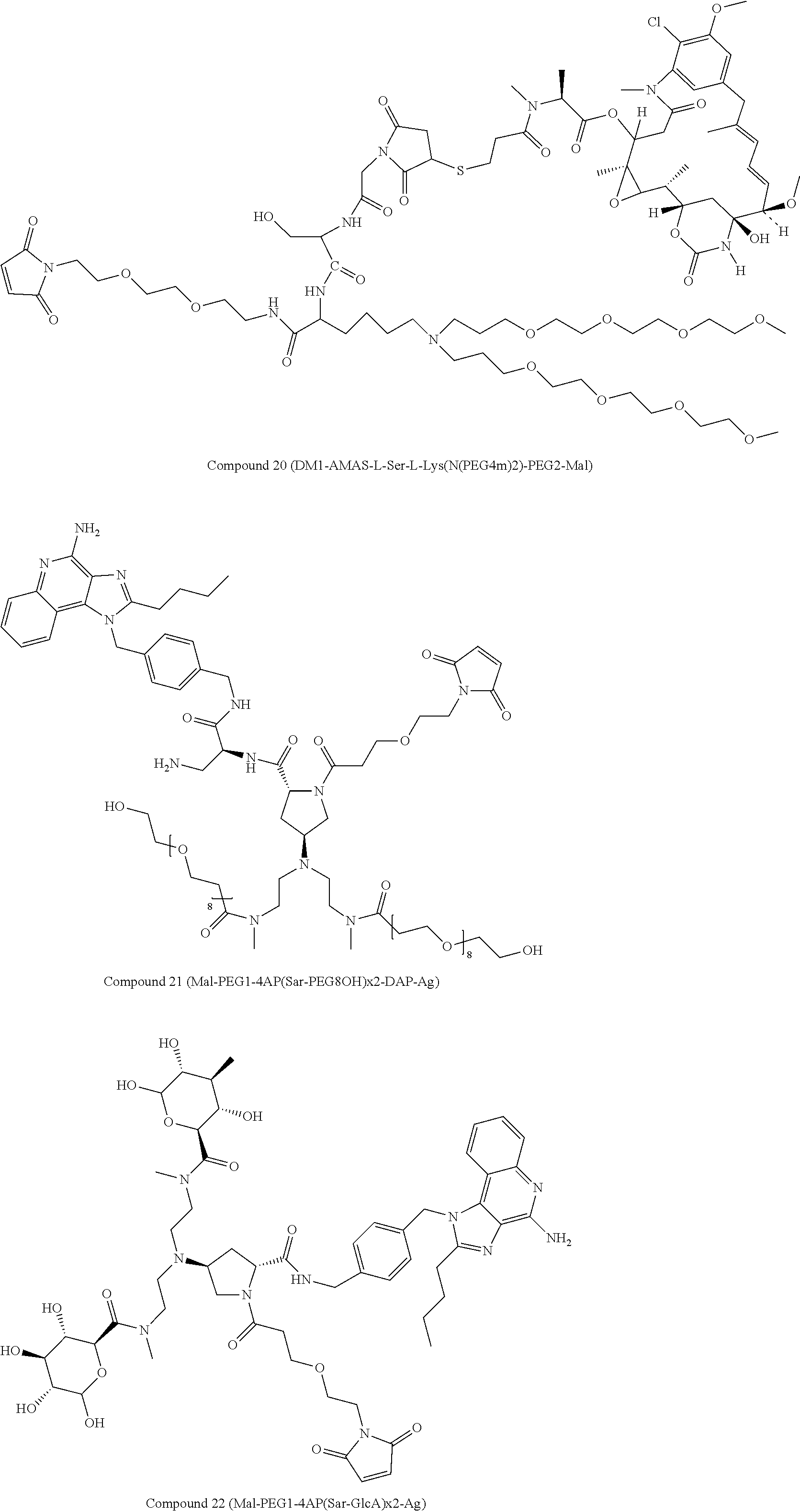

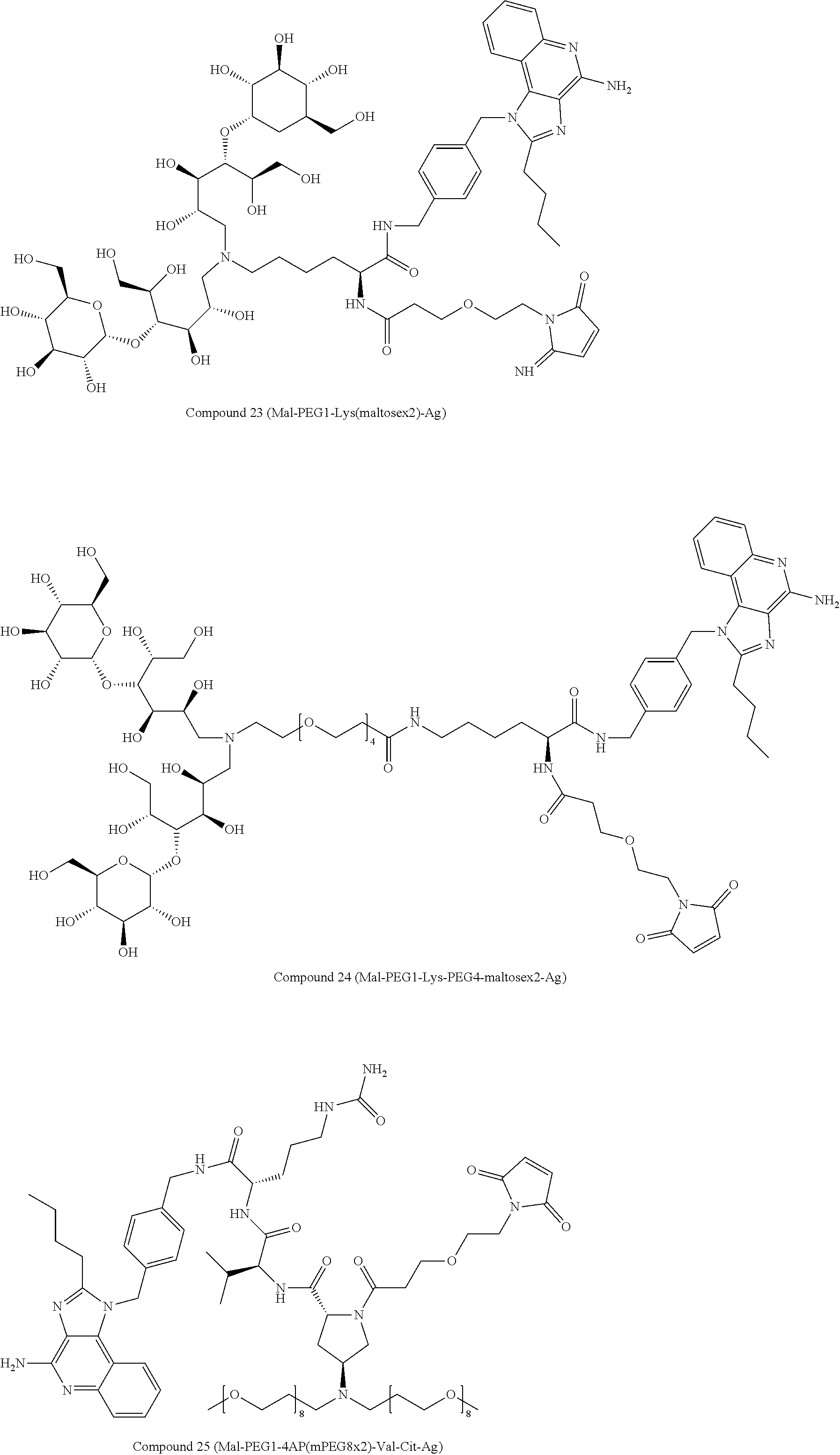

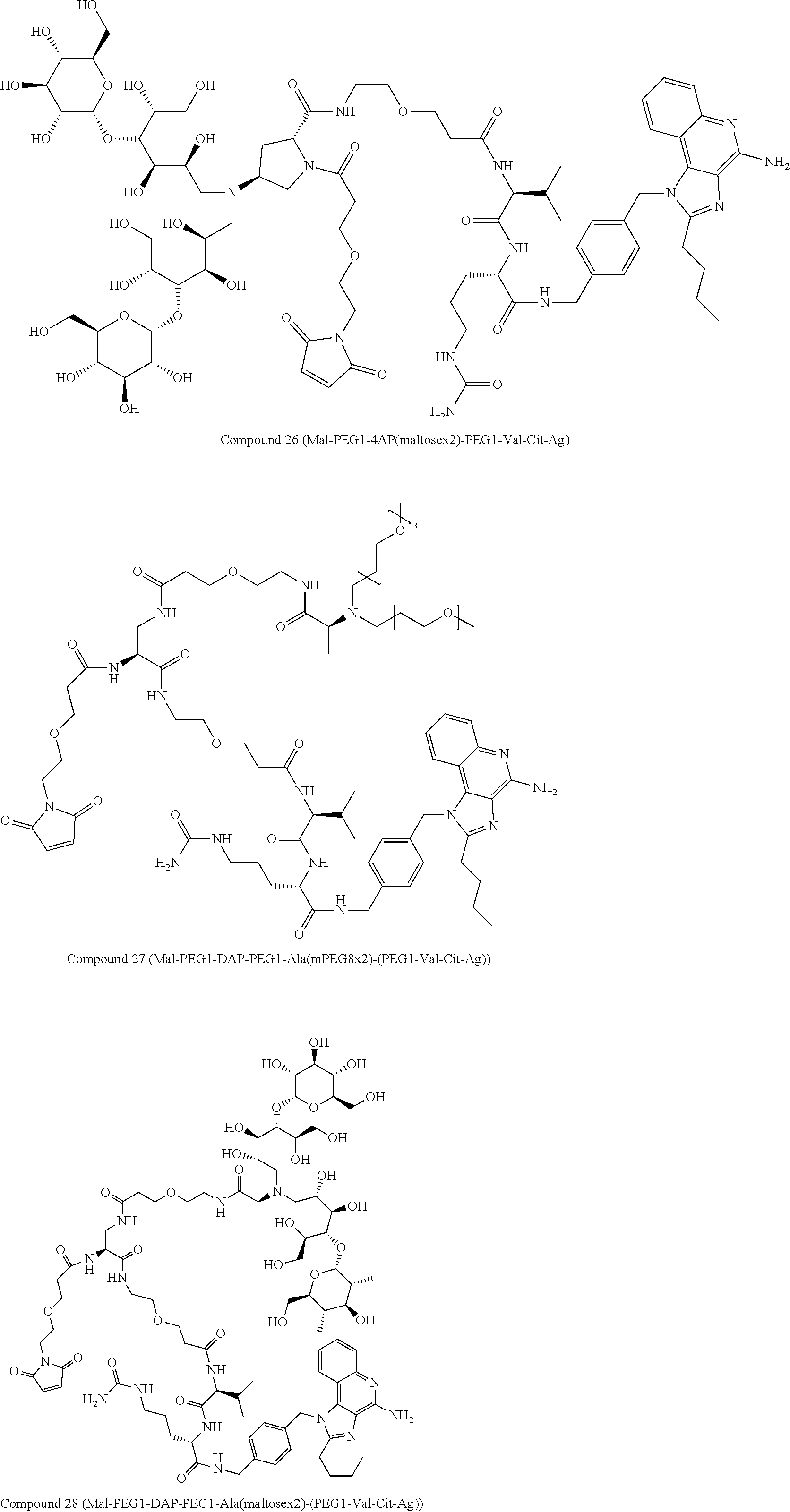

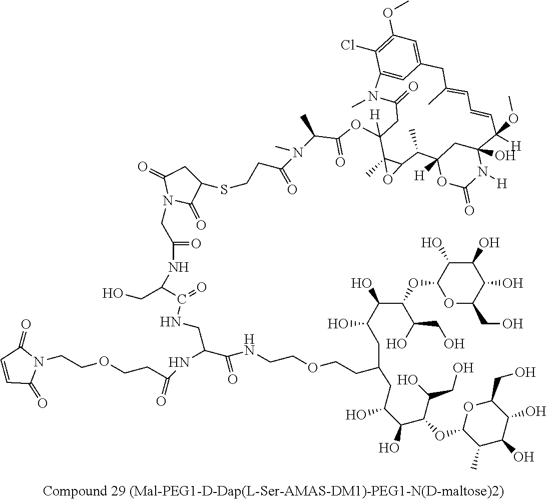

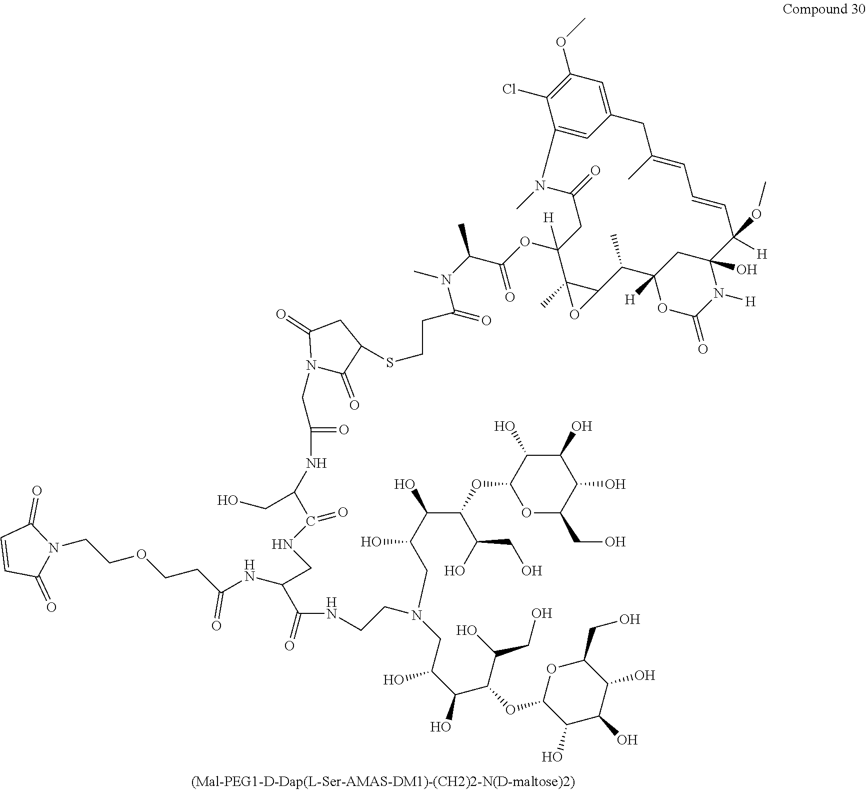

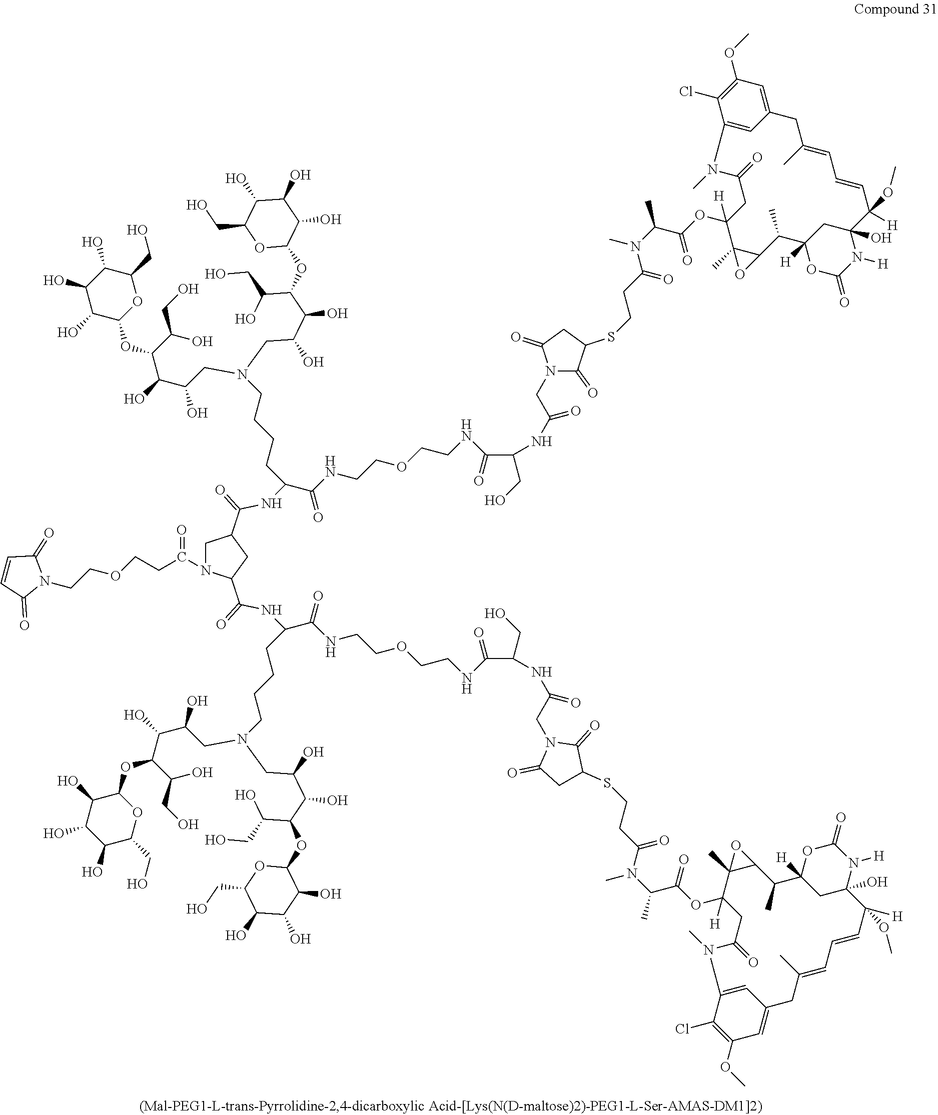

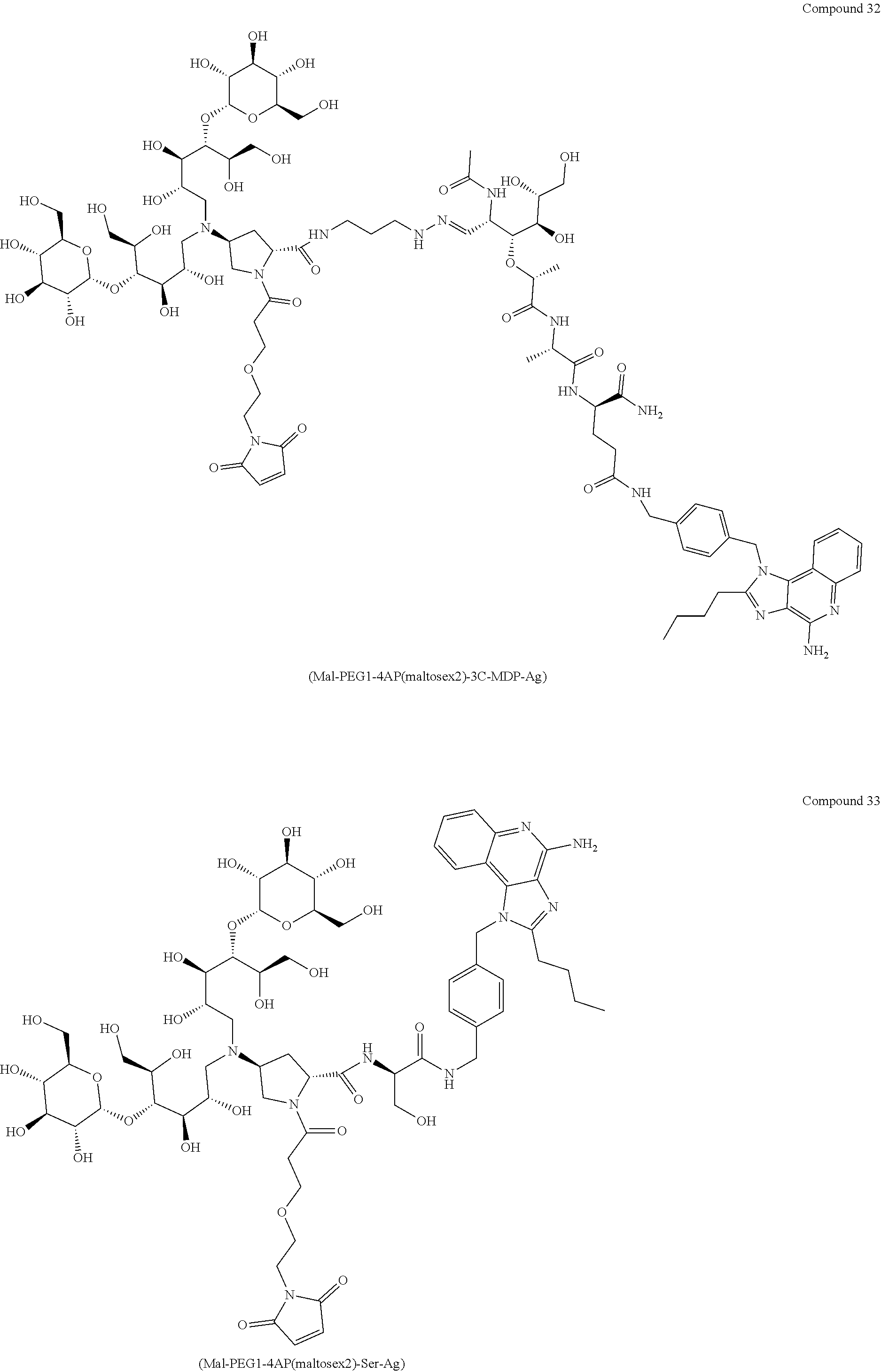

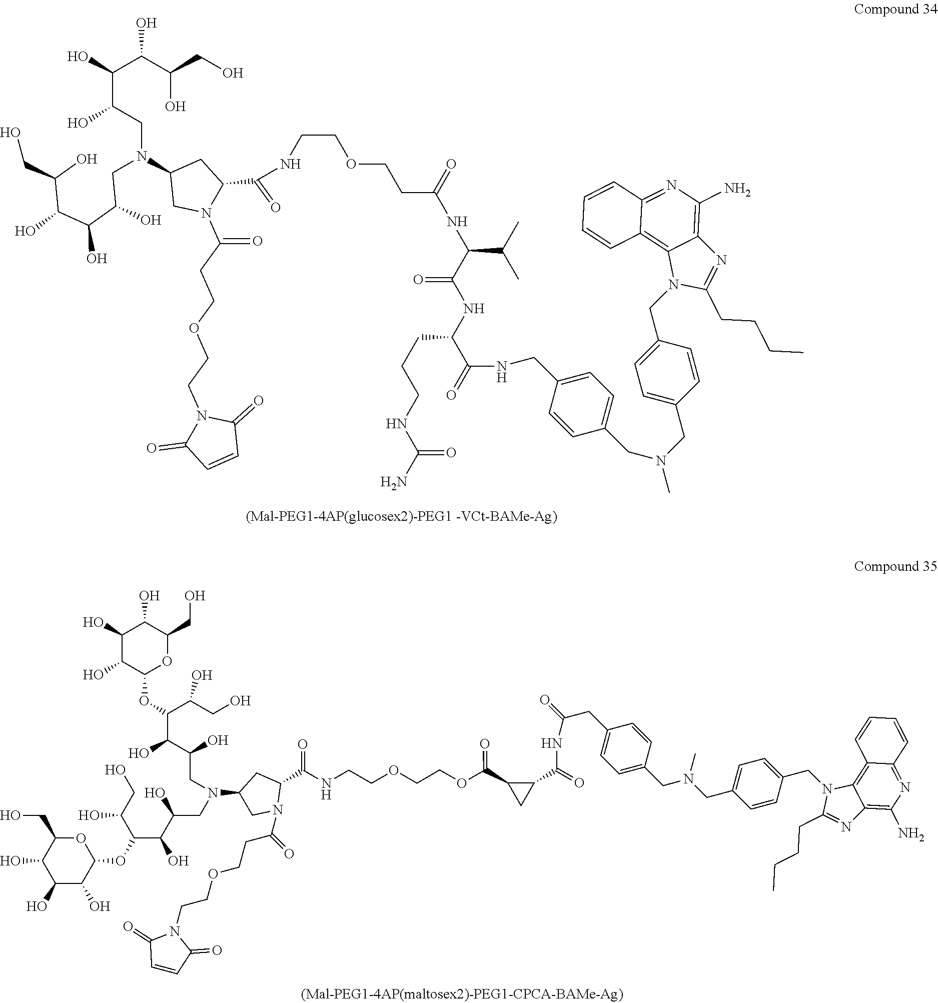

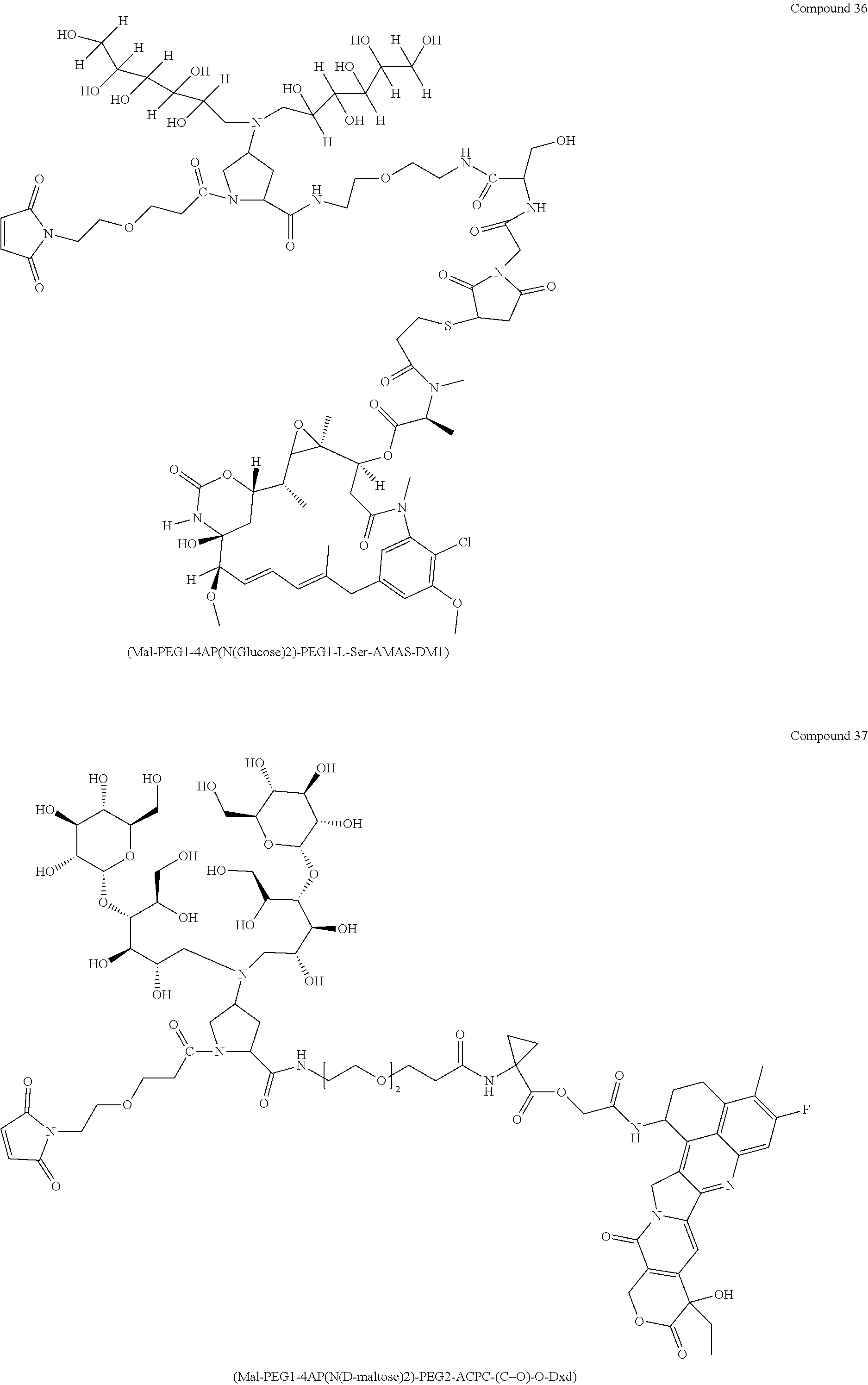

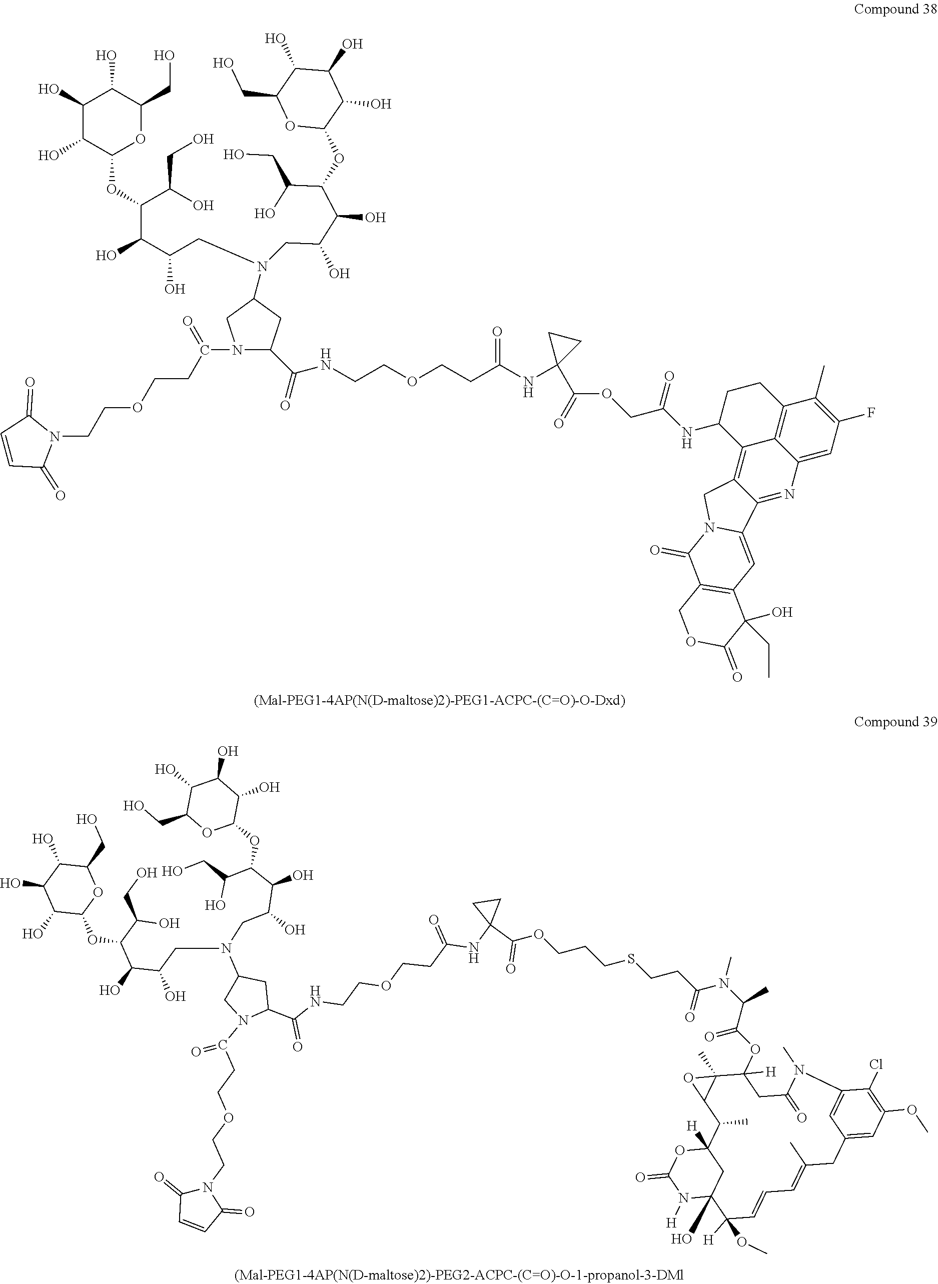

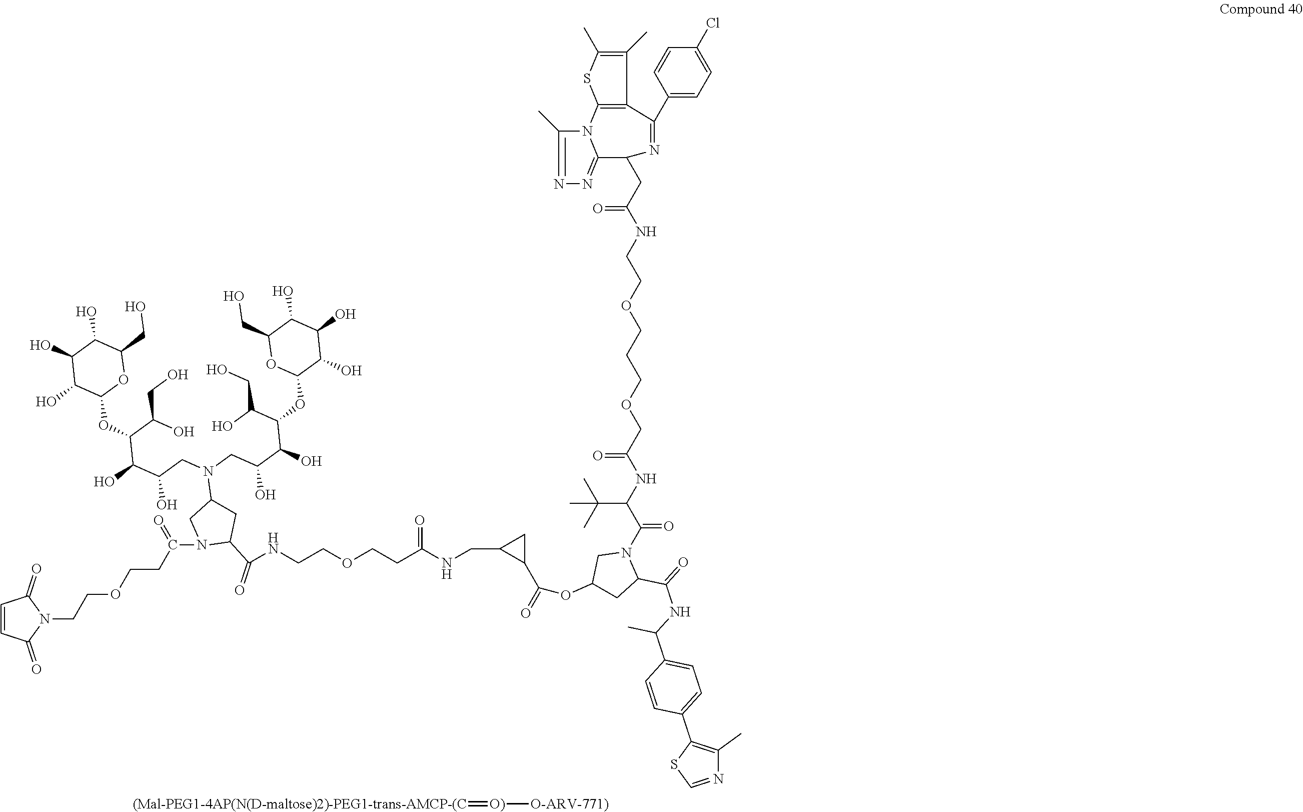

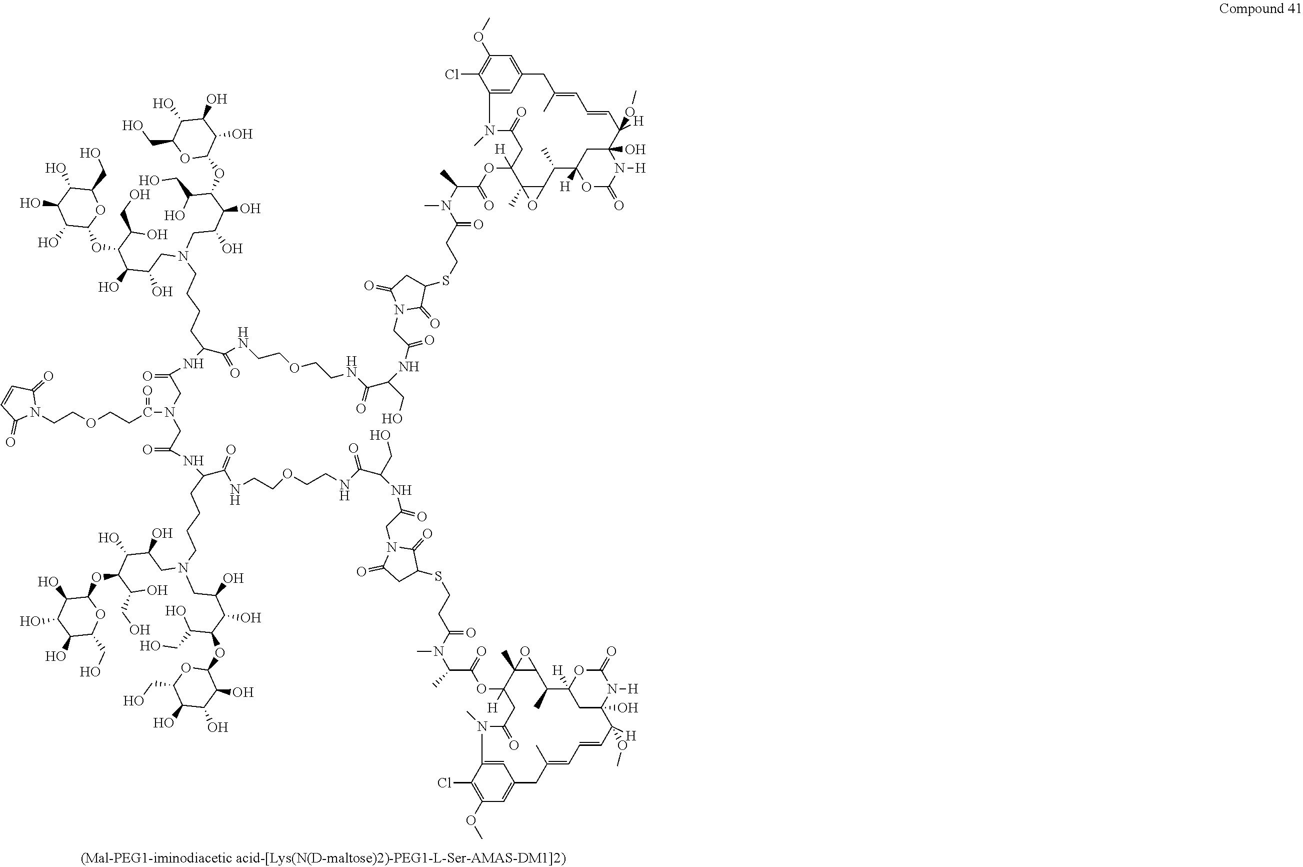

[chemical structure]

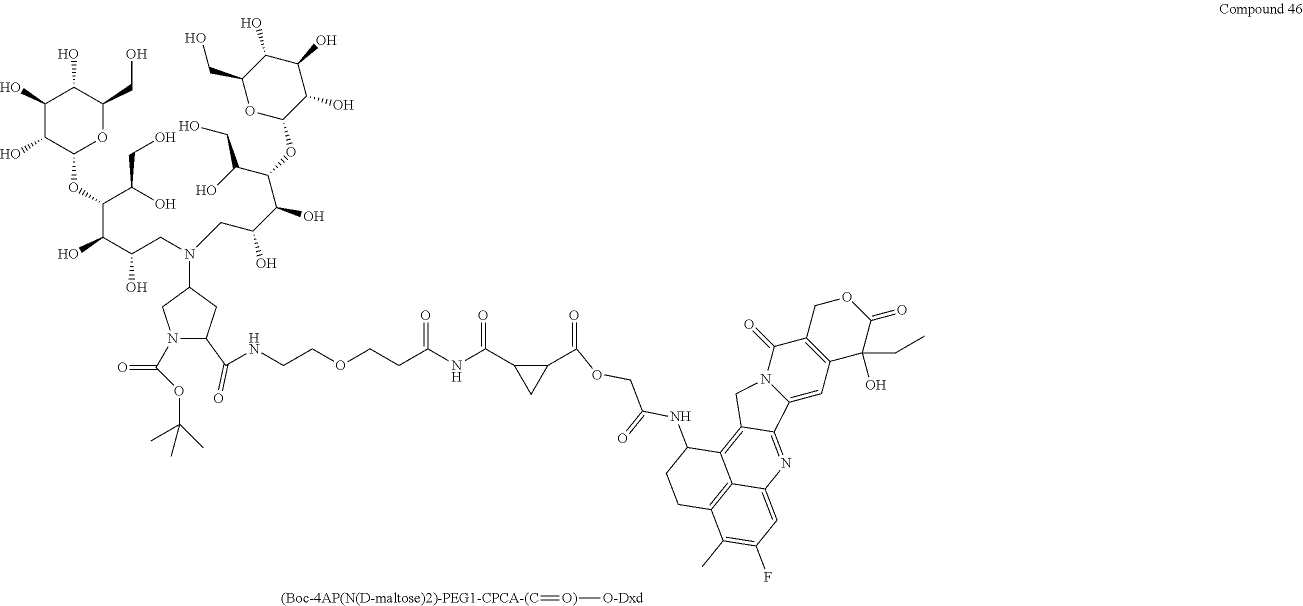

Another aspect of this invention relates to ligand-Drug conjugates each containing a ligand moiety and a moiety derived from any compound described above, in which the ligand is bonded to the compound via a covalent bond formed between a functional group from the ligand and LL in formula I, and the functional group is sulfhydryl, amino, glutamine, formyl, or Keto. Preferred conjugates include one or more of the following features in any combination: (i) the ligand is Trastuzumab, Albumin, Pertuzumab, Sacituzumab, Gemtuzumab, Brentuximab, Inotuzumab, Moxetumomab, Polatuzumab, Enfortumab, Belantamab, Loncastuximab, Tisotumab, Mirvetuximab, Datopotamab, or Telisotuzumab; (ii) the covalent bond is formed between sulfhydryl of the ligand and the maleimide, iodo, or bromo moiety of LL; and (iii) the molar ratio of the ligand to the compound is between 1:1 and 1:20, preferably between 1:2 and 1:16.

Exemplary conjugates include Conjugates 6-32 infra.

An additional aspect of this invention relates to a method of treating cancer comprising administrating to a patient in need thereof an effective amount of any conjugate described above.

Also within the scope of this invention is a pharmaceutical composition containing one of the above-described conjugates and a pharmaceutically acceptable carrier, diluent, or excipient.

Still within the scope of this invention is any of the above conjugates for the manufacture of a medicament for treating cancer.

Further included is a method of preparing a conjugate of this invention, containing the step of reacting a ligand with one of the above-described compounds. The ligand contains one or more of functional groups that are, independently, sulfhydryl, amino, glutamine, formyl or keto.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “halo” or “halogen” herein refers to a fluoro, chloro, bromo, or iodo radical. Examples include a fluoro radical (F), chloro radical (Cl), and a bromo radical (Br).

The term “alkyl” refers to a straight or branched hydrocarbon group, containing 1-20 carbon atoms (e.g., C1-20) and a monovalent radical center derived by the removal of a hydrogen atom from a carbon atom of a parent alkane. Exemplary alkyl groups are methyl, ethyl, n-propyl, isopropyl, n-butyl, iso-butyl, tert-butyl, n-pentyl, and n-hexyl.

The term “alkylcarbonyl” or “carbonyl” refers to alkyl-C(O)—.

The term “haloalkyl” refers to alkyl substituted with one or more halo atoms. Examples include fluoromethyl, difluoromethyl, trifluoromethyl, fluoroethyl (e.g., 1-fluoroetyl and 2-fluoroethyl), difluoroethyl (e.g., 1,1-, 1,2-, and 2,2-difluoroethyl), and trifluoroethyl (e.g., 2,2,2-trifluoroethyl).

The term “heteroalkyl” refers to alkyl having one or more heteroatoms (e.g., O, N, P, and S) that each replaces a carbon atom therein.

The term “alkylene” refers to bivalent alkyl containing two monovalent radical centers derived by the removal of two hydrogen atoms from the same carbon atom or two different carbon atoms of a parent alkane.

The term “heteroalkylene” refers to bivalent heteroalkyl containing two monovalent radical centers.

The term “carboxamide” refers to —C(O)NH2.

The term “amino” refers to a radical derived from amine, which is unsubstituted or substituted with alkyl, aryl, cycloalkyl, heterocycloalkyl, or heteroaryl.

The term “aminoalkyl” refers to NH₂-alkyl, i.e., an alkyl that is substituted with at least one amino group.

The term “alkylamino” refers to alkyl-NH—. Examples of aminoalkyl include aminomethyl and 2-aminoethyl. The term “acylamino” refers to —C(O)—NH—.

The term “aryl” refers to monovalent carbocyclic group containing one or more fused or non-fused phenyl rings. It is understood when multiple rings are employed, the term includes partially unsaturated ring systems. Typical aryl groups include phenyl, biphenyl, 1 or 2-naphthyl, 1,2-dihydronaphthyl, 1,2,3,4-tetrahydronaphthyl, indenyl, indanyl and the like.

The term “arylene” refers to bivalent aryl containing two monovalent radical centers.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include pyridinyl, pyrimidinyl, quinolinyl, isoquinolinyl, indolyl, benzimidazolyl, benzoxazolyl, benzothiophenyl, benzofuranyl, pyrazolyl, triazolyl, oxazolyl, thiadiazolyl, tetrazolyl, oxazolyl, isoxazolyl, carbazolyl, furyl, imidazolyl, thienyl, thiazolyl, and benzothiazolyl.

The term “cycloalkyl” refers to a nonaromatic, saturated or unsaturated monocyclic, bicyclic, tricyclic, or tetracyclic hydrocarbon group containing 3 to 12 carbons (e.g., C3-6 and C3-10). It is understood when multiple rings are employed, the term includes fused, bridged and spiro ring systems. Typical cycloalkyl groups include monocyclic, bicyclic, and spiro rings such as cyclopropyl, cyclobutyl, cyclopentyl, cyclopentenyl, cyclohexyl, cyclohexenyl, cycloheptyl, cyclooctyl, bicyclo[1.1.1]pentyl, bicyclo[2.1.1]hexyl, decahydronaphthalene and the like.

The term “carbocyclo” refers to bivalent cycloalkyl containing two monovalent radical centers.

The term “heterocycloalkyl” refers to a nonaromatic, saturated or unsaturated, 3-8 membered monocyclic, 8-12 membered bicyclic, or 11-14 membered tricyclic ring system having one or more heteroatoms (e.g., O, N, P, and S). Examples include aziridinyl, azetidinyl, pyrrolidinyl, dihydrofuranyl, tetrahydrofuranyl, tetrahydropyranyl, tetrahydrothiophenyl, tetrahydro-2-H-thiopyran-1,1-dioxidyl, piperazinyl, piperidinyl, morpholinyl, imidazolidinyl, azepanyl, dihydrothiadiazolyl, dioxanyl, quinuclidinyl, 2-azaspiro[3.3]heptanyl, and 8-azabicyclo[3.2.1]octanyl. The term “heterocyclo” refers to bivalent heterocycloalkyl containing two monovalent radical centers.

The term “formyl or aldehyde” refers to a —C(O)H group.

The term “keto” refers to a —C(O)— group.

The term “carboxyl” refers to a —C(O)—OH group.

The term “carboxylate” refers to a —O—C(O)-alkyl group.

The term “hydroxyl” refers to an —OH group.

The term “mercapto”, “sulfhydryl” or “thiol” refers to an —SH group.

The term ‘acetyloxy” refers to a —OC(O)CH₃ group.

The term “oxo” refers to a ═O group.

The term “amino acid” refers to any monomeric unit that can be incorporated into a peptide, polypeptide, or protein. Amino acids include natural amino acids and their stereoisomers, as well as non-natural amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Natural amino acids are naturally-occurring α-amino acids encoded by the genetic code, as well as those amino acids that are later modified, e.g., hydroxyproline, α-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a natural amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Non-natural (non-naturally occurring) amino acids include, without limitation, amino acid analogs, amino acid mimetics, synthetic amino acids, and N-substituted amino acids in either the L- or D-configuration that function in a manner similar to the natural amino acids. For example, “amino acid analogs” can be non-natural amino acids that have the same basic chemical structure as natural amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a natural amino acid, e.g., penicillamine. Amino acids may be referred to herein by either the commonly known three letter symbols or by the one-letter symbols recommended by the IUPAC-IUB Biochemical Nomenclature Commission.

The term “amino aldehyde” refers to an aromatic or nonaromatic, saturated or unsaturated, cyclic or non-cyclic hydrocarbon having one or more (e.g., one, two, three, and four) amino groups, one or more (e.g., one, two, three, and four) formyl groups, and at least two heteroatoms (e.g., O, N, P, and S).

The term “amino alcohol” refers to an aromatic or nonaromatic, saturated or unsaturated, cyclic or non-cyclic hydrocarbon having one or more (e.g., one, two, three, and four) amino groups, one or more (e.g., one, two, three, and four) hydroxyl groups, and at least two heteroatoms (e.g., O, N, P, and S).

The term “aminoketo” refers to an aromatic or nonaromatic, saturated or unsaturated, cyclic or non-cyclic hydrocarbon having one or more (e.g., one, two, three, and four) amino groups, one or more (e.g., one, two, three, and four) keto groups, and at least two heteroatoms (e.g., O, N, P, and S).

The term “mercaptocarboxylic acid” refers to an aromatic or nonaromatic, saturated or unsaturated, cyclic or non-cyclic hydrocarbon having one or more (e.g., one, two, three, and four) carboxyl groups, one or more (e.g., one, two, three, and four) sulfhydryl groups, and at least three heteroatoms (e.g., O, N, P, and S).

The term “aminothiol” refers to an aromatic or nonaromatic, saturated or unsaturated, cyclic or non-cyclic hydrocarbon having one or more (e.g., one, two, three, and four) amino groups, one or more (e.g., one, two, three, and four) sulfhydryl groups, and at least two heteroatoms (e.g., O, N, P, and S).

The term “hydroxycarboxylic acid” refers to an aromatic or nonaromatic, saturated or unsaturated, cyclic or non-cyclic hydrocarbon having one or more (e.g., one, two, three, and four) carboxyl groups, one or more (e.g., one, two, three, and four) hydroxyl groups, and at least two heteroatoms (e.g., O, N, P, and S).

The term “diamino dicarboxylic acid” refers to an aromatic or nonaromatic, saturated or unsaturated, cyclic or non-cyclic hydrocarbon having two or more (e.g., two, three, and four) amino groups, two or more (e.g., two, three, and four) carboxyl groups, and at least six heteroatoms (e.g., O, N, P, and S). Examples include 2,5-diaminobenzene-1,4-dicarboxylic acid, 3,6-diaminopyrazine-2,5-dicarboxylic acid, 2,2′-diamino-[1,1′-Biphenyl]-4,4′-dicarboxylic acid, 1,4-diamino-cyclohexane-1,4-dicarboxylic acid, 4,4′-Methylene bisanthranilic acid, 2,5-bis(methylamino)tere-phthalic acid, 3,6-diamino-phthalic acid, 2,5-diaminocyclohexa-1,4-diene-1,4-dicarboxylic acid, 2,3-diaminoterephthalic acid, 2,5-diaminocyclohexane-1,4-dicarboxylic acid, 2,5-diaminoterephthalic acid, 2,5-diaminobenzene-1,3-dicarboxylic acid, 2,5-dihydroxycyclohexa-1,4-diene-1,4-dicarboxylic acid, 2,5-dihydroxy-cyclohexa-1,3-diene-1,4-dicarboxylic acid, 4,6-diaminobenzene-1,3-dicarboxylic acid, 3,6-diaminopyrazine-2,5-dicarboxylic acid, 3,3′-diamino-[1,1′-biphenyl]-4,4′-dicarboxylic acid, 2,2′-diamino-4,4′-biphenyldicarboxylic acid, 2,2′-diamino-4,4′-stilbenedicarboxylic acid, 2,3-diaminosuccinic acid, 2,4-diamino-pentanedioic acid, 2,5-diaminoadipic acid, 2,6-diaminopimelic acid, 2,7-diaminosuberic acid, 2,8-diaminoazelaic acid, 2,9-diaminodecanedioic acid, 2,10-diaminoundecanedioic acid, di-O-methylated ristomycinic acid, cystathionine, lanthionine, and djenkolic acid.

An amide bond refers to either (1) a peptide bond formed between two amino acids, natural or non-natural where said peptide bond is formed between an amine group from one amino acid and a carboxylic acid group from the other amino acid; (2) an isopeptide bond which few enzymes are capable of hydrolyzing (see KW-1017, UniProtKB entries), or (3) an amide bond formed between an amino acid and a non-amino acid compound (such as a bifunctional crosslinker, a polyamine, a monosaccharide, or a diamino dicarboxylic acid).

A glycoside bond refers to one formed between a hemiacetal and a hydroxyl group.

An ester bond refers to one formed between a hydroxyl group and a carboxylic acid group.

An ether bond refers to one having two carbon atoms that are single bonded to an oxygen atom.

A disulfide bond refers to one formed between two thiol groups.

The term “enzymatically cleavable peptide (or amino acid)” refers to a peptide chain (or an amino acid) linking a drug linker and a hydrophilic polymer moiety or a drug moiety via a cleavable bond for the release of hydrophilic polymer or drug, for example upon enzymatic treatment.

The term “self-immolative spacer” refers to a chemical moiety linking to a first trigger with an acid-sensitive (e.g., hydrazone moiety, oxime moiety, carbonate moiety), redox sensitive (e.g., disulfide moiety), hypoxia-sensitive (e.g., azobenzene moiety) or enzymatically cleavable (e.g., amide bond, glycoside bond, arylsulfate bond, pyrophosphate bond or ester bond) component and a second trigger linking to a hydrophilic polymer moiety or a drug moiety, in which both the first and second triggers are capable of spontaneous degradation in response to a specific stimulus.

An antigen is an entity to which a ligand specifically binds.

The term “ligand” used herein covers monoclonal antibodies, bispecific antibodies, trispecific antibodies, PEGylated monoclonal antibodies (Chapman 2002), Fc-fusion proteins (Czajkowsky et al., 2012; Jafari et al., 2017), albumin, PEGylated albumin (Akbarzadehlaleh et al., 2016), and albumin-fusion proteins (Rogers et al., 2015; Wang et al., 2020).

The term monosaccharide as used herein covers monosaccharides and their derivatives. A monosaccharide has the chemical formula: (CH₂O)ₓ, where x≥3. A large number of monosaccharide derivatives are known. Non-limiting examples include glucosamine, sialic acid, galactosamine, ascorbic acid, mannitol, glucuronic acid, muramic acid, and Neuraminic acid.

The term polysaccharide as used herein covers a linear or branched polymer containing 1-20 molecules of monosaccharide(s).

The term PEG as used herein covers a linear or branched polymer of ethylene glycol (—C0-C10 alkylene-(CH2-CH2-O)p-CO—C10 alkylene-, wherein subscript p is an integer selected from 1-50).

Alkyl, alkylene, alkoxy, cycloalkyl, cycloalkylene, heterocyclyl, aryl, aralkyl, heterocyclylalkyl, and heteroaryl mentioned herein include both substituted and unsubstituted moieties, unless specified otherwise. Examples of a substituent include deuterium (D), hydroxyl (OH), halogen (e.g., F, Cl, and Br), amino (NH₂), oxo (═O), cyano (CN), nitro (NO₂), alkyl, alkenyl, alkynyl, alkoxy, cycloalkyl, acylamino, alkylamino, aminoalkyl, haloalkyl (e.g., trifluoromethyl), hydroxyalkyl, heterocyclyl, alkoxycarbonyl, amido, carboxy (COOH), alkanesulfonyl, alkylcarbonyl, alkenylcarbonyl, carbamido, carbamyl, carboxyl, thioureido, thiocyanato, sulfonamido, P(O)₂, P(═O)(alkyl)₂, aryl, arylamino, aralkyl, and heteroaryl. All substitutes can be further substituted.

The term “compound”, when referring to a compound of this invention, also includes its salts, solvates, and prodrugs. The pharmaceutically acceptable salts include those listed in Handbook of Pharmaceutical Salts: Properties, Selection and Use, 2^{nd} Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereometric mixture, a cis-isomer, or a trans-isomer. Compounds of such isomeric forms are within the scope of this invention. They can be present as a mixture or can be isolated using chiral synthesis or chiral separation technologies.

It is understood that compounds of the present invention may exist as stereoisomers. It is further understood that compounds of the present invention include all forms of stereoisomers including enantiomers, diastereomers, and mixtures thereof. Preferred stereoisomers are predominantly one diastereomer. More preferred stereoisomers are predominantly one enantiomer.

It is recognized that one skilled in the art may treat cancer by administering to a patient presently displaying symptoms an effective amount of a conjugate of this invention. Thus, the terms “treatment” and “treating” are intended to refer to therapeutic treatment and prophylactic measures to prevent relapse, wherein the object is to inhibit or slow down (lessen) an undesired physiological change or disorder, such as, for example, the development or spread of cancer. For purposes of this invention, beneficial or desired clinical results include, but are not limited to, alleviation of symptoms, diminishment of extent of disease, stabilized i.e., not worsening) state of disease, delay or slowing of disease progression, amelioration or palliation of the disease state, and remission (whether partial or total), whether detectable or undetectable, “Treatment” can also mean prolonging survival as compared to expected survival if not receiving treatment. Those in need of treatment include those already with the condition or disorder as well as those prone to have the condition or disorder. In the context of cancer, the term “treating” includes any or all of inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

Further, it is recognized that one skilled in the art may treat cancer by administering to a patient at risk of future symptoms an effective amount of the conjugate of this invention and is intended to include prophylactic treatment of such.

As used herein, the term “effective amount” of a compound of any one of formulas I-III refers to an amount that is a dosage, which is effective in treating a disorder, such as the diseases described herein. The attending diagnostician, as one skilled in the art, can readily determine an effective amount by the use of conventional techniques and by observing results obtained under analogous circumstances. In determining an effective amount or dose of a conjugate of this invention, a number of factors are considered, including, but not limited to the conjugate to be administered; the co-administration of other agents, if used; the species of mammal; its size, age, and general health; the degree of involvement or the severity of the disorder, such as cancer; the response of the individual patient; the mode of administration; the bioavailability characteristics of the preparation administered; the dose regimen selected; the use of other concomitant medication; and other relevant circumstances. The combinations of this invention may be administered at therapeutically effective single or divided daily doses. The active components of the combination may be administered in such doses which are therapeutically effective in monotherapy, or in such doses which are lower than the doses used in monotherapy, but when combined result in a desired (joint) therapeutically effective amount.

A conjugate of this invention may be administered alone or in the form of a pharmaceutical composition with pharmaceutically acceptable carriers, diluents or excipients. Such pharmaceutical compositions and processes for making the same are known in the art (See, e.g., Remington: The Science and Practice of Pharmacy, A. Adejare, Editor, 23rd Edition, Academic Press, 2020).

Within this invention it is to be understood that the combinations, compositions, kits, methods, uses or compounds for use according to this invention may envisage the simultaneous, concurrent, sequential, successive, alternate, or separate administration of the active ingredients or components.

The administration of a conjugate of this invention and the at least one other pharmacologically active substance may take place by co-administering the active components or ingredients, such as e.g., by administering them simultaneously, concurrently, sequentially, successively, alternately, or separately, in one single or in two or more separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g., according to the agents used and the condition of the subject.

The elements of the combinations of this invention may be administered (whether dependently or independently) by methods customary to the skilled person, e.g. by oral, enteral, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

Without further elaboration, it is believed that one skilled in the art can, based on the above description, utilize the present invention to its fullest extent. The following examples are to be construed as merely illustrative and not limitative of the remainder of the disclosure in any way whatsoever.

All publications cited herein are hereby incorporated by reference in their entirety.

### BRIEF DESCRIPTION OF DRAWINGS

FIG. 1A shows comparison of tumor inhibition activity among Kadcyla®, Enhertu®, and Conjugates 6 and 20 of this invention in an animal study. Both conjugates contain DM1. Conjugate 6 has 2 glucose moieties. Conjugate 20 has 2 PEG4 moieties. They outperformed Kadcyla® and Enhertu® in inhibiting cancer cells.

FIG. 1B shows change of mouse body weight during treatment with Kadcyla®, Enhertu®, and Conjugates 6 and 20.

FIG. 2A shows comparison of tumor inhibition activity among Kadcyla®, Enhertu®, and Conjugates 6 and 3. Conjugate 6 has two HP moieties. Conjugate 3 has only one HP moiety. Conjugate 6 is more effective than Conjugate 3.

FIG. 2B shows change of mouse body weight during treatment with Kadcyla®, Enhertu®, and Conjugates 6 and 3.

FIG. 3A shows that Deruxtecan (Dxd)-containing Conjugate 32 (with 2 maltose moieties) outperformed Sacituzumab-deruxtecan (a biosimilar to Daiichi's Datroway®) in tumor inhibition activity measured in an animal study.

FIG. 3B shows change of mouse body weight during treatment with Sacituzumab-deruxtecan and Conjugate 32.

FIG. 4A shows that immune-stimulating Conjugate 21 with two HP moieties outperformed Her-T785 in tumor inhibition activity.

FIG. 4B shows change of mouse body weight during treatment with Conjugate 21 and Her-T785.

### DETAILED DESCRIPTION

The compounds of this invention have been carefully designed by including two hydrophilic polymer (HP) moieties to increase half-life, reduce toxicity, reduce immunogenicity, and improve solubility and stability of the ligand drug conjugates of this invention.

One hydrophilic polymer moiety had been incorporated into ADCs (see Greenwald et al., 2003; Fishburn 2008; Schlapschy et al., 2013; Lin et al., 2015; Lyon et al., 2015; Podust et al., 2016; Chan et al., 2017; Hu et al., 2018; Gupta et al., 2019; Hou et al., 2019; Viricel et al., 2019; Tian et al., 2021). Inclusion of a hydrophilic polymer as a component of the drug-linker complex could shield the hydrophobic payload from the exposure to the RES system during circulation, resulting in lower systemic toxicity of and longer pharmacokinetics of ADCs (Lyon et al., 2015, Anami et al., 2018, Viricel et al., 2019, Evans et al., 2022, and Watanabe et al., 2024).

In this disclosure, two hydrophilic polymers, independently selected from a polysaccharide, a PEG, and a derivative thereof, are included in the linker complex for the construction of ligand-drug conjugates with improved activity and reduced toxicity than marketed or clinical-staged ADCs. Further, ADCs with two HPs outperformed its counterpart with only one HP in animal studies. The presence of two hydrophilic polymers in the ligand-drug conjugates not only increases the DAR of the resultant conjugates but also greatly reduces enzymatic destruction and/or cellular clearance of the conjugates in the blood circulatory system. A ligand-drug conjugate containing two hydrophilic polymers shows reduction in toxicity as well as has longer PK and is more efficacious than a conventional LDC.

Accordingly, the linker-drug compounds of the invention each contains two hydrophilic polymers and one or two drug moieties. Referring to formulas I-III above, HP1 and HP2 each are in a parallel orientation with respect to the drug unit. The linker-drug compounds are suitable for preparing ligand-drug conjugates, each of which contains a ligand covalently bound to such a drug-linker. The HP1 and HP2 moieties of the conjugates surprisingly improve pharmacokinetics, bioavailability, and pharmacodynamics as compared to conventional LDCs.

A covalent bond is a hydrazone moiety, an oxime moiety, a carbonate moiety (—O—C(O)—O—), an azobenzene moiety, an amide bond, an arylsulfate bond, a glycoside bond, a beta-glucuronide bond, a beta-galactoside bond, an ester bond, a phosphate bond, a pyrophosphate bond, an ether bond, a thioether bond, a disulfide bond, a C—S bond, a C—O bond, a C—N bond, a C—C bond, an imine bond, a carbamate moiety (—O—C(O)—NH—), a urea moiety (—NH—C(O)—NH—), a 1,2,4-trioxolane (TRX) moiety, a triazole moiety

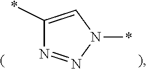 [SMILES: *c1cn(*)nn1]

a moiety derived from strain-promoted azide-alkyne cycloaddition (SPAAC) (Agard, N. J 2006; Lahann 2009), a moiety derived from strain-promoted alkyne-nitrone cycloaddition (SPANC) (MacKenzie et al., 2014), and a moiety derived from the trans-cyclooctene (TCO)-tetrazine click reaction (Liu et al., 2013).

A cleavable bond is a covalent bond that is acid-cleavable (e.g., hydrazone moiety, oxime moiety or carbonate moiety), redox-cleavable (e.g., disulfide bond), hypoxia-cleavable (e.g., an azobenzene moiety) or enzyme-cleavable (e.g., amide bond, glycoside bond, beta-glucuronide bond, beta-galactoside bond, arylsulfate bond, ester bond, phosphate bond, pyrophosphate bond, carbamate moiety, or urea moiety).

A non-cleavable bond, in contrast to a cleavable one, refers to a covalent bond that is not cleavable by acid, redox, hypoxia or enzymes. Non-limiting examples of a non-cleavable bond include C—S bond, C—O bond, C—N bond, C—C bond, ether bond, thioether bond, triazole moiety, moiety derived from strain-promoted azide-alkyne cycloaddition (SPAAC), moiety derived from strain-promoted alkyne-nitrone cycloaddition (SPANC), and a moiety derived from the trans-cyclooctene (TCO)-tetrazine click reaction.

Referring to formulas I-III above, LL contains an amino (NH₂), iodo (I), bromo (Br), maleimide, N-hydroxysuccinimide (NHS), or an aminooxy moiety.

Each of the multifunctional complex, BC, MN and MN1, when present, contains one or more monomeric units, respectively, derived from a natural amino acid, a non-natural amino acid, a monosaccharide, a bi- or multi-functional crosslinker, a polyamine, a polyalcohol, a polycarboxylic acid, a hydroxycarboxylic acid, a diglycolic acid, a glycolic acid, a lactic acid, an iminodiacetic acid, an aminoaldehyde, an aminoalcohol, an aminoketo, a mercaptocarboxylic acid, an aminothiol, penicillamine, 2-(4-aminophenyl)-2-hydroxyacetic acid, pyrrolidine-2,4-dicarboxylic acid, 1-Aminocyclopentane-trans-1,3-dicarboxylic acid, 3,6-bis-(4-aminobutyl)-piperazine-2,5-dione (CDK) and its derivatives, a cyclic peptide, or a diamino dicarboxylic acid. Of note, CDK is a cyclic dipeptide (i.e., cyclic di-lysine).

Each of MN and MN1 is covalently bonded to LL (when BC is a bond), BC, RU1, RU2, RU3, L1 (when RU1 is a bond), L2 (when RU2 is a bond), L3 (when RU3 is a bond), HP1 (when each of RU3 and L3 is a bond), HP2 (when each of RU3 and L3 is a bond), D1 (when each of RU1 and L1 is a bond), and D2 (when each of RU2 and L2 is a bond). Each of the monomeric unit within the corresponding MN and MN1 complex is independently and covalently ligated with each other via a covalent bond so that each of the MN and MN1 complex is a linear oligomer, a branched oligomer (when the number of monomer of the interested complex is equal to or greater than 4), a cyclic oligomer, or a mosaic oligomer which is a cyclic oligomer with one or more ring members having one or more substituents, which can be a monomeric multifunctional compound, a linear oligomer of multifunctional compounds, a branched oligomer of multifunctional compounds, a cyclic oligomer of multifunctional compounds or a mosaic oligomer of multifunctional compounds. A monomeric unit of the MN and MN1 complex exists in residual form (also referred to herein as assembled form).

Ligand Y bonds to the LL by an amide bond (via the exposed lysine or glutamine residue of the ligand), a C—S bond (via either the exposed cysteine residue of the ligand or the sulfhydryl group derived from the modification of non-cysteine residue of the ligand), hydrazone bond or an oxime bond (via the oxidized glycan moiety of the ligand).

A drug moiety can be a polar or conventional drug as a biologically active drug moiety. A polar drug (PD) is prepared by covalently connecting a conventional drug (D) directly or indirectly via a bifunctional linker, to Pc which is a polar, hydrophilic or ionic moiety.

Pc is a moiety derived from a natural amino acid, a non-natural amino acid, a monosaccharide, a polyamine, a polyalcohol, a polycarboxylic acid, a hydroxycarboxylic acid, a glycolic acid, a lactic acid, a diglycolic acid, an iminodiacetic acid, an aminoalcohol, an aminoaldehyde, an aminoketo, a mercaptocarboxylic acid, an aminothiol, a bi- or multi-functional crosslinker, 2-(4-aminophenyl)-2-hydroxyacetic acid, pyrrolidine-2,4-dicarboxylic acid, 1-aminocyclopentane-trans-1,3-dicarboxylic acid, 3,6-bis-(4-aminobutyl)-piperazine-2,5-dione, a cyclic peptide, or a diamino dicarboxylic acid.

Each of the releasable units RU1, RU2, and RU3 with or without a self-immolative spacer, contains either an acid-sensible trigger (e.g., hydrazone, oxime moiety or carbonate moiety), a redox sensible trigger (e.g., disulfide bond), a hypoxia-sensitive trigger (e.g., an azobenzene moiety) or an enzyme-cleavable trigger (e.g., amide bond, glycoside bond, pyrophosphate bond, arylsulfate bond or ester bond).

A single amino acid, an enzyme-cleavable peptide of 2-8 amino acids, a combination of the self-immolative spacer and the enzyme-cleavable amino acid, or a combination of the self-immolative spacer and the enzyme-cleavable peptide are frequently exploited as enzyme-cleavable triggers.

The term “self-immolative spacer” as used herein refers to a chemical moiety having a first acid-, redox-, hypoxia- or enzyme-sensible trigger linking to a releasable unit and a second trigger linking to a hydrophilic polymer moiety or a drug moiety, in which both the first and second triggers are capable of spontaneous degradation in response to a specific stimulus.

Each of D1 and D2 is a drug moiety or its polar drug derivative. Non-limiting examples include mertansine (DM1), ravtansine (DM4), N-methyl-L-Ala-maytansinol, monomethyl auristatin E (MMAE), 7-ethyl-10-hydroxycamptothecin (SN38), PROTAC chemical ARV-771, a TLR7/8 agonist (Ag), an NOD1 agonist, or an NOD2 agonist.

HP can be a pharmaceutically inert moiety (that is, a simple hydrophilic polymer) or a pharmaceutically active moiety (that is, a targeting or druggable hydrophilic polymer). The former includes PEG, glucose, lactose, maltose and the like. The latter includes targeting HPs, such as PEGylated RGD Peptide (Repenko et al., 2018), PEGylated Angiopep-2 (Mei et al., 2014), PEGylated folic acid (Sun et al., 2006), mono- or disaccharides (such as glucose, galactose, mannose, N-acetylneuramic acid, L-fucose, sialic acid, 2-amino-2-deoxy-D-glucose, 2-fluro-2-deoxyglucose, 2,5-anhydro-D-mannitol, Bleomycin disaccharide and the like) (Yu et al., 2013; Yuan et al., 2018) as well as druggable HPs including (1) hydrophilic immune adjuvants of pattern recognition receptors (PRRs) (such as: poly(I:C) (CAS number: 31852-29-6), CpG oligonucleotide, muramyl dipeptide (CAS number: 53678-77-6), Laminarin (CAS Number: 9008-22-4), Nigericin (CAS number: 28643-80-3)); (2) cancer-associated glycoantigens (such as, Globo H (CAS number: 260363-35-7), SSEA-3, SSEA-4) and (3) a conventional drug conjugated with a PEG polymer (such as CL2A-SN-38 (CAS 1279680-68-0), Azide-PEG8-Val-Cit-PABC-Exatecan (Broadpharm catalog number BP-41639), Azide-PEG8-Val-Cit-Doxorubicin (Broadpharm catalog number BP-29761), 7-O-(Amino-PEG4)-paclitaxel (Broadpharm catalog number BP-23995), Azide-PEG8-Val-Cit-PAB-MMAF (Broadpharm catalog number BP-41955), NHS ester-PEG12-Val-Cit-PAB-MMAE (Broadpharm catalog number BP-41288)). Notably, Bleomycin, a disaccharide (the Bleomycin disaccharide)-containing anticancer drug, is a payload with intrinsic both hydrophilic and targeting activities contributed by its own disaccharide. The incorporation of a pharmaceutically active hydrophilic polymer into the drug linker complex can create a ligand-drug conjugate with a variety of distinct activities.

A functional group refers to either an inert group or a reactive group.

An inert group refers to any chemical non-reactive group. Non-limiting examples of inert groups includes: C₁-C₁₀ alkyl, C₁-C₁₀ alkylene, aryl, arylene, C₃-C₈ heterocycle, C₃-C₈ heterocyclo, C₃-C₈ carbocycle, C₃-C₈ carbocyclo, C₁-C₁₀ heteroalkyl, and C₁-C₁₀ heteroalkylene. Said inert group may also be selected from the list of reactive groups.

A reactive group refers to any chemical moiety that is reactive for covalently binding a bindable group. Non-limiting examples of reactive groups include carboxylic acid, amine, aminooxy, hydroxyl, halogen, activated ester such as N-hydroxysuccinimide ester and alkynyl, alkenyl, azide, isocyanate, aldehyde, keto, maleimide, pyridyl disulfide, and thiol.

A bifunctional or multifunctional crosslinker is a PEG-containing or non-PEG-containing chemical reagent that has two or more reactive groups at its ends. These reagents can be used to crosslink two or more molecules together using suitable reactions. Bifunctional or multifunctional crosslinkers include homobifunctional crosslinkers, heterobifunctional crosslinkers, and photoreactive crosslinkers. Homobifunctional (such as glutaraldehyde) crosslinkers are crosslinking agents that have the same functional chemistry at each end of the structure. Heterobifunctional crosslinkers, (such as SMCC (N-succinimidyl 4-(maleimidomethyl)cyclohexane-1-carboxylate) and Sulfo-SMCC) are crosslinking agents that have different reactive groups at each end of the structure. Photoreactive crosslinkers (such as ABH (p-azidobenzoyl hydrazide)) have photoactivatable reactive groups that only become reactive when exposed to UV or visible light. Examples of reactive groups found in a crosslinker include, but are not limited to, amine for conjugation with the carboxylate group of target molecule; NHS-Ester for the amine group of target molecule; maleimide for the thiol group of target molecule; isocyanate for the hydroxyl group of target molecule; alkyne (—C—CH) for the azide group (—N₃) of target molecule; amino, hydrazine or aminooxy for the aldehyde or keto group of target molecule, respectively.

As used herein, the depiction of an asterisk (*) in a chemical formula represents the point of attachment of the group to the corresponding parent formula.

Without further elaboration, it is believed that one skilled in the art can, based on the above description, utilize the present invention to its fullest extent. The following examples are to be construed as merely illustrative and not limitative of the remainder of the disclosure in any way whatsoever.

### EXAMPLES

### Materials and Methods

The following reagents, cell lines and animals were used in (1) preparing compounds of this invention (i.e., linker-drugs with two hydrophilic polymer units of formula I-III) and conjugates of this invention, and (2) biophysical and biochemical assays, cell-based assays and animal studies. Their suppliers are provided below, as well as their CAS registry numbers or catalog numbers (Cat No).

Sodium cyanoborohydride, Sigma-Aldrich, CAS No. 25895-60-7; Nε-(tert-Butoxy-carbonyl)-Nα-[(9H-fluoren-9-ylmethoxy)carbonyl]-L-lysine, Tokyo Chemical Industry Co., Ltd., Cas No. 71989-26-9; Tert-Butyl methyl(2-oxoethyl)carbamate (boc-Sar-aldehyde), Ambeed, CAS No. 123387-72-4; Hydroxy-PEG8-acid, BroadPharm, CAS No. 937188-60-8, lot: B106-140; t-Boc-N-amido-PEG4-NHS ester, BroadPharm, CAS No. 859230-20-9; D-maltose, Sigma-Aldrich, CAS No. 6363-53-7; Mal-PEG1 Acid, BroadPharm, CAS No. 760952-64-5; m-PEG8-aldehyde, BroadPharm, CAS No. 1234369-95-9; Fmoc-N-amido-PEG1-acid, BroadPharm, CAS No. 1654740-73-4; D-Glucuronic acid, Combi-Blocks, CAS No. 6556-12-3; TLR7/8 agonist 1 dihydrochloride (Ag), MedChemExpress, CAS No. 1620278-72-9; Na-Fmoc-NO-Boc-L-2,3-diaminopropionic acid (Fmoc-Dap(Boc)-OH), Tokyo Chemical Industry Co., Ltd., CAS No. 162558-25-0; HATU, Combi-Blocks, CAS No. 148893-10-1; N-Ethyldiiso-propylamine (DIPEA or DIEA), Alfa Aesar, CAS No. 7087-68-5; DMSO, Sigma-Aldrich; Acetonitrile (ACN), J. T. Baker; (2R,4S)-1-Fmoc-4-Boc-amino pyrrolidine-2-carboxylic acid (Fmoc-4AP(Boc)-OH), Combi-Blocks, CAS No. 1820570-42-0; HEPES, Sigma-Aldrich, CAS No. 7365-45-9; Sodium hydroxide (NaOH), Sigma-Aldrich; Trifluoroacetic acid, Sigma-Aldrich, CAS No. 76-05-1; Morpholine, Alfa Aesar; Mal-PEG2-acid, Broadpharm, CAS No. 1374666-32-6; Herceptin (trastuzumab), Roche, Cat No. 000961-1-CTN-05; Sacituzumab, MedChemExpress, HY-P99045, CAS No. 1796566-95-4; DL-Dithiothreitol (DTT), VWR LIFE SCIENCE, CAS No. 3483-12-3; Sephadex™ G25 Fine, Cytiva; Sephadex™ G-100, Cytiva; D-proline, AK Scientific, CAS No. 344-25-2; Boc-Sar-Osu, Combi-Blocks, CAS No. 80621-90-5; Triethylamine, Sigma-Aldrich, CAS No. 121-44-8; N,N-Dimethylformamide (DMF), Sigma-Aldrich, CAS No. 68-12-2; Sucrose, VWR LIFE SCIENCE, CAS No. 57-50-1; Sodium succinate dibasic hexahydrate, Sigma-Aldrich, CAS No. 6106-21-4; Tween-20, VWR LIFE SCIENCE, CAS No. 9005-64-5; Mertansine (DM1), Cayman, CAS: 139504-50-0; Kadcyla, Roche, NDC 50242-088-01; Enhertu, Daiichi-Sankyo AstraZeneca, NDC 65597-406-01; L-Cysteine hydrochloride monohydrate, Sigma, CAS: 7048-04-6; (S)-3-Amino-2-(tert-butoxycarbonylamino)propionic Acid (Boc-Dap-OH), Tokyo Chemical Industry Co., Ltd., CAS: 73259-81-1; N^{α}-(tert-Butoxycarbonyl)-L-lysine (Boc-Lys-OH), Tokyo Chemical Industry Co., Ltd., CAS: 13734-28-6; Serine, Tokyo Chemical Industry Co., Ltd., CAS: 56-45-1; N-Succinimidyl Maleimidoacetate (AMAS), Tokyo Chemical Industry Co., Ltd., CAS: 55750-61-3; Tris(2-carboxyethyl)phosphine (TCEP), GoldBio, CAS: 51805-45-9; 1-(4-Aminobutyl)-2-butyl-1H-imidazo[4,5-c]quinolin-4-amine (T785), Amadis Chemical, CAS No. 313350-31-1; 96-well plate, Nunc, Thermo Fisher, Cat No 167008; Sodium Chloride, Sigma, Cat No S3014; Potassium Chloride, Amresco, Cat No 395; Sodium Phosphate Dibasic Anhydrius, Amresco, Cat No 404; Potassium phosphate monobasic, Sigma, Cat No 795488; Ethylenediaminetetraacetic acid disodium salt dehydrate, Sigma, Cat No E4884; Citric acid, Merck, Cat No 77929; D-(+)-Glucose anhydrous for biochemistry merck CAS: 50-99-7; Maltotriose AK Scientific J93117-25 g CAS: 1109-28-0; H-Ala-OH Ambeed A318333-100 g CAS: 56-41-7; N-(2-Aminoethyl)maleimide Hydrochloride TCI A2436 CAS: 134272-64-3; Fmoc-Gly-Osu combi-block SS0336 CAS: 113484-74-5; Fmoc-Gly-OH, combi-block QA-0010 CAS:29022-11-5; H—N-Me-Ser-OH hydrochloride, combi-block SS-2845, CAS: 141193-65-9; L-Serine combi-block QA-6398 CAS: 56-45-1; D-Serine combi-block OR-0196 CAS: 312-84-5; Fmoc-Ser-OH Ambeed A114508-25 g CAS: 73724-45-5; m-PEG4-aldehyde BROADPHARM BP-21580 CAS: 197513-96-5; t-Boc-N-amido-PEG1-acid BROADPHARM BP-20910 CAS: 1260092-44-1; Mal-PEG1-acid Broadpharm BP-21859 CAS:760952-64-5; Mal-PEG2-amine TFA salt, 99% Broadpharm BP-23313 CAS: 660843-22-1; Formaldehyde solution sigma F8775-500ML CAS: 50-00-0; Fmoc-PEG4-NHS ester Broadpharm BP-20644 CAS: 1314378-14-7; t-Boc-N-amido-PEG4-amine Broadpharm BP-22602 CAS: 811442-84-9; Hydroxy-PEG8-acid, Broadpharm BP-21739 CAS: 937188-60-8; NH2-C2-NH-Boc, ambered, A144595-25 g CAS:57260-73-8; (9H-Fluoren-9-yl)methyl (2-aminoethyl)carbamate hydrobromide (Fmoc-ethylenediamine) Ambeed A1352827 CAS: 352351-55-4; SIA Crosslinker, Broadpharm, BP-22653, CAS: 39028-27-8; Iodoacetic acid, sigma, CAS: 64-69-7; Fmoc-Val-Cit-OH, Broadpharm, BP-23387, CAS:159858-21-6; 2-Iodoethyl ether combi-block OR-3705 CAS: 34270-90-1; Heat-inactivated fetal bovine serum (FBS), GIBCO, Cat No 10437-028; RPMI-1640, GIBCO, Cat No 31800-022; McCoy's 5A, Sigma, Cat No M4892; Cell line NCI-N87, and SK-BR-3 purchased from ATCC; JIMT-1 purchased from Deutsche Sammlung von Mikroorganismen und Zellkulturen (DSMZ, Germany); Boc-ethylenediamine (NH2-C2-NH-Boc), Ambeed, A144595, CAS: 57260-73-8; t-Boc-N-amido-PEG1-amine, Broadpharm, BP-22862, CAS: 127828-22-2; Fmoc-D-Dap(Boc)-OH, Combi-block, SS-2341, CAS:198544-42-2; Fmoc-L-Dap-OH, AK Scientific, cat: Z4318-1 g, CAS No. 181954-34-7; t-Boc-N-amido-PEG1-NHS ester, BroadPharm, CAS No. 1260092-55-4; N-(tert-Butoxycarbonyl)-L-alanine (boc-Ala-OH), Tokyo Chemical Industry Co., Ltd., Cas No. 15761-38-3; Boc-L-propargylglycine (Boc-PGly-OH), Combi-Blocks, CAS 63039-48-5; 3-azidopropionic acid, BroadPharm, CAS No. 18523-47-2; Cupric sulfate pentahydrate (CuSO4.5H2O), Sigma-Aldrich, CAS 7758-99-8; Tris(3-hydroxypropyltriazolylmethyl)amine (THPTA), Sigma-Aldrich, CAS No. 760952-88-3; Sodium L-ascorbate, Tokyo Chemical Industry Co., Ltd., Cas No. 134-03-2; Boc-L-cysteine, Combi-Blocks, CAS No. 20887-95-0; CL2A-SN38, BroadPharm, CAS No. 1279680-68-0; Muramyl dipeptide (MDP), MedChemExpress, CAS No. 53678-77-6; (3-Aminopropyl)-hydrazine, Combi-Blocks, CAS No. 18169-30-7; rel-(1R,2R)-2-(tert-Butoxycarbonyl)cyclo-propane-1-carboxylic acid (CPCA-Otbu), Ambeed, CAS No. 2307773-07-3; Amino-PEG2-t-butyl ester, Broadpharm, BP-20555, CAS No. 756525-95-8; 3-Iodopropan-1-ol, AK Scientific, 9477AB, CAS No. 627-32-7; D-Glucuronic acid sodium salt monohydrate, AK Scientific, X3578, CAS No. 207300-70-7; Dxd, MedChemExpress, HY-13631D, CAS No. 1599440-33-1; Deruxtecan, MedChemExpress, HY-13631E-50 MG, CAS No. 1599440-13-7; ARV-771, A2B Chem LLC, AX63908, CAS No. 1949837-12-0; Boc-trans-AMCP (Trans-cyclopropane-carboxylic acid, 2-[[[(1,1-dimethylethoxy)carbonyl]amino]methyl]-), Combi-block, QV-6335, CAS No. 952708-48-4; Boc-ACPC (1-[(tert-Butoxycarbonyl)amino]cyclopropanecarboxylic acid), Combi-block, SS-0708, CAS No. 88950-64-5; Fmoc-ACPC (1-(Fmoc-amino)cyclo-propanecarboxylic acid), AK Scientific, X3157, CAS No. 126705-22-4; Fmoc-Osu, AK Scientific, M704, CAS No. 82911-69-1; L-trans-Pyrrolidine-2,4-dicarboxylic Acid, Cayman, 24268, CAS No. 64769-66-0; Boc-D-Pro-OH, Combi-blocks, QA-0080, CAS No. 37784-17-1; rel-(1R,2R)-2-(tert-Butoxycarbonyl)cyclopropane-1-carboxylic acid, Ambeed, A996882, CAS No. 2307773-07-3; and (9H-Fluoren-9-yl)methyl (2-(2-aminoethoxy)ethyl)carbamate HCL (Fmoc-N-amido-PEG1-amine), Ambeed, A510063, CAS No. 221352-88-1. Her2/ERBB2 Protein, Human, Recombinant (ECD), Cat. No. 10004-HCCH, Sino Biological. Goat F(ab′)2 Anti-Human IgG Fc (HRP), Cat. No. ab98530, Abcam. 3,3′,5,5′-Tetramethylbenzidine (TMB), Cat. No. 860336-5G, Sigma-Aldrich. BSA, Fraction V, Cat. No. 30063-721, Gibco. Tween 20, Cat. No. 0777-4L, Amresco.

JIMT-1 was maintained in DMEM supplemented with 10% heat-inactivated fetal bovine serum (FBS). NCI-N87 was maintained in RPMI-1640 supplemented with 10% heat-inactivated FBS. SK-BR-3 was maintained in McCoy's 5A supplemented with 10% heat-inactivated FBS. All cell lines were cultured at 37° C. under 5% CO2 atmosphere.

BALB/c mice, and SCID Beige mice were obtained from BioLASCO Co., Ltd (Taiwan). T-DM1 (Trastuzumab emtansine) as a comparative sample was purchased from Shanghai Union Dispensary Co. (Taipei, Taiwan). All animal studies were conducted in accordance with the Animal Care and Use Committee in a facility accredited by the association for Assessment and Accreditation of laboratory Animal Care.

Compounds as prepared were separated and analyzed by chromatography techniques such as high-performance liquid chromatography (HPLC) and mass spectrometer (LC/MS). Compound characterization was performed using LC/MS such as Agilent 6545 Q-TOF LC/MS equipped with an EclipsePlusC18 RRHD column.

Several procedures were followed to prepare exemplary compounds and conjugates of this invention as described below.

### Protocol for Removal of Boc Protection Group (Boc Protocol)

The tert-butoxycarbonyl (Boc) protection group of a compound was removed by incubation with 70% trifluoroacetic acid (TFA) at room temperature for 1 hour (yields about 90-99%). The desired product was purified by HPLC (Agilent® 1260 Infinity® II with ACE C18-300 column), followed by lyophilization with Manifold Lyophilizer (−80° C., 10 mTorr).

### Protocol for Removal of tbu Protection Group (tbu Protocol)

The tert-butyl (tbu) protection group of a compound was removed by incubation with 90% trifluoroacetic acid (TFA) at room temperature for 1 hour (yields about 90-99%). The desired product was purified by HPLC (Agilent® 1260 Infinity® II with ACE C18-300 column), followed by lyophilization with Manifold Lyophilizer (−80° C., 10 mTorr).

### Protocol for Removal of Fmoc Protection Group (Fmoc Protocol)

The fluorenylmethyloxycarbonyl (Fmoc) protection group of a compound was removed by incubation with 12.5% morpholine at room temperature for 20 min (yields about 90-99%). The desired product was purified by HPLC (Agilent® 1260 Infinity® II with ACE® C18-300 column), followed by lyophilization with Manifold Lyophilizer (−80° C., 10 mTorr).

### Protocol for Amide Bond Formation Using NHS Reagent (NHS Protocol)

N-Hydroxysuccinimide (NHS, 65 mM) was mixed with a solution containing an amine (130 mM), 80% dimethylsulfoxide (DMSO), and a 20% 50 mM HEPES buffer (50 mM HEPES, 100 mM NaCl, and 1 mM ethylenediaminetetraacetic acid, i.e., EDTA) at pH 8-9 with agitation at room temperature for 2 hours (yields about 60-95%). The desired product was purified by HPLC, followed by lyophilization with Manifold Lyophilizer (−80° C., 10 mTorr).

### Protocol for Amide Bond Formation Using HATU Reagent (HATU Protocol)

An amine (51.8 mM) was stirred with a carboxylic acid (104 mM), HATU (207 mM), and DIEA (518 mM) in DMF at room temperature for 1 hour (yields about 40-90%). The desired product was purified by HPLC (Agilent® 1260 Infinity® II with ACE® C18-300 column), followed by lyophilization with Manifold Lyophilizer (−80° C., 10 mTorr).

### Protocol for C—N Bond Formation (C—N Bond Protocol)

A primary or secondary amine (50 mM) was stirred with an aldehyde (250 mM) and Sodium cyanoborohydride (200 mM) in DMSO/MEOH (1:1) at 40° C. to 60° C. for 2 hours to 24 hours (60-95% yield). The desired product was purified by HPLC (Agilent® 1260 Infinity® II with ACE® C18-300 column), followed by lyophilization with Manifold Lyophilizer (−80° C., 10 mTorr).

Protocol for C—S bond formation using cesium carbonate reagent (C—S bond protocol)

A thiol (6.82 mM) was stirred with an iodo compound (20.5 mM), tris(2-carboxyethyl)-phosphine (TCEP, 3.41 mM) and cesium carbonate (54.6 mM) in a solution containing 60% DMSO and a 40% 50 mM potassium phosphate buffer (30.8 mM K₂PO₄, 19.2 mM KH2PO4, 100 mM NaCl, and 1 mM EDTA; pH 7) at room temperature for 2 hours (yields about 50-90%). The desired product was purified by HPLC (Agilent® 1260 Infinity II with ACE C18-300 column), followed by lyophilization with Manifold Lyophilizer (−80° C., 10 mTorr).

### Protocol for Maleimide-Thiol Reaction (Maleimide-Thiol Protocol)

Maleimide (35.4 mM) was stirred with a thiol (29.5 mM) in a solution containing 80% DMSO and 20% 50 mM HEPES buffer (pH 6.5) at room temperature for 1 hour (yields about 90-99%). The desired product was purified by HPLC (Agilent® 1260 Infinity® II with ACE® C18-300 column), followed by lyophilization with Manifold Lyophilizer (−80° C., 10 mTorr).

### Protocol for Crosslinking an Amine-Containing Compound and a Thiol-Containing Compound Using SIA Reagent (SIA Protocol)

SIA Cross linker (30 mM) was mixed with a thiol (10 mM), an amine (40 mM) and TCEP (40 mM) in a solution containing 80% DMSO and a 20% 50 mM HEPES buffer (50 mM HEPES, 100 mM NaCl and 1 mM EDTA, pH 8.5) at room temperature for 1 hours (yields about 50-70%). The desired product was purified by HPLC (Agilent® 1260 Infinity II with ACE C18-300 column), followed by lyophilization with Manifold Lyophilizer (−80° C., 10 mTorr).

### Protocol for Disulfide Bond Formation (SPDB Protocol)

An SPDB cross linker was mixed with a thiol in a solution containing 75% DMSO and a 25% 50 mM HEPES buffer (50 mM HEPES, 100 mM NaCl and 1 mM EDTA, pH 8.0) at room temperature for 1 hours. The desired product was purified by HPLC (Agilent® 1260 Infinity II with ACE C18-300 column), followed by lyophilization with Manifold Lyophilizer (−80° C., 10 mTorr).

### Protocol for Oxime Bond Formation (Oxime Protocol)

A ketone was mixed with an aminooxy in a 10 mM sodium phosphate buffer (pH 7.4) containing 3M NaCl at room temperature for 90 min. The desired product was purified by HPLC (Agilent® 1260 Infinity II with ACE C18-300 column), followed by lyophilization with Manifold Lyophilizer (−80° C., 10 mTorr).

### Protocol for Carbamate Bond Formation (Carbamate Protocol)

Step1: The hydroxyl group reacted with 10 equivalents of carbonyldiimidazole (CDI) in DMSO at 40° C. for 1 hour. Then, the intermediate compound R—O-CDI was purified by HPLC (Agilent® 1260 Infinity II with ACE C18-300 column), followed by lyophilization with Manifold Lyophilizer (−80° C., 10 mTorr).

Step2: R—O—CDI reacted with 4 equivalent amino compound and 10 equivalents triethylamine (TEA) in DMSO containing at 40° C. for at least 1 hour. The carbamate product was purified by HPLC (Agilent® 1260 Infinity II with ACE C18-300 column), followed by lyophilization with Manifold Lyophilizer (−80° C., 10 mTorr).

### Protocol for the Carbonate Bond Formation (Carbonate Protocol)

The hydroxyl compound reacted with N,N′-Disuccinimidyl Carbonate (DSC) in DMF or Acetonitrile containing 10 equiv DMAP at room temperature for 2 hours. The carbonate was purified by HPLC (Agilent® 1260 Infinity II with ACE C18-300 column), followed by lyophilization with Manifold Lyophilizer (−80° C., 10 mTorr).

### Protocol for the Ester Bond Formation (Ester Protocol)

The acid compound and hydroxyl compound were mixed with N,N′-Diiso-propylcarbodiimide (DIC) and DMAP in DMF or DMSO at room temperature for at least 1 hour. The ester was purified by HPLC (Agilent® 1260 Infinity II with ACE C18-300 column), followed by lyophilization with Manifold Lyophilizer (−80° C., 10 mTorr).

### Protocol for the Hydrazone Bond Formation (Hydrazone Protocol)

A ketone compound was mixed with hydrazine in a 10 mM sodium phosphate buffer (pH 7.4) containing 3M NaCl, at room temperature for 90 min. The desired product was purified by HPLC (Agilent® 1260 Infinity II with ACE C18-300 column), followed by lyophilization with Manifold Lyophilizer (−80° C., 10 mTorr).

### Protocol for Triazole Formation (Click Protocol)

An alkyne (50 mM) was stirred with an azido (75 mM), a premixed CuSO₄·5H₂O/THPTA (125 mM/150 mM in H2O) and sodium L-ascorbate (200 mM) in DMSO at 35° C. for 2 hours-4 hours (yields about 60-90%). The desired product was purified by HPLC (Agilent® 1260 Infinity® II with ACE® C18-300 column), followed by lyophilization with Manifold Lyophilizer (−80° C., 10 mTorr).

### Preparation and Characterization of Antibody-Drug Conjugates

IgG1 antibody (Ab) molecules (10 mg) were reduced in 0.5 mL of a buffer containing α,α-trehalose dihydrate (9.09) mg, L-histidine HCl (0.225 mg), L-histidine (0.145 mg), polysorbate 20 (0.04 mg), and DTT (0.83 mg). After all accessible disulfides of the antibody were reduced, the buffer was changed to a 50 mM HEPES buffer (50 mM HEPES at pH 8 with 100 mM NaCl and 1 mM EDTA) using G-25 Gel Filtration Columns to remove excess DTT (yields about 80-90%). A Linker-Drug (5-10 molar eq. relative to that of the antibody) was dissolved in DMSO and added to the antibody solution prepared above thereby obtaining a solution containing 2 mg/mL of Ab, 70% 50 mM HEPES buffer (50 mM HEPES pH 7.4, 100 mM NaCl and 1 mM EDTA) and 30% DMSO. The conjugation reaction was performed at room temperature for 1-2 hours. The buffer was subsequently replaced by a solution containing 6% sucrose, 10 mM sodium succinate dibasic hexahydrate, and 0.02% Tween 20 at pH 5 through G-100 Gel Filtration Columns. The protein concentration in the resulting conjugate solution was determined by the BCA protein assay using Herceptin as a standard (yields about 80-90%). The drug/antibody ratio (DAR) was determined with HPLC. Briefly, the weight of the antibody was determined by a fluorescence detector (FLD; excitation at 274 nm and emission at 310 nm), while the weight of the drug was determined by a specific UV absorbance wavelength e.g., 254 nm for DM1. The FLD and UV absorbance of a control (i.e., an antibody solution) was used as a reference. Whole-antibody mass spectrometry provides a second methodology for assessing drug loading homogeneity and DAR of 2×HPsADCs. 2×HPsADCs aggregates were analyzed using a size exclusion column (SEC). Purified 2×HPsADCs were lyophilized and stored at −20° C. until use. Bolt's immune-stimulating ADC, Her-T785, as a comparative sample was prepared as described (Ackerman et al., 2021).

### Preparation of Mouse Serum

Blood was collected from Balb/c mice by submandibular bleeding. Collected blood was allowed to clot at room temperature for 30 minutes, then followed by centrifugation at 15000×g for 20 minutes two times at 4° C. Carefully transferred the supernatant (serum) to a new tube for used immediately.

### Protocol for the Serum Stability Test of Linker-Drugs

A linker-drug stock solution was added into 30 ul of Balb/c mouse serum to reach a final concentration of 1 mM linker-drug in serum, then the mixture was incubated at room temperature. 0.5 ul sample were withdrew from the mixture at the indicated time points for analysis by HPLC (Agilent® 1260 Infinity® II with ACE 5 phenyl-300 column). The serum half-life of each linker-drug compound was calculated by GraphPad Prism 7 program.

In vitro cytotoxicity assays of ADCs In vitro potency was assessed on multiple cancer cell lines: SK-BR3, NCI-N87, JIMT-1, and canpan-1 cells in log-phase growth were seeded for 24 hours in 384 well plates containing 20-μL medium supplemented with 10% FBS. Serial dilutions of ADCs in cell culture media were prepared at 2× working concentrations, and 20 μL of each dilution was added to the 384-well plates. Following addition of APCs, cells were incubated for 6 days at 37° C. Cytotoxicity to tumor cells was assessed by ImageXpress® Micro Imaging system (Molecular Devices). The EC₅₀ value, determined in quadruplicate, is defined as the concentration that achieves half-maximal growth inhibition.

### Pharmacokinetics (PK) Study

### Solutions

PBS: 137 mM NaCl, 2 mM KCl, 10 mM Na₂HPO₄ and 1.8 mM KH₂PO₄.
PBST: 1×PBS, 0.05% Tween 20.
Coating solution: 0.05 M carbonate/bicarbonate buffer, pH 9.6.
Blocking solution: 1×PBS, 1% BSA and 0.05% Tween 20.
Phosphate citrate solution: 100 mM sodium phosphate, pH 5, and 50 mM citrate acid.
TMB solution: 1 mg/ml TMB and 0.0066% H₂O2 in phosphate citrate solution.

### Sample Preparation

Aliquots of an ADC (8 mg/mL in a buffer containing 6% sucrose, 10 mM sodium succinate dibasic hexahydrate and 0.02% Tween 20, pH 5.0) were prepared for intravenous infusion into female Sprague-Dawley rats (6-8 weeks old; LASCO, Taiwan) at a dose of 10 mg/kg. Blood samples were collected at 30 min, 1 hour, 3 hours, 6 hours, 1 day, 3 days, and 7 days post-dosing. Collected blood was allowed to clot at room temperature for 30 minutes, then followed by centrifugation (15000×g) two times at 4° C. for 20 minutes. The supernatant (serum) was transferred to a new tube and stored at −80° C. until analysis.

### ELISA Assay

Recombinant HER2 ECD (at a concentration of 0.25 μg/mL of coating solution and 100 μL per well) was used to coat 96 well-plates at 4° C. overnight. Remove the coating solution and wash the plates with 300 μL PBST three times. Then, the plates were blocked using 300 μL blocking solution at room temperature for 1 hour. Discard the blocking solution and wash the plates with 300 ul PBST three times. The ADC standard curve was established for a concentration from 0.0977 ng/mL to 12.5 ng/mL as follows. Each 100 μL ADC sample of pre-determined concentration was introduced into the HER2 ECD-coated plate and incubated at room temperature for 2 hours. The solutions were discarded. The plate was washed with 300 μL of PBST three times, followed by the addition of 100 μL goat F(ab′)₂ anti-human IgG Fc-HRP (1:10,000 dilution in PBST) per well and incubation at room temperature for 2 hours. The goat antibody solution was removed. The plate was washed with 300 μL of PBST three times. A TMB solution (100 μL) was added to each well at room temperature for 10 minutes. The reaction was stopped by adding 50 μL of 10% H₂SO₄. The OD_{450 nm} absorbance was obtained using a spectrophotometer (Infinite® M200 Pro, TECAN®). The OD_{450 nm} absorbance of blood samples was obtained followed the above protocol for the ADC standard. The PK data was calculated by GraphPad Prism 7 program and PK solver 2.0 using the ADC standard curve as a reference.

### Xenograft Tumor Model Experimental Procedure for Cytotoxic ADCs

Tumor cell lines were purchased from AddexBio (JIMT-1) and grown according to manufacturer's instructions. Cells were harvested when they reached 80-90% confluency by treatment with Trypsin/EDTA solution (Gibco), washed with PBS. 5×10⁶ cells were resuspended in 50 uL culture media and mixed with 50 ul Matrigel Basement Membrane Matrix High Concentration. The mixture was placed on ice for no longer than two hours. 100 μL of the mixture (5×10⁶ cells) were implanted subcutaneously into the right flank of 6-8-week-old female BALB/c Nude (BioLASCO, Taiwan). Tumor size was recorded and estimated using the following formula: (length×width²)/2. Once xenografts reached ˜200 mm³, animals were treated with ADCs. Mice were randomized by tumor size into treatment groups. Cytotoxic ADCs were prepared at a concentration of 2 mg/mL in a buffer (6% sucrose, 10 mM sodium succinate dibasic hexahydrate and 0.02% Tween 20 pH 5.0) and administered at 10 mg/kg (for ADCs of this invention, Kadcyla and Enhertu) by intravenous injection. Mice whose tumors exceeded 2000 mm³ were euthanized as per the IACUC-approved animal protocols.

### Xenograft Tumor Model Experimental Procedure for Immuno-Stimulating ADCs

Tumor cell lines were purchased from ATCC (NCI-N87) and grown according to manufacturer's instructions. Cells were harvested when they reached 80-90% confluency by treatment with Trypsin/EDTA solution (Gibco), washed with PBS. 5×10⁶ cells were resuspended in 50 uL culture media and mixed with 50 ul Matrigel Basement Membrane Matrix High Concentration. The mixture was placed on ice for no longer than two hours. 100 μL of the mixture (5×10⁶ cells) were implanted subcutaneously into the right flank of 6-8-week-old SCID/Beige mice. Mice were treated by intravenous (IV) injection of LDCs.

The synthesis of control compounds 1-5 and exemplary compounds 6-46 of the present invention is shown below.

Control Compound 1 (Boc-D-pro-PEG1-CPCA-(C═O)—O-Dxd) was prepared according to the following protocol:
(i) The ester bond formation between Dxd and CPCA-tbu (rel-(1R,2R)-2-(tert-Butoxycarbonyl)cyclopropane-1-carboxylic acid) followed the ester bond protocol.
(ii) Removal of the tbu protection group of tbu-CPCA-(C═O)—O-Dxd followed the tbu protocol.
(iii) The amide bond formation between t-Boc-N-amido-PEG1-amine and CPCA-(C═O)—O-Dxd followed the HATU protocol.
(iv) Removal of the Boc protection group of t-Boc-N-amido-PEG1-CPCA-(C═O)—O-Dxd followed the Boc protocol.
(v) The amide bond formation between Boc-D-Proline and NH2-PEG1-CPCA-(C═O)—O-Dxd followed the HATU protocol.

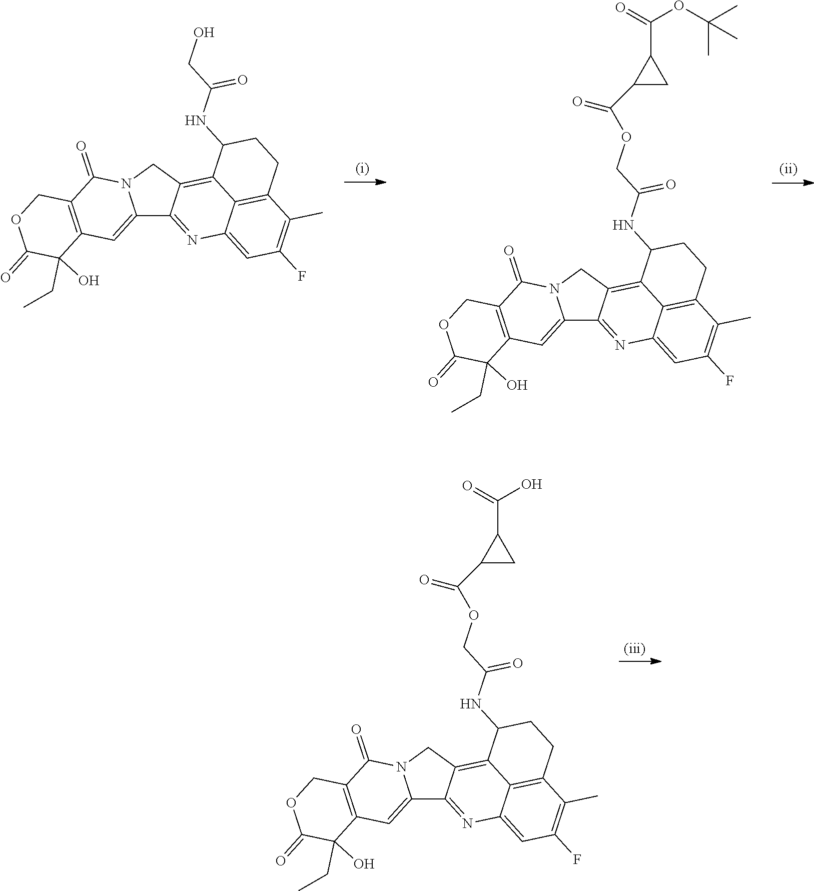 [SMILES: CCC1(O)C(=O)OCc2c1cc1n(c2=O)Cc2c-1nc1cc(F)c(C)c3c1c2C(NC(=O)CO)CC3 ; CCC1(O)C(=O)OCc2c1cc1n(c2=O)Cc2c-1nc1cc(F)c(C)c3c1c2C(NC(=O)COC(=O)C1CC1C(=O)OC(C)(C)C)CC3 ; CCC1(O)C(=O)OCc2c1cc1n(c2=O)Cc2c-1nc1cc(F)c(C)c3c1c2C(NC(=O)COC(=O)C1CC1C(=O)O)CC3]

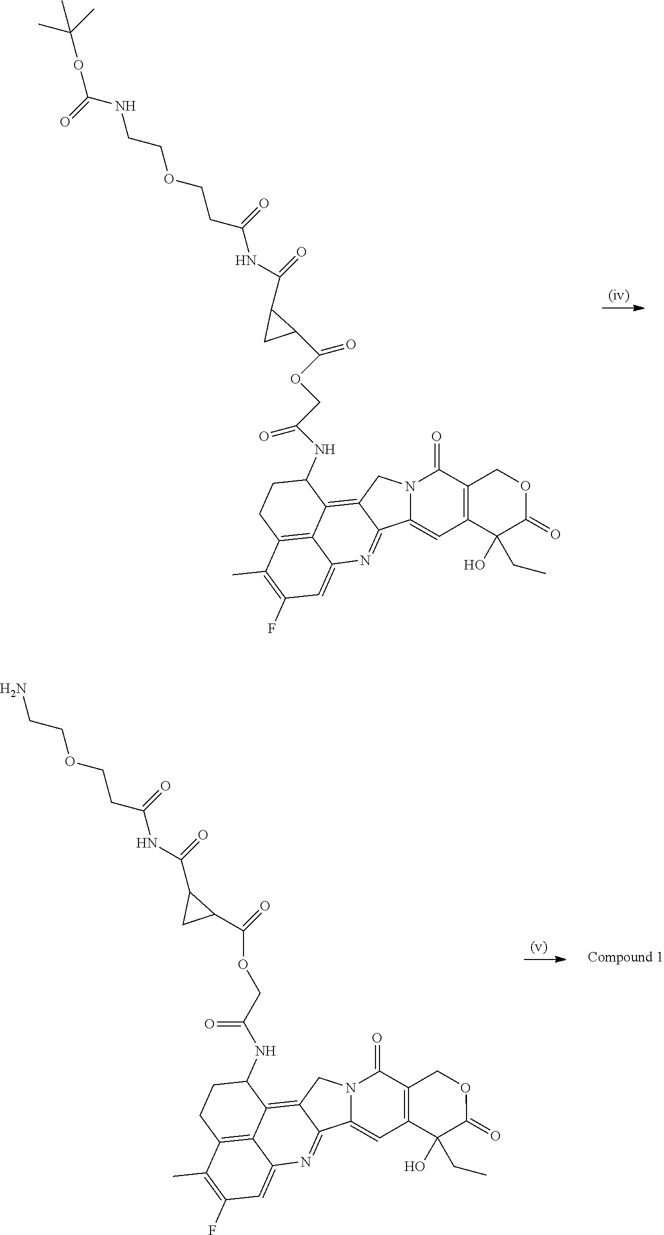 [SMILES: CCC1(O)C(=O)OCc2c1cc1n(c2=O)Cc2c-1nc1cc(F)c(C)c3c1c2C(NC(=O)COC(=O)C1CC1C(=O)NC(=O)CCOCCNC(=O)OC(C)(C)C)CC3 ; CCC1(O)C(=O)OCc2c1cc1n(c2=O)Cc2c-1nc1cc(F)c(C)c3c1c2C(NC(=O)COC(=O)C1CC1C(=O)NC(=O)CCOCCN)CC3]

Control Compound 2 (DM1-AMAS-L-Ser-L-Lys(N(maltotriose)(CH3))-PEG2-Mal) was prepared according to the following protocol:

Part A.
(i) Amide bond formation between L-Serine and N-Succinimidyl Maleimidoacetate (AMAS) using the NHS protocol
(ii) Maleimide-S bond formation between AMAS-L-Ser-OH and DM1 using the maleimide-thiol protocol.

Part B
(i) The C—N bond formation between Boc-Lys(NH2)-OH and maltotriose followed the C—N bond protocol.
(ii) The C—N bond formation between Boc-Lys(NH(maltotriose))-OH and formaldehyde followed the C—N bond protocol.
(iii) The amide bond formation between Boc-Lys(N(maltotriose)(CH3))-OH and mal-PEG2-amine followed the HATU protocol.
(iv) Removal of the Boc protection group of Boc-Lys(N(maltotriose)(CH3))-PEG2-mal followed the Boc protocol.
(v) The amide bond formation between NH2-Lys(N(maltotriose)(CH3))-PEG2-mal and DM1-AMAS-L-Ser followed the HATU protocol.

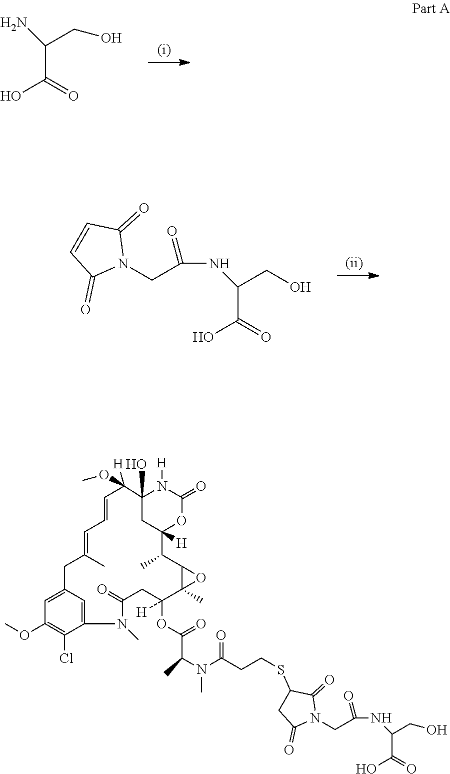 [SMILES: NC(CO)C(=O)O ; O=C(CN1C(=O)C=CC1=O)NC(CO)C(=O)O ; COc1cc2cc(c1Cl)N(C)C(=O)C[C@H](OC(=O)[C@H](C)N(C)C(=O)CCSC1CC(=O)N(CC(=O)NC(CO)C(=O)O)C1=O)[C@]1(C)OC1[C@H](C)[C@@H]1C[C@@](O)(NC(=O)O1)[C@H](OC)/C=C/C=C(\C)C2]

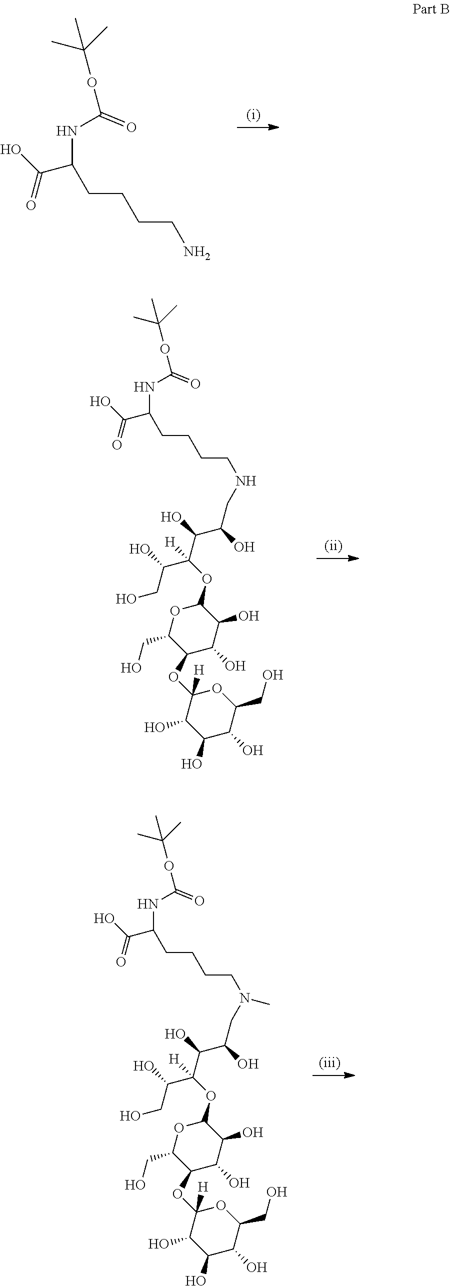

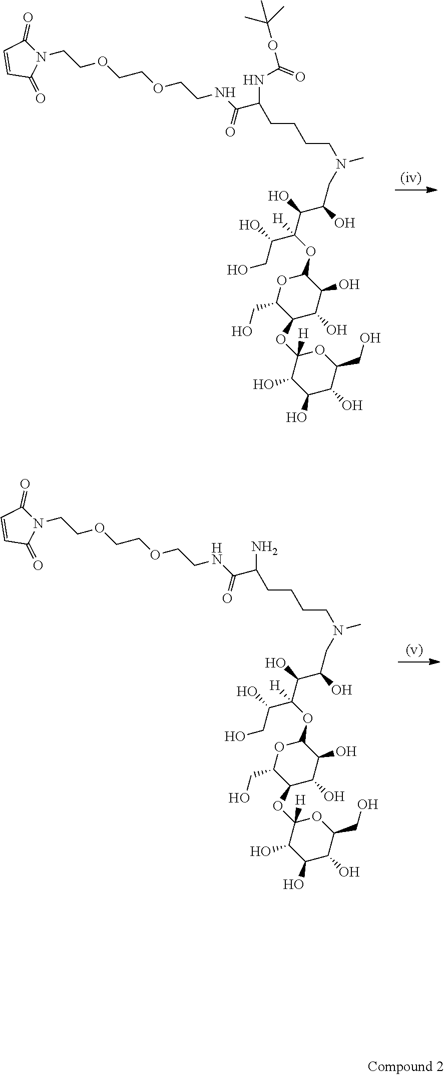

Control Compound 3 (DM1-AMAS-L-Ser-L-Lys(N(D-maltose)(CH3))-PEG2-Mal) was prepared according to the following protocol:

Part A. See the Part A protocol for compound 2 above.

Part B.
(i) The C—N bond formation between Boc-L-Lys(NH2)-OH and D-maltose followed the C—N protocol.
(ii) The C—N bond formation between Boc-L-Lys(NH(D-maltose))-OH and formaldehyde followed the C—N protocol.
(iii) The amide bond formation between Boc-L-Lys(N(D-maltose)(CH3))-OH and Mal-PEG2-amine followed the HATU protocol.
(iv) Removal of the Boc protection group (de-BOC) of Boc-L-Lys(N(D-maltose)(CH3))-PEG2-Mal followed the Boc protocol.
(v) The amide bond formation between NH2-L-Lys(N(D-maltose)(CH3))-PEG2-Mal and DM1-AMAS-L-Ser followed the HATU protocol.

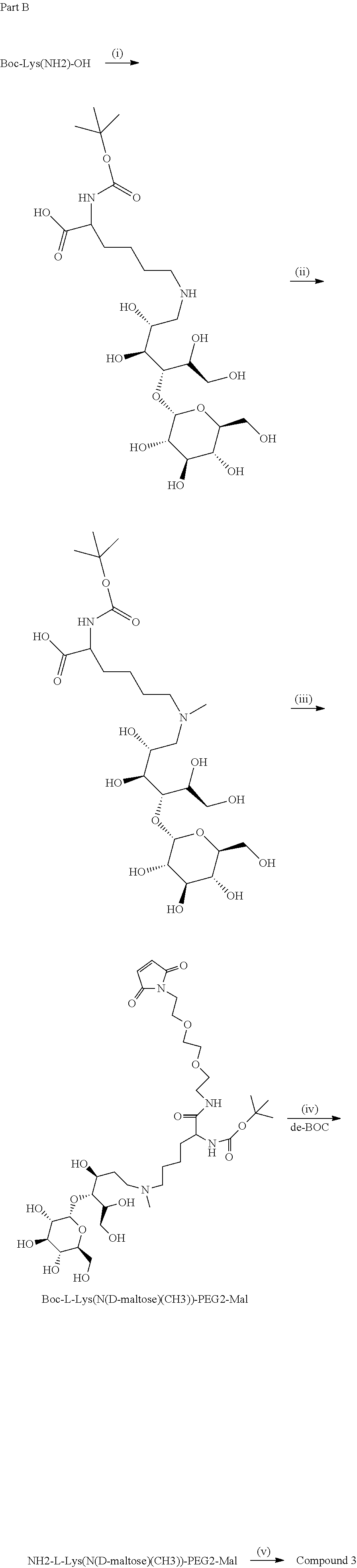

Control Compound 4 (Mal-PEG1-4AP(N(Glucose)(CH3))-PEG1-L-Ser-AMAS-DM1) was prepared according to the following protocol:

Part A. See the Part A protocol for compound 2 above.

Part B.
(i) Removal of the Boc protection group (de-BOC) of Fmoc-4AP(Boc)-OH followed the Boc protocol.
(ii) The C—N bond formation between Fmoc-4AP(NH2)-OH and glucose followed the C—N bond protocol.
(iii) The C—N bond formation (N-Me) between Fmoc-4AP(N(glucose))-OH and formaldehyde followed the C—N bond protocol.
(iv) The amide bond formation between Fmoc-4AP(N(glucose)(CH3))-OH and t-Boc-N-amido-PEG1-amine followed the HATU protocol.
(v) Removal of the Boc protection group of Fmoc-4AP(N(glucose)(CH3))-PEG1-Boc followed the Boc protocol.
(vi) The amide bond formation between Fmoc-4AP(N(glucose)(CH3))-PEG1-NH2 and DM1-AMAS-L-Ser followed the HATU protocol.
(vii) Removal of the Fmoc protection group (de-Fmoc) of Fmoc-4AP(N(glucose)(CH3))-PEG1-Ser-AMAS-DM1 followed the Fmoc protocol.
(viii) The amide bond formation between NH2-4AP(N(glucose)(CH3))-PEG1-Ser-AMAS-DM1 and Mal-PEG1-acid followed the HATU protocol.

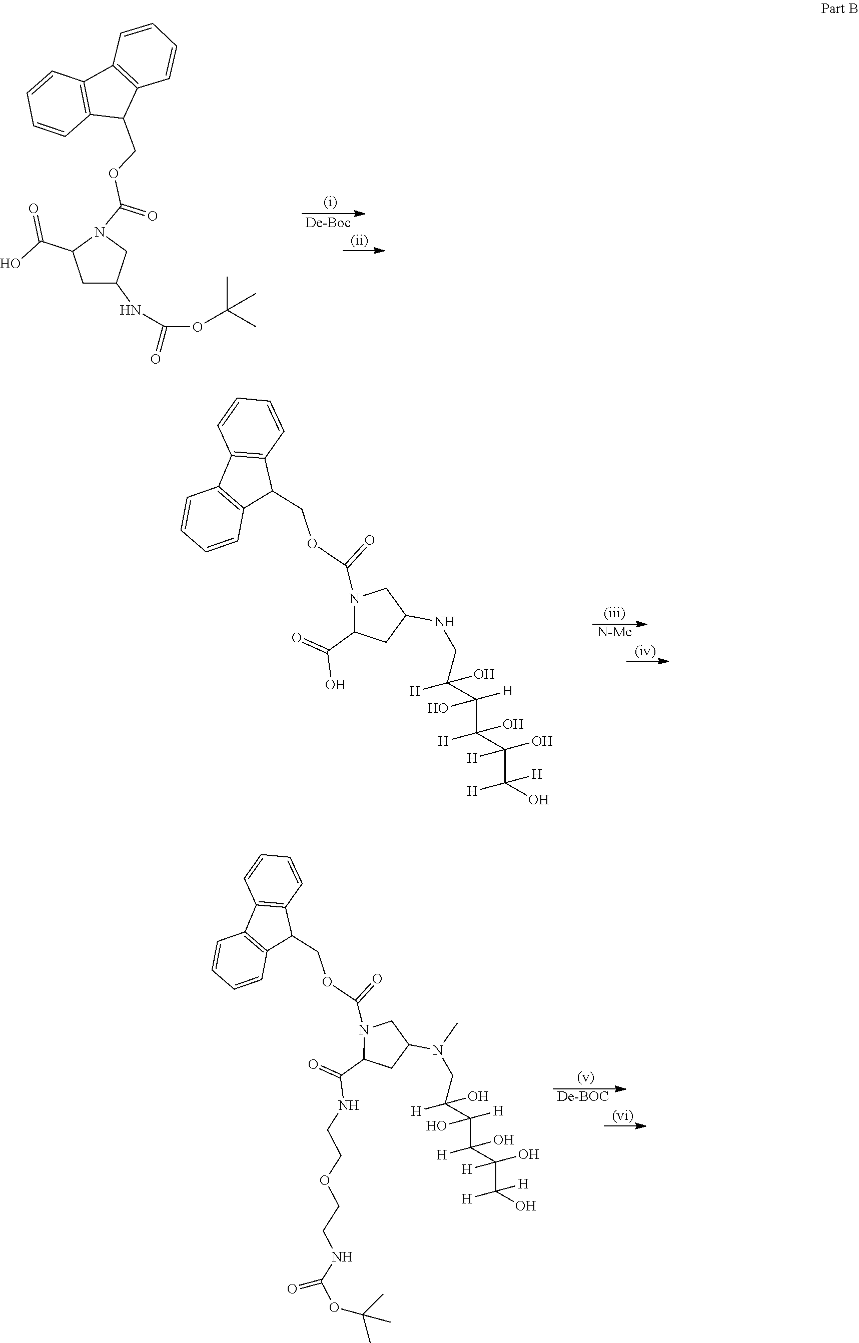 [SMILES: CC(C)(C)OC(=O)NC1CC(C(=O)O)N(C(=O)OCC2c3ccccc3-c3ccccc32)C1 ; "De-Boc" ; O=C(O)C1CC(NCC(O)C(O)C(O)C(O)CO)CN1C(=O)OCC1c2ccccc2-c2ccccc21 ; CN(CC(O)C(O)C(O)C(O)CO)C1CC(C(=O)NCCOCCNC(=O)OC(C)(C)C)N(C(=O)OCC2c3ccccc3-c3ccccc32)C1 ; "De-BOC" ; "N-Me"]

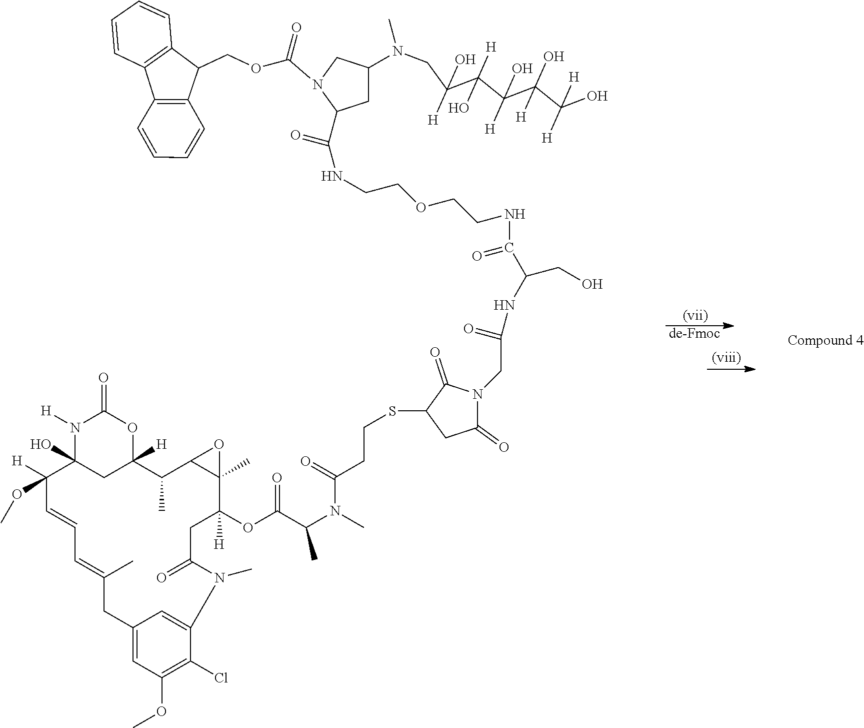 [SMILES: COc1cc2cc(c1Cl)N(C)C(=O)C[C@H](OC(=O)[C@H](C)N(C)C(=O)CCSC1CC(=O)N(CC(=O)NC(CO)C(=O)NCCOCCNC(=O)C3CC(N(C)CC(O)C(O)C(O)C(O)CO)CN3C(=O)OCC3c4ccccc4-c4ccccc43)C1=O)[C@]1(C)OC1[C@H](C)[C@@H]1C[C@@](O)(NC(=O)O1)[C@H](OC)/C=C/C=C(\C)C2 ; "de-Fmoc"]

Control Compound 5 (Mal-PEG1-4AP(GlcMe)-PEG1-VCt-BAMe-Ag) was prepared according to the following protocol:

Part A
(i) The C—N bond formation between tert-Butyl 4-formylbenzylcarbamate and Ag followed the C—N bond protocol.
(ii) The methylation (N-Me) of boc-BA-Ag followed the C—N bond protocol.
(iii) Removal of the Boc (de-BOC) protection group of boc-BAMe-Ag followed the Boc protocol.

Part B
(i) The amide bond formation between of Fmoc-PEG1-COOH and Val-Cit-OH followed the HATU protocol.
(ii) The amide bond formation between of Fmoc-PEG1-Val-Cit-OH and BAMe-Ag followed the HATU protocol.
(iii) Removal of the Fmoc protection group of Fmoc-PEG1-Val-Cit-BAMe-Ag followed the Fmoc protocol.

Part C
(i) The removal of the Boc protection group of Fmoc-4AP(boc)-OH followed the Boc protocol.
(ii) The C—N bond formation between Fmoc-4AP(NH2)-OH and glucose followed the C—N bond protocol.
(iii) The methylation of Fmoc-4AP(Glc)-OH followed the C—N bond protocol.
(iv) The amide bond formation between Fmoc-4AP(GlcMe)-OH and NH2-PEG1-Val-Cit-BAMe-Ag followed the HATU protocol.
(v) Removal of the Fmoc protection group of Fmoc-4AP(GlcMe)-PEG1-Val-Cit-BAMe-Ag followed the Fmoc protocol.
(vi) The amide bond formation between NH2-4AP(GlcMe)-PEG1-Val-Cit-BAMe-Ag and Mal-PEG1-acid followed the HATU protocol.

ESI-MS m/z calculated for C68H97N14O15 [M+H]+: 1349.7; found 1349.7.

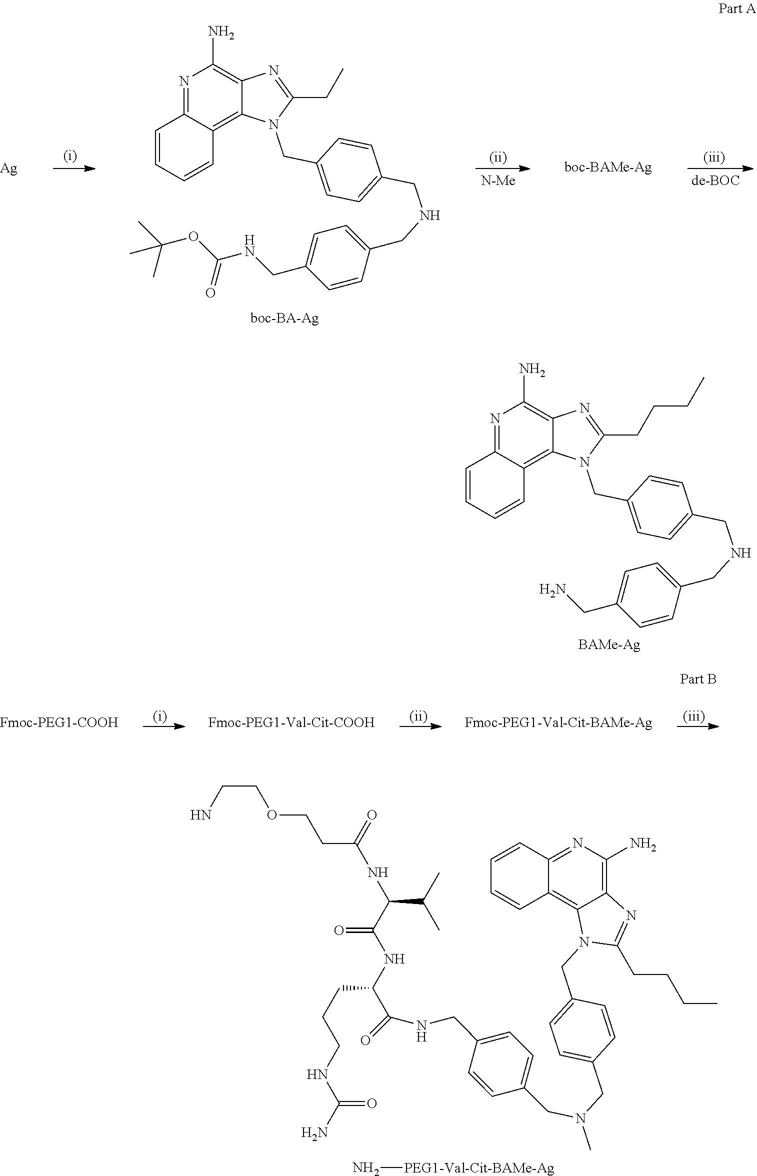

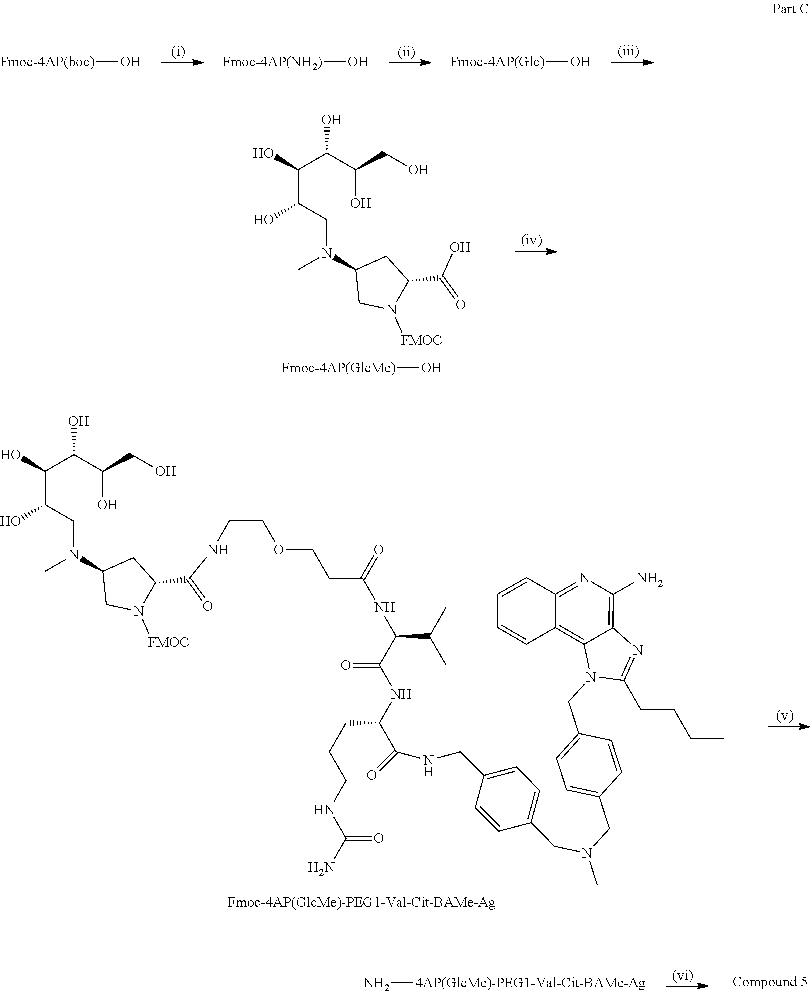

Compound 6 (DM1-AMAS-L-Ser-L-Lys(N(glucose)2)-PEG2-Mal) was prepared according to the following protocol:

Part A, see the Part A protocol for compound 2 above.

Part B.
(i) The C—N bond formation between Boc-L-Lys(NH2)-OH and glucose followed the C—N bond protocol.
(ii) The amide bond formation (Mal-PEG) between Boc-L-Lys(N(glucose)2)-OH and Mal-PEG2-amine followed the HATU protocol.
(iii) Removal of the Boc protection group (de-BOC) of Boc-L-Lys(N(glucose)2)-PEG2-Mal followed the Boc protocol.
(iv) The amide bond formation (DM1) between NH2-L-Lys(N(glucose)2)-PEG2-Mal and DM1-AMAS-L-Ser followed the HATU protocol.

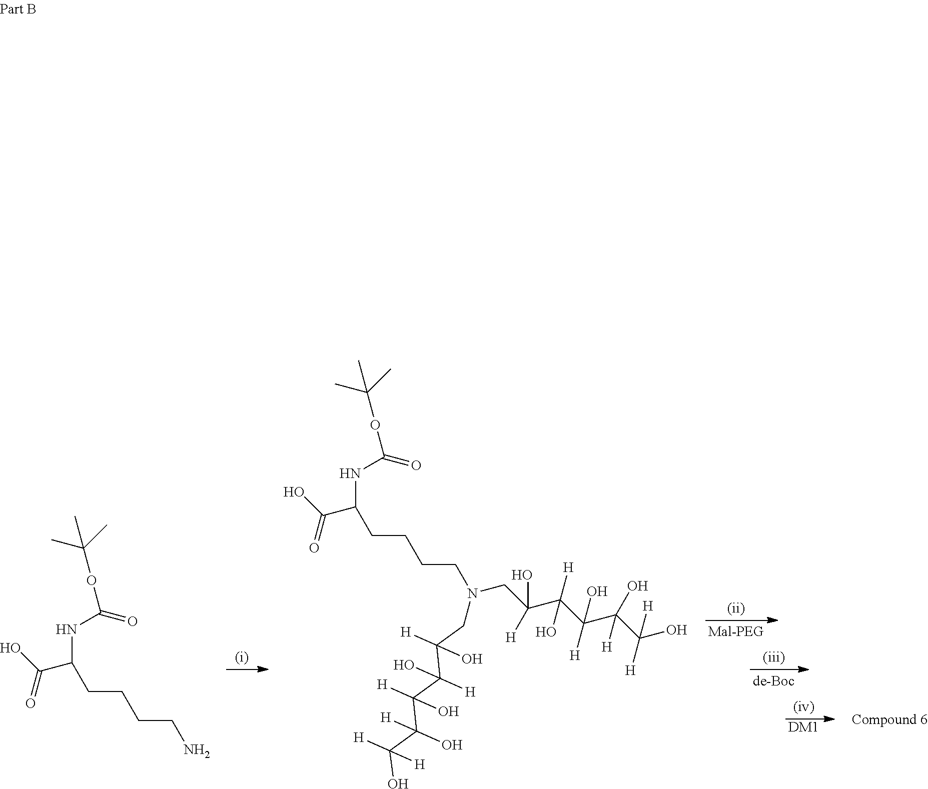

Compound 7 (DM1-AMAS-L-Ser-L-Lys(N(D-maltose)2)-PEG2-Mal) was prepared according to the following protocol:

Part A, See the Part A protocol for compound 2 above.

Part B.
(i) The C—N bond formation between Boc-L-Lys(NH2)-OH and D-maltose followed the C—N bond protocol.
(ii) The amide bond formation (Mal-PEG) between Boc-L-Lys(N(D-maltose)2)-OH and Mal-PEG2-amine followed the HATU protocol.
(iii) Removal of the Boc protection group (de-BOC) of Boc-L-Lys(N(D-maltose)2)-PEG2-Mal followed the Boc protocol.
(iv) The amide bond formation (DM1) between NH2-L-Lys(N(D-maltose)2)-PEG2-Mal and DM1-AMAS-L-Ser followed the HATU protocol.

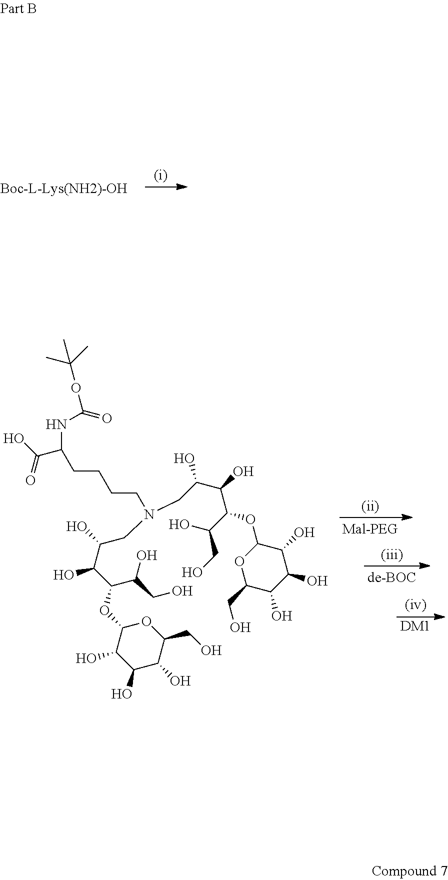

Compound 8 (DM1-AMAS-L-Ser-L-Lys(N(D-maltose)2)-(CH2)2-Mal) was prepared according to the following protocol:

Part A, see the Part A protocol for compound 2 above.

Part B
(i) See Part B, step (i) for Compound 7.
(ii) The amide bond formation (Mal-C2-NH) between Boc-L-Lys(N(D-maltose)2)-OH and Mal-(CH2)2-amine followed the HATU protocol.
(iii) Removal of the Boc protection group (de-BOC) of Boc-L-Lys(N(D-maltose)2)-(CH2)2-Mal followed the Boc protocol.
(iv) The amide bond formation (DM1) between NH2-L-Lys(N(D-maltose)2)-(CH2)2-Mal and DM1-AMAS-L-Ser followed the HATU protocol.

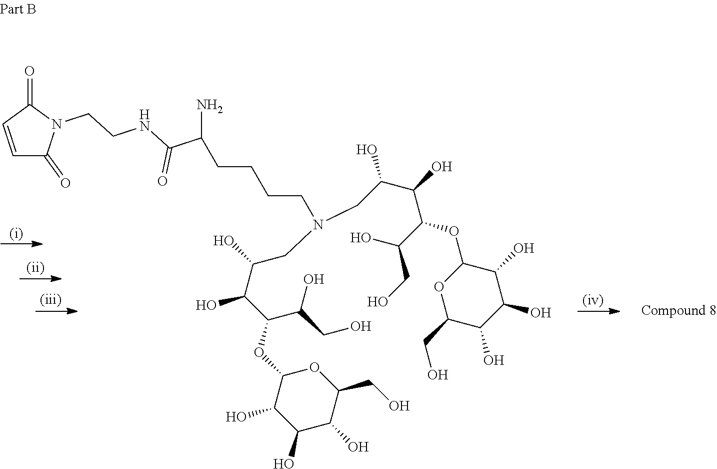

Compound 9 (DM1-AMAS-L-Ser-L-Lys(Gly-N(D-maltose)2)-(CH2)2-Mal) was prepared according to the following protocol:

Part A, see the Part A protocol for compound 2 above.

Part B
(i) The amide bond formation between Boc-L-Lys(NH2)-OH and Fmoc-Gly-Osu followed the NHS protocol.
(ii) Removal of the Fmoc protection group (de-Fmoc) of Boc-L-Lys(Gly-Fmoc)-OH followed the Fmoc protocol.
(iii) The C—N bond formation (D-maltose) between Boc-L-Lys(Gly-NH2)-OH and D-maltose followed the C—N bond protocol.
(iv) The amide bond formation (Mal-C2-NH) between Boc-L-Lys(Gly-N(D-maltose)2)-OH and Mal-(CH2)2-amine followed the HATU protocol.
(v) Removal of the Boc protection group (de-BOC) of Boc-L-Lys(Gly-N(D-maltose)2)-(CH2)2-Mal followed the Boc protocol.
(vi) The amide bond formation (DM1) between NH2-L-Lys(Gly-N(D-maltose)2)-(CH2)2-Mal and DM1-AMAS-L-Ser followed the HATU protocol.

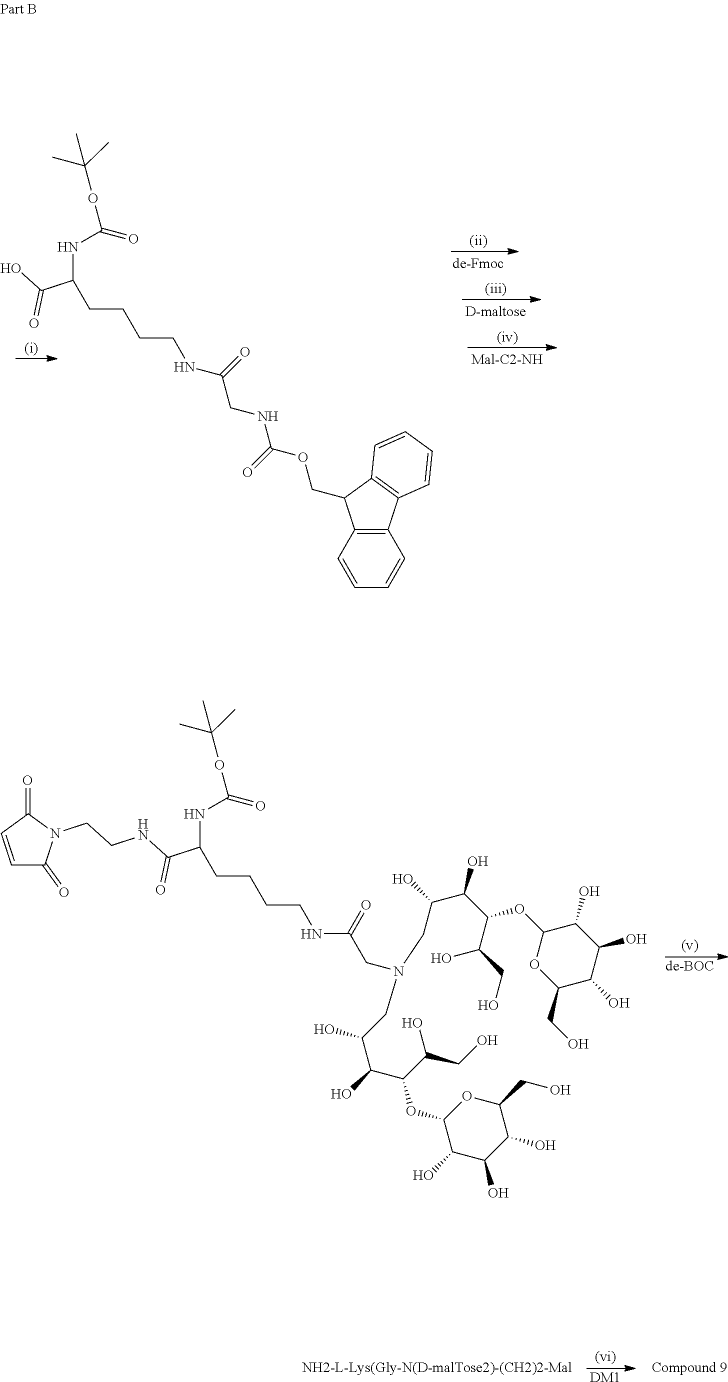

Compound 10 (DM1-AMAS-L-Ser-L-Lys(N(PEG4m)2)-(CH2)2-Mal) was prepared according to the following protocol:

Part A, see the Part A protocol for compound 2 above.

Part B
(i) The C—N bond formation (PEG4) between Boc-L-Lys(NH2)-OH and m-PEG4-aldehyde followed the C—N bond protocol.
(ii) The amide bond formation (Mal-C2-NH) between Boc-L-Lys(N(PEG4m)2)-OH and Mal-(CH2)2-amine followed the HATU protocol.
(iii) Removal of the Boc protection group (de-BOC) of Boc-L-Lys(N(PEG4m)2)-(CH2)2-Mal followed the Boc protocol.
(iv) The amide bond formation between (DM1) NH2-L-Lys(N(PEG4m)2)-(CH2)2-Mal and DM1-AMAS-L-Ser followed the HATU protocol.

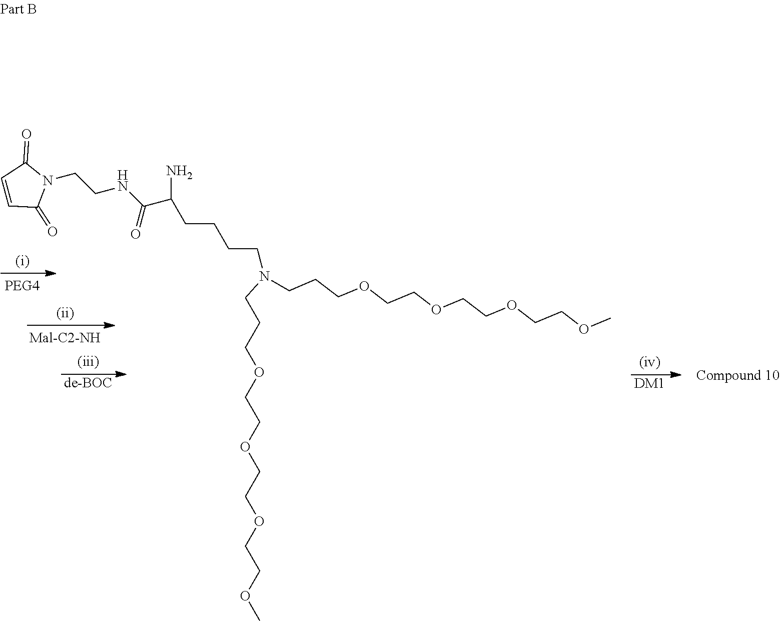

Compound 11 (DM1-AMAS-L-Ser-L-Lys(PEG4-N(D-maltose)2)-(CH2)2-Mal) was prepared according to the following protocol:

Part A, see the Part A protocol for compound 2 above.

Part B
(i) The amide bond formation (Fmoc-PEG4-NHS) between Boc-L-Lys(NH2)-OH and Fmoc-PEG4-NHS followed the NHS protocol.
(ii) Removal of the Fmoc protection group (de-Fmoc) of Boc-L-Lys(PEG4-Fmoc)-OH followed the Fmoc protocol.
(iii) The C—N bond formation (D-maltose) between Boc-L-Lys(PEG4-NH2)-OH and D-maltose followed the C—N bond protocol.
(iv) The amide bond formation (Mal-C2-NH) between Boc-L-Lys(PEG4N(D-maltose)2)-OH and Mal-(CH2)2-amine followed the HATU protocol.
(v) Removal of the Boc protection group (de-BOC) of Boc-L-Lys(PEG4N(D-maltose)2)-(CH2)2-Mal followed the Boc protocol.
(vi) The amide bond formation (DM1) between NH2-L-Lys(PEG4N(D-maltose)2)-(CH2)2-Mal and DM1-AMAS-L-Ser followed the HATU protocol.

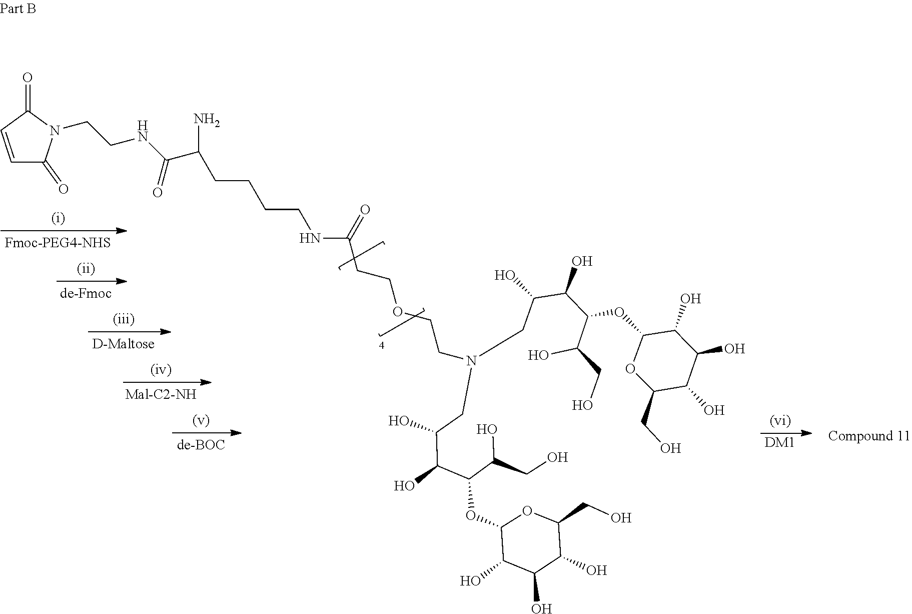

Compound 12 (DM1-AMAS-L-Ser-L-Lys(PEG4-N(D-maltose)2)-PEG2-Mal) was prepared according to the following protocol:

Part A, see the Part A protocol for compound 2 above.

Part B, following protocols similar to the Part B protocols for compound 11.
(i) The amide bond formation (Fmoc-PEG4-NHS) between Boc-L-Lys(NH2)-OH and Fmoc-PEG4-NHS followed the NHS protocol.
(ii) Removal of the Fmoc protection group (de-Fmoc) of Boc-L-Lys(PEG4-Fmoc)-OH followed the Fmoc protocol.
(iii) The C—N bond formation (D-Maltose) between Boc-L-Lys(PEG4-NH2)-OH and D-maltose followed the C—N bond protocol.
(iv) The amide bond formation (Mal-PEG2) between Boc-L-Lys(PEG4N(D-maltose)2)-OH and Mal-PEG2-amine followed the HATU protocol.
(v) Removal of the Boc protection group (de-Boc) of Boc-L-Lys(PEG4N(D-maltose)2)-PEG2-Mal followed the Boc protocol.
(vi) The amide bond formation (DM1) between NH2-L-Lys(PEG4N(D-maltose)2)-PEG2-Mal and DM1-AMAS-L-Ser followed the HATU protocol.

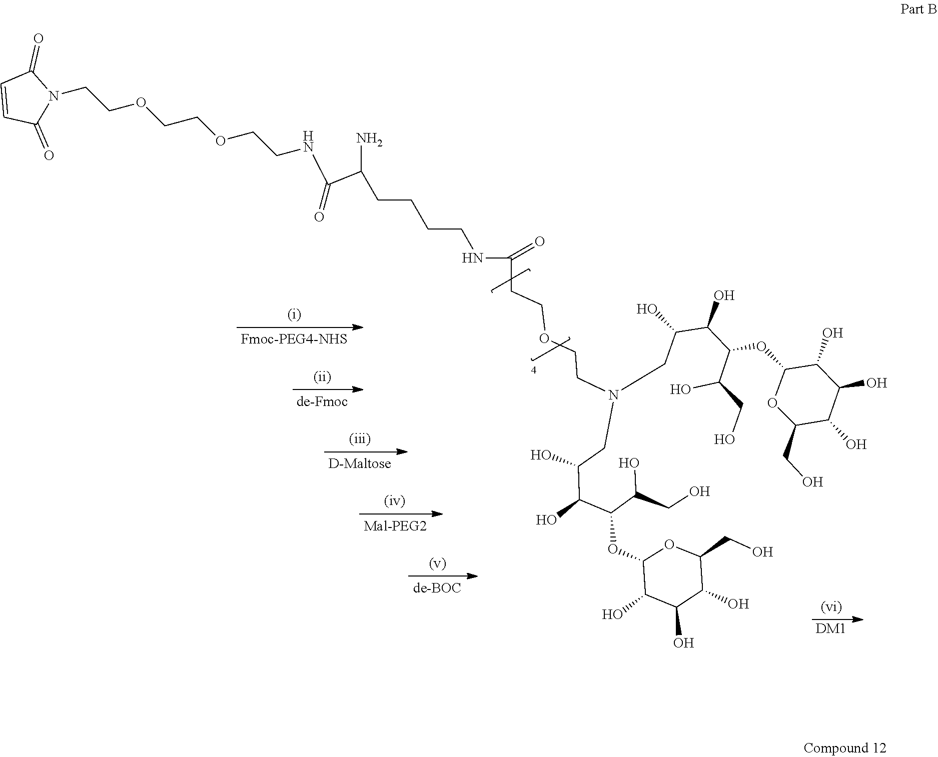

Compound 13 (DM1-AMAS-L-Ser-L-Dap(PEG4-N(D-maltose)2)-(CH2)2-Mal) was prepared according to the following protocol:

Part A, see the Part A protocol for compound 2 above.

Part B, following protocols similar to the Part B protocols for compound 11.
(i) The amide bond formation (Fmoc-PEG4-NHS) between Boc-L-Dap(NH2)-OH and Fmoc-PEG4-NHS followed the NHS protocol.
(ii) Removal of the Fmoc protection group (de-Fmoc) of Boc-L-Dap(PEG4-Fmoc)-OH followed the Fmoc protocol.
(iii) The C—N bond formation (D-maltose) between Boc-L-Dap(PEG4-NH2)-OH and D-maltose followed the C—N bond protocol.
(iv) The amide bond formation (Mal-C2-NH) between Boc-L-Dap(PEG4N(D-maltose)2)-OH and Mal-(CH2)2-amine followed the HATU protocol.
(v) Removal of the Boc protection group (de-BOC) of Boc-L-Dap(PEG4N(D-maltose)2)-(CH2)2-Mal followed the Boc protocol.
(vi) The amide bond formation (DM1) between NH2-L-Dap(PEG4N(D-maltose)2)-(CH2)2-Mal and DM1-AMAS-L-Ser followed the HATU protocol.

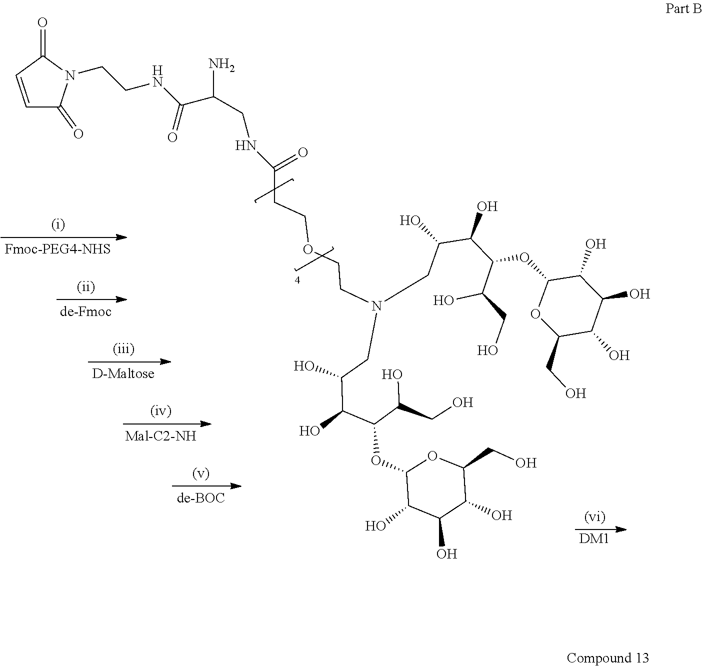

Compound 14 (Mal-PEG1-4AP(PEG1-L-Ser-AMAS-DM1)-PEG4-N(maltose)2) was prepared according to the following protocol:

Part A, see the Part A protocol for compound 2 above.

Part B
(i) The C—N bond formation (D-maltose) between Boc-PEG4-NH and D-maltose followed the C—N bond protocol.
(ii) Removal of the Boc protection group (de-BOC) of Boc-PEG4-N(D-maltose)2 followed the Boc protocol.
(iii) Removal of the Boc protection group (de-BOC) of Fmoc-4AP(Boc)-OH followed the Boc protocol.
(iv) The amide bond formation (BOC-PEG1) between Fmoc-4AP(NH2)-OH and Boc-PEG1-acid followed the HATU protocol.
(v) The amide bond formation (N-PEG4-N(D-maltose)) between Fmoc-4AP(PEG1-Boc)-OH and NH2-PEG4-N(D-maltose)2 followed the HATU protocol.
(vi) Removal of the Boc protection group (de-BOC) of Fmoc-4AP(PEG1-Boc)-PEG4-N(D-maltose)2 followed the Boc protocol.
(vii) The amide bond formation (DM1) between Fmoc-4AP(PEG1-NH2)-PEG4-N(D-maltose)2 and DM1-AMAS-L-Ser followed the HATU protocol.
(viii) Removal of the Fmoc protection group (de-Fmoc) of Fmoc-4AP(PEG1-L-Ser-AMAS-DM1)-PEG4-N(D-maltose)2 followed the Fmoc protocol.
(ix) The amide bond formation (Mal-PEG1) between NH2-4AP(PEG1-L-Ser-AMAS-DM1)-PEG4-N(D-maltose)2 and Mal-PEG1-acid followed the HATU protocol.

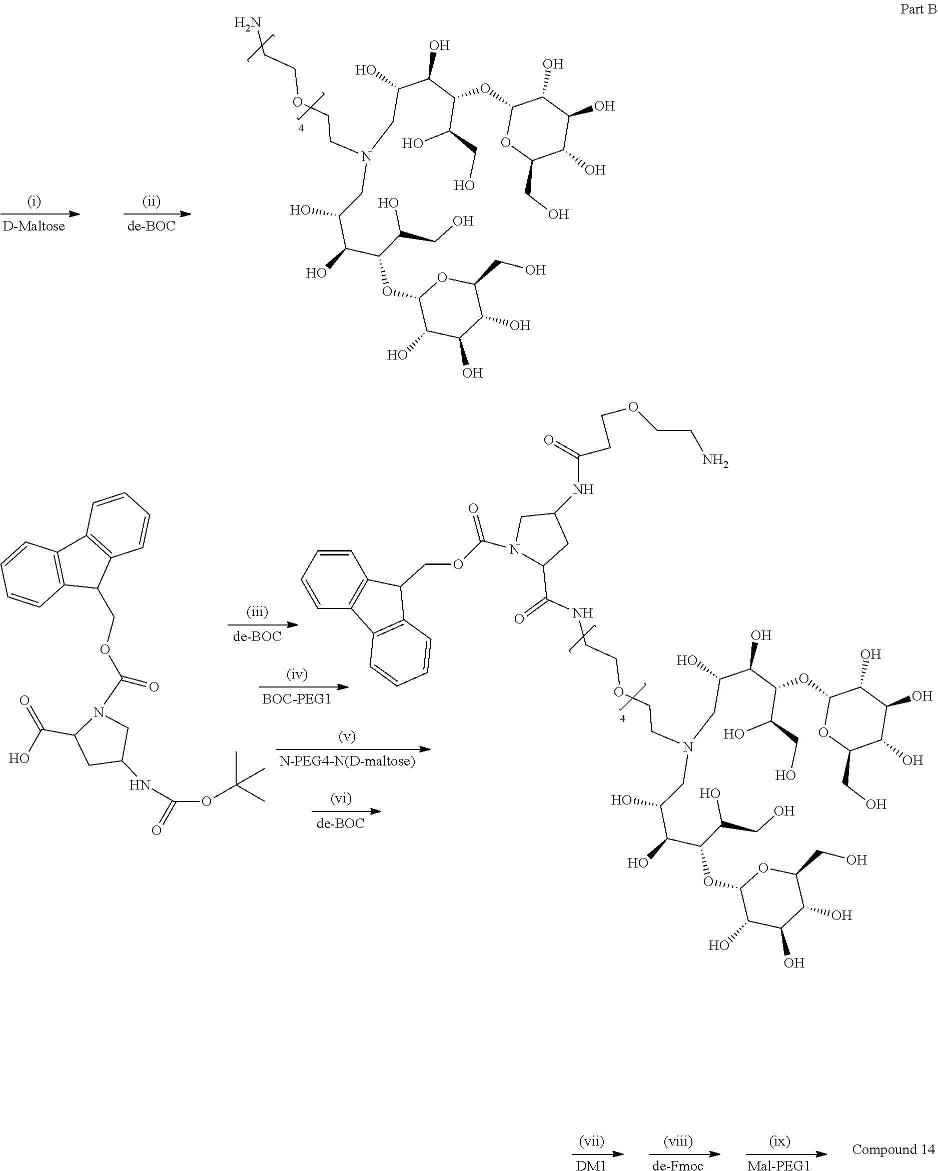

Compound 15 (DM1-AMAS-L-Ser-L-Lys(N(maltotriose)2)-PEG2-Mal) was prepared according to the following protocol:

Part A, see the Part A protocol for compound 2 above.

Part B
(i) The C—N(maltotriose) bond formation between Boc-L-Lys-OH and maltotriose followed the C—N bond protocol.
(ii) The amide bond formation (Mal-PEG2-NH) between Boc-L-Lys(N(maltotriose)2)-OH and Mal-PEG2-amine followed the HATU protocol.
(iii) Removal of the Boc protection group (de-BOC) of Boc-L-Lys(N(maltotriose)2)-PEG2-Mal followed the Boc protocol.
(iv) The amide bond formation (DM1) between NH2-L-Lys(N(maltotriose)2)-PEG2-Mal and DM1-AMAS-L-Ser followed the HATU protocol.

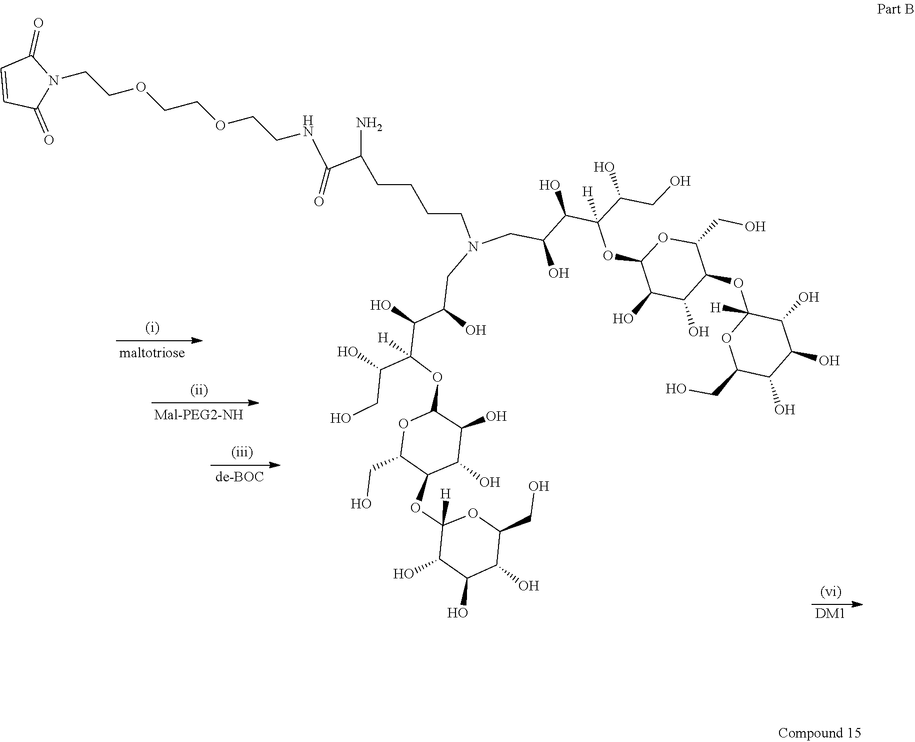

Compound 16 (Mal-PEG1-4AP(PEG1-Ser-AMAS-DM1)-(CH2)2-Gly-N(D-maltose)2) was prepared according to the following protocol:

Part A, see the Part A protocol for compound 2 above.

Part B
(i) The amide bond formation (N-BOC ethylenediamine, EDA-BOC) between Fmoc-Gly-OH and NH2-C2-NH-Boc followed the HATU protocol.
(ii) Removal of the Fmoc protection group (de-Fmoc) of Boc-(CH2)2-Gly-Fmoc followed the Fmoc protocol.
(iii) The C—N bond (D-maltose) formation between Boc-(CH2)2-Gly-NH2 and D-maltose followed the C—N bond protocol.
(iv) Removal of the Boc protection group (de-BOC) of Boc-(CH2)2-Gly-N(D-maltose)2 followed the Boc protocol.
(v) Removal of the Boc protection group (de-BOC) of Fmoc-4AP(Boc)-OH followed the Boc protocol.
(vi) The amide bond formation (N-BOC PEG1) between Fmoc-4AP(NH2)-OH and t-Boc-N-amido-PEG1-acid followed the HATU protocol.
(vii) The amide bond formation (D-Maltose) between Fmoc-4AP(PEG1-Boc)-OH and NH2-(CH2)2-Gly-N(D-maltose)2 followed the HATU protocol.
(viii) Removal of the Boc protection group (de-BOC) of Fmoc-4AP(PEG1-Boc)-(CH2)2-Gly-N(D-maltose)2 followed the Boc protocol.
(ix) The amide bond formation (DM1) between Fmoc-4AP(PEG1-NH2)-(CH2)2-Gly-N(D-maltose)2 and DM1-L-AMAS-Ser followed the HATU protocol.
(x) Removal of the Fmoc protection group (de-Fmoc) of Fmoc-4AP(PEG1-L-Ser-AMAS-DM1)-(CH2)2-Gly-N(D-maltose)2 followed the Fmoc protocol.
(xi) The amide bond formation (Mal-PEG1) between NH2-4AP(PEG1-L-Ser-AMAS-DM1)-(CH2)2-Gly-N(D-maltose)2 and Mal-PEG1-acid followed the HATU protocol.

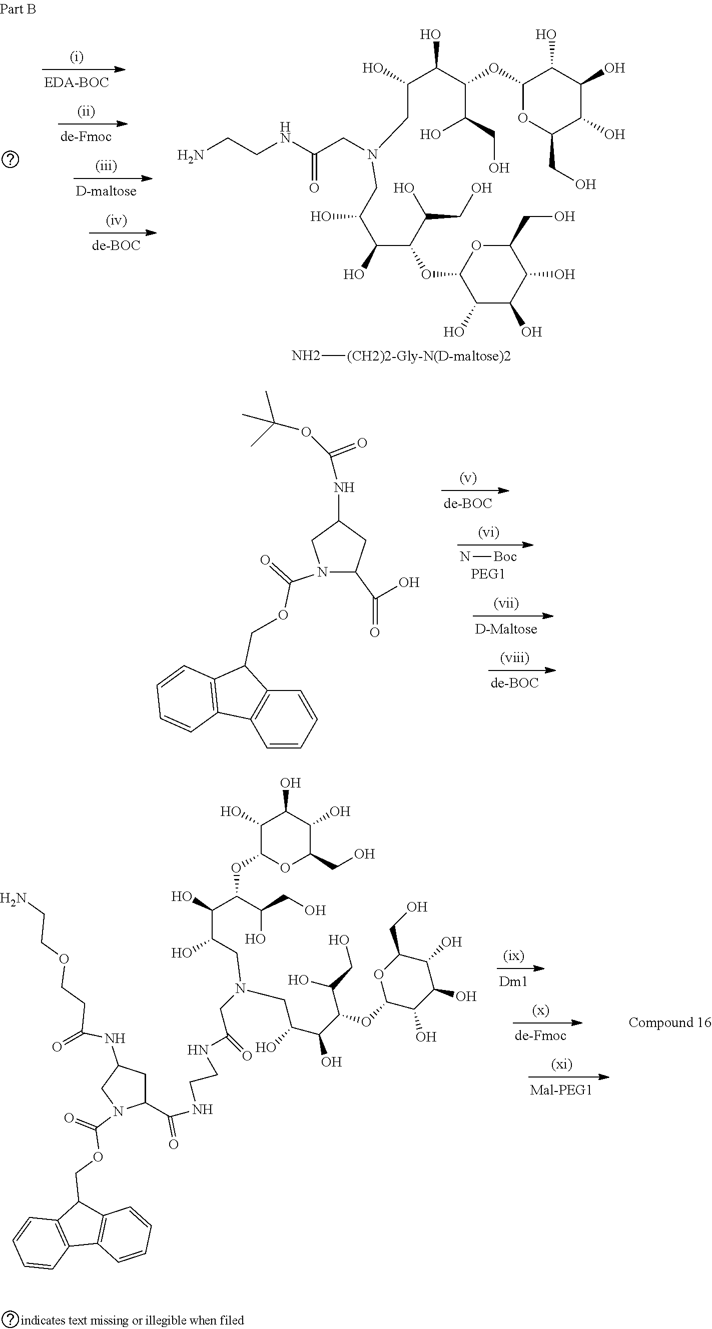

Compound 17 (Mal-PEG1-4AP(PEG1-L-Ser-AMAS-DM1)-(CH2)2-(Sar-PEG8-OH)2) was prepared according to the following protocol:

Part A, see the Part A protocol for compound 2 above.

Part B
(i) The C—N bond formation between Fmoc-ethylenediamine and Boc-Sar-aldehyde followed the C—N bond protocol.
(ii) Removal of the Boc protection group of Fmoc-(CH2)2-N(Sar-Boc)2 followed the Boc protocol.
(iii) The amide bond formation between Fmoc-(CH2)2-N(Sar-NH)2 and Hydroxy-PEG8-acid followed the HATU protocol.
(iv) Removal of the Fmoc protection group of Fmoc-(CH2)2-N(Sar-PEG8-OH)2 followed the Fmoc protocol.
(v) Removal of the Boc protection group (de-BOC) of Fmoc-4AP(Boc)-OH followed the Boc protocol.
(vi) The amide bond formation (N—BOC-PEG1) between Fmoc-4AP(NH2)-OH and t-Boc-N-amido-PEG1-acid followed the HATU protocol.
(vii) The amide bond formation between Fmoc-4AP(PEG1-Boc)-OH and NH2-(CH2)2-N(Sar-PEG8-OH)2 followed the HATU protocol.
(viii) Removal of the Boc protection group of Fmoc-4AP(PEG1-Boc)-(CH2)2-N(Sar-PEG8-OH)2 followed the Boc protocol.
(ix) The amide bond formation between Fmoc-4AP(PEG1-NH2)-(CH2)2-N(Sar-PEG8-OH)2 and DM1-AMAS-L-Ser followed the HATU protocol.
(x) Removal of the Fmoc protection group of Fmoc-4AP(PEG1-L-Ser-AMAS-DM1)-(CH2)2-N(Sar-PEG8-OH)2 followed the Fmoc protocol.
(xi) The amide bond formation between NH2-4AP(PEG1-L-Ser-AMAS-DM1)-(CH2)2-N(Sar-PEG8-OH)2 and Mal-PEG1-acid followed the HATU protocol.

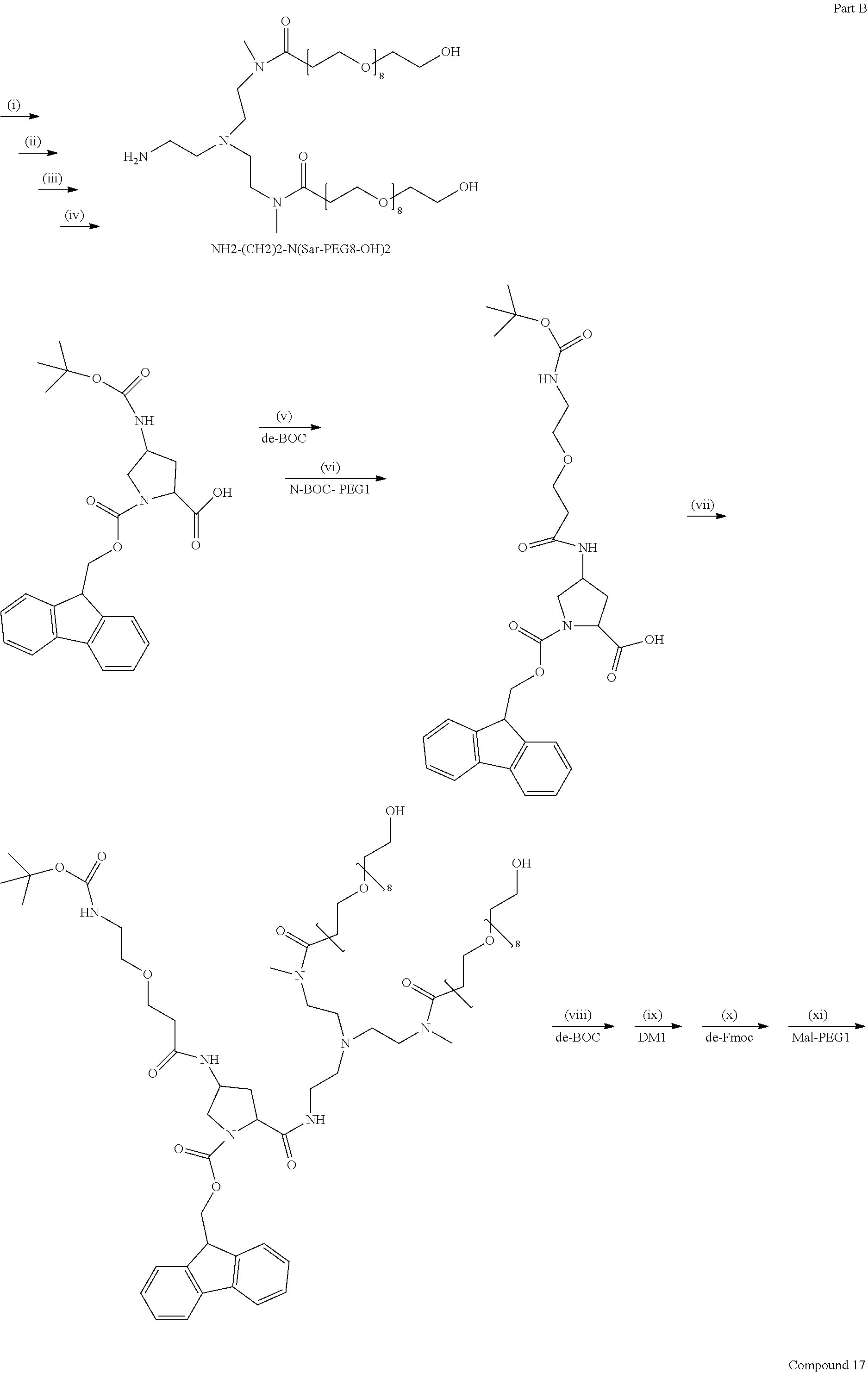

Compound 18 (Mal-PEG1-4AP(N(maltose)2)-VCt-Cys-C2OC2-DM1) was prepared according to the following protocol:

Part A.
(i) The C—S bond formation between DM1 and 2-Iodoethyl ether followed the C—S protocol.
(ii) The C—S bond formation between DM1-C2OC2-I and Cysteine followed the C—S protocol.

Part B
(i) The amide bond formation between Fmoc-Val-Cit-OH and Cys-C2OC2-DM1 followed the HATU protocol.
(ii) Removal of the Fmoc protection group of Fmoc-Val-Cit-Cys-C2OC2-DM1 followed the Fmoc protocol.
(iii) Removal of the Boc protection group of Fmoc-4AP(Boc)-OH followed the Boc protocol.
(iv) The C—N bond formation between Fmoc-4AP(NH2)-OH and D-maltose followed the C—N bond protocol.
(v) The amide bond formation between Fmoc-4AP(N(D-maltose)2)-OH and NH2-Val-Cit-Cys-C2OC2-DM1 followed the HATU protocol.
(vi) Removal of the Fmoc protection group of Fmoc-4AP(N(D-maltose)2)-Val-Cit-Cys-C20C2-DM1 followed the Fmoc protocol.
(vii) The amide bond formation between NH2-4AP(N(D-maltose)2)-Val-Cit-Cys-C20C2-DM1 and Mal-PEG1-acid followed the HATU protocol.

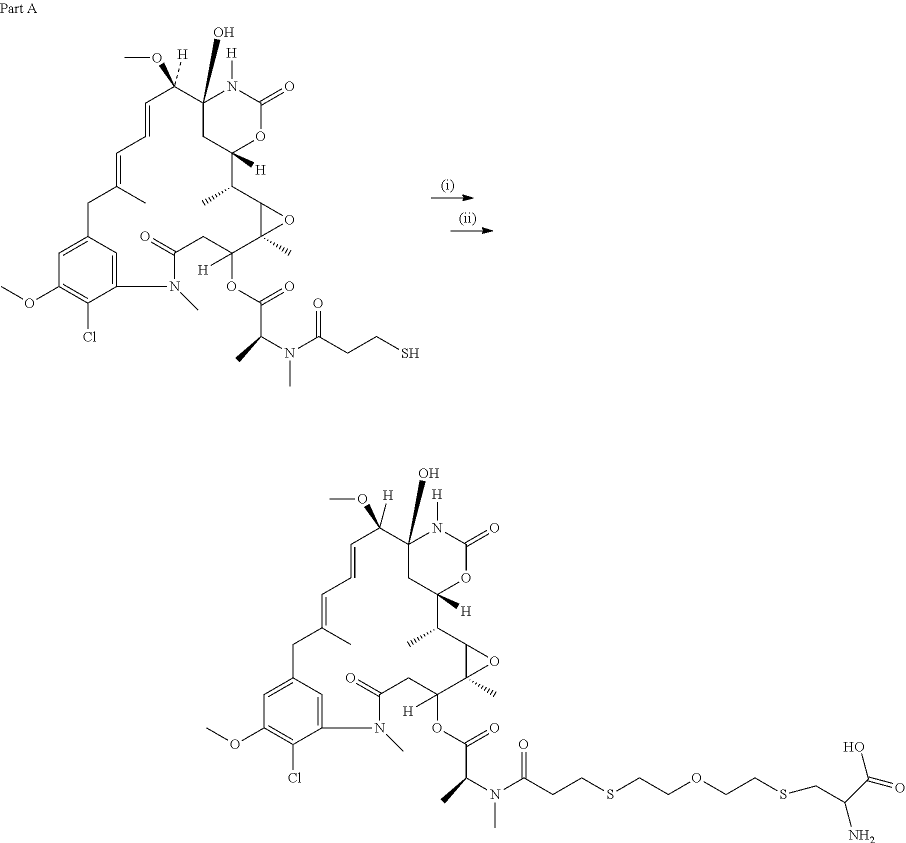 [SMILES: COc1cc2cc(c1Cl)N(C)C(=O)CC(OC(=O)[C@H](C)N(C)C(=O)CCS)[C@]1(C)OC1[C@H](C)[C@@H]1C[C@@](O)(NC(=O)O1)[C@H](OC)/C=C/C=C(\C)C2 ; COc1cc2cc(c1Cl)N(C)C(=O)CC(OC(=O)[C@H](C)N(C)C(=O)CCSCCOCCSCC(N)C(=O)O)[C@]1(C)OC1[C@H](C)[C@@H]1C[C@@](O)(NC(=O)O1)[C@H](OC)/C=C/C=C(\C)C2]

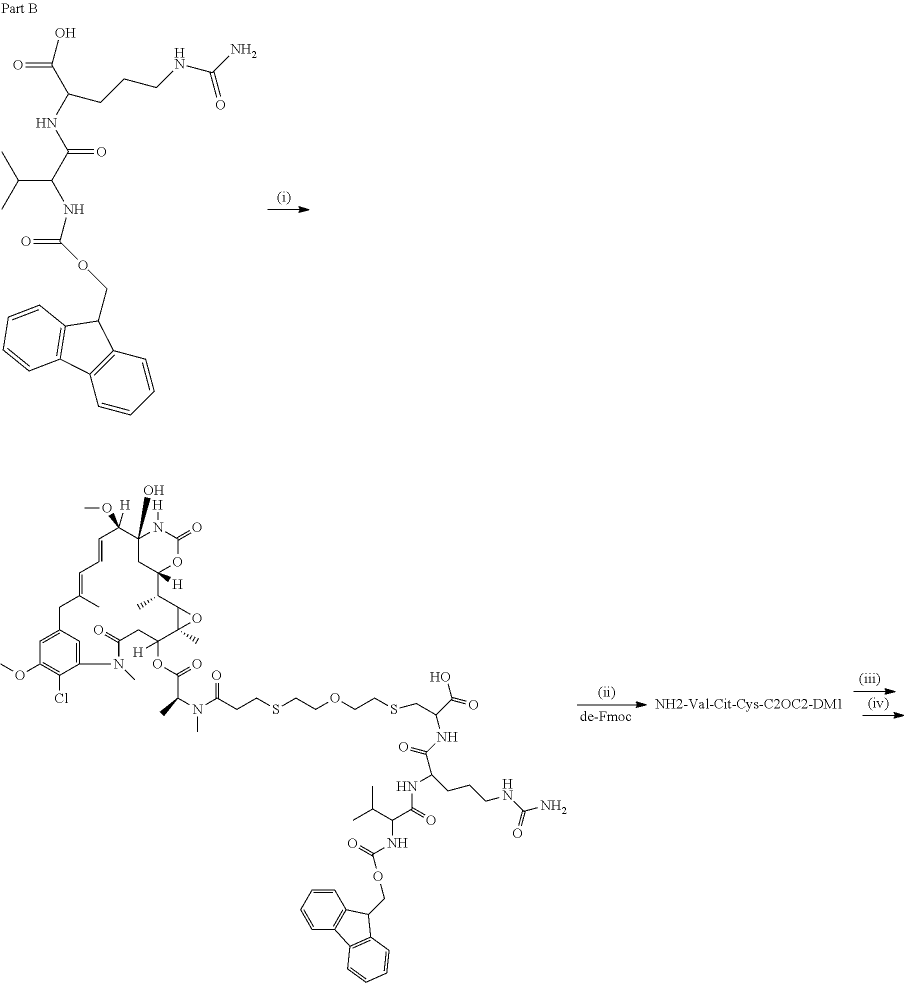 [SMILES: CC(C)C(NC(=O)OCC1c2ccccc2-c2ccccc21)C(=O)NC(CCCNC(N)=O)C(=O)O ; COc1cc2cc(c1Cl)N(C)C(=O)CC(OC(=O)[C@H](C)N(C)C(=O)CCSCCOCCSCC(NC(=O)C(CCCNC(N)=O)NC(=O)C(NC(=O)OCC1c3ccccc3-c3ccccc31)C(C)C)C(=O)O)[C@]1(C)OC1[C@H](C)[C@@H]1C[C@@](O)(NC(=O)O1)[C@H](OC)/C=C/C=C(\C)C2]

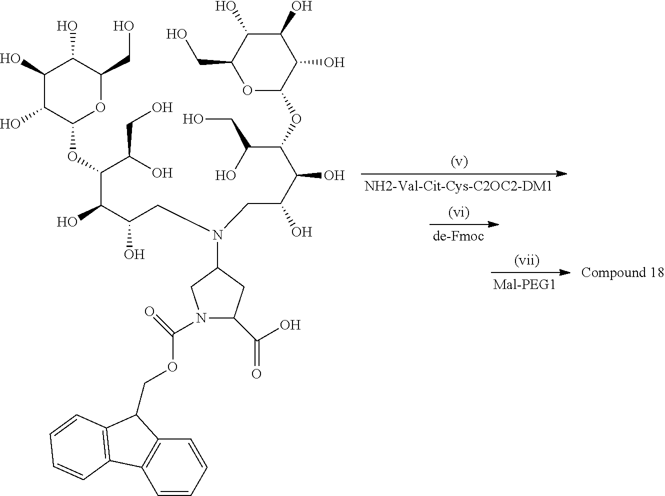

Compound 19 (Mal-PEG1-4AP(N(maltose)2)-Ala-Cys-C2OC2-DM1) was prepared according to the following protocol:

Part A, see the Part A protocol for compound 18.

Part B
(i) Removal of the Boc protection group (de-BOC) of Fmoc-4AP(Boc)-OH followed the Boc protocol.
(ii) The C—N bond formation (D-maltose) between Fmoc-4AP(NH2)-OH and D-maltose followed the C—N bond protocol.
(iii) The amide bond formation between Fmoc-4AP(N(D-maltose)2)-OH and Alanine (Ala) followed the HATU protocol.
(iv) The amide bond formation (CysDM1) between Fmoc-4AP(N(D-maltose)2)-Ala-OH and Cys-C2OC2-DM1 followed the HATU protocol.
(v) Removal of the Fmoc protection group (de-Fmoc) of Fmoc-4AP(N(D-maltose)2)-Ala-Cys-C20C2-DM1 followed the Fmoc protocol.
(vi) The amide bond formation (Mal-PEG1) between NH2-4AP(N(D-maltose)2)-Ala-Cys-C20C2-DM1 and Mal-PEG1-acid followed the HATU protocol.

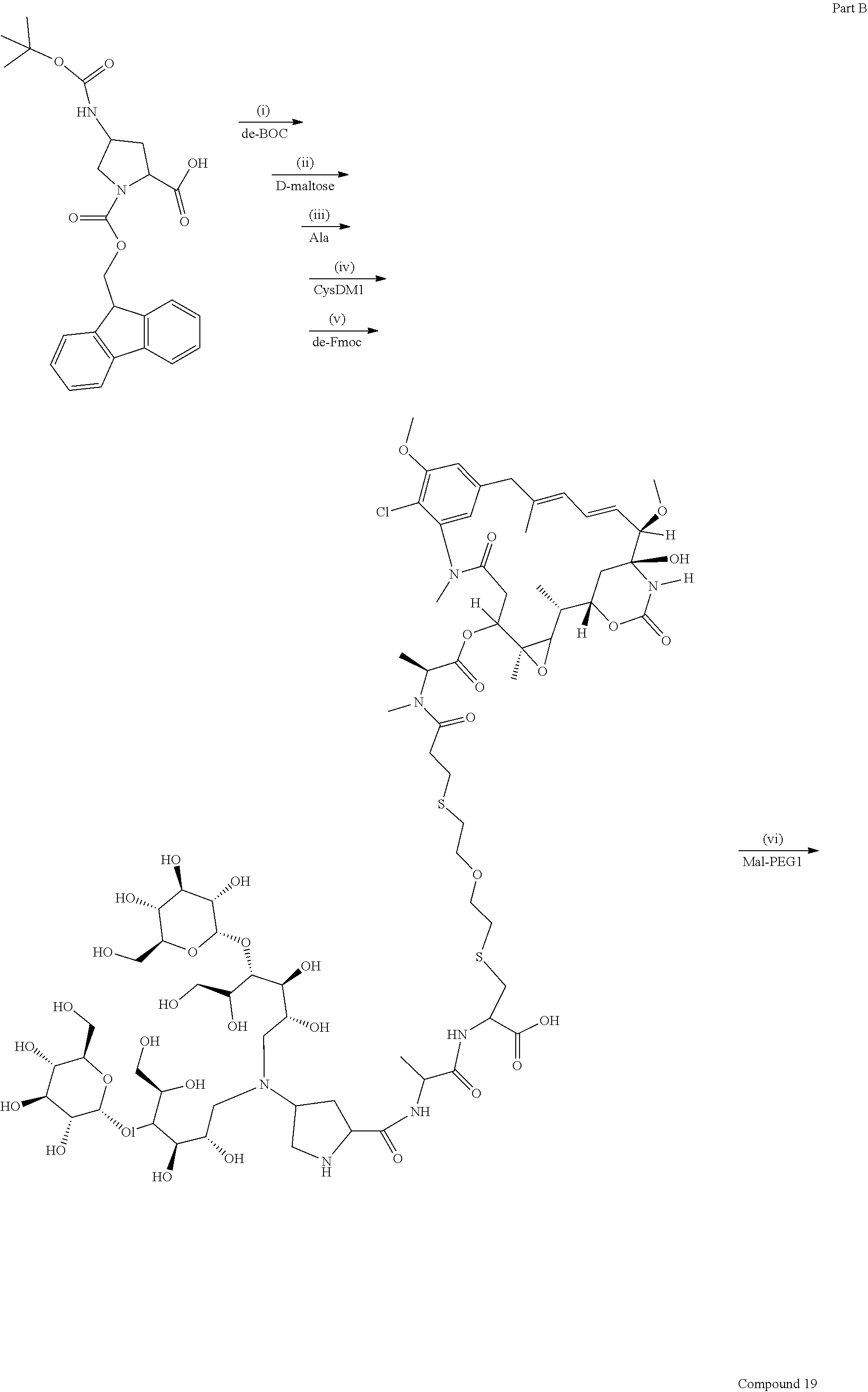

Compound 20 (DM1-AMAS-L-Ser-L-Lys(N(PEG4m)2)-PEG2-Mal) was prepared according to the following protocol:

Part A, see the Part A protocol for compound 2 above.

Part B.
(i) The C—N bond formation between Boc-L-Lys(NH2)-OH and m-PEG4-aldehyde followed the C—N bond protocol.
(ii) The amide bond formation (Mal-PEG2) between Boc-L-Lys(N(PEG4m)2)-OH and Mal-PEG2-amine TFA salt followed the HATU protocol.
(iii) Removal of the Boc protection group (de-BOC) of Boc-L-Lys(N(PEG4m)2)-PEG2-Mal followed the Boc protocol.
(iv) The amide bond formation (DM1) between NH2-L-Lys(N(PEG4m)2)-PEG2-Mal and DM1-AMAS-L-Ser followed the HATU protocol

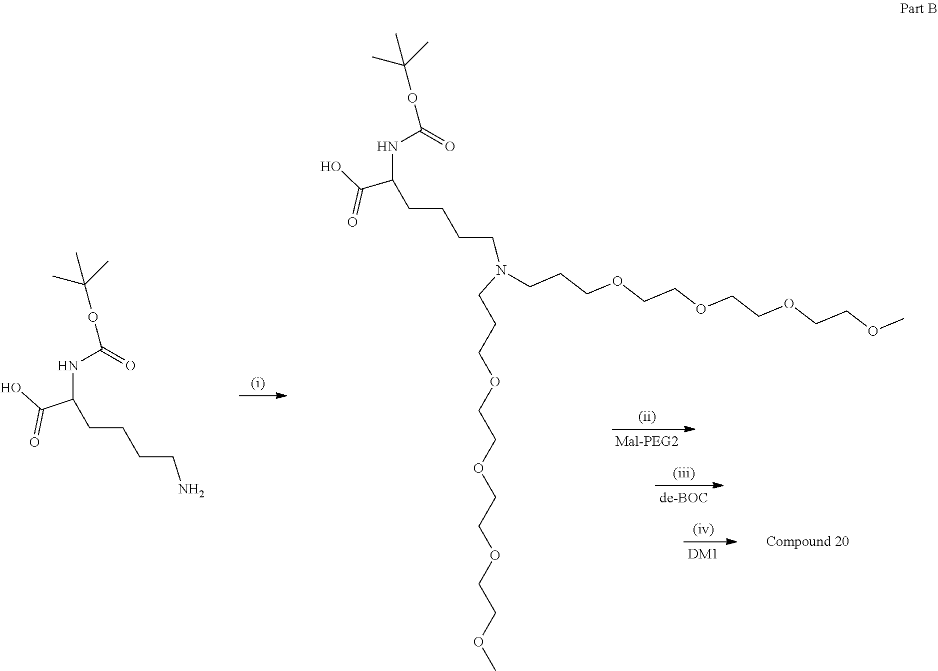

Compound 21 (Mal-PEG1-4AP(Sar-PEG80H)×2-DAP-Ag) was prepared according to the following protocol:
(i) The amide bond formation between Fmoc-DAP(boc)-OH and TLR7/8 agonist Ag followed the HATU protocol.
(ii) Removal of the Fmoc protection group (de-Fmoc) of Fmoc-DAP(boc)-Ag followed the Fmoc protocol.
(iii) The C—N bond formation between Fmoc-4AP(NH2)-OH and tert-Butyl methyl(2-oxoethyl)carbamate (boc-Sar-aldehyde) followed the C—N bond protocol.
(iv) Removal of the Boc protection group (de-BOC) of (Fmoc-4AP(Boc-Sar)×2-OH) followed the Boc protocol.
(v) The amide bond formation (PEG8) between (Fmoc-4AP(NH2-Sar)×2-OH) and Hydroxy-PEG8-acid followed the HATU protocol.
(vi) The amide bond formation between Fmoc-4AP(Sar-PEG80H)×2-OH and NH2-DAP(boc)-Ag followed the HATU protocol.
(vii) Removal of the Fmoc protection group (de-Fmoc) of Fmoc-4AP(Sar-PEG80H)×2-DAP(boc)-Ag followed the Fmoc protocol.
(viii) The amide bond formation (Mal-PEG1) between NH2-4AP(Sar-PEG80H)×2-DAP(boc)-Ag and Mal-PEG1-acid followed the HATU protocol.
(ix) Removal of the Boc protection group (de-BOC) of MP1A-4AP(Sar-PEG80H)×2-DAP(boc)-Ag followed the Boc protocol.

ESI-MS m/z calculated for C83H135N12O26 [M+H]+: 1716; found 1716.

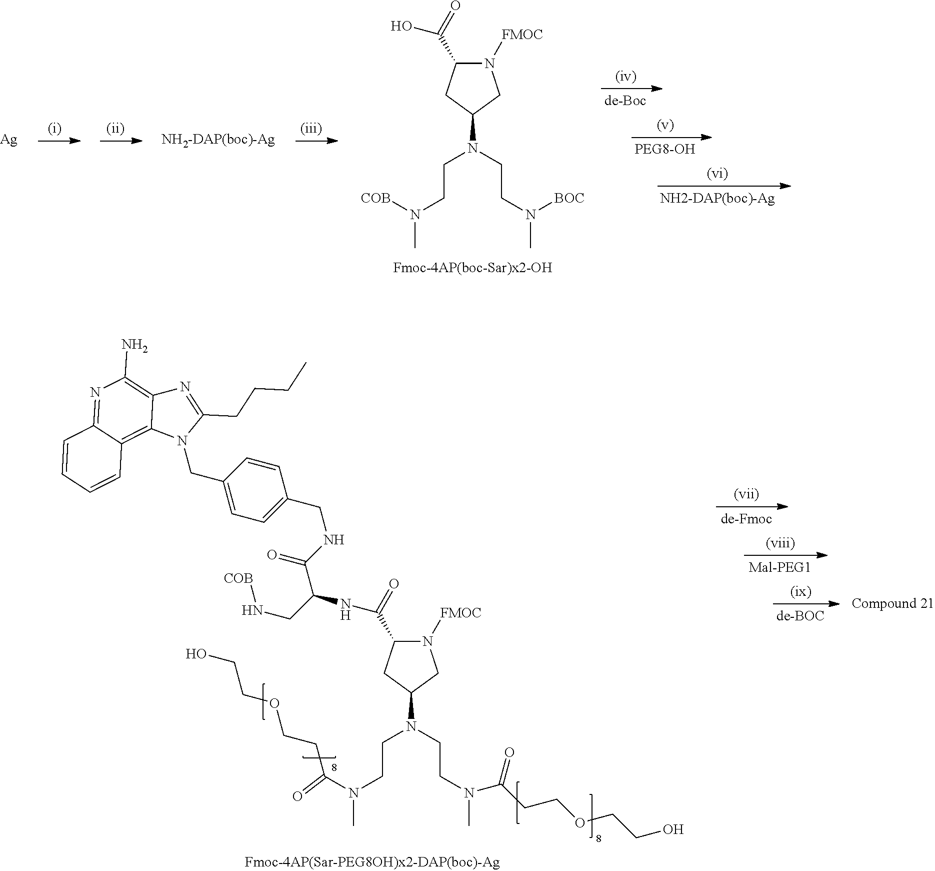

Compound 22 (Mal-PEG1-4AP(Sar-GlcA)×2-Ag) was prepared according to the following protocol:
(i) The C—N bond formation between Fmoc-4AP(NH2)-OH and tert-Butyl methyl(2-oxoethyl)carbamate (Boc-Sar-aldehyde) followed the C—N bond protocol.
(ii) The amide bond formation between Fmoc-4AP(Boc-Sar)×2-OH and TLR7/8 agonist Ag followed the HATU protocol.
(iii) Removal of Boc protection (de-BOC) of Fmoc-4AP(Boc-Sar)×2-Ag followed the Boc protocol.
(iv) The amide bond formation between Fmoc-4AP(NH2-Sar)×2-Ag and glucuronic acid (GlcA) followed the HATU protocol.
(v) Removal of the Fmoc protection group (de-Fmoc) of Fmoc-4AP(GlcA-Sar)×2-Ag followed the Fmoc protocol.
(vi) The amide bond formation (Mal-PEG1) between NH2-4AP(GlcA-Sar)×2-Ag and Mal-PEG1-acid followed the HATU protocol.

ESI-MS m/z calculated for C54H73N10O17 [M+H]+: 1133.5; found 1133.5.

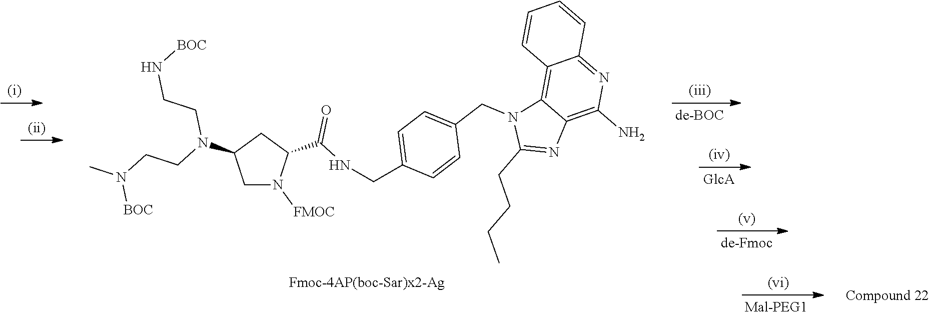

Compound 23 (Mal-PEG1-Lys(maltose×2)-Ag) was prepared according to the following protocol:
(i) Removal of the Boc protection group of Fmoc-Lys(Boc)-OH followed the Boc protocol.
(ii) The C—N bond formation between Fmoc-Lys(NH2)-OH and maltose followed the C—N bond protocol.
(iii) The amide bond formation (Ag) between Fmoc-Lys(maltose×2)-OH and TLR7/8 agonist Ag followed the HATU protocol.
(iv) Removal of the Fmoc protection group (de-Fmoc) of Fmoc-Lys(maltose×2)-Ag followed the Fmoc protocol.
(v) The amide bond formation (Mal-PEG1) between NH2-Lys(maltose×2)-Ag and Mal-PEG1-acid followed the HATU protocol.

ESI-MS m/z calculated for C61H91N8O25 [M+H]+: 1335.6; found 1335.6.

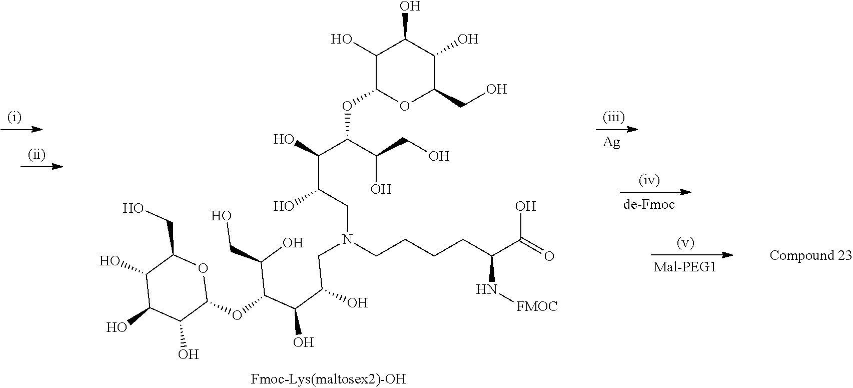

Compound 24 (Mal-PEG1-Lys-PEG4-maltose×2-Ag) was prepared according to the following protocol:
(i) The amide bond formation between Fmoc-Lys(NH2)-OH and t-Boc-N-amido-PEG4-NHS ester followed the NHS protocol.
(ii) Removal of the Boc protection group of Fmoc-Lys(PEG4-boc)-OH followed the Boc protocol.
(iii) The C—N bond formation between Fmoc-Lys(PEG4-NH2)-OH and maltose followed the C—N bond protocol.
(iv) The amide bond formation between Fmoc-Lys(PEG4-maltose×2)-OH and TLR7/8 agonist Ag followed the HATU protocol.
(v) Removal of the Fmoc protection group of Fmoc-Lys(PEG4-maltose×2)-Ag followed the Fmoc protocol.
(vi) The amide bond formation between NH2-Lys(PEG4-maltose×2)-Ag and Mal-PEG1-acid followed the HATU protocol.

ESI-MS m/z calculated for C72H112N9O30 [M+H]+: 1582.7; found 1582.7.

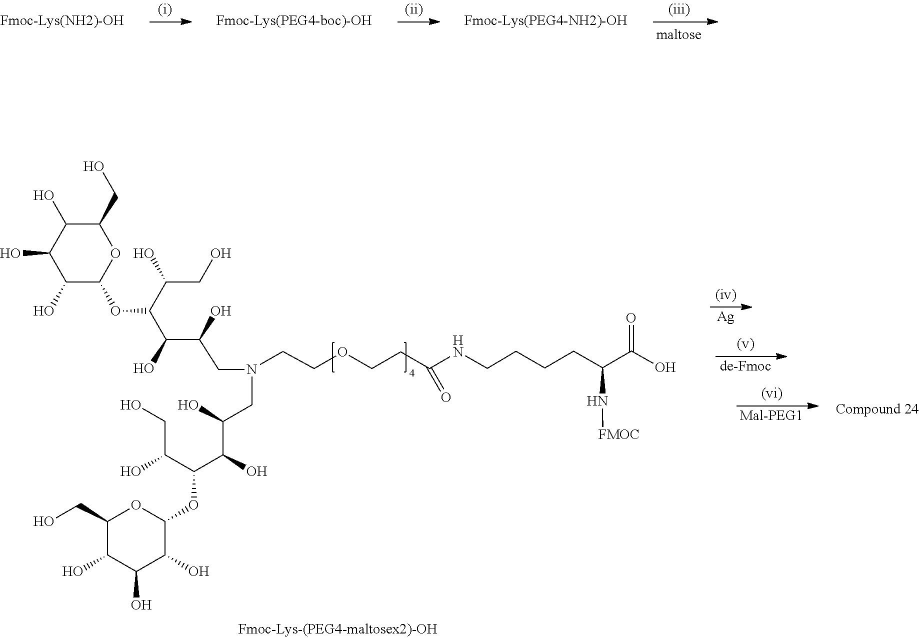

Compound 25 (Mal-PEG1-4AP(mPEG8×2)-Val-Cit-Ag) was prepared according to the following protocol: Part A
(i) The amide bond formation between Fmoc-Val-Cit-OH and TLR7/8 agonist Ag followed the HATU protocol.
(ii) Removal of the Fmoc protection group of Fmoc-Val-Cit-Ag followed the Fmoc protocol.

Part B
(i) The C—N bond formation between Fmoc-4AP(NH2)-OH and m-PEG8-aldehyde followed the C—N bond protocol.
(ii) The amide bond formation between Fmoc-4AP(mPEG8×2)-OH and NH2-Val-Cit-Ag followed the HATU protocol.
(iii) Removal of the Fmoc protection group of Fmoc-4AP(mPEG8×2)-Val-Cit-Ag followed the Fmoc protocol.
(iv) The amide bond formation between NH2-4AP(mPEG8×2)-Val-Cit-Ag and Mal-PEG1-acid followed the HATU protocol.

ESI-MS m/z calculated for C83H135N12O24 [M+H]+: 1684; found 1684.

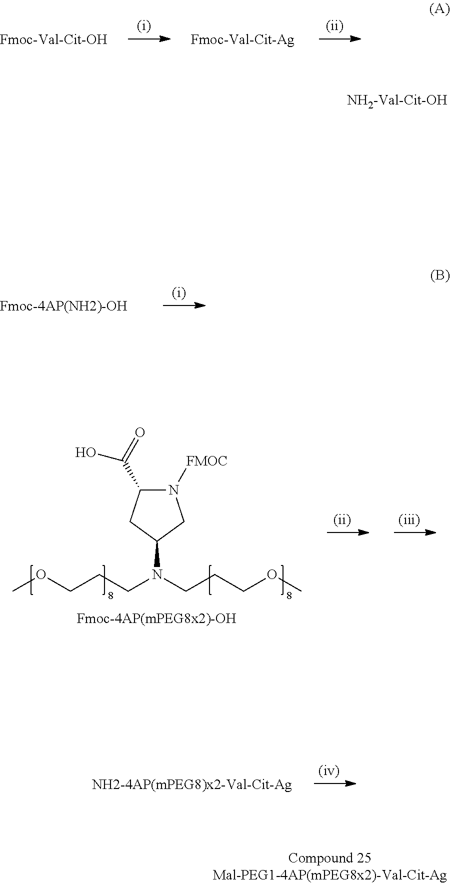

Compound 26 (Mal-PEG1-4AP(maltose×2)-PEG1-Val-Cit-Ag) was prepared according to the following protocol:

Part A
(i) The amide bond formation between NH2-Val-Cit-Ag and Fmoc-N-amido-PEG1-acid followed the HATU protocol.
(ii) Removal of the Fmoc protection group of Fmoc-PEG1-Val-Cit-Ag followed the Boc protocol.

Part B
(i) Removal of Boc protection group of Fmoc-4AP(boc)-OH followed the Boc protocol.
(ii) The C—N bond formation between Fmoc-4AP(NH2)-OH and maltose followed the C—N bond protocol.
(iii) The amide bond formation between Fmoc-4AP(maltose×2)-OH and NH2-PEG1-Val-Cit-Ag followed the HATU protocol.
(iv) Removal of the Fmoc protection group of Fmoc-4AP(maltose×2)-PEG1-Val-Cit-Ag followed the Fmoc protocol.
(v) The amide bond formation between NH2-4AP(maltose×2)-PEG1-Val-Cit-Ag and Mal-PEG1-acid followed the HATU protocol.

ESI-MS m/z calculated for C76H116N13O30 [M+H]+: 1690.8; found 1690.8.

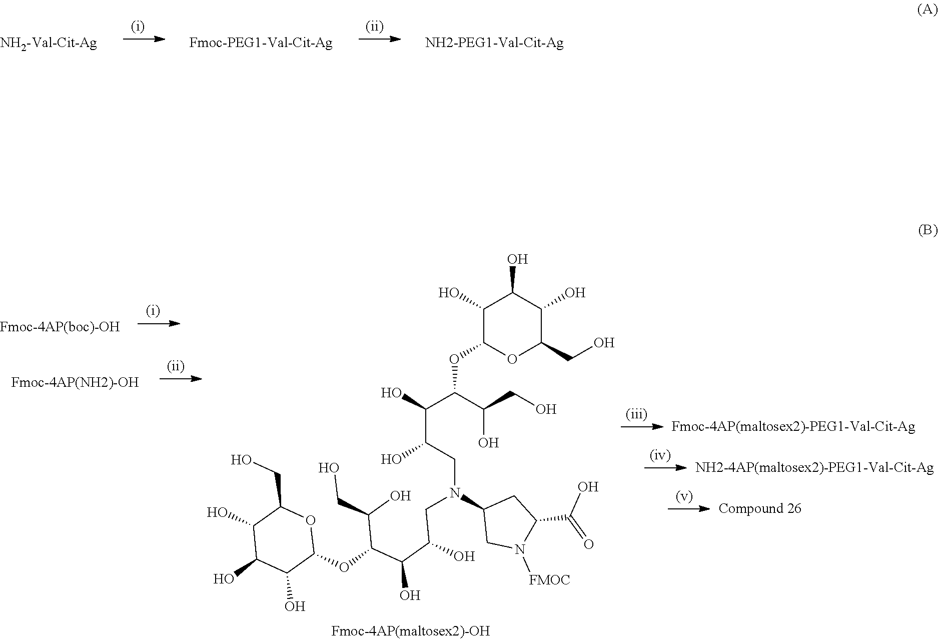

Compound 27 (Mal-PEG1-DAP-PEG1-Ala(mPEG8×2)-(PEG1-Val-Cit-Ag)) was prepared according to the following protocols.
(i) The amide bond formation between Fmoc-DAP(NH2)-OH and t-Boc-N-amido-PEG1-NHS ester (boc-PEG1-NHS) followed the NHS protocol.
(ii) Removal of the Boc protection group of Fmoc-DAP(boc-PEG1)-OH followed the Boc protocol.
(iii) The amide bond formation between Fmoc-DAP(PEG1-NH2)-OH and N-(tert-Butyloxycarbonyl)-L-alanine (boc-Ala-OH) followed the HATU protocol.
(iv) Removal of the Boc protection group of Fmoc-DAP(PEG1-Ala-boc)-OH followed the Boc protocol.
(v) The C—N bond formation between Fmoc-DAP(PEG1-Ala-NH2)-OH and mPEG8-aldehyde followed the C—N bond protocol.
(vi) The amide bond formation between Fmoc-DAP(PEG1-Ala-mPEG8×2)-OH and NH2-PEG1-Val-Cit-Ag followed the HATU protocol.
(vii) Removal of the Fmoc protection group of Fmoc-DAP(PEG1-Ala-mPEG8×2)-PEG1-Val-Cit-Ag followed the Fmoc protocol.
(viii) The amide bond formation between NH2-Fmoc-DAP(PEG1-Ala-mPEG8×2)-PEG1-Val-Cit-Ag and Mal-PEG1-acid followed the HATU protocol.

ESI-MS m/z calculated for C94H156N15O29 [M+H]+: 1959.1; found 1959.1.

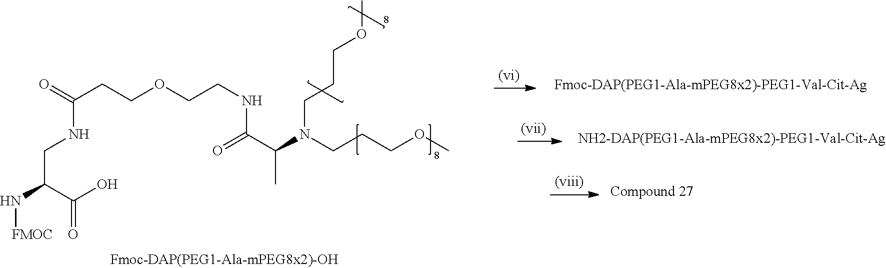

Compound 28 (Mal-PEG1-DAP-PEG1-Ala(maltose×2)-(PEG1-Val-Cit-Ag)) was prepared according to the following protocols.
(i) The C—N bond formation between Fmoc-DAP(PEG1-Ala-NH2)-OH and maltose followed the C—N bond protocol.
(ii) The amide bond formation between Fmoc-DAP(PEG1-Ala-maltose×2)-OH and NH2-PEG1-Val-Cit-Ag followed the HATU protocol.
(iii) Removal of the Fmoc protection group of Fmoc-DAP(PEG1-Ala-maltose×2)-PEG1-Val-Cit-Ag followed the Fmoc protocol.
(iv) The amide bond formation between NH2-DAP(PEG1-Ala-maltose×2)-PEG1-Val-Cit-Ag and Mal-PEG1-acid followed the HATU protocol.

ESI-MS m/z calculated for C82H128N15O33 [M+H]+: 1850.9; found 1850.9.

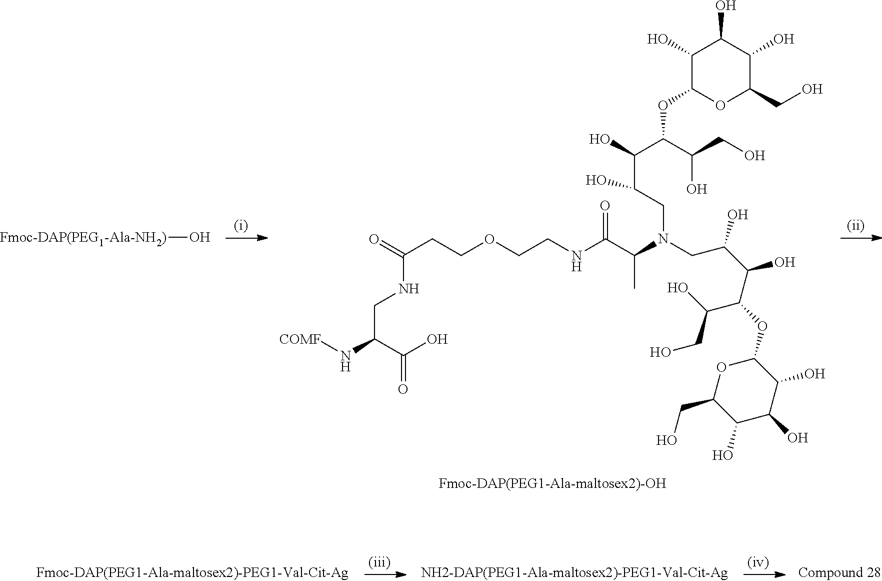

Compound 29 (Mal-PEG1-D-Dap(L-Ser-AMAS-DM1)-PEG1-N(D-maltose)2) was prepared according to the following protocols.

Part A, see the Part A protocol for compound 2 above.

Part B. NH2-PEG1-N(D-maltose)2 synthesis.
(i) The C—N bond formation between t-Boc-N-amido-PEG1-amine and D-maltose followed the C—N bond protocol.
(ii) Removal of the Boc protection group of t-Boc-N-amido-PEG1-N(D-maltose)2 followed the Boc protocol.

Part C. Mal-PEG1-D-Dap(L-Ser-AMAS-DM1)-PEG1-N(D-maltose)2
(i) The amide bond formation between Fmoc-D-Dap(Boc)-OH and NH2-PEG1-N(D-maltose)2 followed the HATU protocol.
(ii) Removal of the Boc protection group of Fmoc-D-Dap(Boc)-PEG1-N(D-maltose)2 followed the Boc protocol.
(iii) The amide bond formation (DM1) between Fmoc-D-Dap(NH2)-PEG1-N(D-maltose)2 and DM1-AMAS-L-Ser followed the HATU protocol.
(iv) Removal of the Fmoc protection group (de-Fmoc) of Fmoc-D-Dap(L-Ser-AMAS-DM1)-PEG1-N(D-maltose)2 followed the Fmoc protocol.
(v) The amide bond formation (Mal-PEG1) between NH2-D-Dap(L-Ser-AMAS-DM1)-PEG1-N(D-maltose)2 and Mal-PEG1-acid followed the HATU protocol.

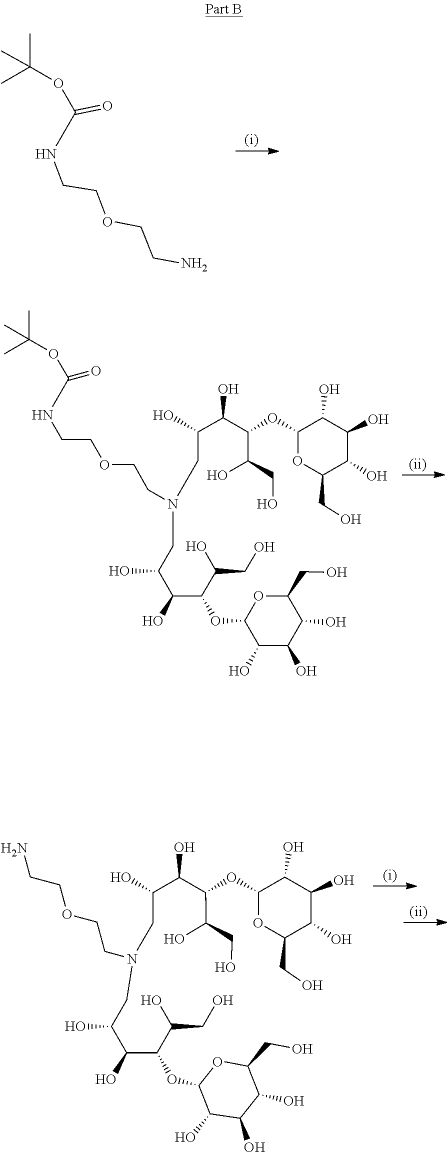

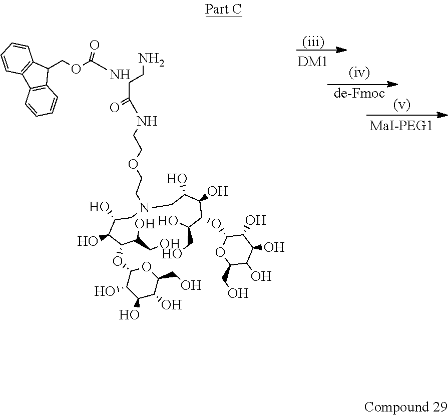

Compound 30 (Mal-PEG1-D-Dap(L-Ser-AMAS-DM1)-(CH2)2-N(D-maltose)2) was prepared according to the following protocols.

Part A, see the Part A protocol for compound 2 above.

Part B. NH2-(CH2)2-N(D-maltose)2 synthesis.
(i) The C—N bond formation between Boc-ethylenediamine (Boc-(CH2)2-NH2) and D-maltose followed the C—N bond protocol.
(ii) Removal of the Boc protection group of Boc-(CH2)2-N(D-maltose)2 followed the Boc protocol.

Part C. Mal-PEG1-D-Dap(L-Ser-AMAS-DM1)-(CH2)2-N(maltose)2
(i) The amide bond formation between Fmoc-D-Dap(Boc)-OH and NH2-(CH2)2-N(D-maltose)2 followed the HATU protocol.
(ii) Removal of the Boc protection group of Fmoc-D-Dap(Boc)-(CH2)2-N(D-maltose)2 followed the Boc protocol.
(iii) The amide bond formation between Fmoc-D-Dap(NH2)-(CH2)2-N(D-maltose)2 and DM1-AMAS-L-Ser followed the HATU protocol.
(iv) Removal of the Fmoc protection group of Fmoc-D-Dap(L-Ser-AMAS-DM1)-(CH2)2-N(D-maltose)2 followed the Fmoc protocol.
(v) The amide bond formation between NH2-D-Dap(L-Ser-AMAS-DM1)-(CH2)2-N(D-maltose)2 and Mal-PEG1-acid followed the HATU protocol.

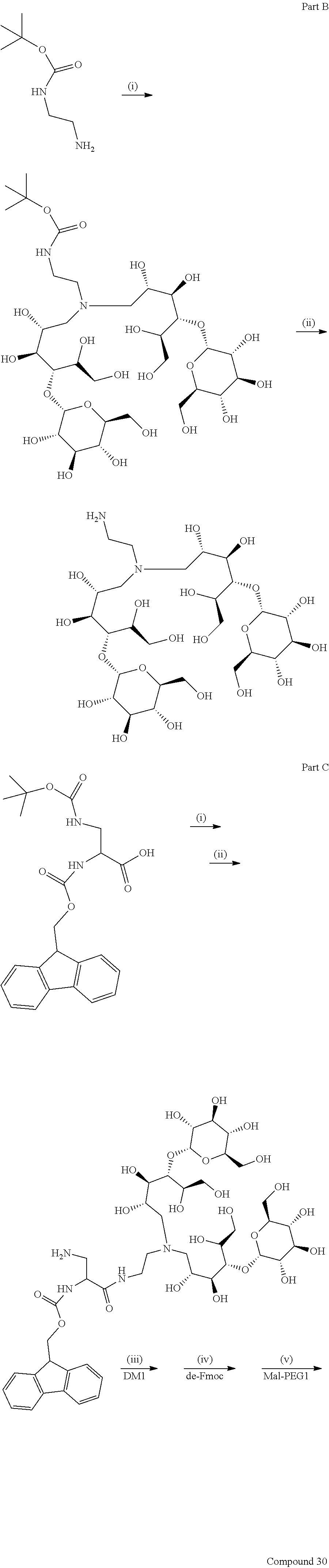

Compound 31 (Mal-PEG1-L-trans-Pyrrolidine-2,4-dicarboxylic Acid-[Lys(N(D-maltose)2)-PEG1-L-Ser-AMAS-DM1]2) was prepared according to the following protocol:

Part A, see the Part A protocol for compound 2 above.

Part B.
(i) The amide bond formation between L-trans-Pyrrolidine-2,4-dicarboxylic Acid and Fmoc-Osu followed the NHS protocol.

Part C.
(i) Removal of the Boc protection group of Fmoc-Lys(Boc)-OH followed the Boc protocol.
(ii) The C—N bond formation between Fmoc-Lys(NH2)-OH and D-maltose followed the C—N bond protocol.
(iii) The amide bond formation between Fmoc-Lys(N(D-maltose)2)-OH and t-Boc-N-amido-PEG1-amine followed the HATU protocol.
(iv) Removal of the Boc protection group of Fmoc-Lys(N(D-maltose)2)-PEG1-Boc followed the Boc protocol.
(v) The amide bond formation between Fmoc-Lys(N(D-maltose)2)-PEG1-NH2 and DM1-AMAS-L-Ser followed the HATU protocol.
(vi) Removal of the Fmoc protection group of Fmoc-Lys(N(D-maltose)2)-PEG1-L-Ser-AMAS-DM1 followed the Fmoc protocol.
(vii) The amide bond formation (di-amide) between NH2-Lys(N(D-maltose)2)-PEG1-L-Ser-AMAS-DM1 and Fmoc-L-trans-Pyrrolidine-2,4-dicarboxylic Acid followed the HATU protocol.
(viii) Removal of the Fmoc protection group of Fmoc-L-trans-Pyrrolidine-2,4-dicarboxylic Acid-[Lys(N(D-maltose)2)-PEG1-L-Ser-AMAS-DM1]2 followed the Fmoc protocol.
(ix) The amide bond formation between NH2-L-trans-Pyrrolidine-2,4-dicarboxylic Acid-[Lys(N(D-maltose)2)-PEG1-L-Ser-AMAS-DM1]2 and Mal-PEG1-acid followed the HATU protocol.

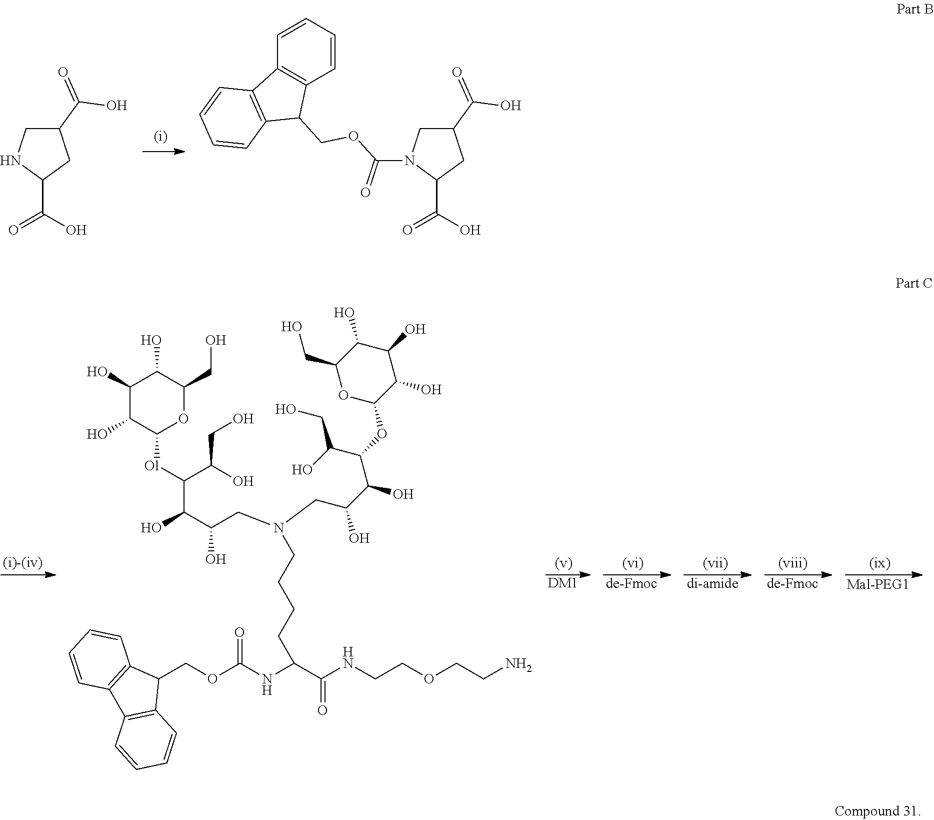

Compound 32 (Mal-PEG1-4AP(maltose×2)-3C-MDP-Ag) was prepared according to the following protocol:

Part A.
(i) The amide bond formation between Ag and muramyl dipeptide (MDP) followed the HATU protocol.

Part B.
(i) The amide bond formation between Fmoc-4AP(maltose×2)-OH and (3-Aminopropyl)hydrazine followed the HATU protocol.
(ii) The hydrazone bond formation of Fmoc-4AP(maltose×2)-3C-hydrazine and MDP-Ag followed the hydrazone protocol.
(iii) Removal of the Fmoc protection group of Fmoc-4AP(maltose×2)-3C-MDP-Ag followed the Fmoc protocol.
(iv) The amide bond formation of NH2-4AP(maltose×2)-3C-MDP-Ag and Mal-PEG1-acid followed the HATU protocol.

ESI-MS m/z calculated for C82H126N15O34 [M+H]+: 1864.9; found 1864.9.

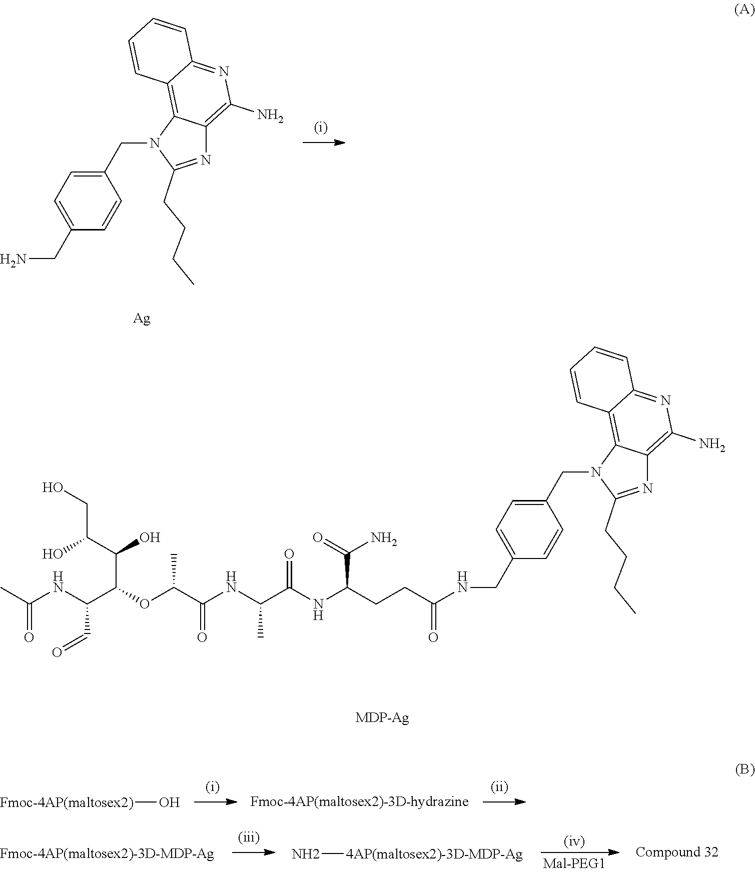

Compound 33 (Mal-PEG1-4AP(maltose×2)-Ser-Ag) was prepared according to the following protocol:

Part A.
(i) The amide bond formation between of Ag and N-boc-D-serine followed the HATU protocol.
(ii) The removal of the Boc protection group of Boc-Ser-Ag followed the Boc protocol.

Part B.
(i) The removal of the Boc protection group of Fmoc-4AP(boc)-OH followed the Boc protocol.
(ii) The C—N bond formation between Fmoc-4AP(NH2)-OH and maltose followed the C—N bond protocol.
(iii) The amide bond formation between Fmoc-4AP(maltose×2)-OH and NH2-Ser-Ag followed the HATU protocol.
(iv) Removal of the Fmoc protection group of Fmoc-4AP(maltose×2)-Ser-Ag followed the Fmoc protocol.
(v) The amide bond formation between NH2-4AP(maltose×2)-Ser-Ag and Mal-PEG1-acid followed the HATU protocol.

ESI-MS m/z calculated for C63H92N9O27 [M+H]+: 1406.6; found 1406.6.

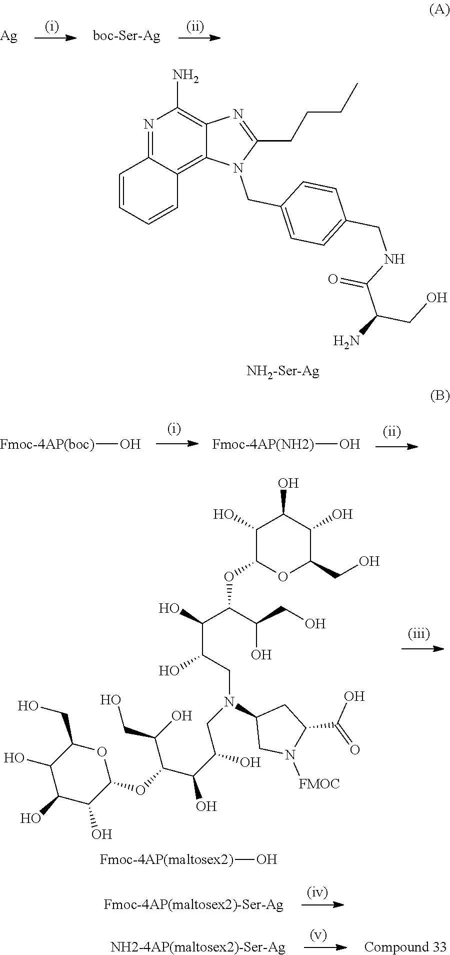

Compound 34 (Mal-PEG1-4AP(glucose×2)-PEG1-VCt-BAMe-Ag) was prepared according to the following protocol:

Part A.
(i) The C—N bond formation between tert-Butyl 4-formylbenzylcarbamate and Ag followed the C—N bond protocol.
(ii) The methylation of boc-BA-Ag followed the C—N bond protocol.
(iii) Removal of the Boc protection group of boc-BAMe-Ag followed the Boc protocol.

Part B.
(i) The amide bond formation between of Fmoc-PEG1-COOH and Val-Cit-OH followed the HATU protocol.
(ii) The amide bond formation between of Fmoc-PEG1-Val-Cit-OH and BAMe-Ag followed the HATU protocol.
(iii) Removal of the Fmoc protection group of Fmoc-PEG1-Val-Cit-BAMe-Ag followed the Fmoc protocol.

Part C.
(i) The removal of the Boc protection group of Fmoc-4AP(boc)-OH followed the Boc protocol.
(ii) The C—N bond formation between Fmoc-4AP(NH2)-OH and glucose followed the C—N bond protocol.
(iii) The amide bond formation between Fmoc-4AP(glucose×2)-OH and NH2-PEG1-Val-Cit-BAMe-Ag followed the HATU protocol.
(iv) Removal of the Fmoc protection group of Fmoc-4AP(glucose×2)-PEG1-Val-Cit-BAMe-Ag followed the Fmoc protocol.
(v) The amide bond formation between NH2-4AP(glucose×2)-PEG1-Val-Cit-BAMe-Ag and Mal-PEG1-acid followed the HATU protocol.

ESI-MS m/z calculated for C73H107N14O20 [M+H]+: 1499.8; found 1499.8.

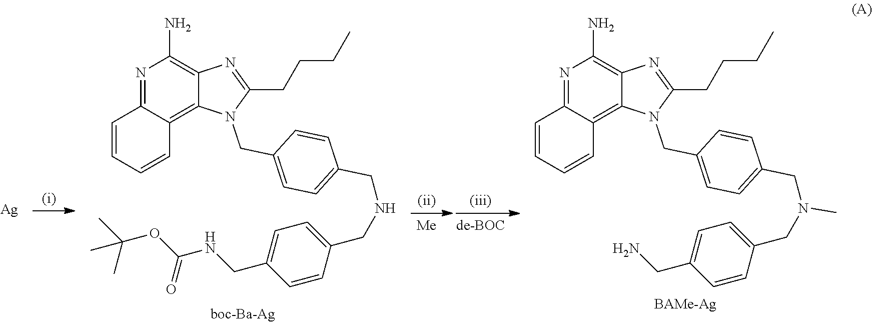

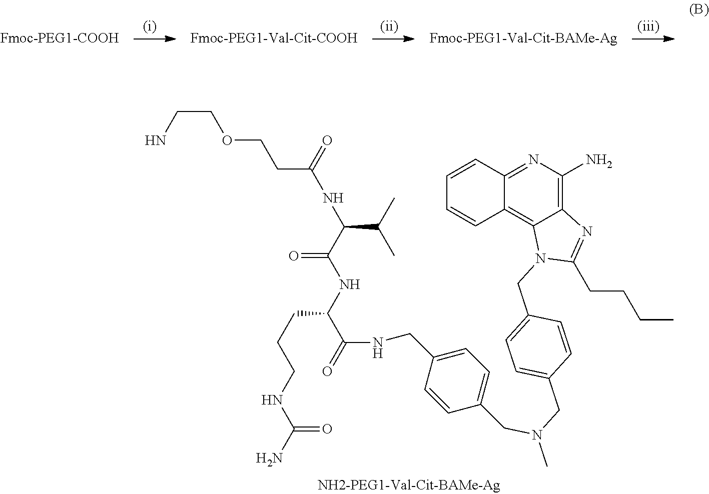 [SMILES: CCCCc1nc2c(N)nc3ccccc3c2n1Cc1ccc(CN(C)Cc2ccc(CNC(=O)[C@H](CCCNC(N)=O)NC(=O)[C@@H](NC(=O)CCOCCN)C(C)C)cc2)cc1]

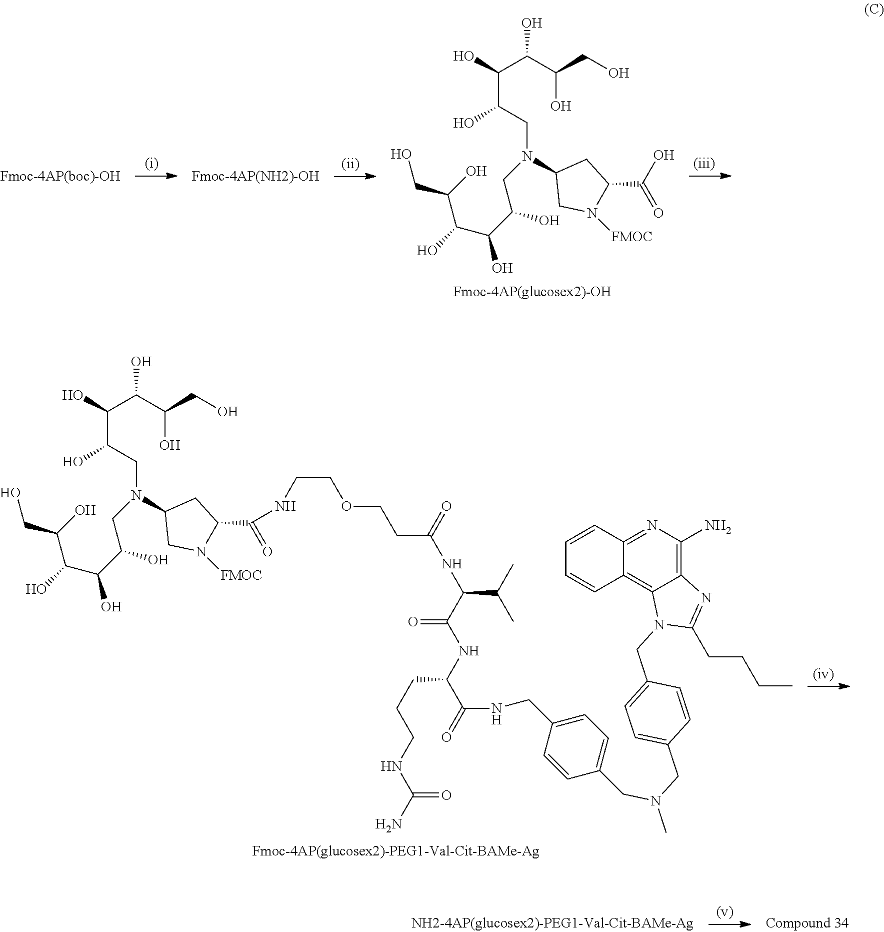

Compound 35 (Mal-PEG1-4AP(maltose×2)-PEG1-CPCA-BAMe-Ag) was prepared according to the following protocol:

Part A, See the Part A protocol for compound 34.

Part B.
(i) The amide bond formation between of Fmoc-PEG1-OH and rel-(1R,2R)-2-(tert-Butoxycarbonyl)cyclopropane-1-carboxylic acid (CPCA-Otbu) followed the ester bond protocol.
(ii) Removal of the tbu protection group of Fmoc-PEG1-CPCA-Otbu followed the boc protocol.
(iii) The amide bond formation between of Fmoc-PEG1-CPCA-OH and BAMe-Ag followed the HATU protocol.
(iv) Removal of the Fmoc protection group of Fmoc-PEG1-CPCA-BAMe-Ag followed the Fmoc protocol.

Part C.
(i) The C—N bond formation between Fmoc-4AP(NH2)-OH and maltose followed the C—N bond protocol.
(ii) The amide bond formation between Fmoc-4AP(maltose×2)-OH and NH2-PEG1-CPCA-BAMe-Ag followed the HATU protocol.
(iii) Removal of the Fmoc protection group of Fmoc-4AP(maltose×2)-PEG1-CPCA-BAMe-Ag followed the Fmoc protocol.
(iv) The amide bond formation between NH2-4AP(maltose×2)-PEG1-CPCA-BAMe-Ag and Mal-PEG1-acid followed the HATU protocol.

ESI-MS m/z calculated for C79H111N10O30 [M+H]+: 1679.7; found 1679.7.

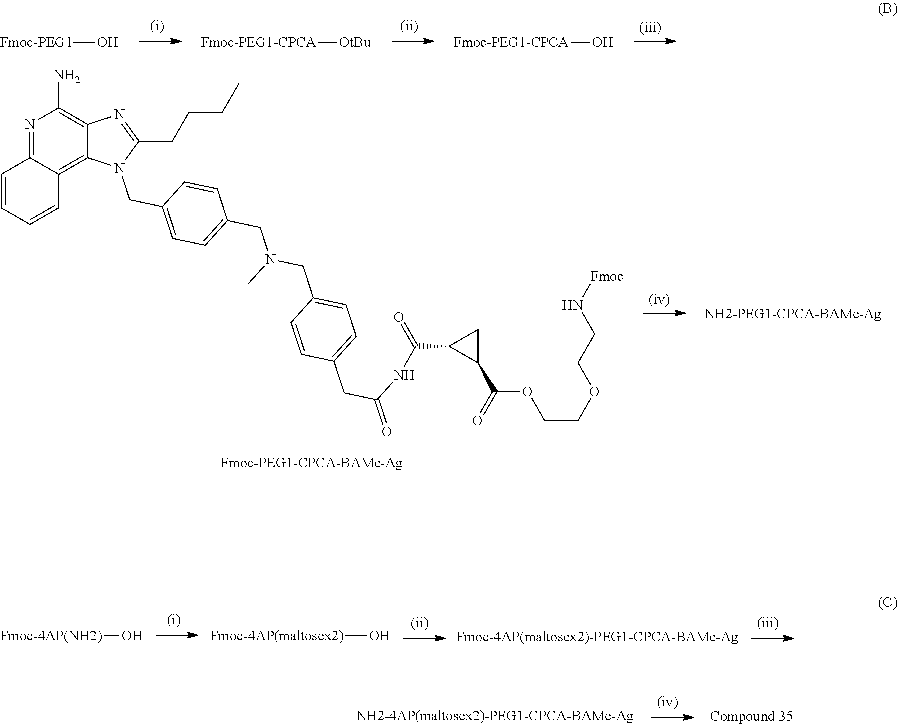

Compound 36 (Mal-PEG1-4AP(N(Glucose)2)-PEG1-L-Ser-AMAS-DM1) was prepared according to the following protocol:

Part A, see the Part A protocol for compound 2 above.

Part B.
(i) Removal of the Boc protection group of Fmoc-4AP(Boc)-OH followed the Boc protocol.
(ii) The C—N bond formation between Fmoc-4AP(NH2)-OH and glucose followed the C—N bond protocol.
(iii) The amide bond formation between Fmoc-4AP(N(glucose)2)-OH and t-Boc-N-amido-PEG1-amine followed the HATU protocol.
(iv) Removal of the Boc protection group of Fmoc-4AP(N(glucose)2)-PEG1-Boc followed the Boc protocol.
(v) The amide bond formation between Fmoc-4AP(N(glucose)2)-PEG1-NH2 and DM1-AMAS-L-Ser followed the HATU protocol.
(vi) Removal of the Fmoc protection group of Fmoc-4AP(N(glucose)2)-PEG1-L-Ser-AMAS-DM1 followed the Fmoc protocol.
(vii) The amide bond formation between NH2-4AP(N(glucose)2)-PEG1-L-Ser-AMAS-DM1 and Mal-PEG1-acid followed the HATU protocol.

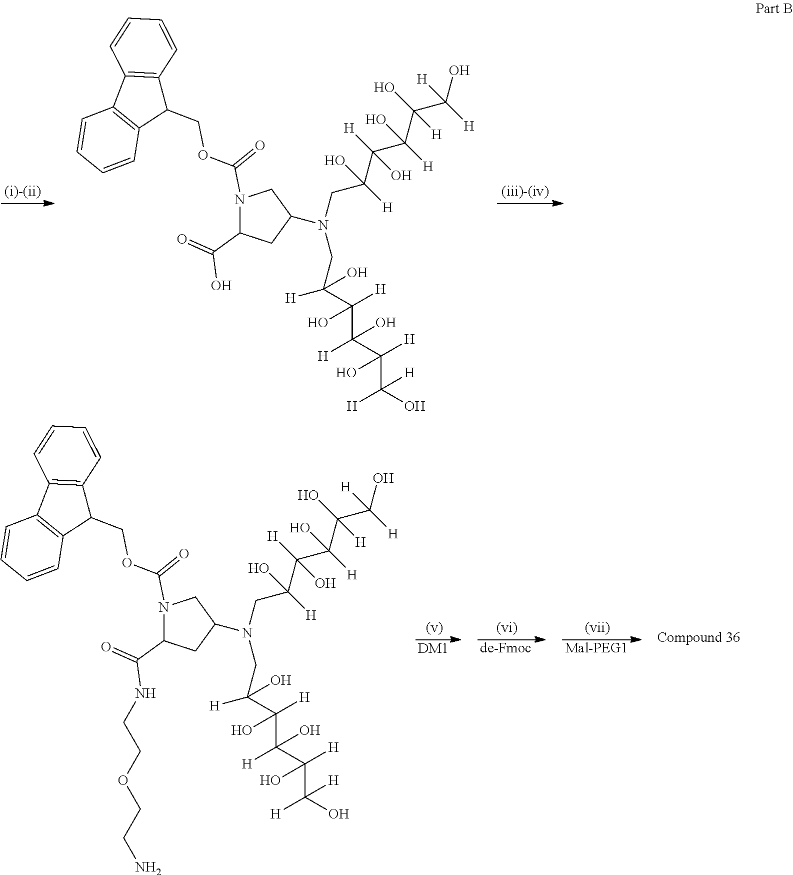

Compound 37 (Mal-PEG1-4AP(N(D-maltose)2)-PEG2-ACPC-(C═O)—O-Dxd) was prepared according to the following protocol:

Part A.
(i) The ester bond formation between Dxd and Boc-ACPC followed the ester bond protocol.
(ii) Removal of the Boc protection group of Boc-ACPC-(C═O)—O-Dxd followed the Boc protocol.

Part B.
(i) The C—N bond formation between Boc-4AP(NH2)-OH and D-maltose followed the C—N bond protocol.
(ii) The amide bond formation between Boc-4AP(N(D-maltose)2)-OH and amino-PEG2-acid followed the HATU protocol.
(iii) The amide bond formation (ACPC-Dxd) between Boc-4AP(N(D-maltose)2)-PEG2-acid and NH2-ACPC-(C═O)—O-Dxd followed the HATU protocol.
(iv) Removal of the Boc protection group of Boc-4AP(N(D-maltose)2)-PEG2-ACPC-(C═O)—O-Dxd followed the Boc protocol.
(v) The amide bond formation between NH2-4AP(N(D-maltose)2)-PEG2-ACPC-(C═O)—O-Dxd and Mal-PEG1-acid followed the HATU protocol.

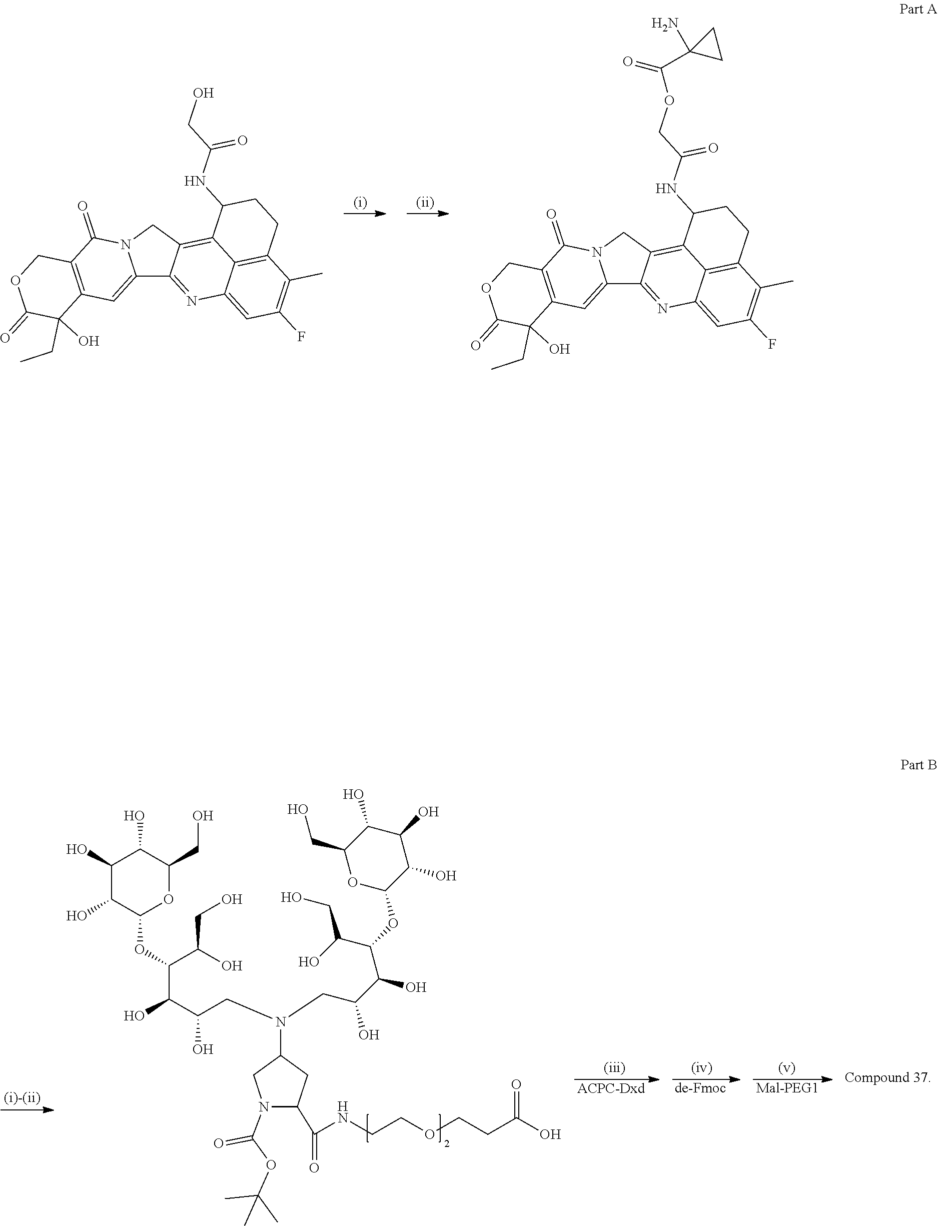

Compound 38 (Mal-PEG1-4AP(N(D-maltose)2)-PEG1-ACPC-(C═O)—O-Dxd) was prepared according to the following protocol:

Part A.
(i)-(ii) See the Part A protocol for Compound 37 above.
(iii) The amide bond formation between NH2-ACPC-(C═O)—O-Dxd and Boc-PEG1-acid followed the HATU protocol.
(iv) Removal of the Boc protection group of Boc-PEG1-ACPC-(C═O)—O-Dxd followed the Boc protocol.

Part B.
(i) The C—N bond formation between Boc-4AP(NH2)-OH and D-maltose followed the C—N bond protocol.
(ii) The amide bond formation (PEG1-ACPC-Dxd) between Boc-4AP(N(D-maltose)2)-OH and NH2-PEG1-ACPC-(C═O)—O-Dxd followed the HATU protocol.
(iii) Removal of the Boc protection group of Boc-4AP(N(D-maltose)2)-PEG1-ACPC-(C═O)—O-Dxd followed the Boc protocol.
(iv) The amide bond formation between NH2-4AP(N(D-maltose)2)-PEG1-ACPC-(C═O)—O-Dxd and Mal-PEG1-acid followed the HATU protocol.

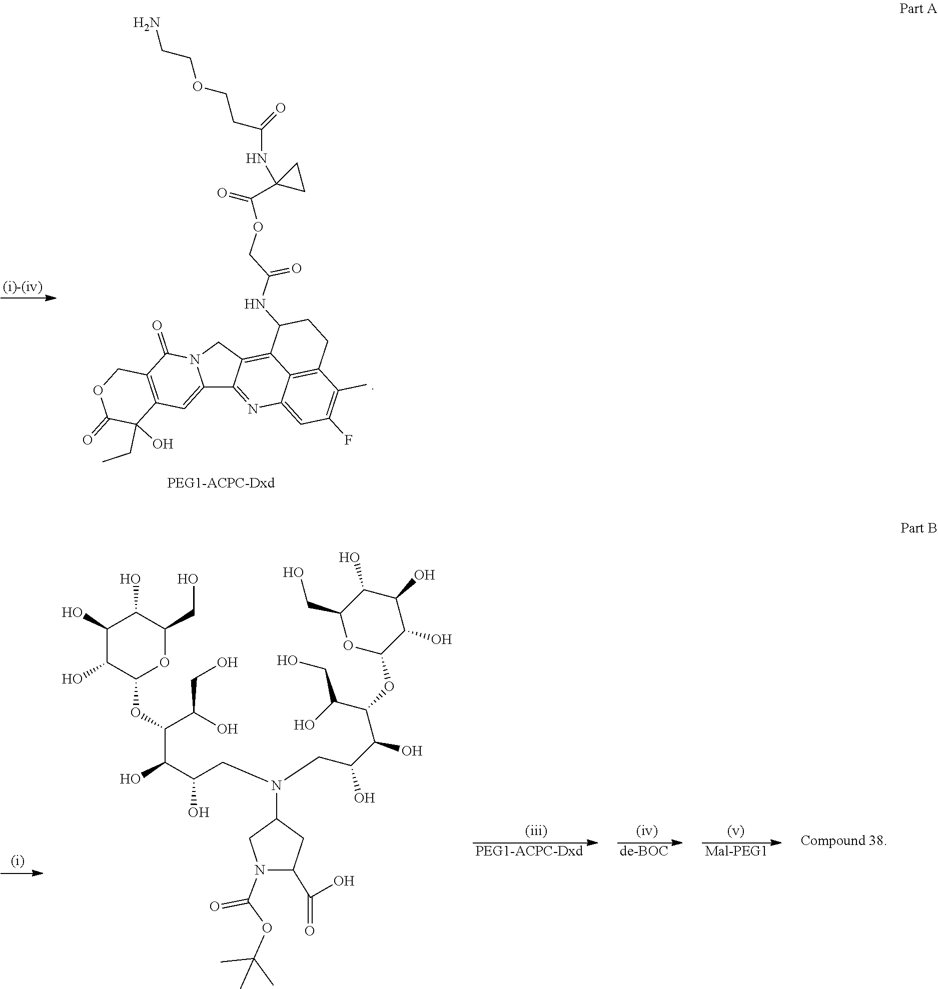

Compound 39 (Mal-PEG1-4AP(N(D-maltose)2)-PEG2-ACPC-(C═O)—O-1-propanol-3-DM) was prepared according to the following protocol:

Part A.
(i) The C—S bond formation (PrOH) between DM1 and 3-iodopropan-1-ol followed the C—S protocol.
(ii) The ester bond formation (ACPC-DM1) between DM1-3-propan-1-ol and Fmoc-ACPC followed the ester bond protocol.
(iii) Removal of the Fmoc protection group of Fmoc-ACPC-(C═O)—O-1-propanol-3-DM1 followed the Fmoc protocol.

Part B.
(i) Removal of the Boc protection group of Fmoc-4AP(Boc)-OH followed the Boc protocol.
(ii) The C—N bond formation between Fmoc-4AP(NH2)-OH and D-maltose followed the C—N bond protocol.
(iii) The amide bond formation between Fmoc-4AP(N(D-maltose)2)-OH and amino-PEG2-t-butyl ester followed the HATU protocol.
(iv) Removal of the tbu protection group of Fmoc-4AP(N(D-maltose)2)-PEG2-Otbu followed the tbu protocol.
(v) The amide bond formation (ACPC-DM1) between Fmoc-4AP(N(D-maltose)2)-PEG2-acid and NH2-ACPC-(C═O)—O-1-propanol-3-DM1 followed the HATU protocol.
(vi) Removal of the Fmoc protection group of Fmoc-4AP(N(D-maltose)2)-PEG2-ACPC-(C═O)—O-1-propanol-3-DM1 followed the Fmoc protocol.
(vii) The amide bond formation between NH2-4AP(N(D-maltose)2)-PEG2-ACPC-(C═O)—O-1-propanol-3-DM1 and Mal-PEG1-acid followed the HATU protocol.

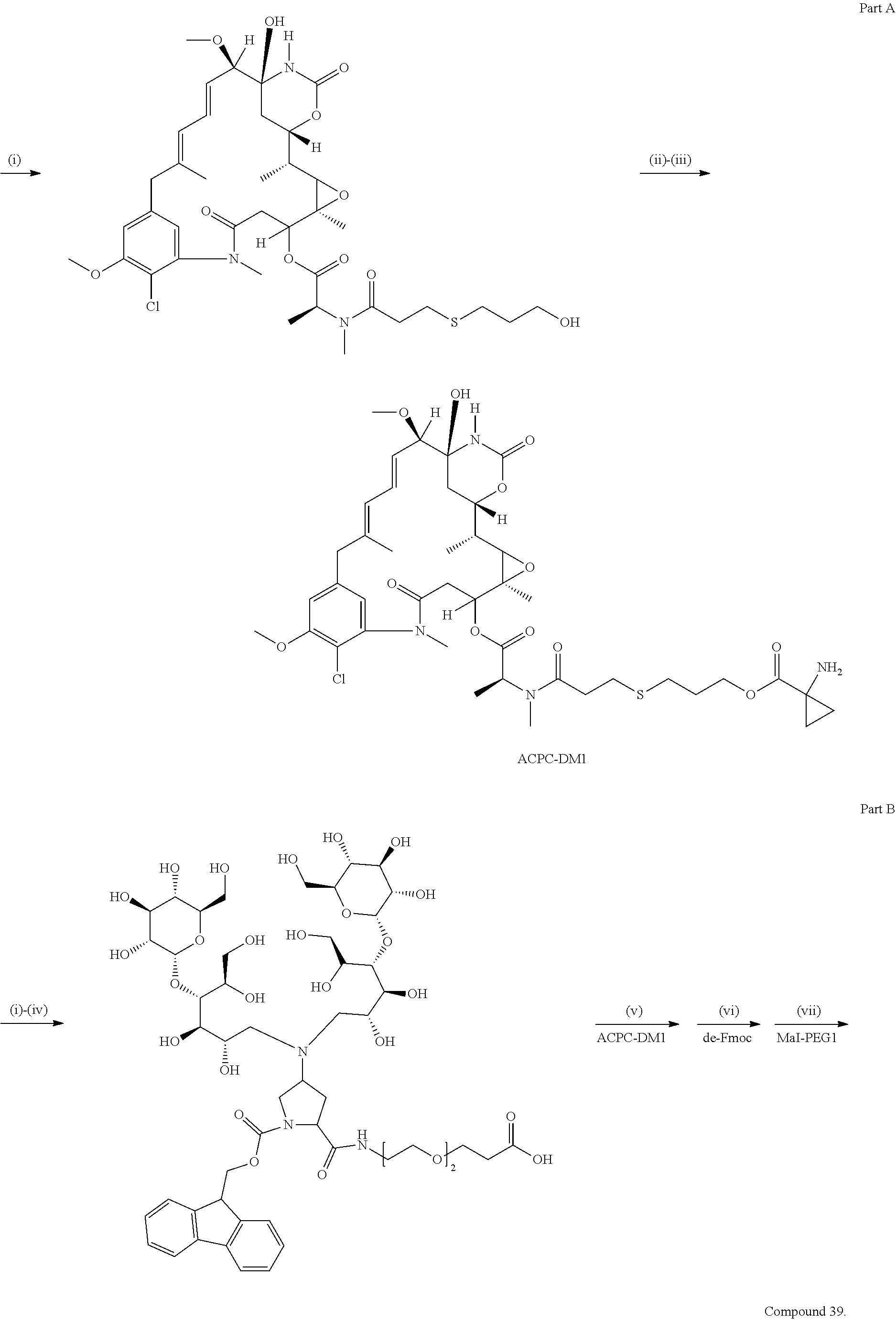

Compound 40 (Mal-PEG1-4AP(N(D-maltose)2)-PEG1-trans-AMCP-(C═O)—O-ARV-771) was prepared according to the following protocol:

Part A.
(i) The ester bond formation between ARV-771 and Boc-trans-AMCP followed the ester bond protocol.
(ii) Removal of the Boc protection group of Boc-trans-AMCP-(C═O)—O-ARV-771 followed the Boc protocol.

Part B.
(i) The C—N bond formation between Boc-4AP(NH2)-OH and D-maltose followed the C—N bond protocol.
(ii) The amide bond formation between Boc-4AP(N(D-maltose)2)-OH and amino-PEG1-acid followed the HATU protocol.
(iii) The amide bond formation between Boc-4AP(N(D-maltose)2)-PEG1-acid and NH2-trans-AMCP-(C═O)—O-ARV-771 followed the HATU protocol.
(iv) Removal of the Boc protection group of Boc-4AP(N(D-maltose)2)-PEG1-trans-AMCP-(C═O)—O-ARV-771 followed the Boc protocol.
(v) The amide bond formation between NH2-4AP(N(D-maltose)2)-PEG1-trans-AMCP-(C═O)—O-ARV-771 and Mal-PEG1-acid followed the HATU protocol.

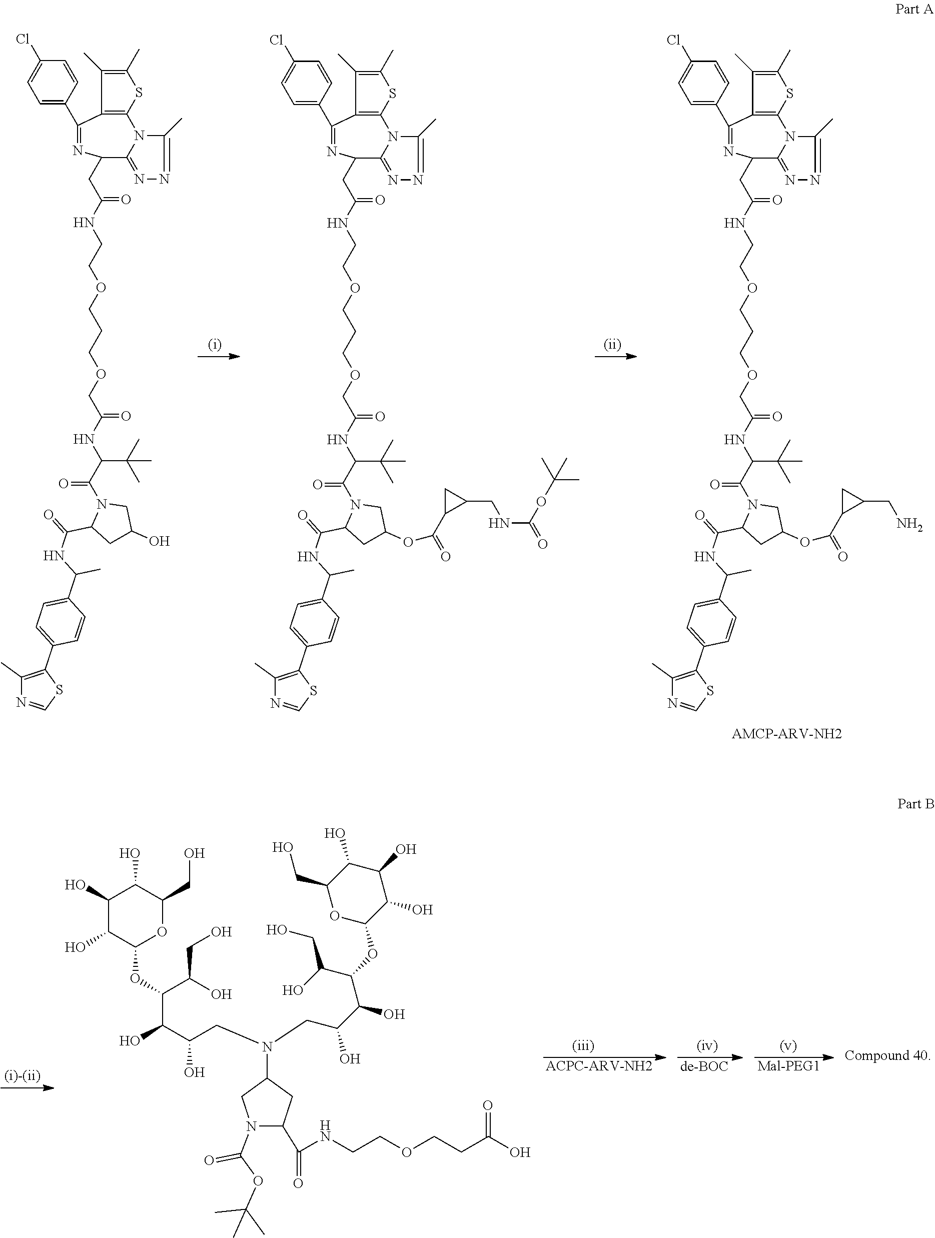

Compound 41 (Mal-PEG1-iminodiacetic acid-[Lys(N(D-maltose)2)-PEG1-L-Ser-AMAS-DM1]2) was prepared according to the following protocol: Part A, see the Part A protocol for compound 2 above.

Part B.
(i) Removal of the Boc protection group of Fmoc-Lys(Boc)-OH followed the Boc protocol.
(ii) The C—N bond formation between Fmoc-Lys(NH2)-OH and D-maltose followed the C—N bond protocol.
(iii) The amide bond formation between Fmoc-Lys(N(D-maltose)2)-OH and t-Boc-N-amido-PEG1-amine followed the HATU protocol.
(iv) Removal of the Boc protection group of Fmoc-Lys(N(D-maltose)2)-PEG1-Boc followed the Boc protocol.
(v) The amide bond formation (DM1) between Fmoc-Lys(N(D-maltose)2)-PEG1-NH2 and DM1-AMAS-L-Ser followed the HATU protocol.
(vi) Removal of the Fmoc protection group of Fmoc-Lys(N(D-maltose)2)-PEG1-L-Ser-AMAS-DM1 followed the Fmoc protocol.
(vii) The amide bond formation between NH2-Lys(N(D-maltose)2)-PEG1-L-Ser-AMAS-DM1 and Fmoc-iminodiacetic acid followed the HATU protocol.
(viii) Removal of the Fmoc protection group of Fmoc-iminodiacetic acid-[Lys(N(D-maltose)2)-PEG1-L-Ser-AMAS-DM1]2 followed the Fmoc protocol.
(ix) The amide bond formation between NH2-iminodiacetic acid-[Lys(N(D-maltose)2)-PEG1-L-Ser-AMAS-DM1]2 and Mal-PEG1-acid followed the HATU protocol.

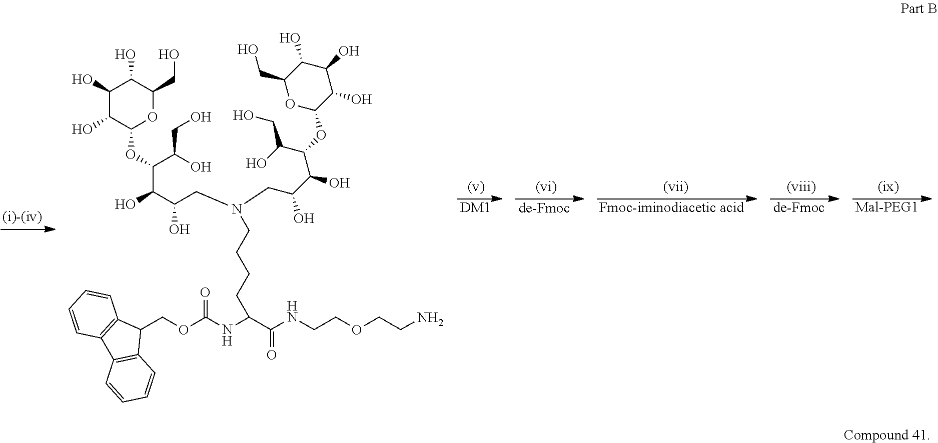

Compound 42 (Mal-PEG1-Lys(PEG1-L-Ser-AMAS-DM1)-Lys(N(D-maltose)2)-PEG1-L-Ser-AMAS-DM1) was prepared according to the following protocol:

Part A, see the Part A protocol for compound 2 above.

Part B.
(i) Removal of the Boc protection group of Fmoc-Lys(Boc)-OH followed the Boc protocol.
(ii) The amide bond formation between Fmoc-Lys(NH2)-OH and t-Boc-N-amido-PEG1-NHS followed the NHS protocol.
(iii) Removal of the Boc protection group of Fmoc-Lys(PEG1-Boc)-OH followed the Boc protocol.
(iv) The amide bond formation between Fmoc-Lys(PEG1-NH2)-OH and DM1-AMAS-L-Ser followed the NHS protocol.

Part C.
(i) Removal of the Boc protection group of Fmoc-Lys(Boc)-OH followed the Boc protocol.
(ii) The C—N bond formation between Fmoc-Lys(NH2)-OH and D-maltose followed the C—N bond protocol.
(iii) The amide bond formation between Fmoc-Lys(N(D-maltose)2)-OH and t-Boc-N-amido-PEG1-amine followed the HATU protocol.
(iv) Removal of the Boc protection group of Fmoc-Lys(N(D-maltose)2)-PEG1-Boc followed the Boc protocol.
(v) The amide bond formation between Fmoc-Lys(N(D-maltose)2)-PEG1-NH2 and DM1-AMAS-L-Ser followed the HATU protocol.
(vi) Removal of the Fmoc protection group of Fmoc-Lys(N(D-maltose)2)-PEG1-L-Ser-AMAS-DM1 followed the Fmoc protocol.
(vii) The amide bond formation between NH2-Lys(N(D-maltose)2)-PEG1-L-Ser-AMAS-DM1 and Fmoc-Lys(PEG1-L-Ser-AMAS-DM1)-OH followed the HATU protocol.
(viii) Removal of the Fmoc protection group of Fmoc-Lys(PEG1-L-Ser-AMAS-DM1)-Lys(N(D-maltose)2)-PEG1-L-Ser-AMAS-DM1 followed the Fmoc protocol.
(ix) The amide bond formation between NH2-Lys(PEG1-L-Ser-AMAS-DM1)-Lys(N(D-maltose)2)-PEG1-L-Ser-AMAS-DM1 and Mal-PEG1-acid followed the HATU protocol.

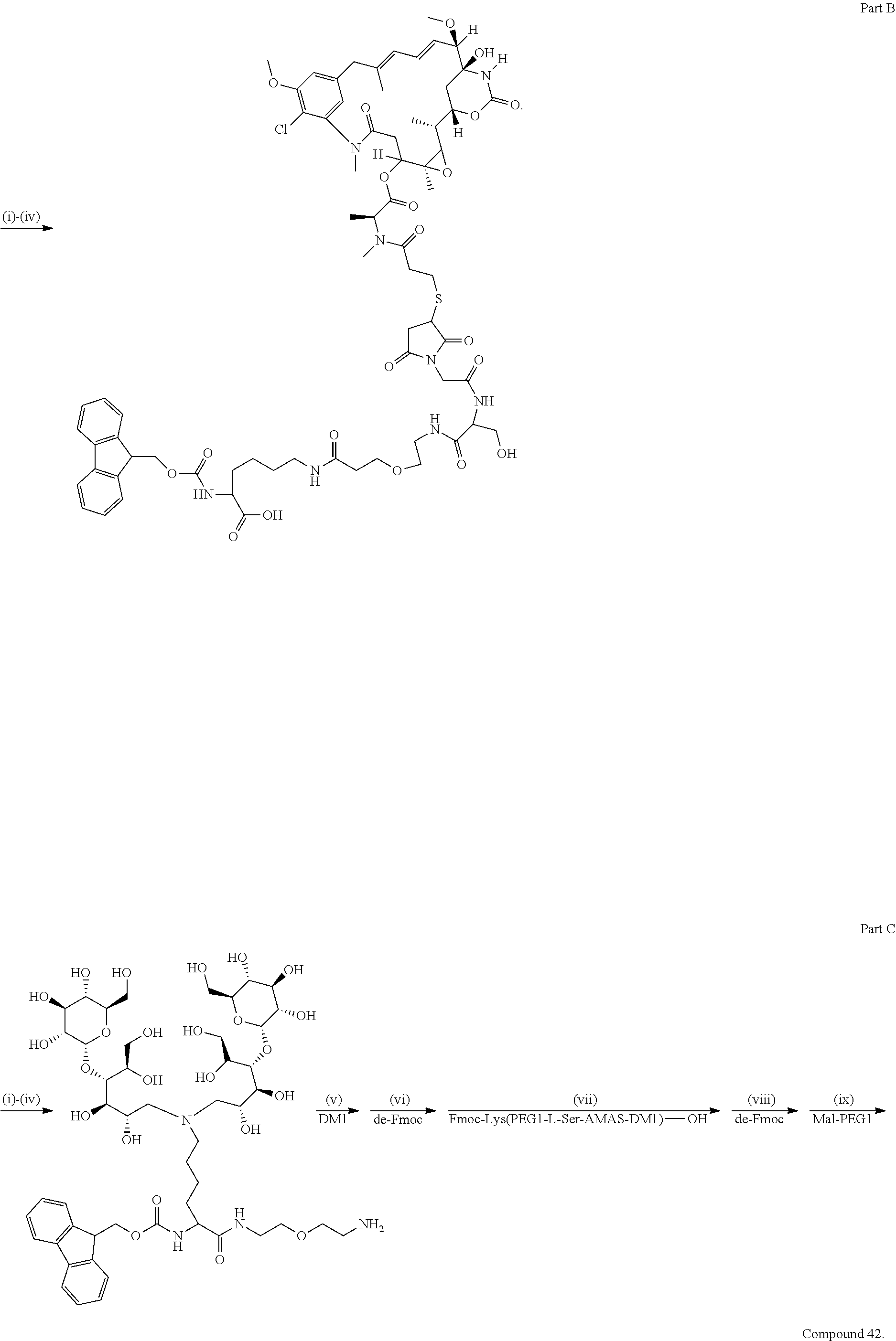

Compound 43 (Mal-PEG1-4AP(N(D-maltose)2)-PEG2-AMCP-(C═O)—O-Dxd) was prepared according to the following protocol:

Part A.
(i) The ester bond formation between Dxd and Boc-AMCP followed the ester bond protocol.
(ii) Removal of the Boc protection group of Boc-AMCP-(C═O)—O-Dxd followed the Boc protocol.

Part B.
(i) The C—N bond formation between Boc-4AP(NH2)-OH and D-maltose followed the C—N bond protocol.
(ii) The amide bond formation between Boc-4AP(N(D-maltose)2)-OH and amino-PEG2-acid followed the HATU protocol.
(iii) The amide bond formation between Boc-4AP(N(D-maltose)2)-PEG2-acid and NH2-trans-AMCP-(C═O)—O-Dxd followed the HATU protocol.
(iv) Removal of the Boc protection group of Boc-4AP(N(D-maltose)2)-PEG2-trans-AMCP-(C═O)—O-Dxd followed the Boc protocol.
(v) The amide bond formation between NH2-4AP(N(D-maltose)2)-PEG2-trans-AMCP-(C═O)—O-Dxd and Mal-PEG1-acid followed the HATU protocol.

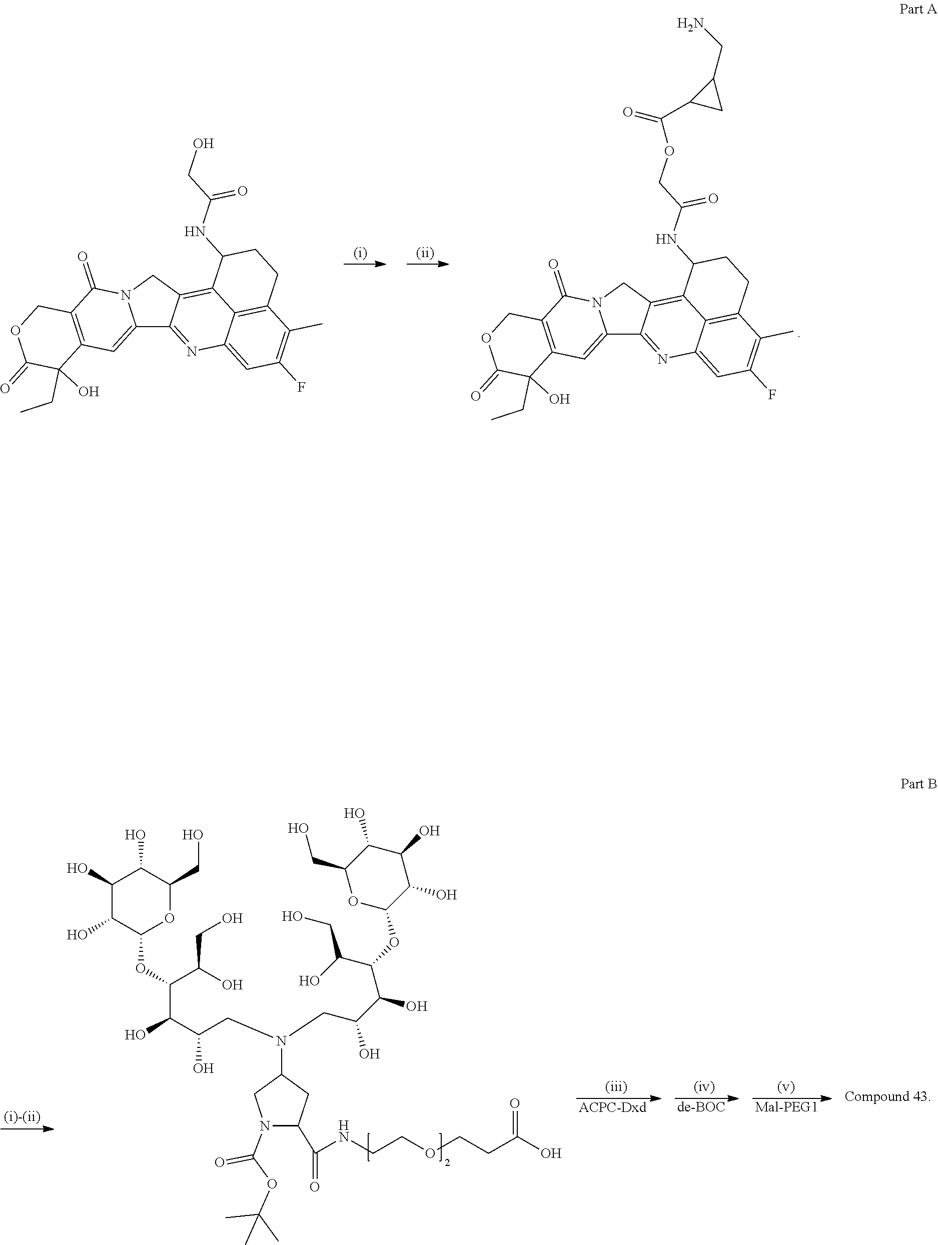

Compound 44 (Mal-PEG1-4AP(N(D-maltose)2)-PEG2-Val-Cit-PAB-MMAE) was prepared according to the following protocol:
(i) Removal of the Boc protection group of Fmoc-4AP(Boc)-OH followed the Boc protocol.
(ii) The C—N bond formation between Fmoc-4AP(NH2)-OH and D-maltose followed the C—N bond protocol.
(iii) The amide bond formation between Fmoc-4AP(N(D-maltose)2)-OH and amino-PEG2-t-butyl ester followed the HATU protocol.
(iv) Removal of the tbu protection group of Fmoc-4AP(N(D-maltose)2)-PEG2-Otbu followed the tbu protocol.
(v) The amide bond formation between Fmoc-4AP(N(D-maltose)2)-PEG2-acid and Val-Cit-PAB-MMAE followed the HATU protocol.
(vi) Removal of the Fmoc protection group of Fmoc-4AP(N(D-maltose)2)-PEG2-Val-Cit-PAB-MMAE followed the Fmoc protocol.
(vii) The amide bond formation between NH2-4AP(N(D-maltose)2)-PEG2-Val-Cit-PAB-MMAE and Mal-PEG1-acid followed the HATU protocol.

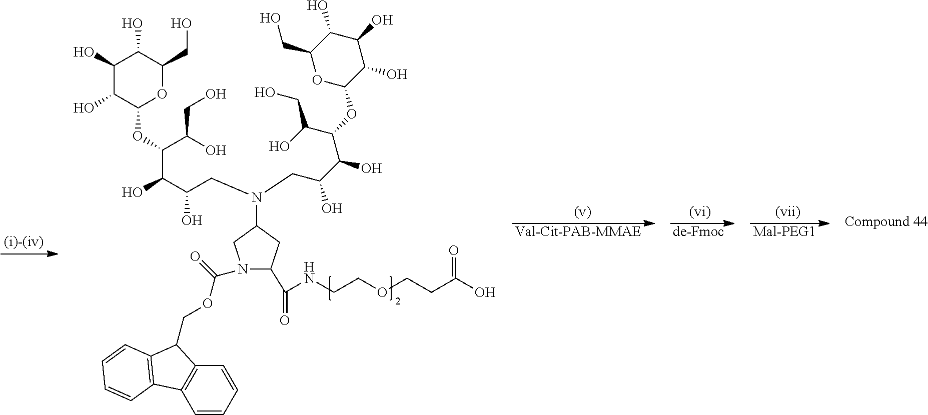

Compound 45 (Mal-PEG1-Dap(glucuronic acid)-Dap(glucuronic acid)-PEG1-L-Ser-AMAS-DM1) was prepared according to the following protocol:

Part A, see the Part A protocol for compound 2 above.

Part B.
(i) The amide bond formation between Fmoc-Dap(Boc)-OH and H-Dap(Boc)-OH followed the HATU protocol.
(ii) Removal of the Boc protection group of Fmoc-Dap(Boc)-Dap(Boc)-OH followed the Boc protocol.
(iii) The amide bond formation between Fmoc-Dap(NH2)-Dap(NH2)-OH and glucuronic acid followed the HATU protocol.
(iv) The amide bond formation between Fmoc-Dap(glucuronic acid)-Dap(glucuronic acid)-OH and t-Boc-N-amido-PEG1-amine followed the HATU protocol.
(v) Removal of the Boc protection group of Fmoc-Dap(glucuronic acid)-Dap(glucuronic acid)-PEG1-Boc followed the Boc protocol.
(vi) The amide bond formation between Fmoc-Dap(glucuronic acid)-Dap(glucuronic acid)-PEG1-NH2 and DM1-AMAS-L-Ser followed the HATU protocol.
(vii) Removal of the Fmoc protection group of Fmoc-Dap(glucuronic acid)-Dap(glucuronic acid)-PEG1-L-Ser-AMAS-DM1 followed the Fmoc protocol.
(viii) The amide bond formation between NH2-Dap(glucuronic acid)-Dap(glucuronic acid)-PEG1-L-Ser-AMAS-DM1 and Mal-PEG1-acid followed the HATU protocol.

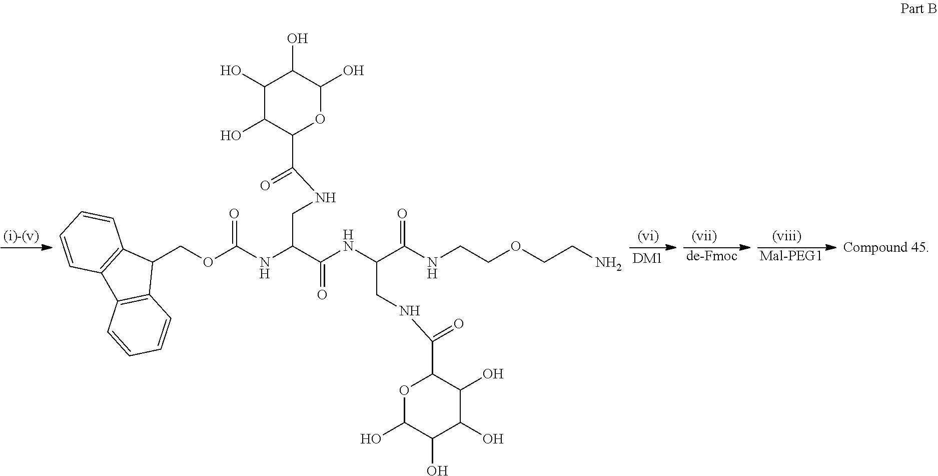

Compound 46 (Boc-4AP(N(D-maltose)2)-PEG1-CPCA-(C═O)—O-Dxd) was prepared according to the following protocol:

Part A.
(i) The ester bond formation between Dxd and CPCA-tbu (rel-(1R,2R)-2-(tert-Butoxycarbonyl)cyclopropane-1-carboxylic acid) followed the ester bond protocol.
(ii) Removal of the tbu protection group of tbu-CPCA-(C═O)—O-Dxd followed the tbu protocol.

Part B.
(i) The C—N bond formation between Boc-4AP(NH2)-OH and D-maltose followed the C—N bond protocol.
(ii) The amide bond formation between Boc-4AP(N(D-maltose)2)-OH and Fmoc-N-amido-PEG1-amine [(9H-Fluoren-9-yl)methyl (2-(2-aminoethoxy)ethyl)carbamate]followed the HATU protocol.
(iii) Removal of the Fmoc protection group of Boc-4AP(N(D-maltose)2)-PEG1-Fmoc followed the Fmoc protocol.
(iv) The amide bond formation between Boc-4AP(N(D-maltose)2)-PEG1-NH2 and CPCA-(C═O)—O-Dxd followed the HATU protocol.

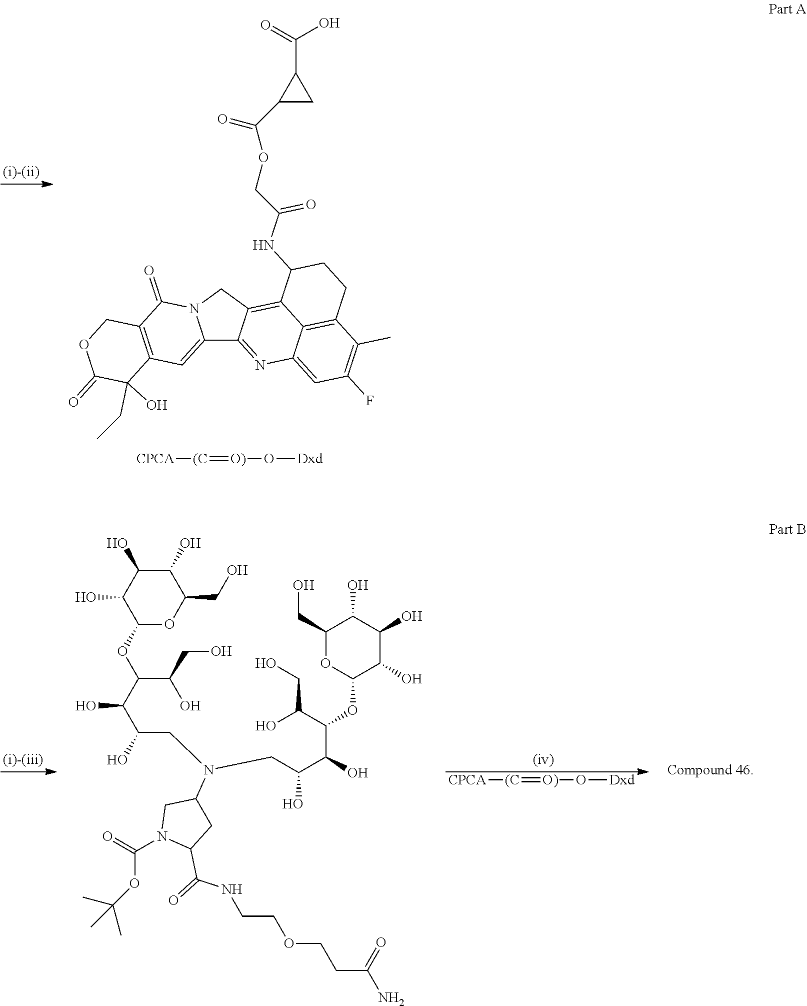

Another aspect of this invention relates to ligand-drug conjugates each containing a ligand moiety and a moiety derived from any one of the compounds described above. The ligand is bonded to the compound via a covalent bond formed between a functional group from the ligand and LL in formula I, and the functional group is sulfhydryl, amino, glutamine, formyl, or Keto.

The exemplary ligand is Trastuzumab and Sacituzumab. The covalent bond is formed between sulfhydryl of the ligand and the maleimide moiety of LL.

Typically, the molar ratio of the ligand to the compound is between 1:1 and 1:20, preferably between 1:2 and 1:16.

Control Conjugates 1-5 and exemplary conjugates 6-32 of this invention were prepared from a ligand and a drug linker using one of the protocols described above are shown below.

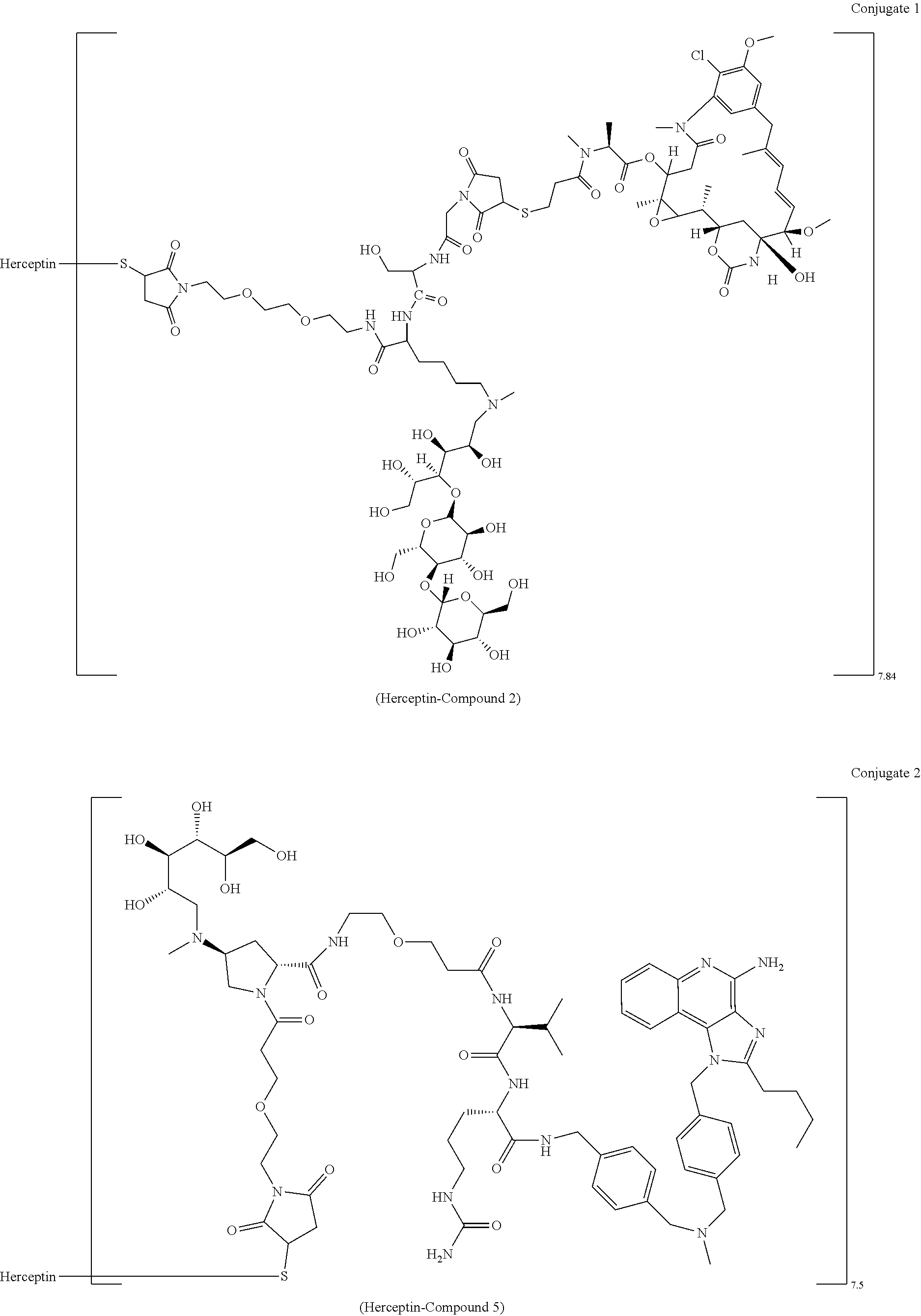

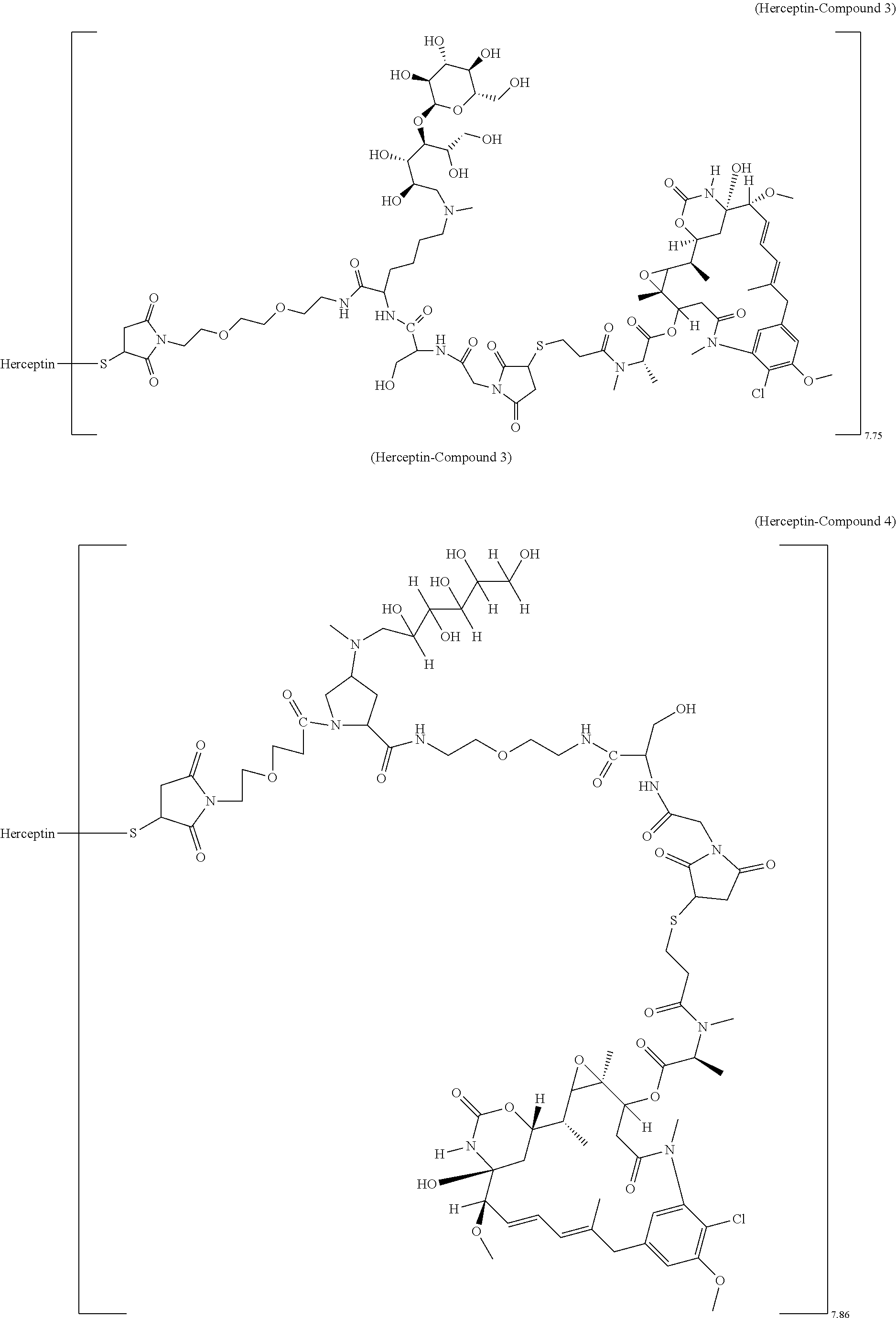

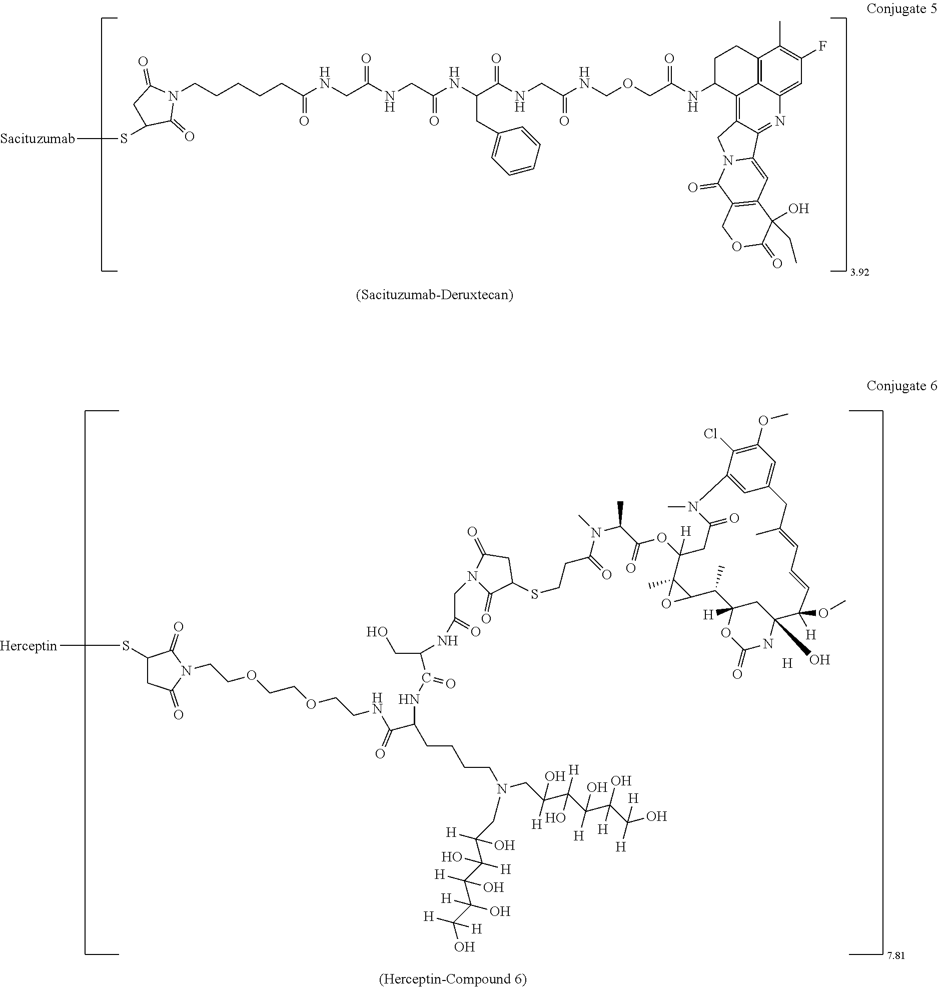

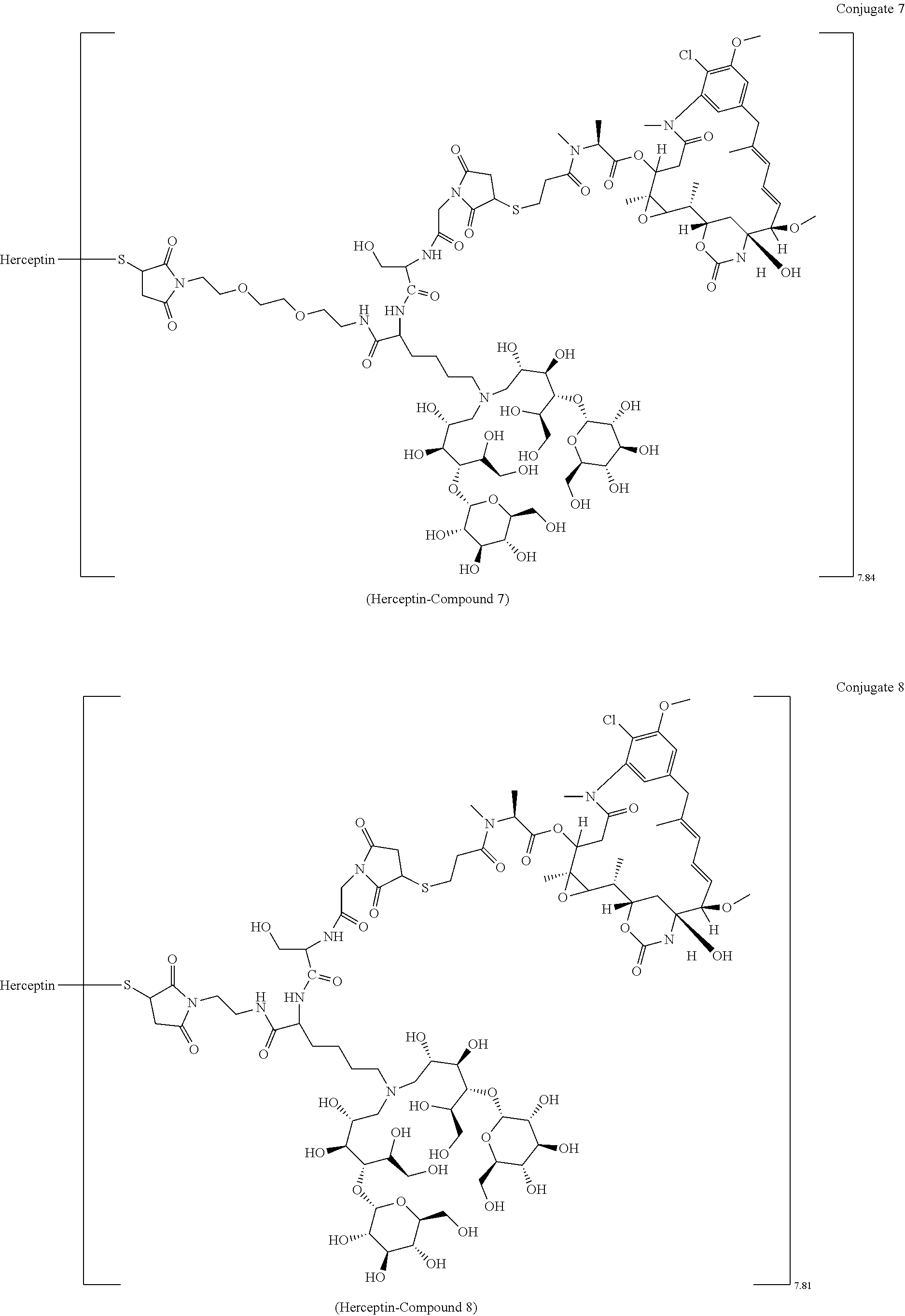

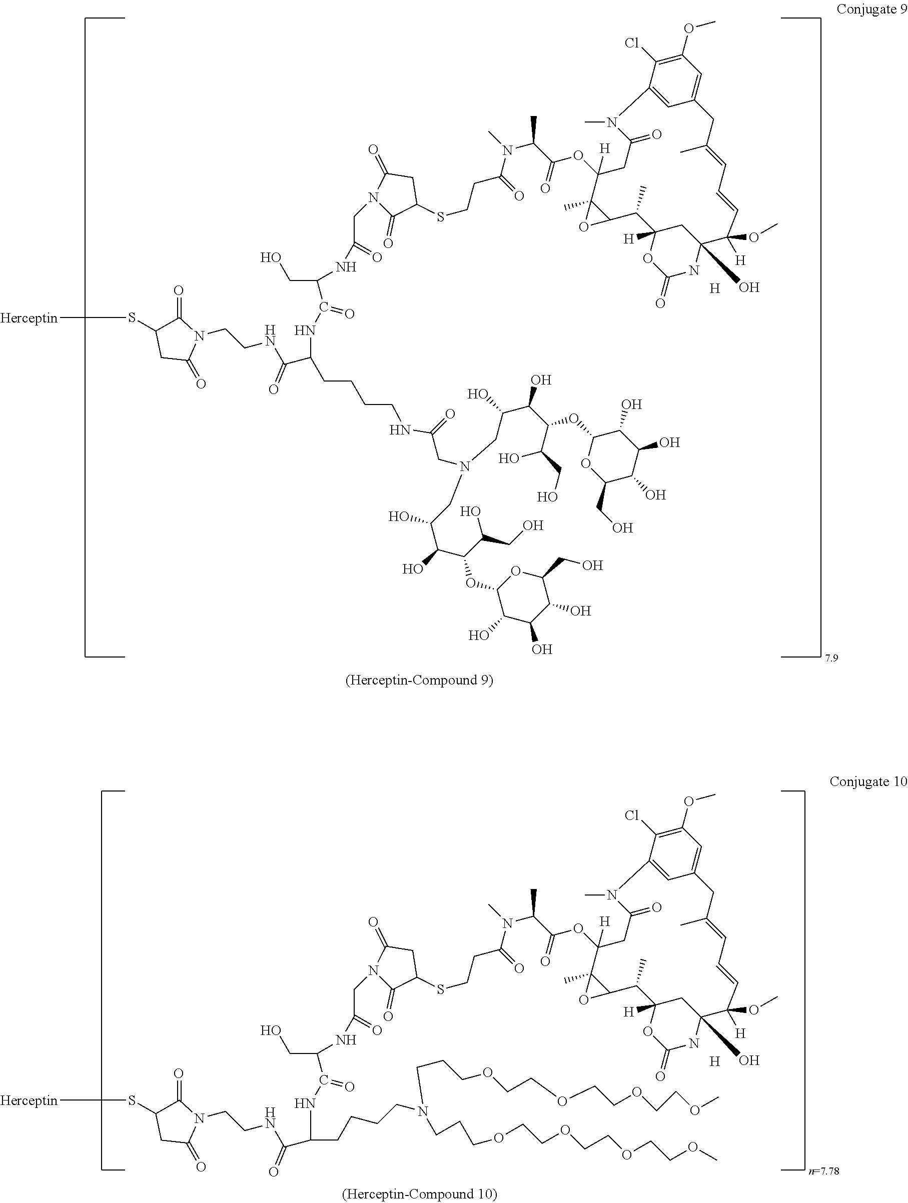

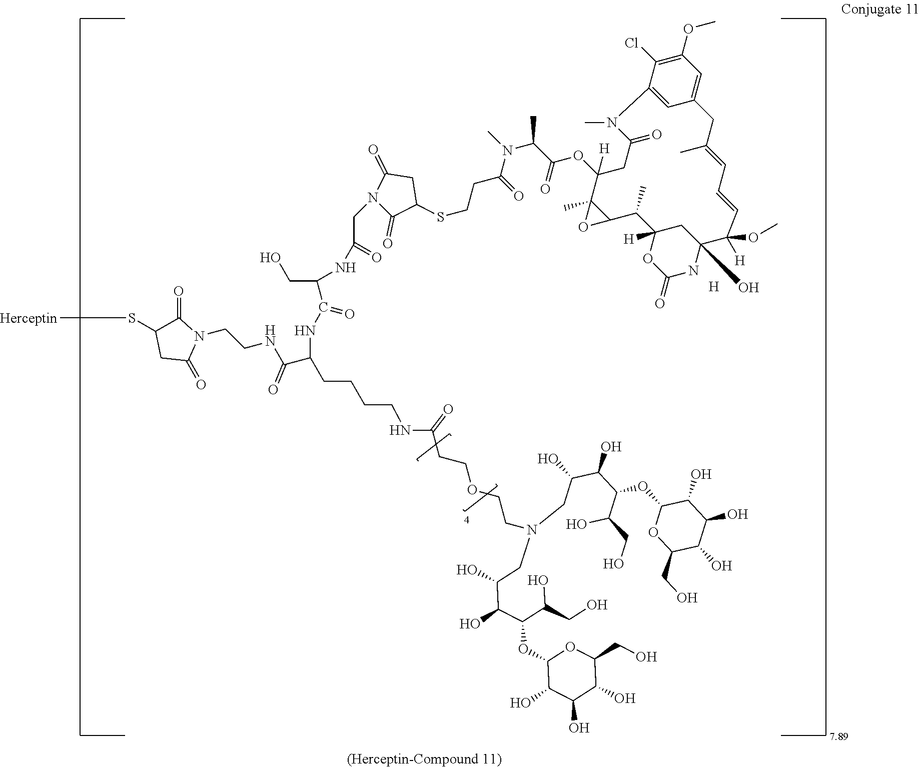

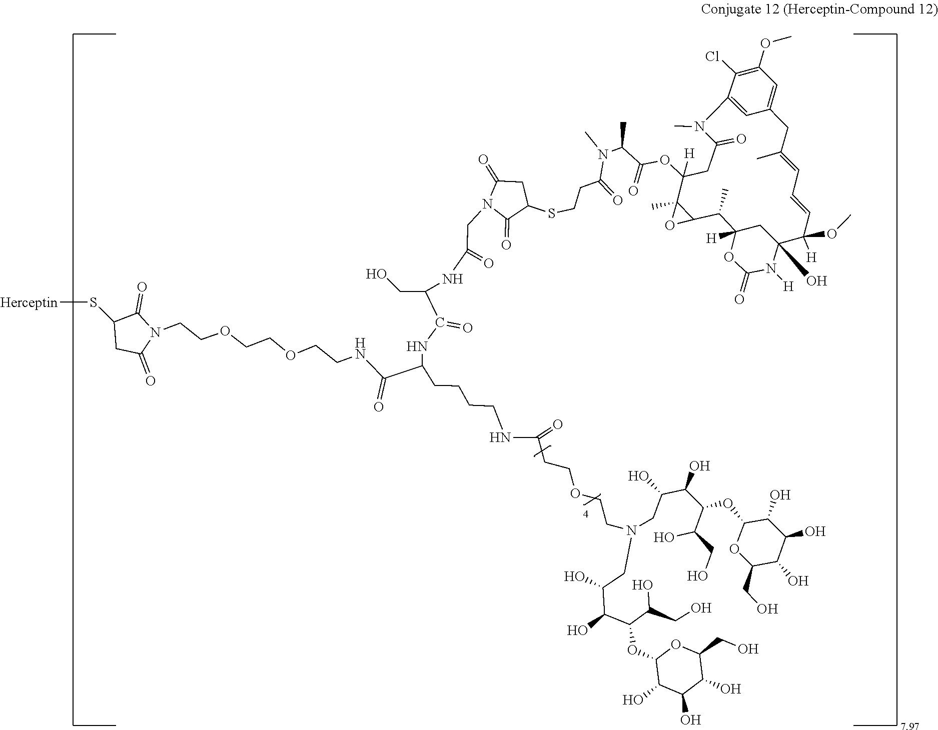

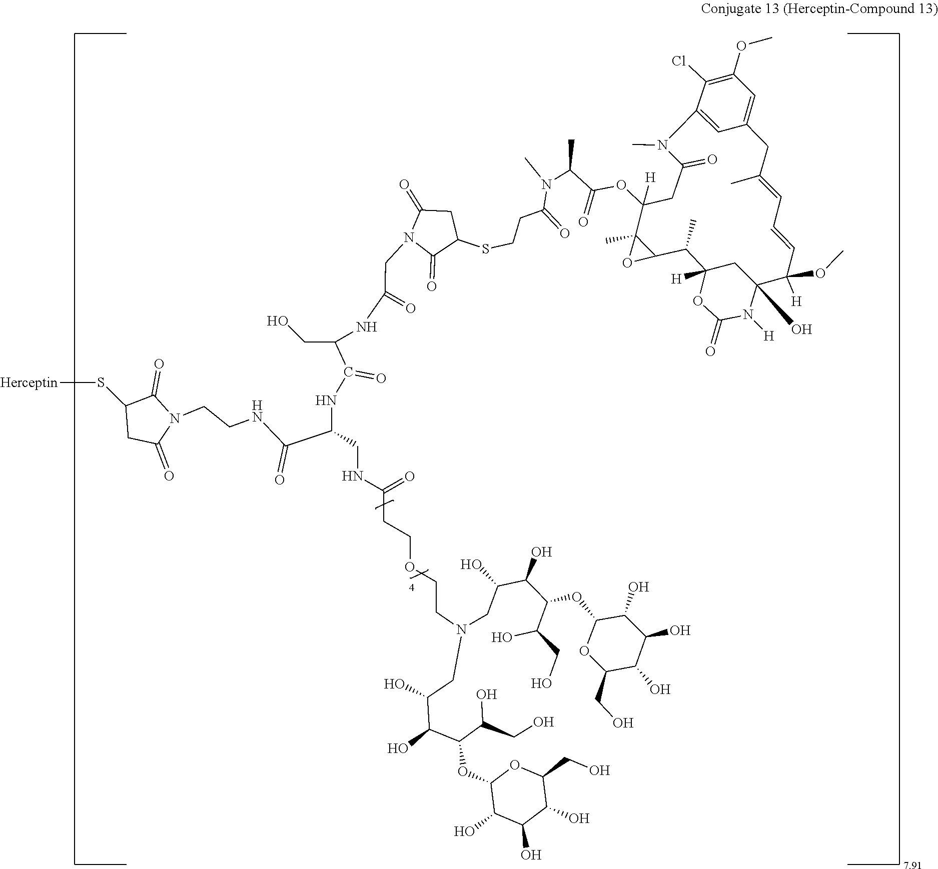

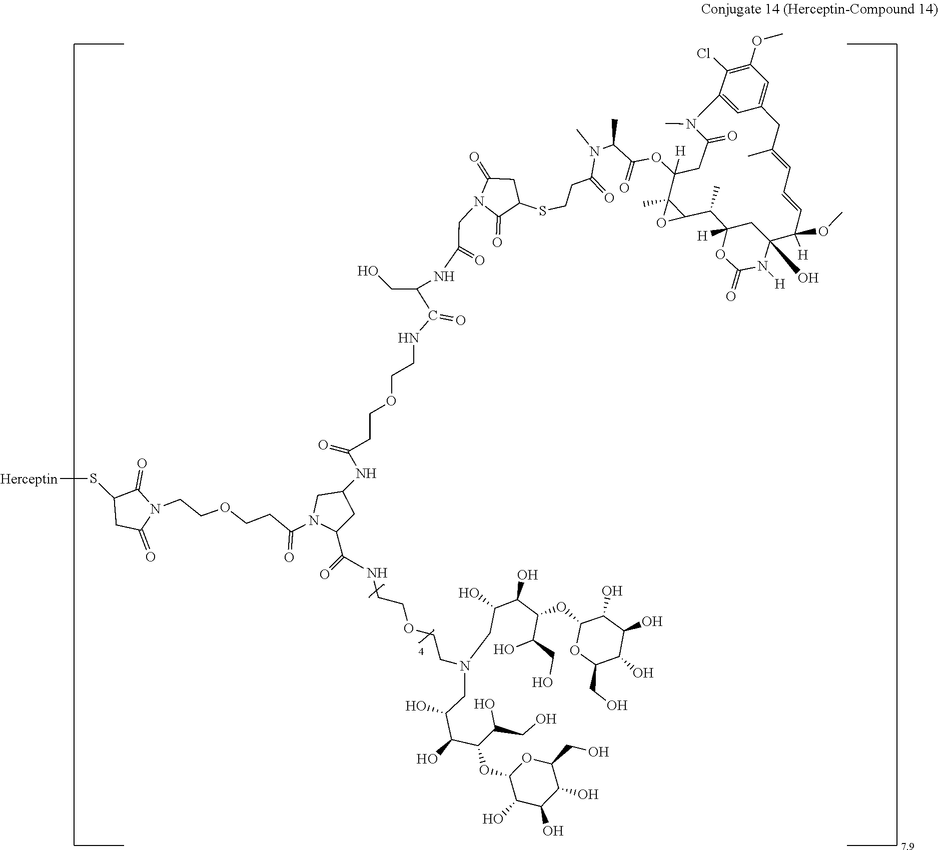

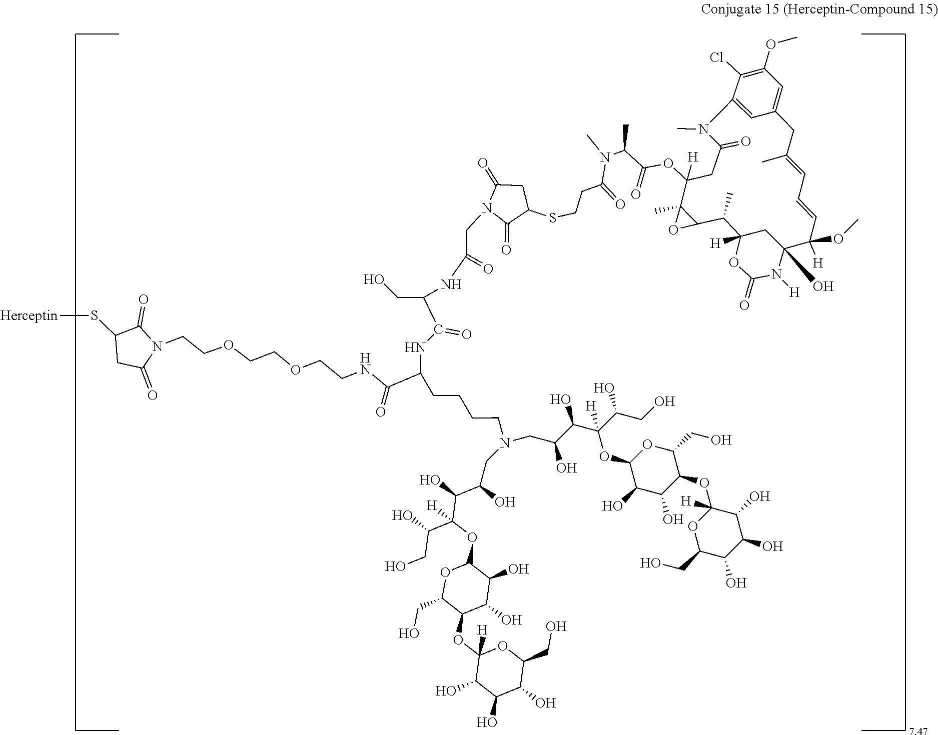

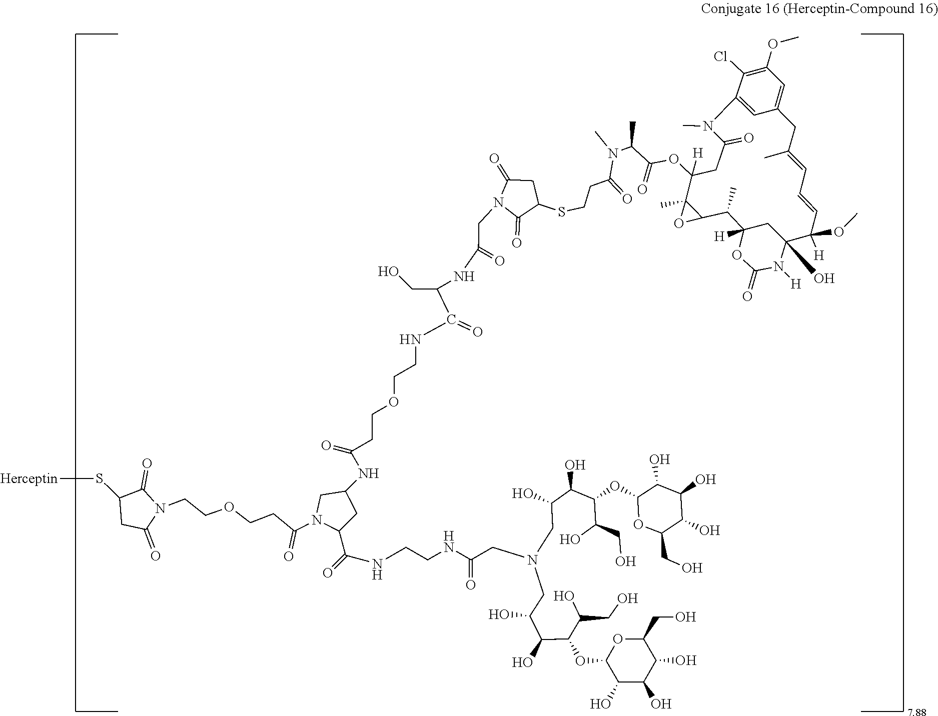

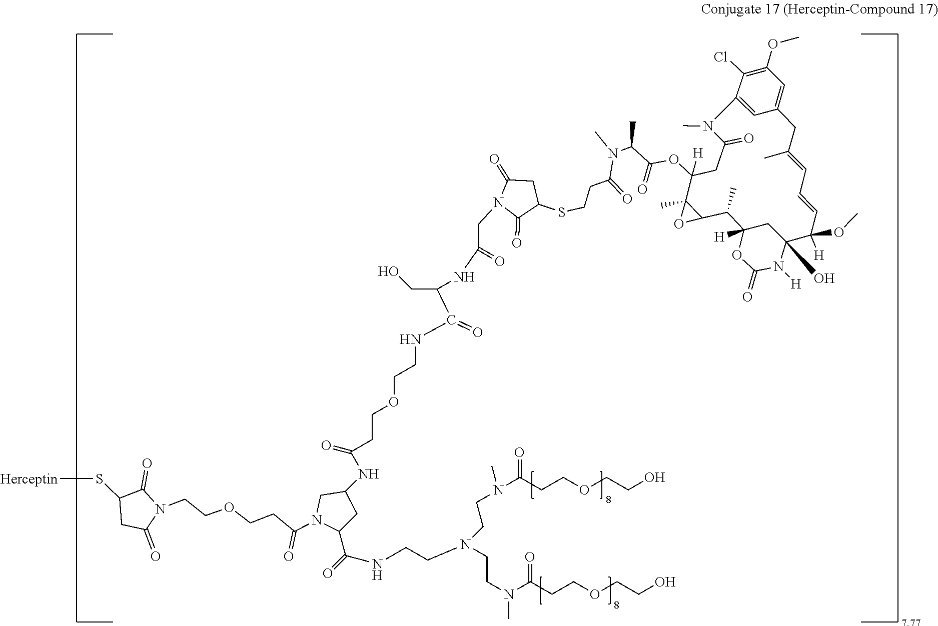

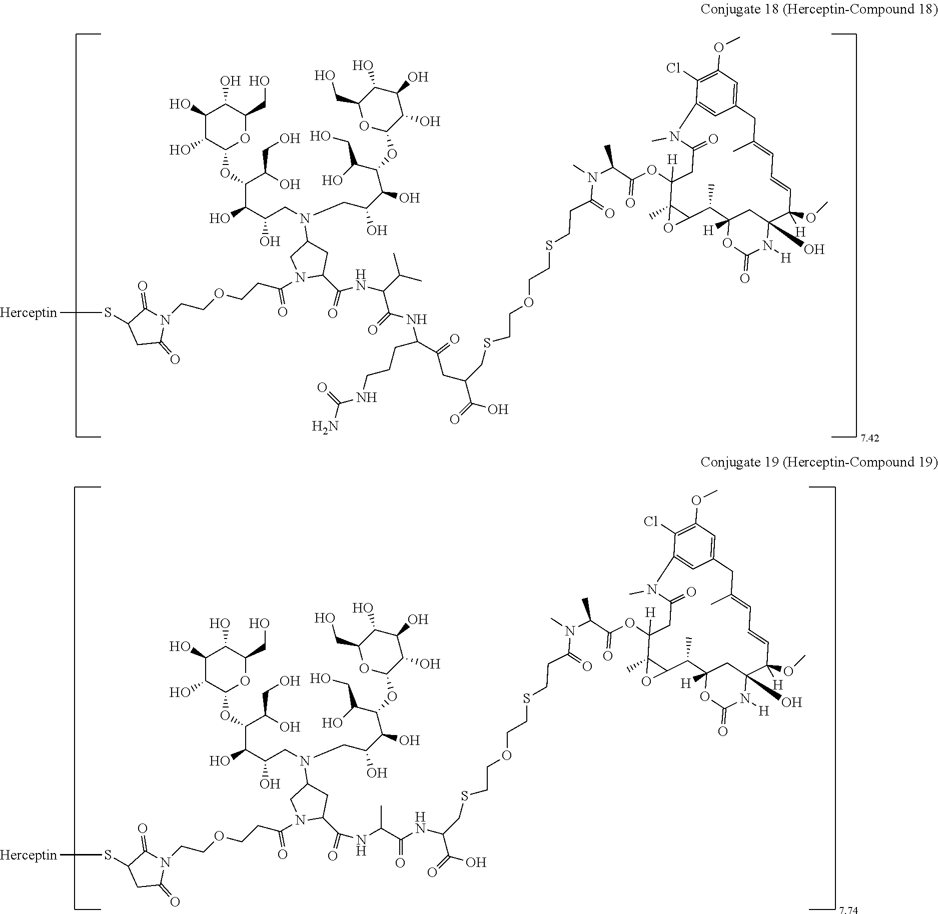

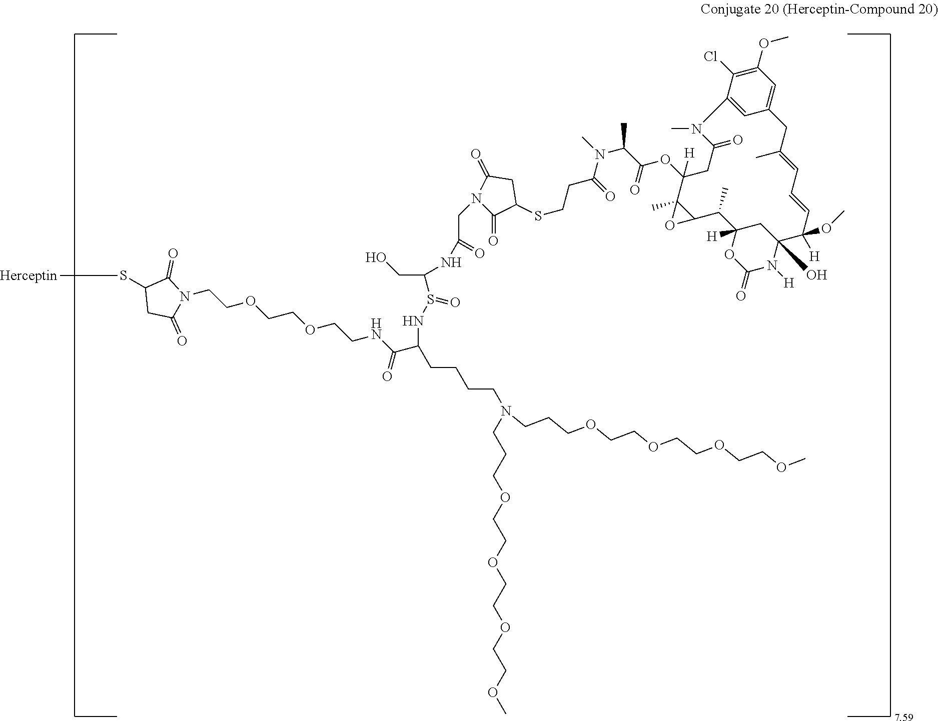

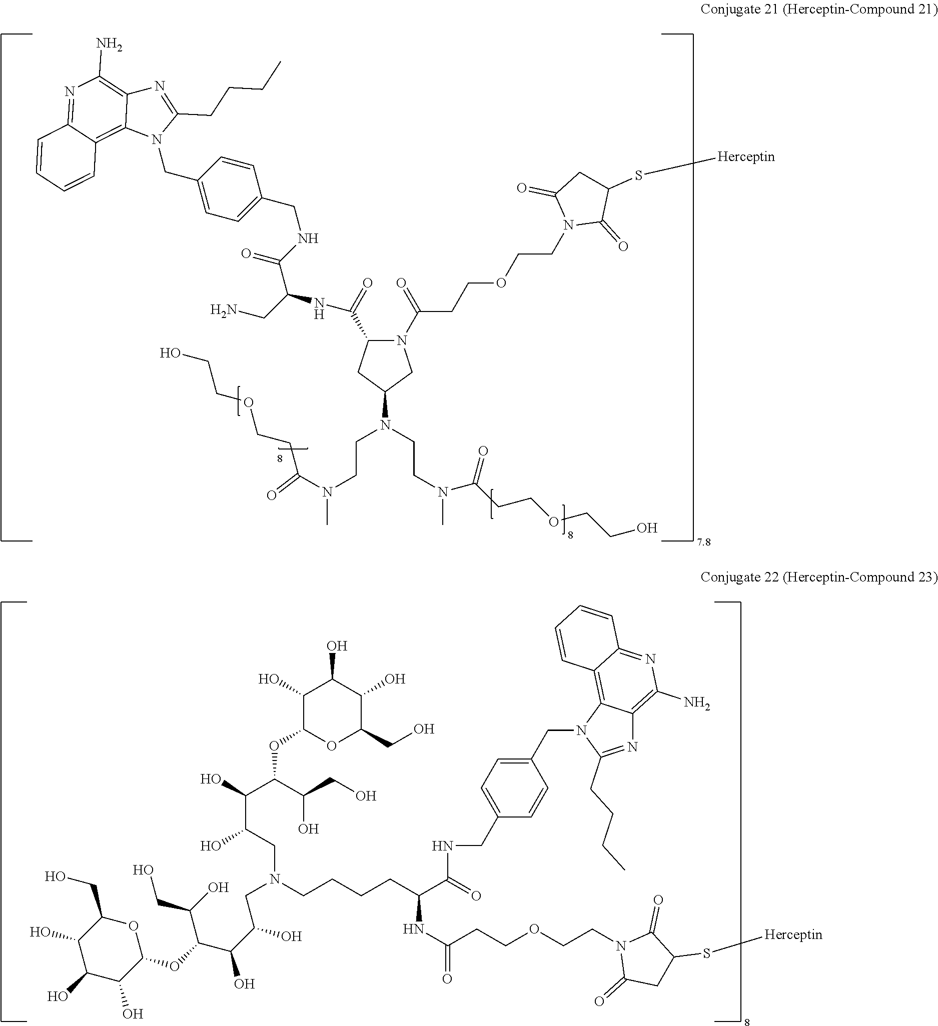

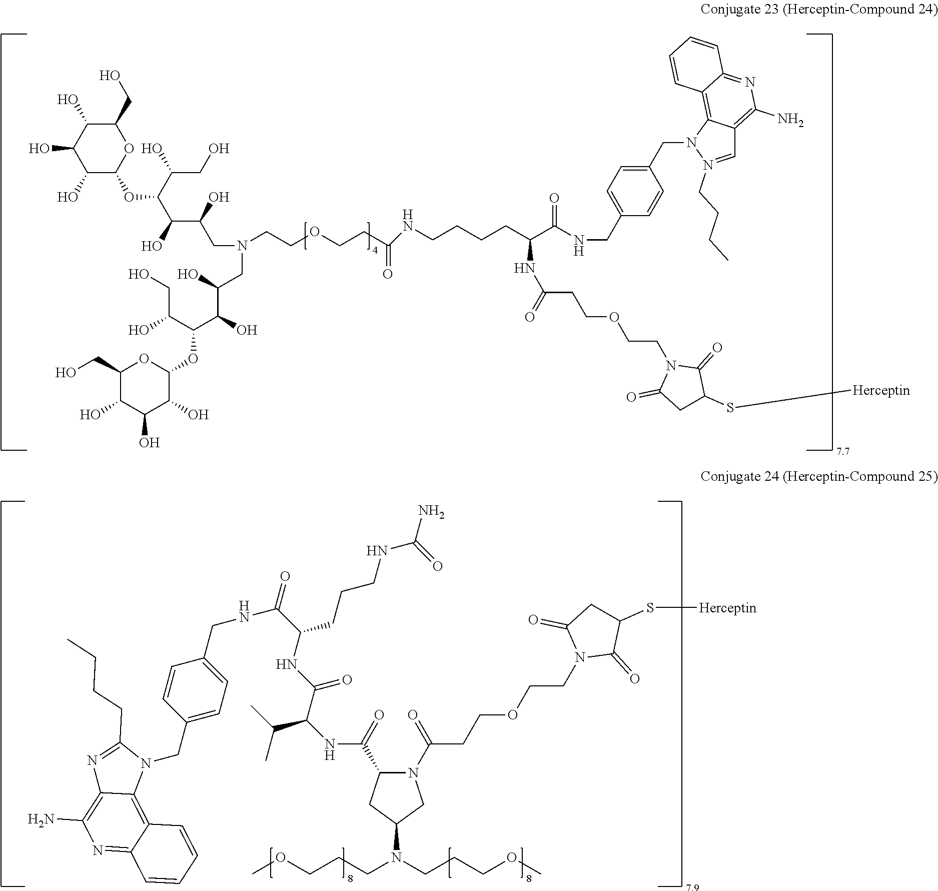

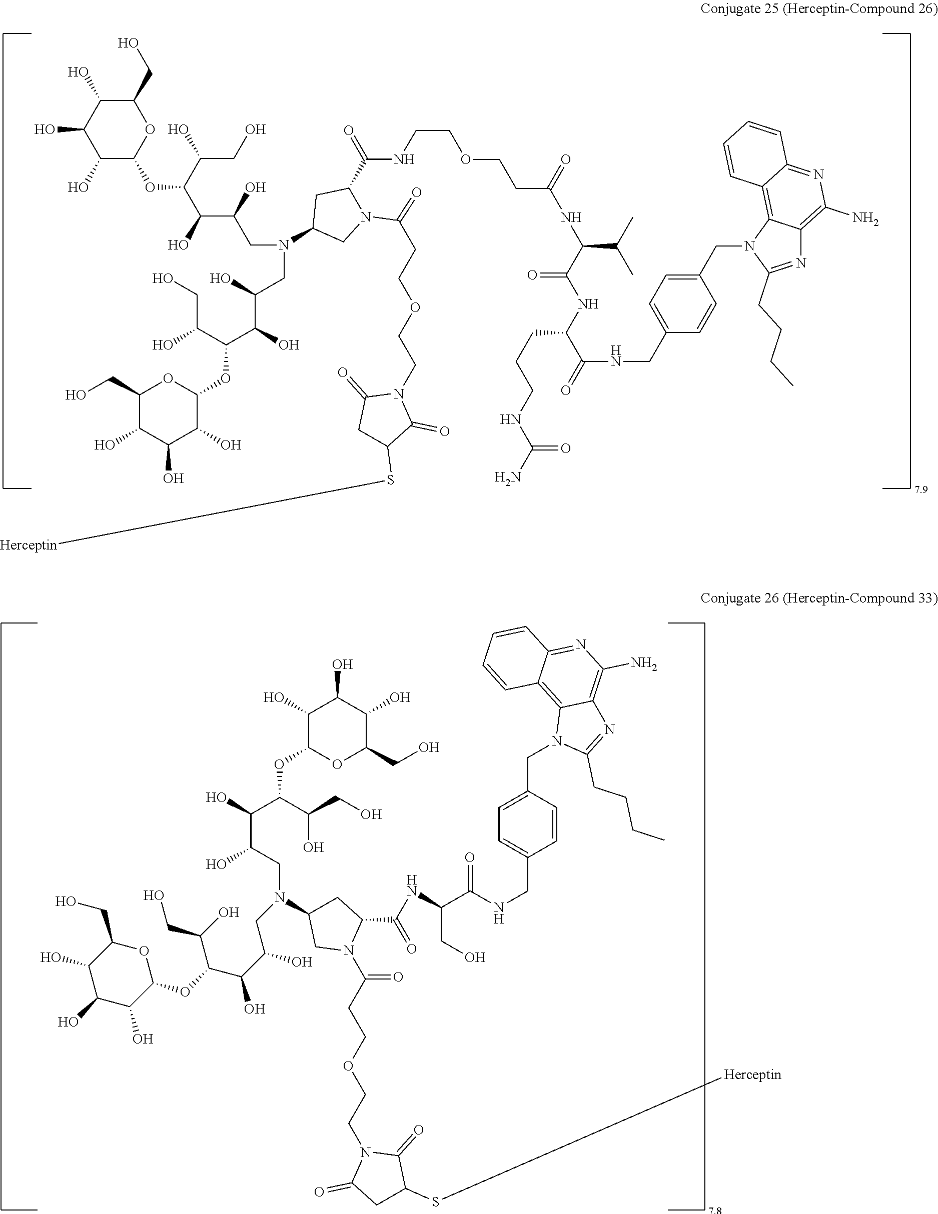

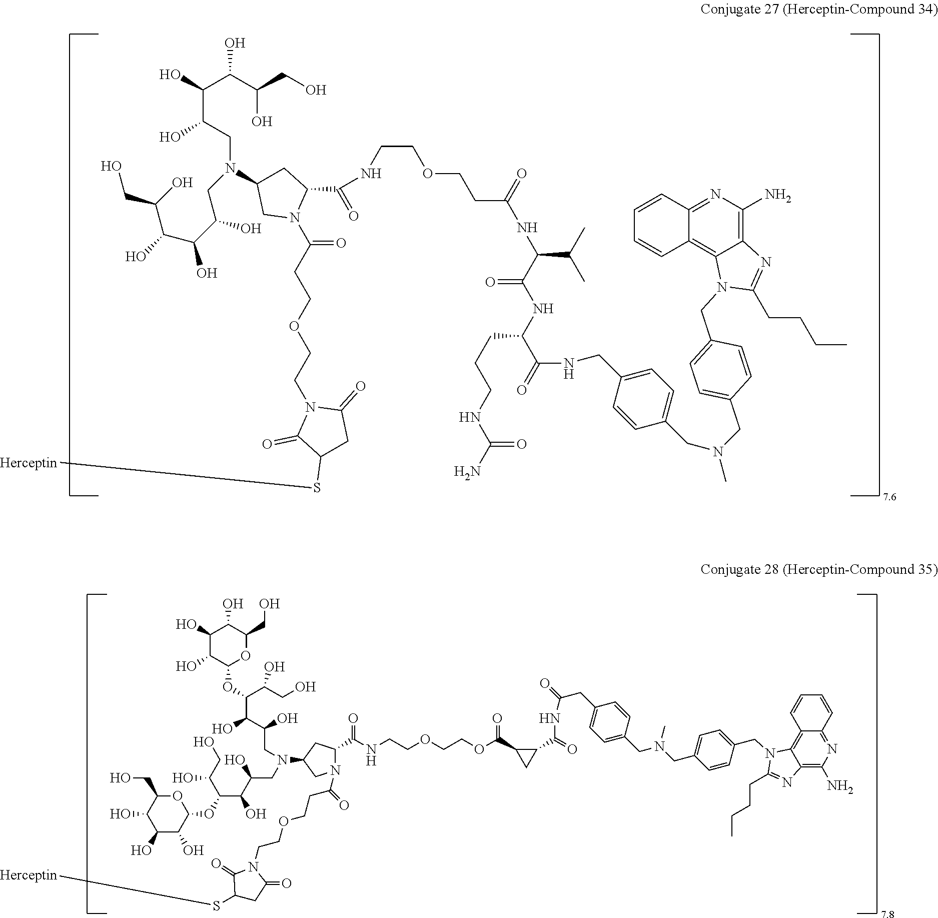

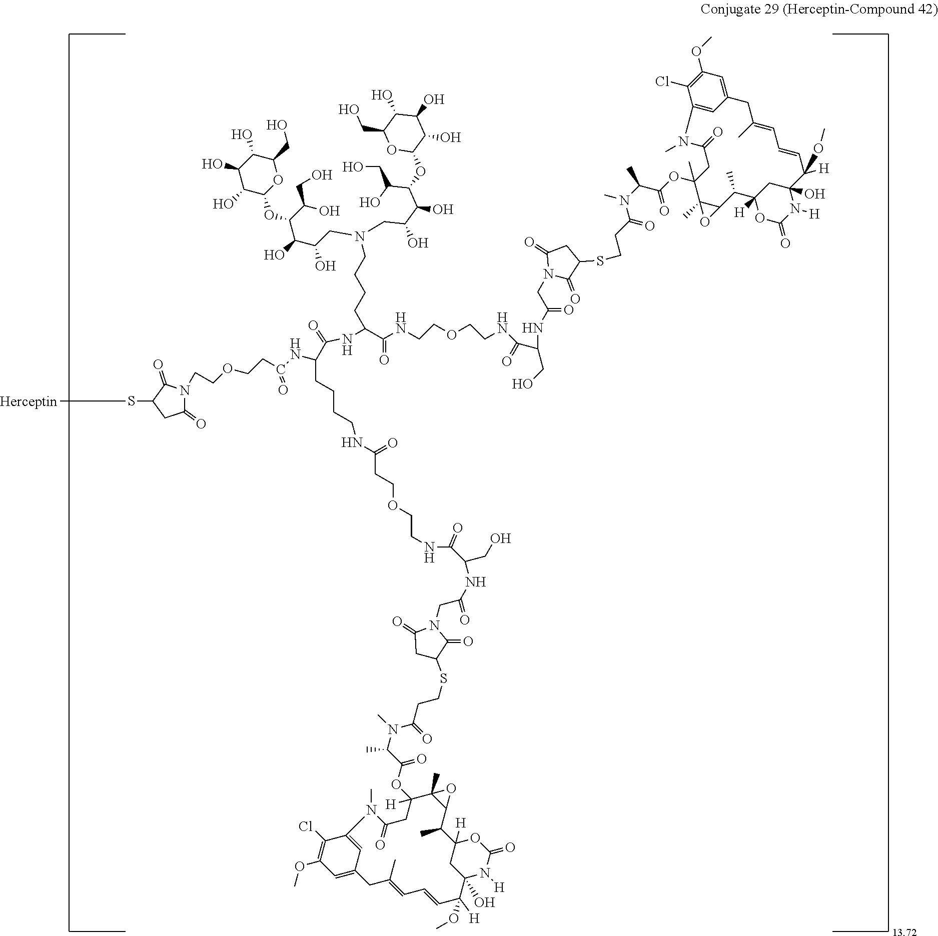

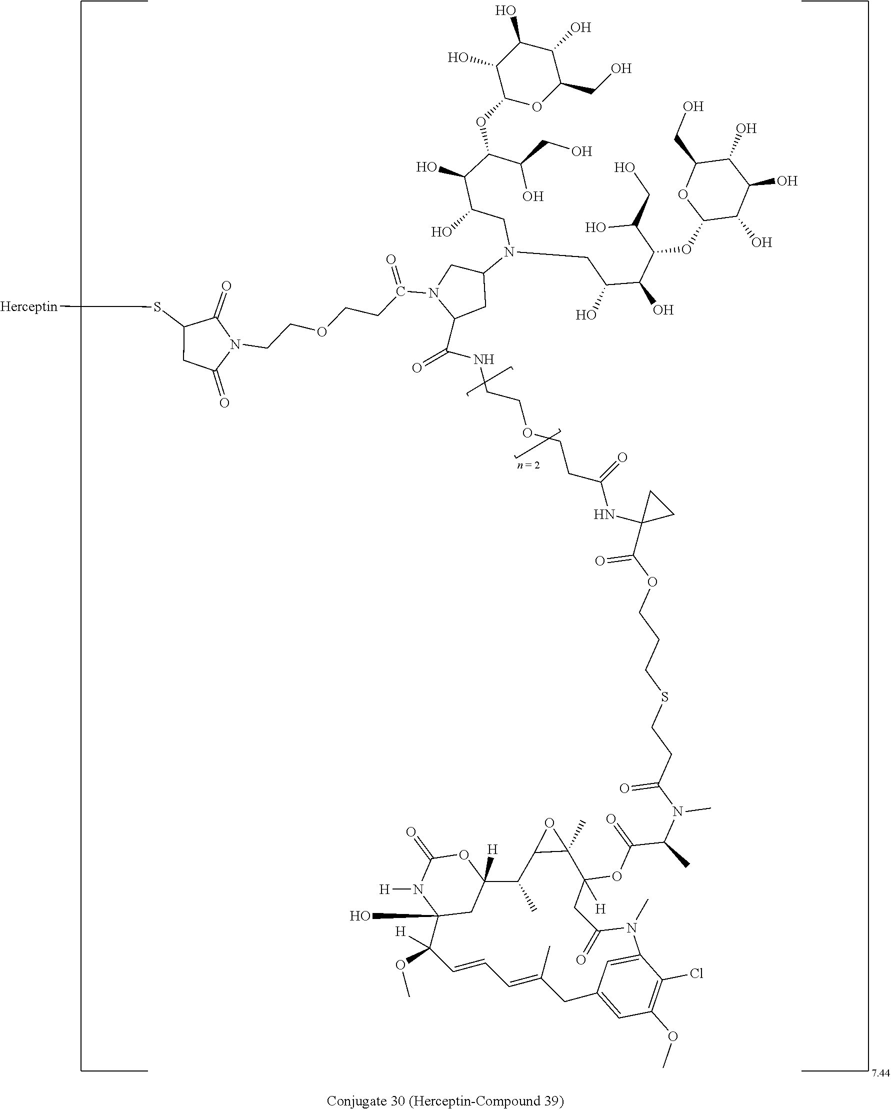

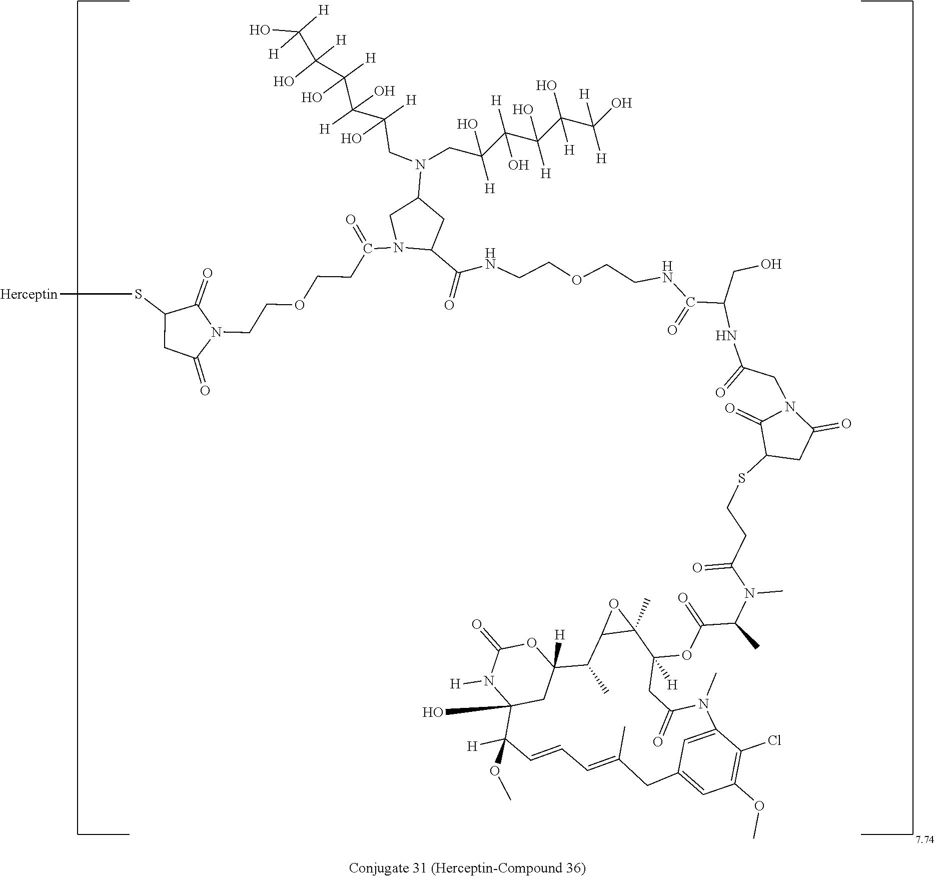

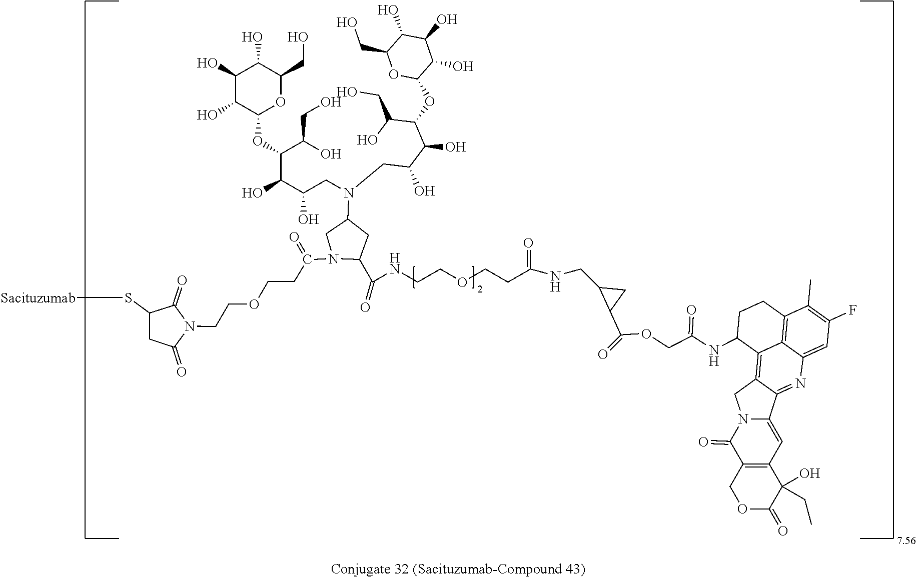

Also within the scope of this invention is a method of treating cancer including the step of administrating to a patient in need thereof an effective amount of any one of the conjugates described above.

The method can be used to treat any cancer. Nonlimiting examples include hematopoietic cancers such as, for example, lymphomas (Hodgkin Lymphoma and Non-Hodgkin Lymphomas) and leukemias and solid tumors. Examples of hematopoietic cancers include, follicular lymphoma, anaplastic large cell lymphoma, mantle cell lymphoma, acute myeloblastic leukemia, chronic myelocytic leukemia, chronic lymphocytic leukemia, diffuse large B cell lymphoma, and multiple myeloma. Examples of solid tumors include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

### Two HPs Greatly Augmented the Serum Stability of Linker-Drug Complex

The experiment was performed as described in the section of Materials and Methods. Two linker-drug complexes (Compound 1 and 46, respectively) were prepared with an ester bond as the fifth connector, i.e., the bond between linker and payload. Compound 1 contains no HP, while 46 two HPs, each consisting of maltose. As shown in Table 1 below, the stability of 1 and 46 in mouse serum greatly differed. Two HPs augmented the half-life of Compound 46 more than 7 folds, compared to that of Compound 1.

TABLE 1
Half-life of Compound 1 and 46.

| Linker-drug Complex | t_{1/2} (hours) |
|---|---|
| Compound 1 | 2.6 |
| Compound 46 | 18.8 |

Two HPs reduced ADC Aggregation

Conjugate 3 (DAR-7.75 with one HP moiety), Conjugate 6 (DAR˜7.81 with two HP moieties), and trastuzumab emtansine (Kadcyla, DAR˜3.4 with no HP moiety) were analyzed for aggregation.

The experiment was performed as described in the section of Materials and Methods. A 10 ul of each ADC sample (5 mg/ml) was injected into an HPLC instrument (Agilent® 1260 Infinity® II, Column: Agilent AdvanceBio® SEC 300A 2.7 m and Mobile phase: PBS). Aggregation was measured by peak areas of aggregated ADC multimers.

As shown in Table 2 below, Trastuzumab emtansine was the most prone to aggregation (1.6% aggregation). Conjugate 3 had an aggregation of 0.5%. Conjugate 6 showed the least aggregation, i.e., 0.3%.

TABLE 2
Aggregation analysis of Kadcyla, Conjugates 3 and 6

| ADC | Aggregation (%) |
|---|---|
| Kadcyla | 1.6123 |
| Conjugate 3 | 0.4939 |
| Conjugate 6 | 0.3246 |

Two HPs increased the cytotoxic activities of ADCs in cancer cells Conjugates 6 and Conjugate 20 contain the chemotherapy drug DM1. Conjugates 6 has two glucose moieties as the HPs. Conjugate 20 has two PEG4 moieties as the HPs. The cytotoxic activities of Conjugates 6, Conjugate 20 and two commercial ADCs, namely trastuzumab emtansine (Kadcyla®, Genentech, California) and trastuzumab deruxtecan (Enhertu®, Daiichi Sankyo, New Jersey) were compared in three cell lines: N87, SKBR3, and JIMT-1, each having different expression levels of HER2. The experiment was performed as described in the section of Materials and Methods.

The IC50 values were calculated and shown in Table 3 below.

Conjugates 6 and 20 were each more effective in inhibiting all three cancer cell lines than trastuzumab deruxtecan. When compared to trastuzumab emtansine, the two conjugates were more effective in inhibiting JIMT-1. Remarkably, Conjugates 6 and 20 showed from 10 to 100 folds stronger cytotoxic activities toward JIMT-1 cells than trastuzumab deruxtecan.

TABLE 3
IC₅₀ of Kadcyla, Enhertu, Conjugates 6
and 20 obtained from cell-based assays.

| | IC₅₀ (mol/L) | | | |
|---|---|---|---|---|
| Cell line | Kadcyla | Enhertu | Conjugate 6 | Conjugate 20 |
| N87 | — | 2.12E−10 | 3.04E−11 | 3.07E−11 |
| SKBR3 | — | 1.27E−10 | 1.62E−11 | 2.20E−11 |
| JIMT-1 | 2.67E−09 | 6.34E−07 | 2.72E−10 | 5.99E−10 |

### Two HPs Prolonged PK of ADC in Rat

It is known that a trastuzumab-based ADC with DM1 as the payload could not have a DAR more than 4. Remarkably, with the help of two HPs, the DM1-containing Conjugate 6 could reached a DAR about 7.81. Thus, it would be informative to study its PK profile. The experiment was performed as described in the section of Materials and Methods. As shown in Table 4, Conjugate 6 exhibited a longer PK than that of Kadcyla.

TABLE 4
PK of Kadcyla and Conjugate 6 in rat

| | Kadcyla | Conjugate 6 |
|---|---|---|
| AUC₀₋ₜ (ug/ml*d) | 500.97 ± 35.85 | 701.95 ± 91.63 |

### Two HPs Increased the Potency of ADCs in Animal Studies

The experiment was performed as described in the section of Materials and Methods. The antitumor activity of a single intravenous injection (10 mg/kg at day zero) was obtained for each compound using the JIMT-1 xenograft model. Tumor volume, calculated as length×width²/2, was obtained in predetermined days.

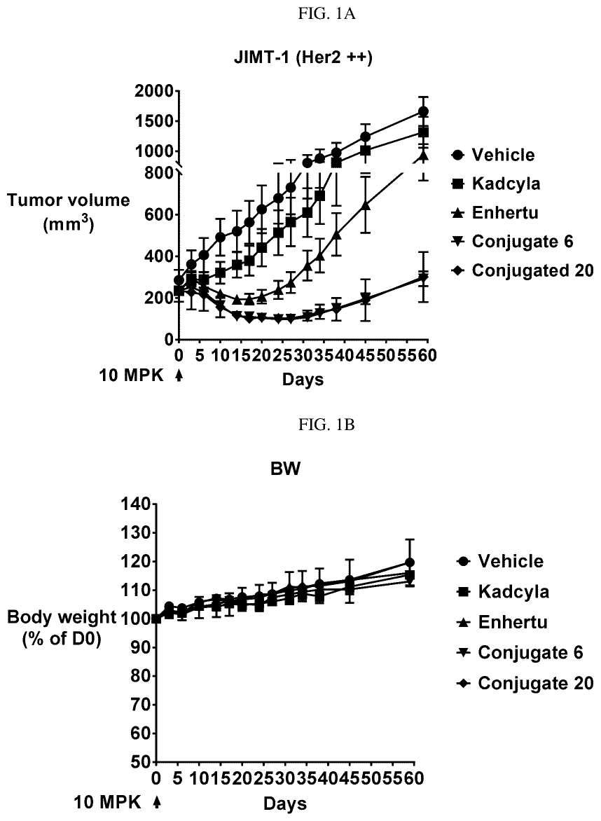

As shown in FIG. 1A, it was observed that the tumor volume decreased dramatically in the groups treated with Conjugate 6 and Conjugate 20 while both groups treated with trastuzumab emtansine and trastuzumab deruxtecan showed significant tumor growth.

Mouse body weight (calculated as percentage of that at day 0) during the period time of drug treatment demonstrated that tolerability of Conjugate 6 and Conjugate 20 was similar to marketed ADCs (Kadcyla® and Enhertu®) in JIMT-1 xenograft model (see FIG. 1), despite the fact that Conjugates 6 (DAR˜7.81) and 20 (DAR˜7.59) each contain more than two-fold of DM1 payload than Kadcyla (DAR-3.4).

Two HPs were Better than One HP in Augmenting the Cytotoxic Activity of DM1-Containing ADCs

The experiment was performed as described in the section of Materials and Methods. Conjugates 3 and 6 and trastuzumab emtansine were evaluated. Note that Conjugate 3 contains DM1 and a single maltose moiety, while Conjugate 6 containing DM1 and two parallel glucose moieties.

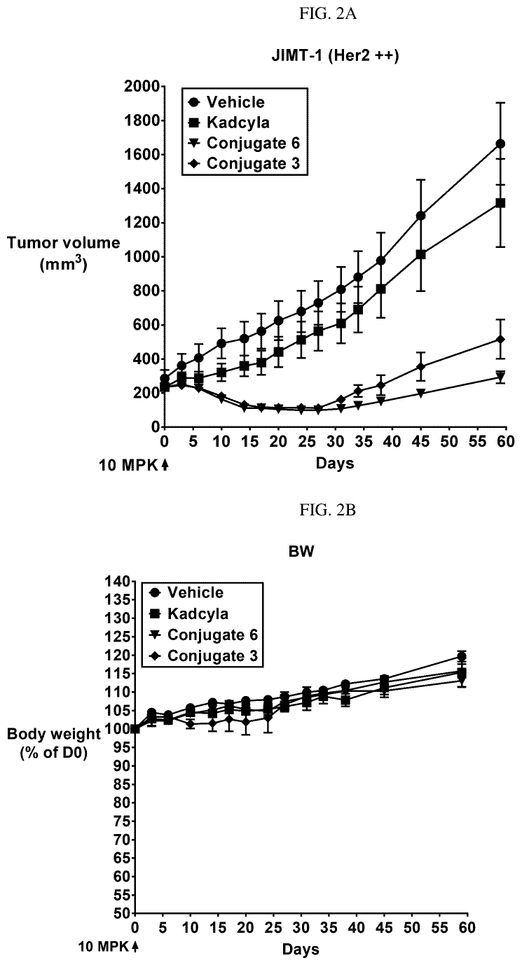

Each ADC sample was injected intravenously once (10 mg/kg at day zero). As shown in FIG. 2A, Conjugate 6 exhibited a higher antitumor activity than Conjugate 3, despite the fact that both Conjugates 3 (with one maltose moiety as its single HP) and 6 (with two glucose moieties as its parallel HPs) contain the same total number of glucose molecules.

Mouse body weight during the period time of drug treatment demonstrated that tolerability of Conjugate 6 (DAR˜7.81) and Conjugate 3 (DAR˜7.75) was similar to that of Kadcyla® (DAR-3.4) in JIMT-1 xenograft model. See FIG. 2B.

### Augmentation of ADC's Cytotoxicity by Two HPs was Antibody- and Payload-Independent

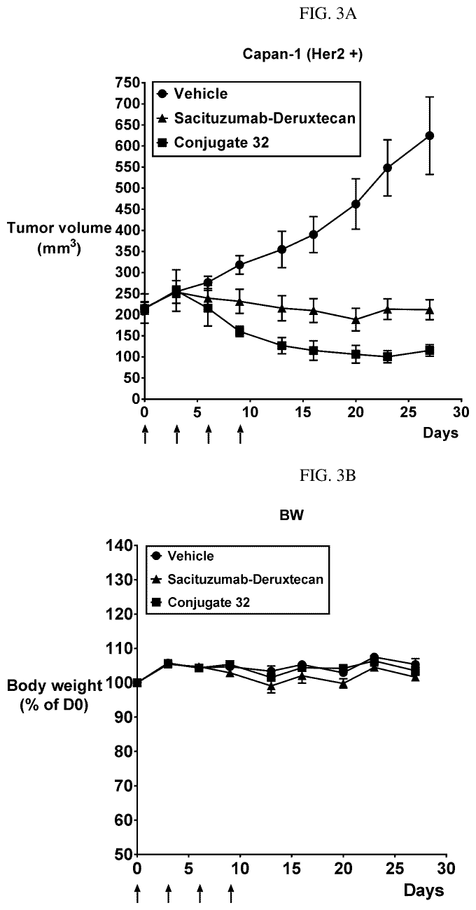

The experiment was performed as described in the section of Materials and Methods. Conjugate 32 is a Sacituzumab-based ADC containing chemotherapy drug deruxtecan (Dxd) and two maltose moieties as the HPs. The antitumor activity of four intravenous injections (12.5 mg/kg at day 0, 3, 6 and 9) of Conjugate 32 and Sacituzumab-deruxtecan (a biosimilar to Daiichi's Datroway) was compared using the Capan-1 xenograft model. Tumor volume, calculated as length×width²/2, was obtained on predetermined days. As shown in FIG. 3A, Conjugate 32 was more efficacious in inhibiting tumor growth than Sacituzumab-deruxtecan. Besides, mouse body weight (calculated as percentage of that at day 0) during the period time of drug treatment demonstrated that tolerability of Conjugate 32 was similar to that of Sacituzumab-deruxtecan. See FIG. 3B.

Furthermore, the observation that, compared to their counterparts without HP, two HPs-containing Conjugates 6 and Conjugate 20 (both consisting of trastuzumab and emtansine, see FIG. 1A) as well as Conjugate 32 (consisting of sacituzumab and deruxtecan, see FIG. 3A) all showed a higher cytotoxicity strongly supported the notion that augmentation of ADC's cytotoxicity by two HPs was antibody- and payload-independent.

### Two HPs Also Augmented the Antitumor Activity of an Immunostimulating ADC

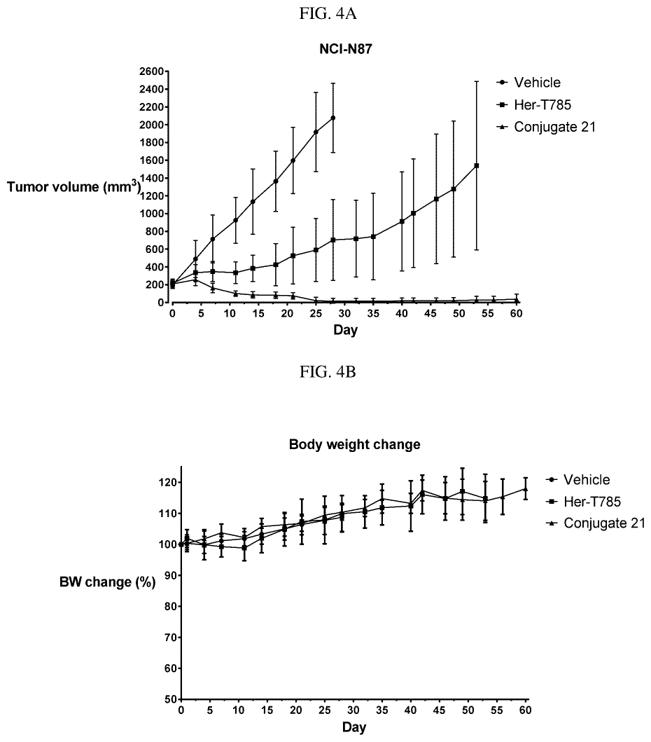

The experiment was performed as described in the section of Materials and Methods. Conjugate 21 and Her-T785 each were injected intravenously once (5 mg/kg at day 0). Tumor volume, calculated as length×width²/2, was obtained in predetermined days. As shown in FIG. 4A, Conjugate 21 eliminated the tumor mass while Her-T785 only moderately inhibited tumor growth.

Mouse body weight (calculated as percentage of that at day 0) during the period time of drug treatment demonstrated that tolerability of Conjugate 21 was similar to that of Her-T785 in the NCI-N87 xenograft model. See FIG. 4B.

### OTHER EMBODIMENTS

All of the features disclosed in this specification may be combined in any combination. Each feature disclosed herein may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the conjugates of this invention also can be made, screened for their efficacy in treating cancer.

What is claimed is:

1. A compound of formula I: 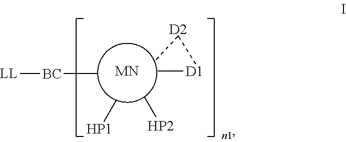
in which
LL is a ligand linker containing a coupling moiety capable of reacting with a ligand via a sulfhydryl, amino, glutamine, formyl, or keto group contained in the ligand,
BC is a bridging complex containing 1 to 20 monomeric units,
MN is a multifunctional linker complex containing 1 to 20 monomeric units,
D1 is a first drug moiety,
D2 is absent or a second drug moiety,
HP1 is a first hydrophilic polymer moiety,
HP2 is a second hydrophilic polymer moiety,
LL is bonded to BC or MN via a first connector,
BC is bonded MN via a second connector,
HP1 is bonded to MN via a third connector,
HP2 is bonded to MN via a fourth connector,
D1 is bonded to MN via a fifth connector,
D2 when present is bonded to MN or D1 via a sixth connector,
One of [symbol] is a covalent bond and the other [symbol] is absent;
n1 is 1 or 2, and
each of the first to sixth connectors independently is selected from the group consisting of a hydrazone moiety, an oxime moiety, a carbonate moiety (—O—C(O)—O—), an azobenzene moiety, an amide bond, an arylsulfate bond, a glycoside bond, a beta-glucuronide bond, a beta-galactoside bond, an ester bond, a phosphate bond, a pyrophosphate bond, an ether bond, a thioether bond, a disulfide bond, a C—S bond, a C—O bond, a C—N bond, a C—C bond, an imine bond, a carbamate moiety (—O—C(O)—NH—), a urea moiety (—NH—C(O)—NH—), a 1,2,4-trioxolane (TRX) moiety, a triazole moiety 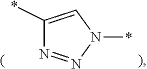 [SMILES: *c1cn(*)nn1]
a moiety derived from strain-promoted azide-alkyne cycloaddition (SPAAC), a moiety derived from strain-promoted alkyne-nitrone cycloaddition (SPANC), and a moiety derived from the trans-cyclooctene (TCO)-tetrazine click reaction.

2. The compound of claim 1, wherein the compound is a compound of formula II: 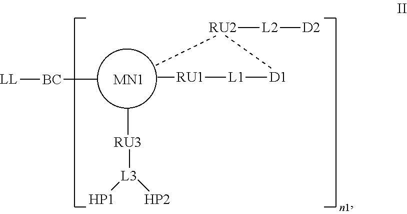
in which
MN1 is a multifunctional linker complex containing 1 to 20 monomeric units,
RU1 is a bond or a first releasable unit with or without a self-immolative spacer,
RU2 is a bond or a second releasable unit with or without a self-immolative spacer;
one of [symbol] is a covalent bond and the other [symbol] is absent;
RU3 is a bond or a third releasable unit with or without a self-immolative spacer,
L1 is a bond or a first bifunctional crosslinker,
L2 is a bond or a second bifunctional crosslinker,
L3 is a bond or a multifunctional crosslinker,
RU1 when present is bonded to MN1 via a seventh connector,
L1 when present is bonded to RU1 or MN1 via an eighth connector,
D1 is bonded to L1, RU1, or MN1 via the fifth connector,
RU2 when present is bonded to MN1 or D1 via a ninth connector,
L2 when present is bonded to RU2, MN1 or D1 via a tenth connector,
D2 when present is bonded to L2, RU2, MN1 or D1 via the sixth connector,
RU3 when present is bonded to MN1 via an eleventh connector,
L3 when present is bonded to RU3 or MN1 via a twelfth connector,
HP1 is bonded to L3, RU3, or MN1 via the third connector,
HP2 is bonded to L3, RU3, or MN1 via the fourth connector,
each of the seventh to twelfth connectors independently is selected from the group consisting of a hydrazone moiety, an oxime moiety, a carbonate moiety (—O—C(O)—O—), an azobenzene moiety, an amide bond, an arylsulfate bond, a glycoside bond, a beta-glucuronide bond, a beta-galactoside bond, an ester bond, a phosphate bond, a pyrophosphate bond, an ether bond, a thioether bond, a disulfide bond, a C—S bond, a C—O bond, a C—N bond, a C—C bond, an imine bond, a carbamate moiety (—O—C(O)—NH—), a urea moiety (—NH—C(O)—NH—), a 1,2,4-trioxolane (TRX) moiety, a triazole moiety 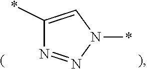 [SMILES: *c1cn(*)nn1]
a moiety derived from strain-promoted azide-alkyne cycloaddition (SPAAC), a moiety derived from strain-promoted alkyne-nitrone cycloaddition (SPANC), and a moiety derived from the trans-cyclooctene (TCO)-tetrazine click reaction; and at least one of the third to twelfth connectors is a releasable connector selected from the group consisting of a hydrazone moiety, an oxime moiety, a carbonate moiety, an azobenzene moiety, a disulfide bond, an amide bond, an arylsulfate bond, a glycoside bond, a beta-glucuronide bond, a beta-galactoside bond, an ester bond, a phosphate bond, a pyrophosphate bond, a carbamate moiety, a urea moiety, and a 1,2,4-trioxolane (TRX) moiety.

3. The compound of claim 1, wherein the compound is a compound of formula III: 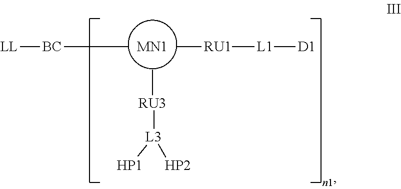
in which
MN1 is a multifunctional linker complex containing 1 to 20 monomeric units,
RU1 is a bond or a first releasable unit with or without a self-immolative spacer,
RU3 is a bond or a third releasable unit with or without a self-immolative spacer,
L1 is a bond or a first bifunctional crosslinker,
L3 is a bond or a multifunctional crosslinker,
RU1 when present is bonded to MN1 via a seventh connector,
L1 when present is bonded to RU1 or MN1 via an eighth connector,
D1 is bonded to L1, RU1, or MN1 via the fifth connector,
RU3 when present is bonded to MN1 via an eleventh connector,
L3 when present is bonded to RU3 or MN1 via a twelfth connector,
HP1 is bonded to L3, RU3, or MN1 via the third connector,
HP2 is bonded to L3, RU3, or MN1 via the fourth connector,
each of the seventh, eighth, eleventh and twelfth connectors independently is selected from the group consisting of a hydrazone moiety, an oxime moiety, a carbonate moiety (—O—C(O)—O—), an azobenzene moiety, an amide bond, an arylsulfate bond, a glycoside bond, a beta-glucuronide bond, a beta-galactoside bond, an ester bond, a phosphate bond, a pyrophosphate bond, an ether bond, a thioether bond, a disulfide bond, a C—S bond, a C—O bond, a C—N bond, a C—C bond, an imine bond, a carbamate moiety (—O—C(O)—NH—), a urea moiety (—NH—C(O)—NH—), a 1,2,4-trioxolane (TRX) moiety, a triazole moiety 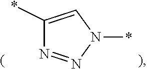 [SMILES: *c1cn(*)nn1]
a moiety derived from strain-promoted azide-alkyne cycloaddition (SPAAC), a moiety derived from strain-promoted alkyne-nitrone cycloaddition (SPANC), and a moiety derived from the trans-cyclooctene (TCO)-tetrazine click reaction; and at least one of the third, fourth, fifth, seventh, eighth, eleventh and twelfth connectors is a releasable connector selected from the group consisting of a hydrazone moiety, an oxime moiety, a carbonate moiety, an azobenzene moiety, a disulfide bond, an amide bond, an arylsulfate bond, a glycoside bond, a beta-glucuronide bond, a beta-galactoside bond, an ester bond, a phosphate bond, a pyrophosphate bond, a carbamate moiety, a urea moiety, and a 1,2,4-trioxolane (TRX) moiety.

4. The compound of claim 2, wherein BC, MN or MN1 contains at least 2 monomeric units each being bonded to its adjacent monomeric unit via a covalent bond.

5. The compound of claim 2, wherein at least one of the fifth, seventh and eighth connectors is a releasable connector, and at least one of the sixth, ninth and tenth connectors is a releasable connector.

6. The compound of claim 2, wherein HP1 and HP2 are connected to a single monomeric unit contained in MN or MN1.

7. The compound of claim 6, wherein HP1 and HP2 are each connected to the monomeric unit via a C—N bond.

8. The compound of claim 1, wherein each of HP1 and HP2, independently, is a pharmaceutically active moiety.

9. The compound of claim 2, wherein LL contains amino (NH₂), iodo (I), bromo (Br), a maleimide moiety, an N-hydroxysuccinimide (NHS) moiety, or an aminooxy moiety; and LL is bonded to BC, MN or MN1 via an amide bond, an arylsulfate bond, a glycoside bond, a beta-glucuronide bond, a beta-galactoside bond, an ester bond, a phosphate bond, a pyrophosphate bond, an ether bond, a thioether bond, a disulfide bond, a C—S bond, a C—O bond, a C—N bond, a C—C bond, an imine bond, a carbamate moiety (—O—C(O)—NH—), a urea moiety (—NH—C(O)—NH—), a 1,2,4-trioxolane (TRX) moiety, a triazole moiety 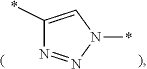 [SMILES: *c1cn(*)nn1]
a moiety derived from strain-promoted azide-alkyne cycloaddition (SPAAC), a moiety derived from strain-promoted alkyne-nitrone cycloaddition (SPANC), or a moiety derived from the trans-cyclooctene (TCO)-tetrazine click reaction.

10. The compound of claim 9, wherein LL is 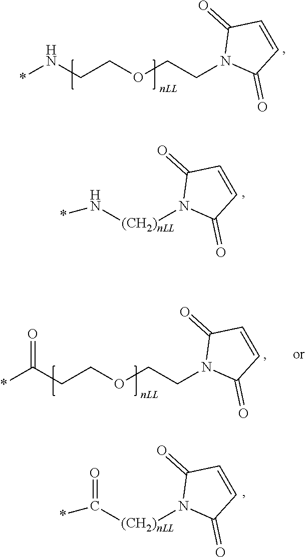
in which nLL is an integer from 0 to 10, preferably 1 or 2.

11. The compound of claim 10, wherein LL is

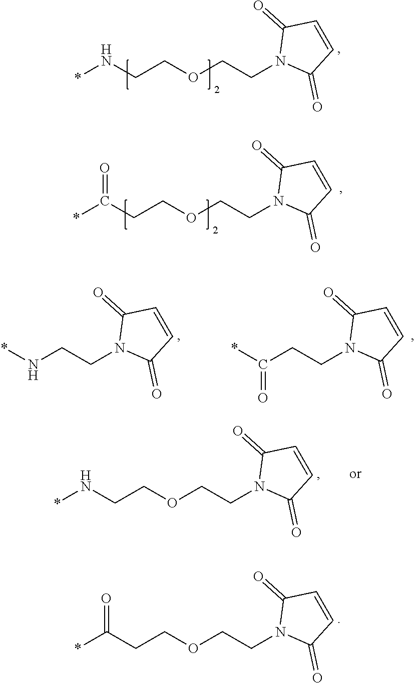

12. The compound of claim 2, wherein each of BC, MN, MN1, L1, L2, and L3, independently, contains one or more monomeric units derived from the group consisting of a natural amino acid, a non-natural amino acid, a monosaccharide, a bi- or multi-functional crosslinker, a polyamine, a polyalcohol, a polycarboxylic acid, a hydroxycarboxylic acid, a glycolic acid, a lactic acid, a diglycolic acid, an iminodiacetic acid, an aminoaldehyde, an aminoketo, an aminoalcohol, a mercaptocarboxylic acid, an aminothiol, a penicillamine, 2-(4-aminophenyl)-2-hydroxyacetic acid, pyrrolidine-2,4-dicarboxylic acid, 1-aminocyclopentane-trans-1,3-dicarboxylic acid, 3,6-bis-(4-aminobutyl)piperazine-2,5-dione, a cyclic peptide, and a diamino dicarboxylic acid.

13. The compound of claim 12, wherein each of BC, MN, and MN1 independently, is an oligomer containing 1 to 10 monomeric unit selected from the group consisting of:

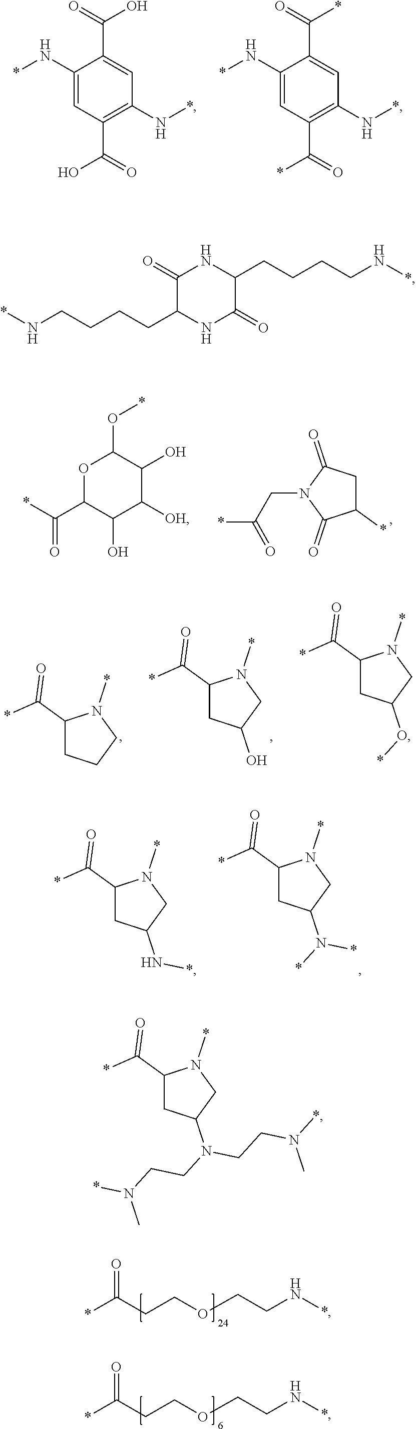

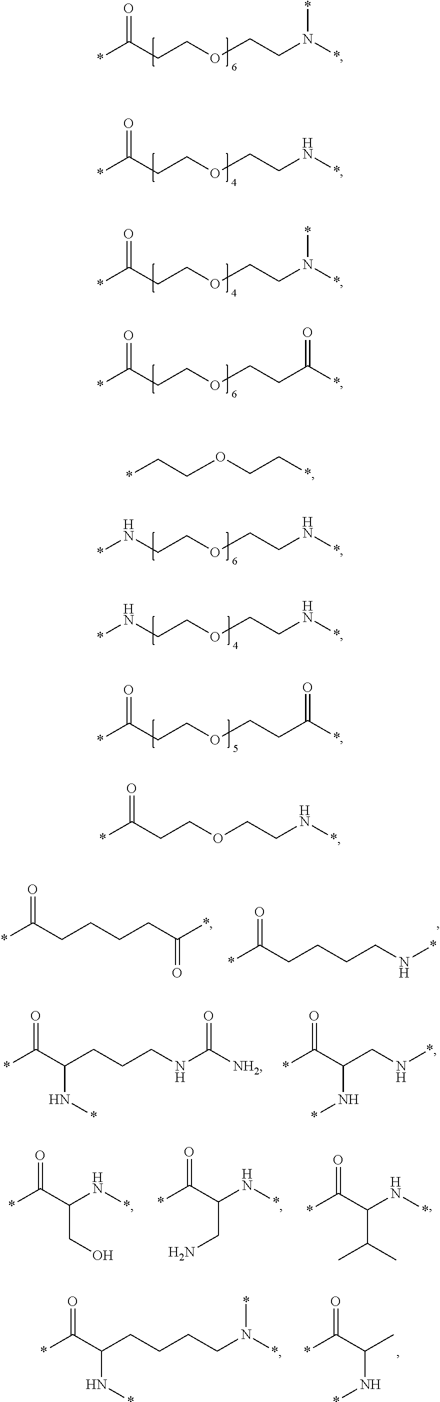

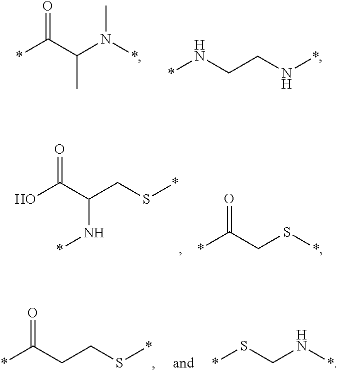

14. The compound of claim 13, wherein each of BC, MN and MN1, independently, is the oligomer containing 1 to 8 monomeric units selected from the group consisting of:

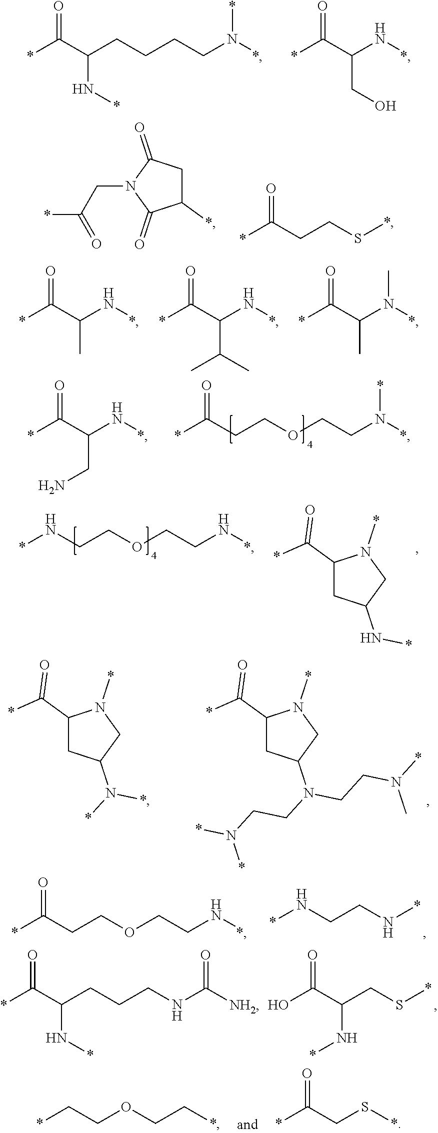

15. The compound of claim 1, wherein each of HP1 and HP2, independently, is a monosaccharide moiety, an oligosaccharide moiety, or a polyethylene glycol moiety.

16. The compound of claim 15, wherein each of HP1 and HP2, independently, is a glucose moiety, a galactose moiety, a mannose moiety, a ribose moiety, a maltose moiety, a lactose moiety, a cellobiose moiety, a maltotriose moiety, a cellotriose moiety, a bleomycin moiety, or a polyethylene glycol moiety containing 1 to 10 ethylene glycol units.

17. The compound of claim 2, wherein each of RU1, RU2, and RU3, independently, is a serine moiety, a PEG moiety, a valine moiety, a citrulline moiety, an alanine moiety, an asparagine moiety, a 2,3-diaminopropionic acid (DAP) moiety, 4-aminobenzyl alcohol, 4-aminobenzyl-amine, an alanine-alanine-asparagine moiety, a glycine-glycine-phenylalanine-glycine moiety or any combination thereof.

18. The compound of claim 1, wherein each of D1 and D2 is a drug moiety derived from mertansine (DM1), ravtansine (DM4), N-methyl-L-Ala-maytansinol, monomethyl auristatin E, 7-ethyl-10-hydroxycampto-thecin (SN38), ARV-771, TLR7/8 agonist Ag, NOD1 agonist or NOD2 agonist, or any combination thereof.

19. The compound of claim 1, wherein the compound is one of Compounds 6-46.

20. The compound of claim 1, wherein the compound is a compound selected from the group consisting of Compounds 6, 20, 21, 34, 35, 40, 43 and 45.

21. A ligand-drug conjugate comprising a ligand moiety and a moiety derived from a compound of claim 1, wherein the ligand is bonded to the compound via a covalent bond formed between a functional group from the ligand and LL in formula I, and the functional group is sulfhydryl, amino, glutamine, formyl, or keto.

22. The ligand-drug conjugate of claim 21, wherein the ligand is Trastuzumab, albumin, Pertuzumab, or Sacituzumab and the covalent bond is formed between sulfhydryl of the ligand and the maleimide, iodo, or bromo moiety of LL.

23. The ligand-drug conjugate of claim 21, wherein the molar ratio of the ligand to the compound is between 1:1 and 1:20, preferably between 1:2 and 1:16.

24. The ligand-drug conjugate of claim 21, wherein the conjugate is one of Conjugates 6-32.

25. A method of treating cancer comprising administrating to a patient in need thereof an effective amount of the conjugate of claim 21.

26. A pharmaceutical composition comprising the conjugate of claim 21, and a pharmaceutically acceptable carrier, diluent, or excipient.

27. A method of preparing a conjugate of claim 21, comprising reacting a ligand with a compound of claim 1, wherein the ligand contains one or more of functional groups that are, independently, sulfhydryl, amino, glutamine, formyl, or keto.
